(19) United States
(12) Patent Application Publication
(10) Pub. No.: US 2024/0408198 A1
(43) Pub. Date: Dec. 12, 2024

(54) THERAPEUTIC COMPOSITIONS AND METHODS FOR ALLOGENEIC HEMATOPOIETIC STEM CELL TRANSPLANTATION

(71) Applicant: ORCA BIOSYSTEMS, INC., Menlo Park, CA (US)

(72) Inventors: Nathaniel Fernhoff, Menlo Park, CA (US); Ivan Dimov, Menlo Park, CA (US); Catherine Yin, Menlo Park, CA (US); Scott Killian, Menlo Park, CA (US); Alan Marmelstein, Menlo Park, CA (US)

(21) Appl. No.: 18/655,170

(22) Filed: May 3, 2024

Related U.S. Application Data
(63) Continuation of application No. PCT/US2022/048896, filed on Nov. 3, 2022.
(60) Provisional application No. 63/275,894, filed on Nov. 4, 2021.

(51) Int. Cl. A61K 39/00; C07K 16/28

(52) U.S. Cl. CPC A61K 39/4611; A61K 39/4621; C07K 16/2866; C07K 16/2896

(57) ABSTRACT

Embodiments of the disclosure provide compositions, kits, and methods for allogeneic hematopoietic cell transplantation (alloHCT) to patients. In an embodiment, a therapeutic composition for alloHCT includes at least first and second populations of isolated CD45+ cells (ICC). At least a portion of the CD45+ cells in the first population may have an antibody bound to a marker on the cell surface which is used to separate CD34+ cells from a mixture of nucleated cells (MNC) from donor or invitro produced blood. The MNC may comprise various cell types in various amounts, for example, about 70% CD34+ cells, less than about 5% CD3+ cells and less about 20% granulocytes. The ICC's in the second population include regulatory T cells which are typically at least about 50% of the population. Embodiments of the disclosure are particularly useful for treatment of hematologic cancers (e.g., leukemia, lymphoma), sickle cell anemia, GVHD, autoimmune and other diseases.

### CROSS-REFERENCE

This application is a continuation of International Application No. PCT/US2022/48896, filed Nov. 3, 2022, which claims the benefit of priority of U.S. Provisional Application Nos. 63/275,894, filed Nov. 4, 2021, the contents of which is incorporated herein by reference in its entirety for all purposes.

### BACKGROUND

Patients with hematologic malignancies such as leukemia and lymphoma beyond first remission or with refractory relapse are rarely cured with standard chemotherapy. Myeloablative allogeneic hematopoietic cell transplantation (alloHCT) is associated with improved survival in these patients. Myeloablative alloHCT is a procedure in which the patient undergoes chemotherapy or radiation to ablate or destroy tissue in the bone causing the malignancy. They then receive hematopoietic stem and progenitor cells (HSPC) and other cells from a donor's blood. However, alloHCT has a major drawback in that it often results in graft versus host disease (GVHD). GVHD is a condition in which the transplanted donor peripheral blood stem cells view the patient's body as foreign, and the donated cells attack the patient's tissue (e.g., skin, GI tissue, liver tissue, and lung tissue) resulting in a number of complications, many of which can be serious (e.g., rash, nausea, jaundice, GI issues, and shortness of breath) and which result in morbidity and mortality. Thus, there is a need for improved methods and therapies for hematopoietic cell transplantation which have a reduced incidence and severity of GVHD including, reduced morbidity and mortality.

### SUMMARY

Embodiments of the disclosure provide therapeutic compositions for the treatment of one or more diseases or conditions where the compositions comprise one or more cells populations including, for example, populations of hematopoietic stem and progenitor cells (HSPC's); populations of regulatory T cells (Tregs) and populations of conventional T cells (Tcons). The diseases and conditions treated by embodiments of the therapeutic compositions described herein may include one or more of leukemia, lymphoma and other forms of stem cell- and/or hematologic based cancer, non-malignant hematologic conditions (e.g., sickle cell anemia) various autoimmune conditions (e.g., multiple sclerosis) and graft versus host disease resulting from organ transplants (e.g., bone marrow transplant).

In some aspects, provided herein are methods of treating a disease or condition in a human subject. In some embodiments, the method may comprise: obtaining isolated regulatory T cells (Tregs). In some embodiments, the obtaining Tregs may comprise: contacting a donor cell sample may comprise Tregs with an amount of an anti-human CD25 affinity reagent such that less than 85% of the CD25 polypeptides expressed on the surface of the Tregs in the donor cell sample are occupied (or bound to) by the affinity reagent. In some embodiments, the method further comprises isolating Tregs from (a)(i) occupied by the affinity reagent; thereby obtaining isolated Tregs. In some embodiments, the method further comprises administering the isolated Tregs to the human subject. In some embodiments, the isolated Tregs exhibit pSTAT5 activity according to an in vitro assay.

In some embodiments, less than 80% of the CD25 polypeptides on the Tregs are occupied by the affinity reagent. In some embodiments, less than 75% of the CD25 polypeptides on the Tregs are occupied by the affinity reagent. In some embodiments, at least 30% of the CD25 polypeptides on the Tregs are occupied by the affinity reagent. In some embodiments, at least 40% of the CD25 polypeptides on the Tregs are occupied by the affinity reagent. In some embodiments, at least 50% of the CD25 polypeptides on the Tregs are occupied by the affinity reagent. In some embodiments, from about 30% to about 80% of the CD25 polypeptides on the Tregs are occupied by the affinity reagent. In some embodiments, about 50% to about 75% of the CD25 polypeptides on the Tregs are occupied by the affinity reagent.

In some embodiments, the isolated Tregs exhibit at least 10% of the pSTAT5 activity in an in vitro assay as compared to Tregs that do not have CD25 polypeptides occupied by the affinity reagent. In some embodiments, the isolated Tregs exhibit at least 20% of the pSTAT5 activity in an in vitro assay as compared to Tregs that do not have CD25 polypeptides occupied by the affinity reagent. In some embodiments, the isolated Tregs exhibit at least 40% of the pSTAT5 activity in an in vitro assay as compared to Tregs that do not have CD25 polypeptides occupied by the affinity reagent. In some embodiments, the isolated Tregs exhibit at least 50% of the pSTAT5 activity in an in vitro assay as compared to Tregs that do not have CD25 polypeptides occupied by the affinity reagent.

In some embodiments, the method further may comprise isolating hematopoietic stem cells (HSCs) or hematopoietic stem and progenitor cells (HSPCs) from the donor cell sample. In some embodiments, the isolating HSCs or for HSPCs may comprise contacting the donor cell sample with an anti-human CD34 affinity reagent and isolating cells that have CD34s occupied by the anti-human CD34 affinity reagent, thereby obtaining isolated HSCs or isolated HSPCs. In some embodiments, the method further may comprise administering the isolated HSCs or isolated HSPCs to the human subject.

In some embodiments, at least 2×10⁶ HSCs or at least 2×10⁶ HSPCs per kilogram of the human subject's actual body weight or ideal body weight are administered to the human subject. In some embodiments, from about 5×10⁵ to about 2×10⁷ HSCs or from about 5×10⁵ to about 2×10⁷ HSPCs per kilogram of the human subject's actual body weight or ideal body weight are administered to the human subject.

In some embodiments, at least 1×10⁶ Tregs per kilogram of the human subject's actual body weight or ideal body weight are administered to the human subject. In some embodiments, from about 5×10⁵ to about 5×10⁶ Tregs per kilogram of the human subject's actual body weight or ideal body weight are administered to the human subject.

In some embodiments, the method may comprise contacting the donor cell sample with an anti-human CD3 affinity reagent. In some embodiments, the method may comprise quantifying an amount of CD3 positive conventional T cells (Tcons) in the donor cell sample by identifying the presence of CD3s on Tcons that are occupied by the anti-human CD3 affinity reagent. In some embodiments, the human subject may be further administered a cell population may comprise from about 5×10⁵ to about 5×10⁶ Tcons per kilogram of actual body weight or ideal body weight of said human subject.

In some embodiments, the method may comprise administering a single GVHD prophylactic agent to the human subject. In some embodiments, the single GVHD prophylactic agent may be tacrolimus or sirolimus. In some embodiments, the tacrolimus may be formulated for oral administration or for intravenous administration. In some embodiments, the tacrolimus may be administered in an amount to maintain a target blood level of at least about 3 ng/ml for about 20 or more days post-transplant of any cell populations. In some embodiments, the tacrolimus may be administered in an amount to maintain a target blood level of at least about 4 ng/ml for about 40 or more days post-transplant of any cell population. In some embodiments, the tacrolimus may be administered for at least about 60 days after administering any cell population.

In some embodiments, the method may comprise administering a conditioning regimen to the human subject. In some embodiments, the conditioning regimen may be administered from about two days to about ten days before transplanting any cell population. In some embodiments, the conditioning regimen may be a myeloablative conditioning regimen. In some embodiments, the conditioning regimen may comprise at least three conditioning reagents, wherein at least one conditioning reagent may comprise thiotepa. In some embodiments, the conditioning regimen may comprise one or more doses of busulfan, fludarabine, and thiotepa.

In some embodiments, the donor cell sample may be a blood sample. In some embodiments, the donor cell sample may be a mobilized peripheral blood sample.

In some embodiments, the contacting Tregs with an affinity reagent may comprise: contacting a donor cell sample may comprise Tregs with an amount of an anti-human CD25 affinity reagent such that less than 85% of the CD25 polypeptides expressed on the surface of the Tregs in the donor cell sample are occupied (or bound to) by the affinity reagent according to an assay. The assay may comprise: (1) measuring the amount of background fluorescence from a first sample of a cell population, wherein the background fluorescence is emitted at a wavelength corresponding to a first fluorophore and wherein a Mean Fluorescence Intensity Zero (MFI-0) value is assigned the measured amount of background fluorescence; (2) contacting a second sample of a cell population with an anti-CD25 antibody which is conjugated to the first fluorophore, wherein the contacting occurs at a concentration, temperature, and time sufficient to achieve at least 90% saturation; (3) measuring the amount of fluorescence emitted from the cells of step (2), wherein a Mean Fluorescence Intensity Saturation (MFI-sat) value is assigned the measured amount of fluorescence; (4) contacting simultaneously a third sample of a cell population with the anti-CD25 antibody conjugated to the first fluorophore and an affinity reagent which lacks a fluorophore and under the contacting conditions used in step (2); (5) measuring the amount of fluorescence emitted from the cells of step (4), wherein a Mean Fluorescence Intensity X (MFI-x) value is assigned the measured amount of fluorescence; and (6) quantifying the percentage of receptor occupancy according to the following equation: % Receptor Occupancy=(MFI-x−MFI-0)/(MFI-sat−MFI-0)×100%.

In some embodiments, the pSTAT5 activity may be measured by intracellular cytokine staining, wherein the assay may comprise: (1) obtaining a cell having been exposed to an affinity reagent; (2) contacting the cell with IL-2 at a concentration, temperature and time sufficient for the IL-2 to interact with its receptor on the cell and activate IL-2 signaling by the cell; (3) contacting the cell with each of: a first antibody conjugated with a first fluorophore and directed against CD3, a second antibody conjugated with a second fluorophore and directed against CD4, a third antibody conjugated with a third fluorophore and directed against CD25, a fourth antibody conjugated with a fourth fluorophore and directed against CD127, and a fifth antibody conjugated with a fifth fluorophore and directed against pSTAT5. In some cases, each of the first, second, third, fourth, and fifth fluorophore are different fluorophores; (4) subjecting the cell of step (3) to flow cytometry which may be gated for CD3+CD4+CD25+CD127− and collecting the CD3+CD4+CD25+CD127− cells; (5) detecting the amount of fluorescence from the fifth fluorophores and detecting the amount of florescence from the first, the second, and/or the third fluorophores in the collected cells of step (5); and (6) calculating the percentage of the collected cells which have fluorescence from the first, the second, and/or the third fluorophores and which have fluorescence from the fifth fluorophore, wherein this percentage represents the fraction of cells having receptors occupied by the affinity reagent.

In some embodiments, the affinity reagent blocks IL2 signaling in the Tregs. In some embodiments, the affinity reagent blocks pSTAT5 activity in the Tregs. In some embodiments, the Tregs comprise at least 1×10⁶ Tregs. In some embodiments, the pSTAT5 is phosphorylated at tyrosine 694.

In some aspects, provided herein are methods of treating a disease or condition in a human subject. In some embodiments, the method may comprise: obtaining isolated regulatory T cells (Tregs); wherein the obtaining Tregs may comprise: contacting a donor cell sample may comprise Tregs with an amount of an anti-human CD25 affinity reagent such that more than 85% of the CD25 polypeptides expressed on the surface of the Tregs in the donor cell sample are occupied (or bound to) by the affinity reagent; isolating Tregs from (a)(i) obtained by the affinity reagent; thereby obtaining isolated Tregs; and administering the isolated Tregs to the human subject. In some embodiments, the isolated Tregs exhibit pSTAT5 activity according to an in vitro assay. In some embodiments, the affinity reagent does not block IL2 signaling in the Tregs.

In some aspects, provided here in are kits. In some embodiments, described herein is a kit for use in preparation of a therapeutic composition. The kit may comprise: an anti-human CD25 affinity reagent; and instructions for use of (a) to isolate regulatory T cells (Tregs) from a donor cell sample. In some embodiments, the instructions may include directions to isolate the Tregs which have the anti-human CD25 affinity reagent such that less than 85% of CD25 polypeptides expressed on the surface of the Tregs in the donor cell sample are occupied by the anti-human CD25 affinity reagent.

In some embodiments, the kit may comprise instructions for use of the kit to prepare a therapeutic composition. In some embodiments, the kit may comprise instructions to isolate a cell population of Tregs. In some embodiments, the kit may comprise an anti-human CD34 affinity reagent and instructions to isolate a cell population of CD34 positive hematopoietic stem and progenitor cells (HSPCs). In some embodiments, the kit may comprise a means to isolate CD25 positive cells and/or CD34 positive cells.

In some embodiments, the means may comprise a sorting column, such as a magnetized column.

In some embodiments, the instructions include directions to isolate Tregs such that less than 80% of CD25 polypeptides expressed on the surface of the Tregs in the donor cell sample are occupied by the anti-human CD25 affinity reagent. In some embodiments, the instructions include directions to isolate Tregs such that less than 75% of CD25 polypeptides expressed on the surface of the Tregs in the donor cell sample are occupied by the anti-human CD25 affinity reagent. In some embodiments, the instructions include directions to isolate Tregs such that at least 30% of CD25 polypeptides expressed on the surface of the Tregs in the donor cell sample are occupied by the anti-human CD25 affinity reagent. In some embodiments, the instructions include directions to isolate Tregs such that at least 40% of CD25 polypeptides expressed on the surface of the Tregs in the donor cell sample are occupied by the anti-human CD25 affinity reagent. In some embodiments, the instructions include directions to isolate Tregs such that from about 30% to 80% of CD25 polypeptides expressed on the surface of the Tregs in the donor cell sample are occupied by the anti-human CD25 affinity reagent. In some embodiments, the instructions include directions to isolate Tregs such that from 50% to 75% of CD25 polypeptides expressed on the surface of the Tregs in the donor cell sample are occupied by the anti-human CD25 affinity reagent.

In some embodiments, the kit may comprise one or more buffer solutions and containers for the buffer solutions. In some embodiments, the buffer solution contains about 2.5 volume percent human serum albumin and about 1 millimolar Ethylenediaminetetraacetic acid (EDTA). In some embodiments, the buffer solution may be a phosphate buffer. In some embodiments, the buffer solution has a pH of about 7.2. In some embodiments, the kit may comprise a plurality of immunoglobulin molecules suspended in the buffer solution. In some embodiments, the plurality of immunoglobulin molecules may comprise IVgG. In some embodiments, the affinity reagents are conjugated to a fluorophore for optical sorting.

In some embodiments, an amount of anti-human CD25 affinity reagents in the kit may be selected to process greater than about 8 liters of donor blood. In some embodiments, an amount of anti-human CD34 affinity reagents in the kit may be selected to process greater than about 8 liters of donor blood. In some embodiments, an amount of anti-human CD25 affinity reagents in the kit may be selected to process greater than about 15 liters of donor blood. In some embodiments, an amount of anti-human CD34 affinity reagents in the kit may be selected to process greater than about 15 liters of donor blood.

In some embodiments, the instructions for use are stored on a network, a computer network, the Internet or the cloud. In some embodiments, the instructions for use include information unique to a batch of anti-human CD25 affinity reagents or a batch of anti-human CD34 affinity reagents used in the kit, wherein the information may be used by an instrument or device in the preparation of the at least one therapeutic composition to optimize a parameter of the at least one therapeutic composition. In some embodiments, the parameter may be a purity, concentration, or dose of Tregs in the therapeutic composition.

In some embodiments, the kit further may comprise a GVHD prophylactic agent. In some embodiments, the GVHD prophylactic agent may be tacrolimus or sirolimus. In some embodiments, the tacrolimus may be formulated for oral administration or for intravenous administration. In some embodiments, instructions for use include directions such that the tacrolimus may be administered in an amount to maintain a target blood level of at least about 3 ng/ml for about 20 or more days post-transplant of any cell population. In some embodiments, instructions for use include directions such that the tacrolimus may be administered in an amount to maintain a target blood level of at least about 4 ng/ml for about 40 or more days post-transplant of any cell population. In some embodiments, instructions for use include directions for tacrolimus administration for at least about 60 days after administering any cell population.

In some embodiments, the kit further may comprise one or more conditioning reagents for a myeloablative conditioning regimen. In some embodiments, the kit may comprise three conditioning reagents, wherein at least one conditioning reagent may comprise thiotepa. In some embodiments, the conditioning reagents may comprise one or more doses of busulfan, fludarabine, and thiotepa.

In some embodiments, the kit further may comprise an anti-human CD3 affinity reagent. In some embodiments, the instructions for use comprise directions to quantify an amount of conventional T cells (Tcons) in the donor cell sample.

In some embodiments, the instructions include directions to detect pSTAT5 activity in the Tregs in an in vitro assay. In some embodiments, the Tregs exhibit at least 10% of the pSTAT5 activity after isolating Tregs with the kit in an in vitro assay as compared to Tregs that do not have CD25 polypeptides occupied by the anti-human CD25 affinity reagent. In some embodiments, the Tregs exhibit at least 20% of the pSTAT5 activity after isolating Tregs with the kit in an in vitro assay as compared to Tregs that do not have CD25 polypeptides occupied by the anti-human CD25 affinity reagent. In some embodiments, the Tregs exhibit at least 40% of the pSTAT5 activity after isolating Tregs with the kit in an in vitro assay as compared to Tregs that do not have CD25 polypeptides occupied by the anti-human CD25 affinity reagent. In some embodiments, the Tregs exhibit at least 50% of the pSTAT5 activity after isolating Tregs with the kit in an in vitro assay as compared to Tregs that do not have CD25 polypeptides occupied by the anti-human CD25 affinity reagent.

It shall be understood that different aspects and/or embodiments of the present disclosure can be appreciated individually, collectively, or in combination with each other. Any description herein concerning a specific composition and/or method apply to and may be used for any other specific composition and/or method as disclosed herein. Additionally, any composition disclosed herein is applicable to any herein-disclosed method. In other words, any aspect or embodiment described herein can be combined with any other aspect or embodiment as disclosed herein

### INCORPORATION BY REFERENCE

All publications, patents, and patent applications mentioned in this specification are herein incorporated by reference to the same extent as if each individual publication, patent, or patent application was specifically and individually indicated to be incorporated by reference. The following patent documents are incorporated herein by reference in their entireties and for any purposes: WO2018170335; WO2019157158; WO2019157158; WO2020047508; WO2021050597; WO2021127276; WO2021178566; WO2022098926; and WO2022098926.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-B illustrate the schematics of the transplant according to the methods described herein (identified as High-Precision Orca-T or OrcaT) and the differences compared to a standard of care (SOC) cohort (identified as Conventional Transplant or SOC).

FIG. 1C illustrates a schematic of graft production and administration.

FIG. 2A illustrates the weight of patients enrolled in the study disclosed in the Examples.

FIGS. 2B-2C illustrate the HSPC and Treg cell dose administered to the patients enrolled in the study disclosed in the Examples.

FIG. 2D illustrates the purity of Treg cells administered to the patients enrolled in the study disclosed in the Examples.

FIG. 3A shows the time to platelet engraftment in the study group (identified as Orca-T) and the standard of care (SOC) cohort.

FIGS. 3B-3L illustrate engraftment of various cell populations in the patients in the study group disclosed in the Examples. The figures also illustrate the levels of each cell type in the donors before sample collection. Boxplots where shown: boxes show the 75th, 50th, and 25th percentiles; whiskers show the 90th and 10th percentiles. X-axes nomenclature: the leading number (e.g. 01, 02, 025, . . . ) are mentioned for ordering; following the underscore, Dscrn=healthy donor pre-G-CSF mobilization, Rscrn=recipient within 1 month prior to conditioning, apher=healthy donor blood draw at the time of apheresis, d028=recipient day 28 post-transplant, d056-d365=recipient days post-transplant. N's shown indicate the sample sizes for each timepoint. Symbols indicate values for individual measurements. Cell numbers×10³ per uL of blood are equivalent to ×1,000 cells per uL of blood.

FIG. 3M-3N show the timeline of lymphocyte and monocyte engraftment in a subset of the study group (Orca-T) and the standard of care cohort.

FIG. 30 shows representative flow cytometry data for the frequency of CD3+CD4+ T cells that were Tregs in two subjects compared to a healthy control. In the healthy control, 3.72% of circulating CD3+CD4+ T cells were Tregs (CD25+CD127dim). In the two graft recipients, 28.1% and 23.7% of CD3+CD4+ T cells were Tregs on day +28, 32.3% and 17.8% on day +56, and 19.2% and 20.7% on day +100 post-transplant.

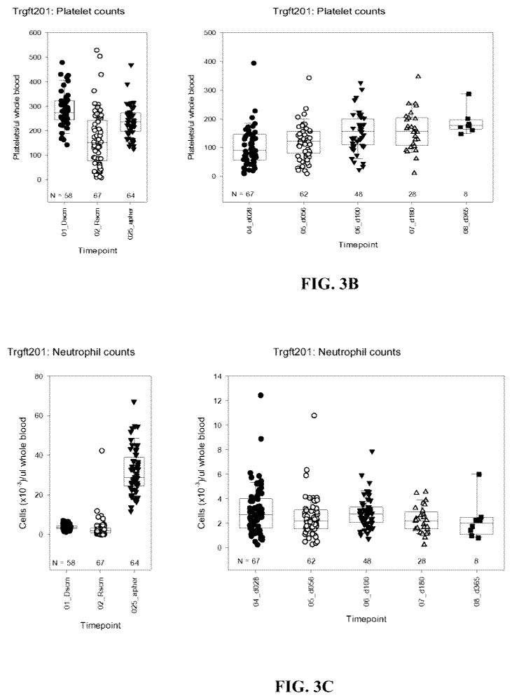

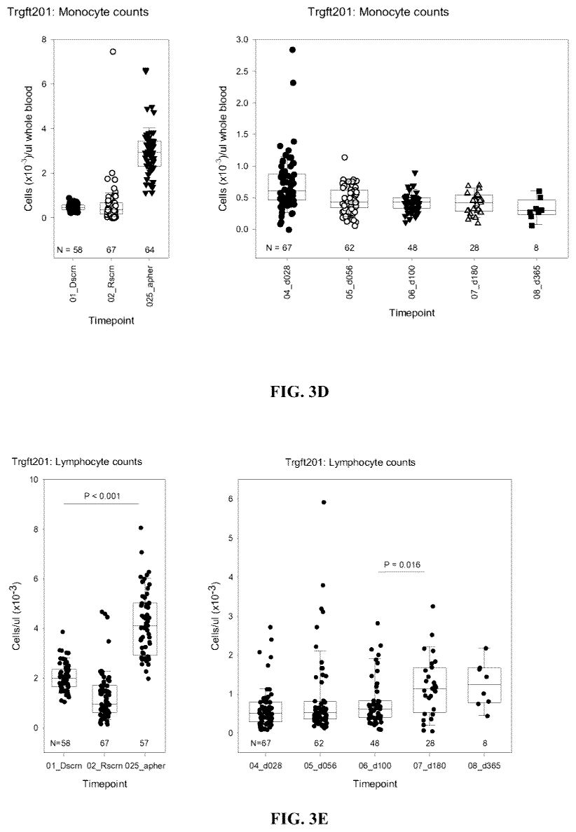

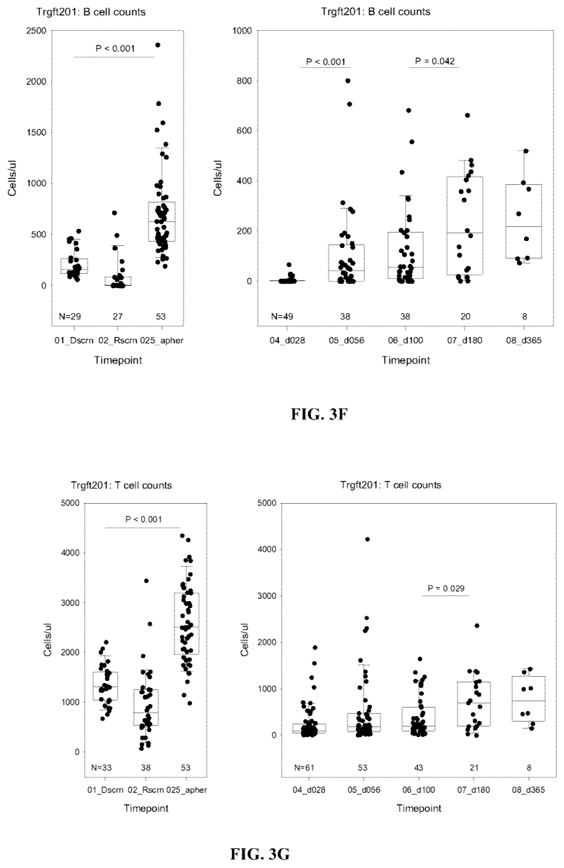

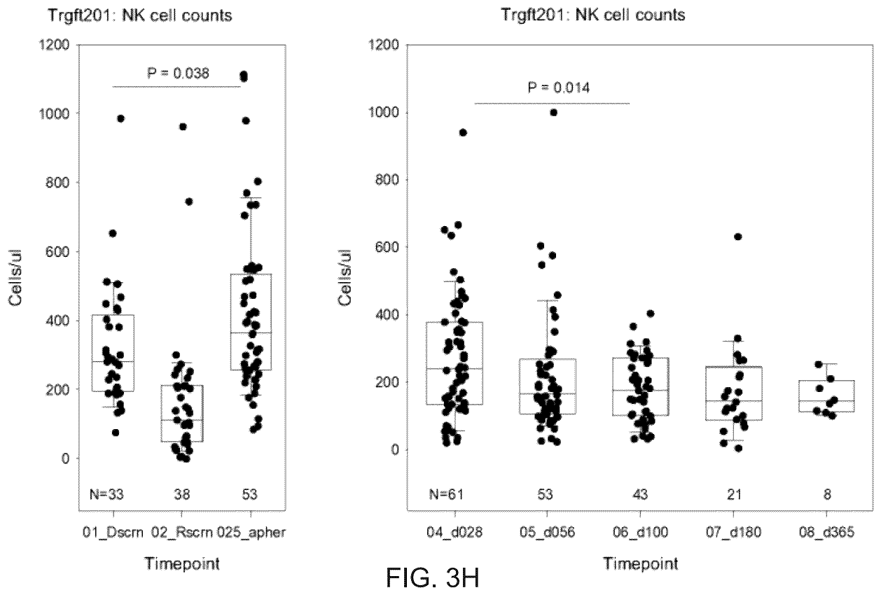

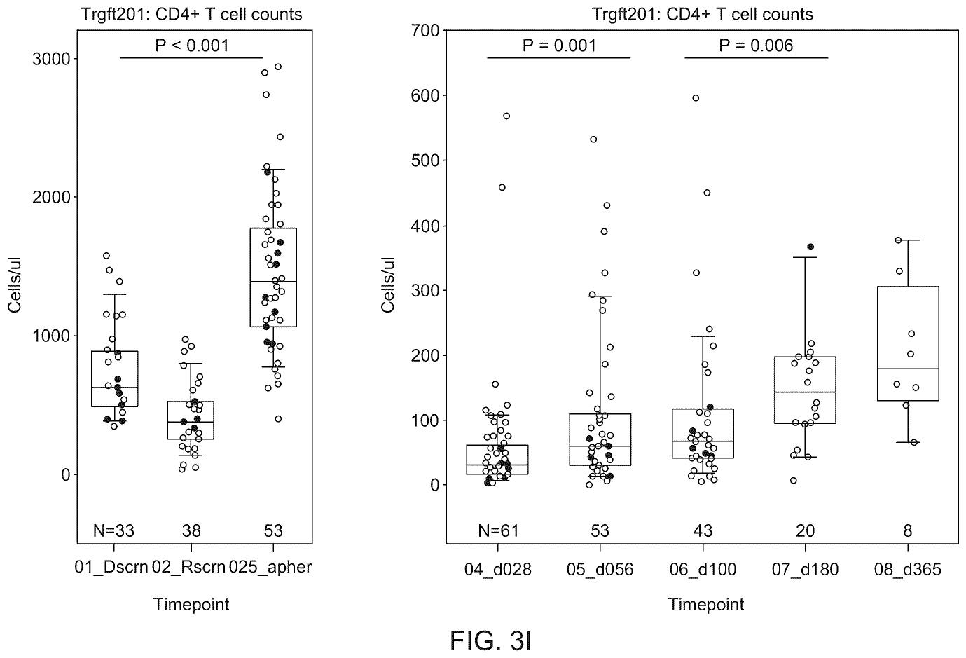

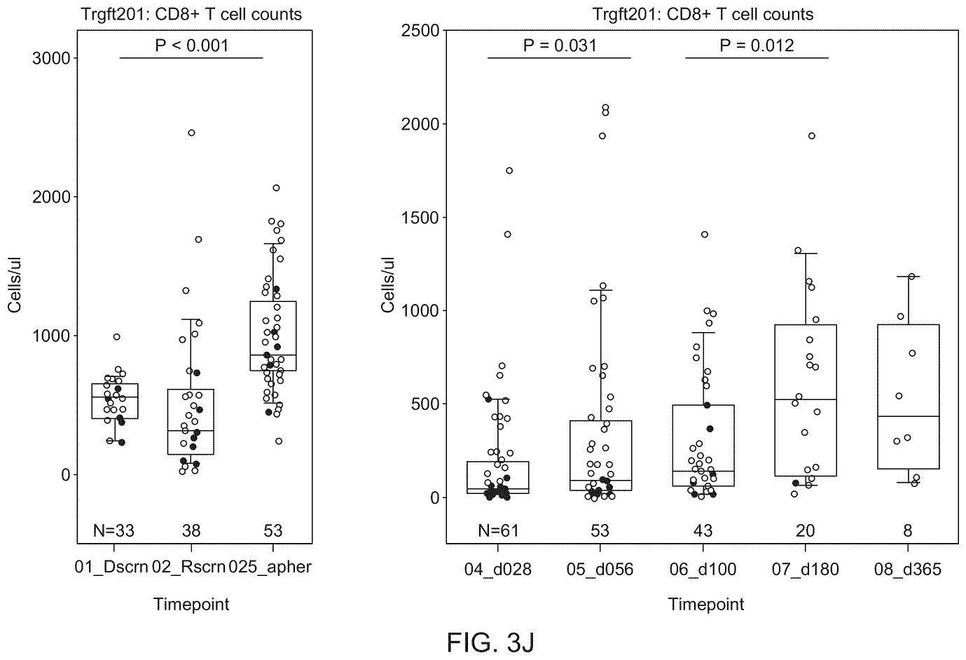

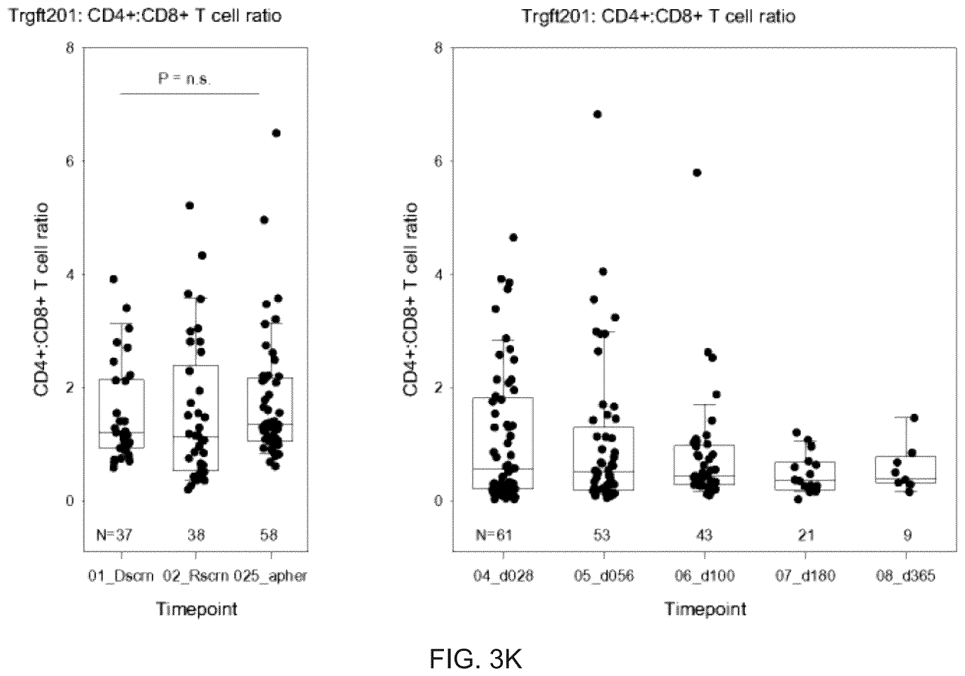

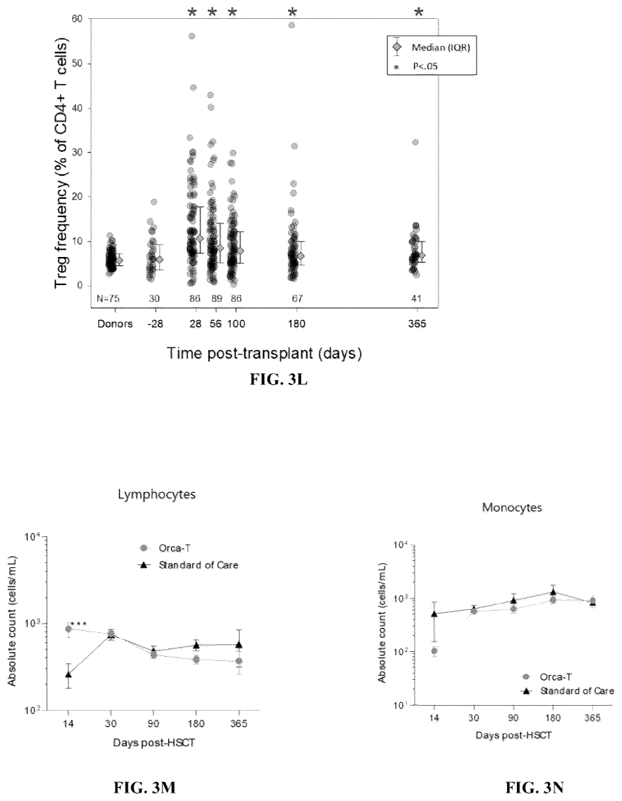

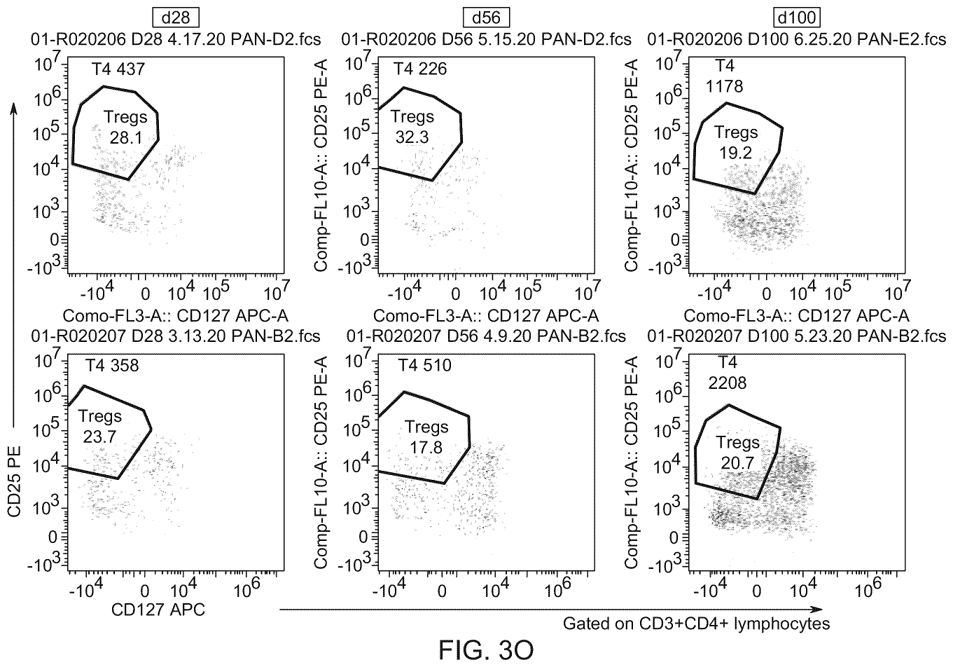

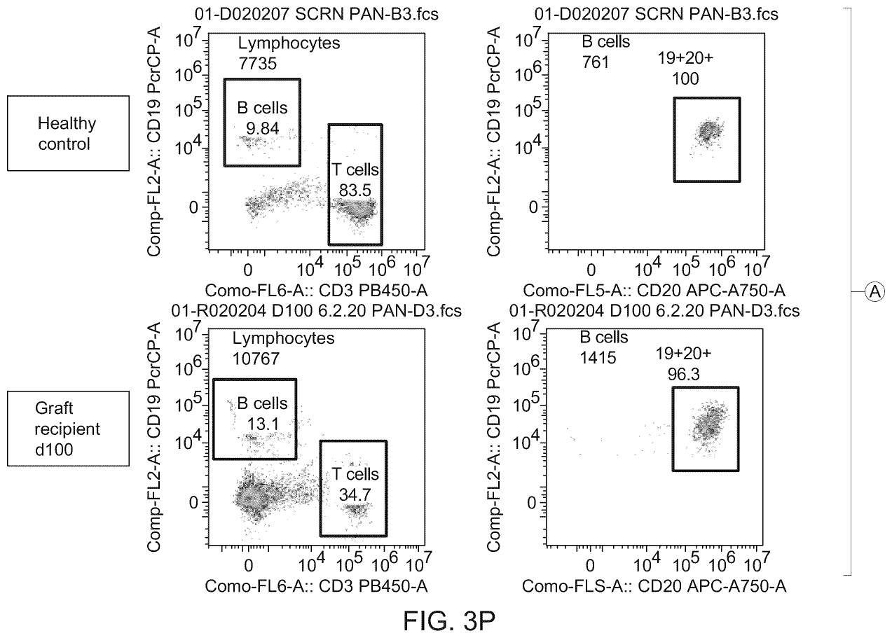

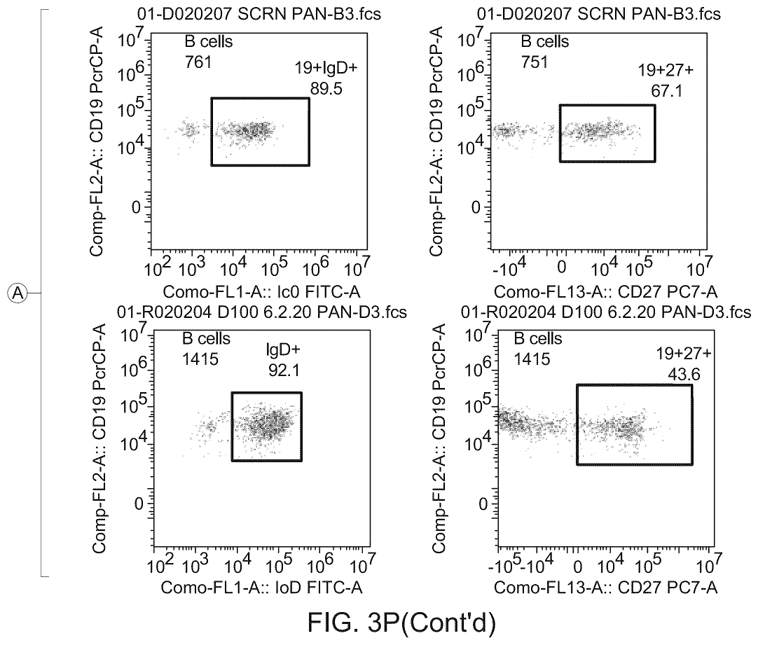

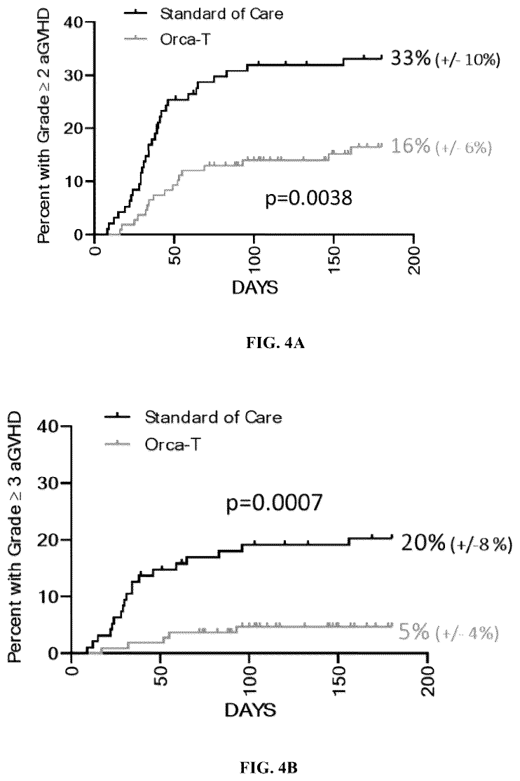

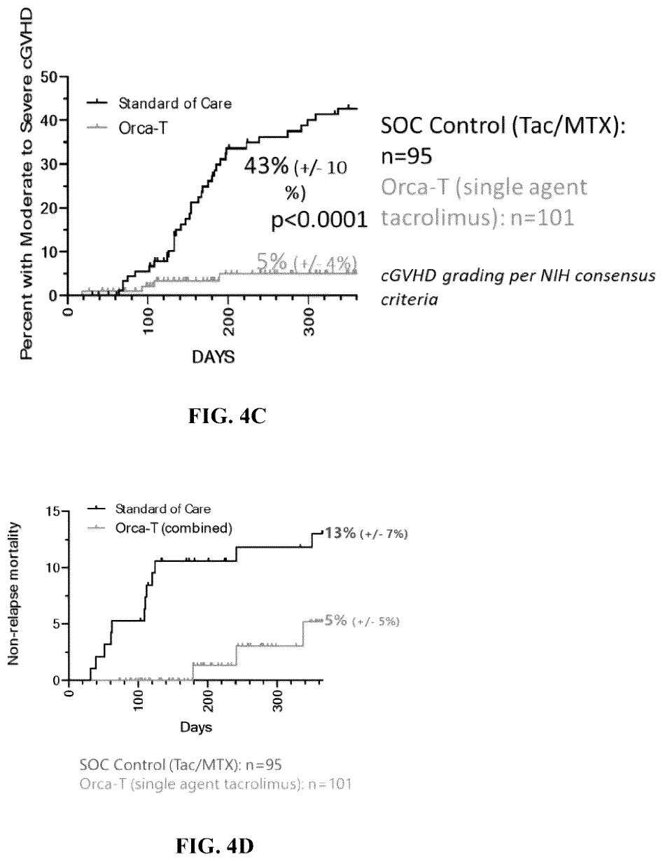

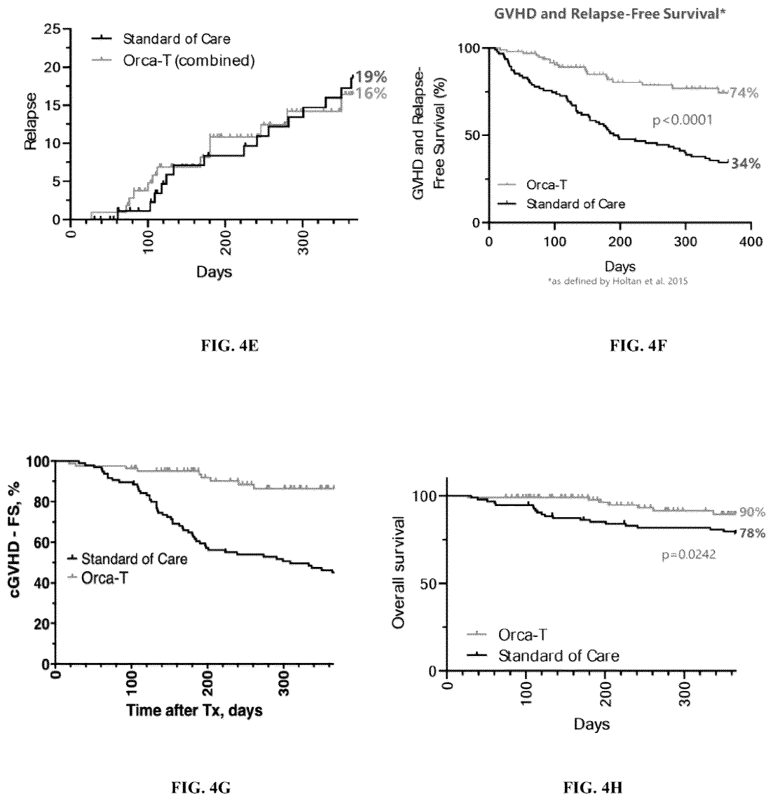

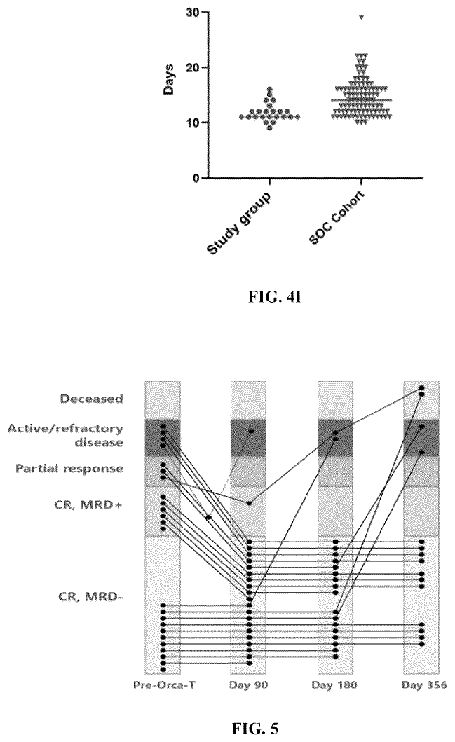

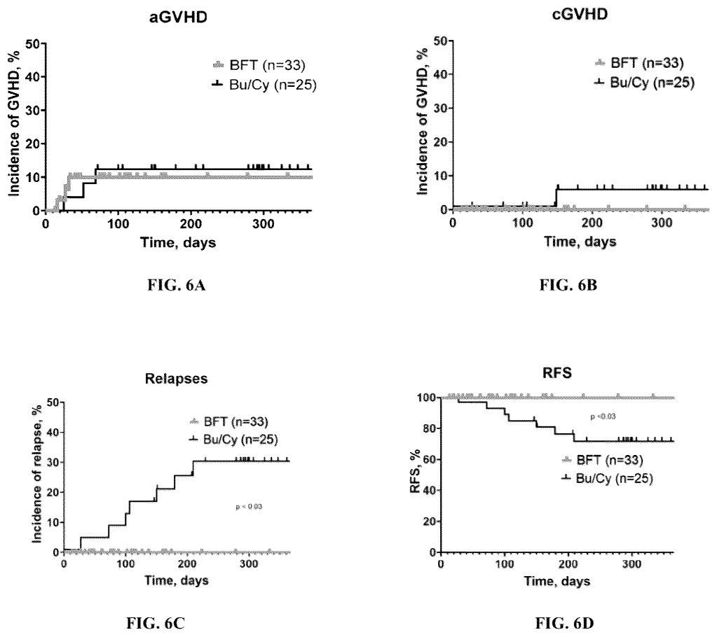

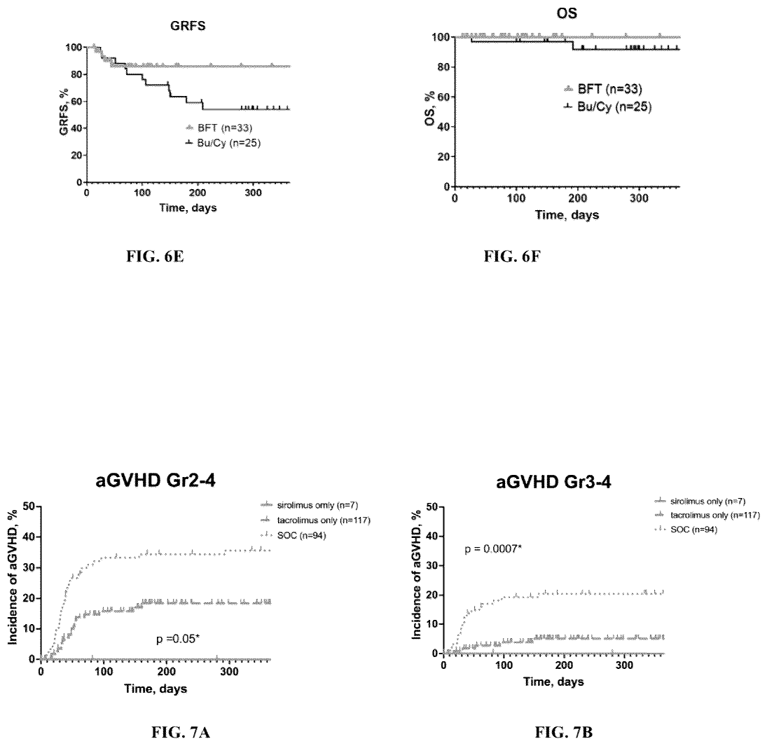

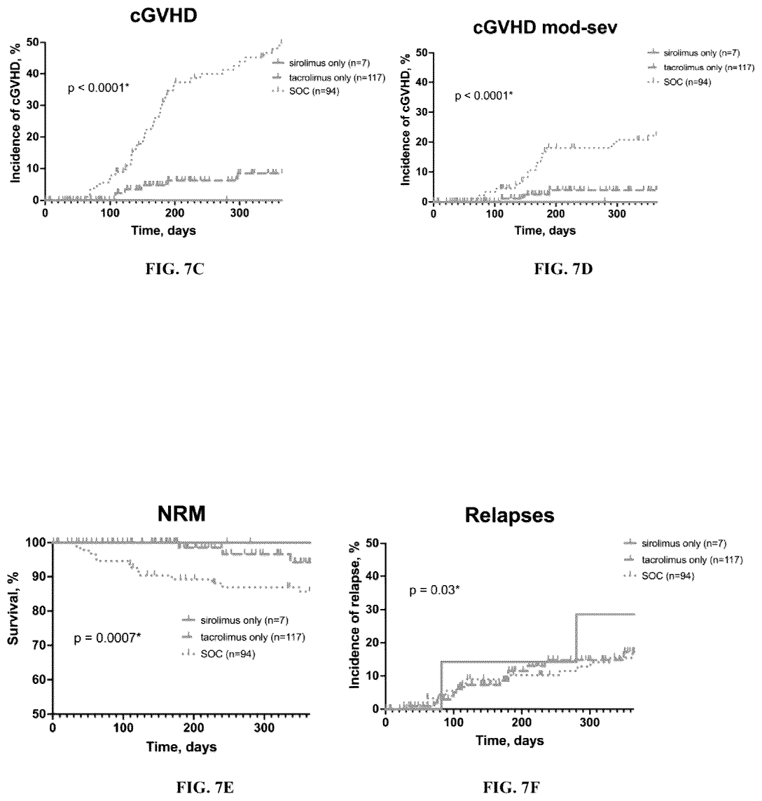

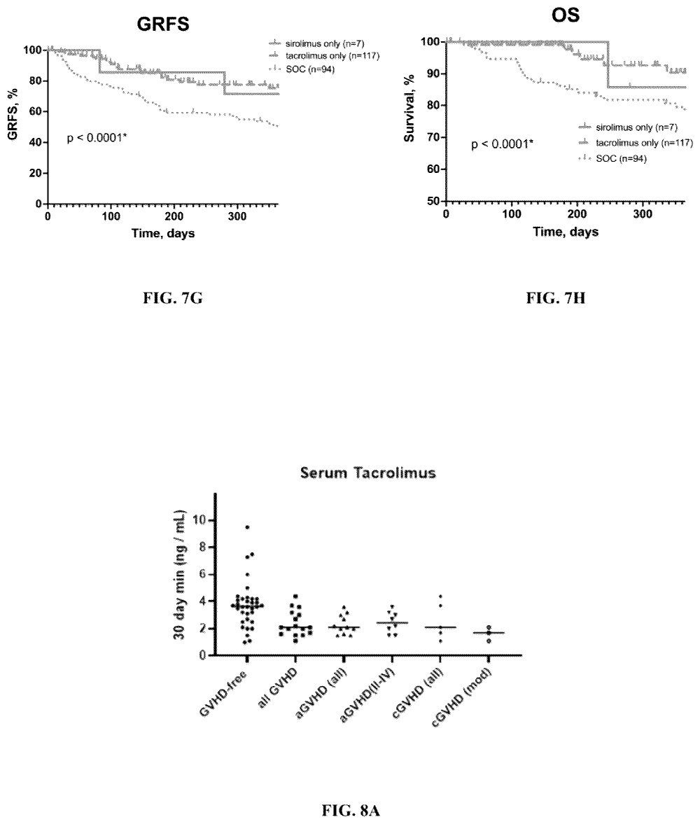

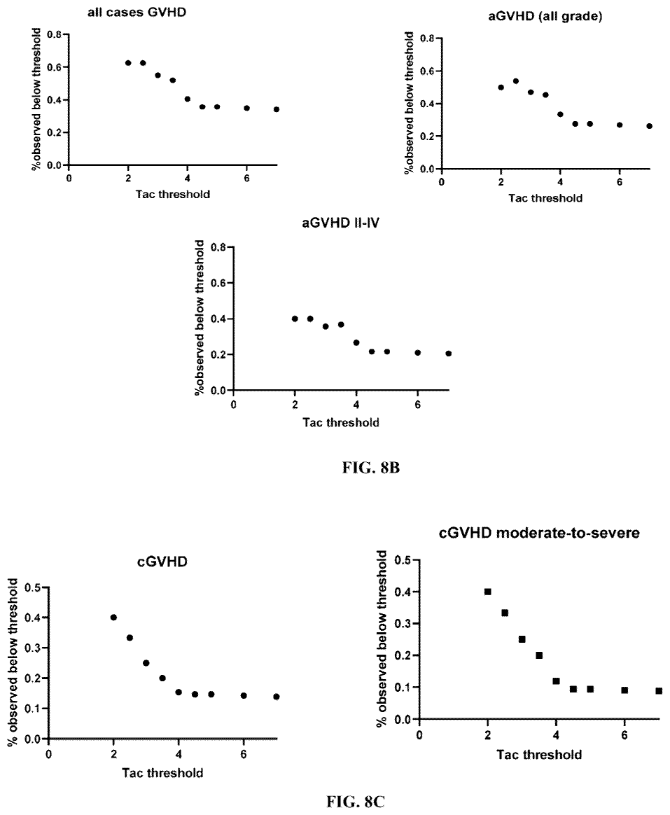

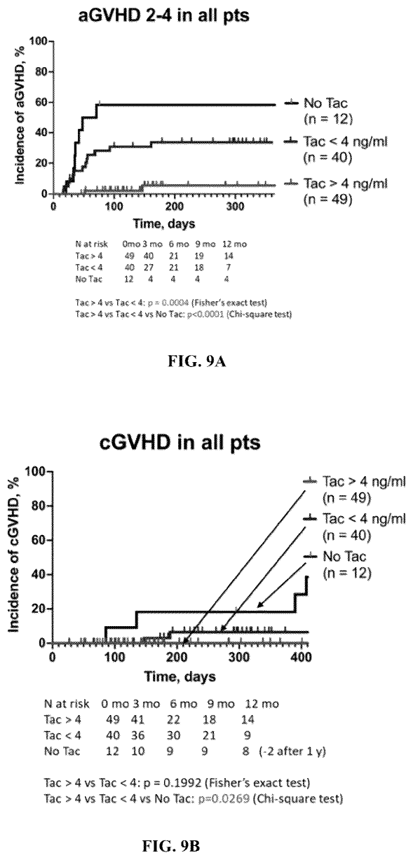

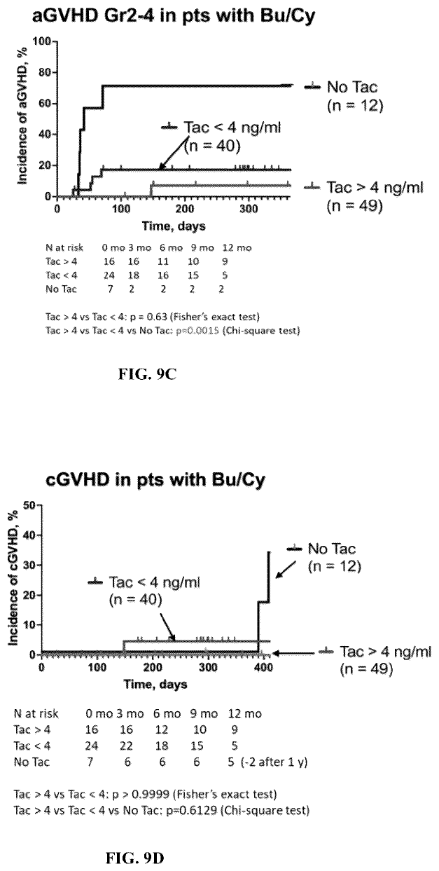

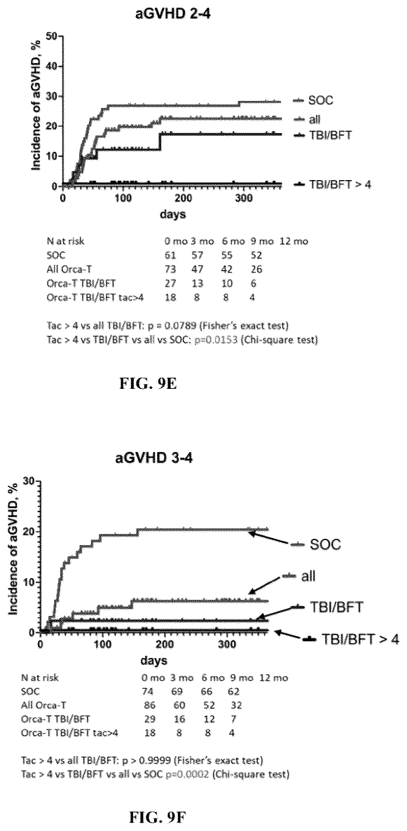

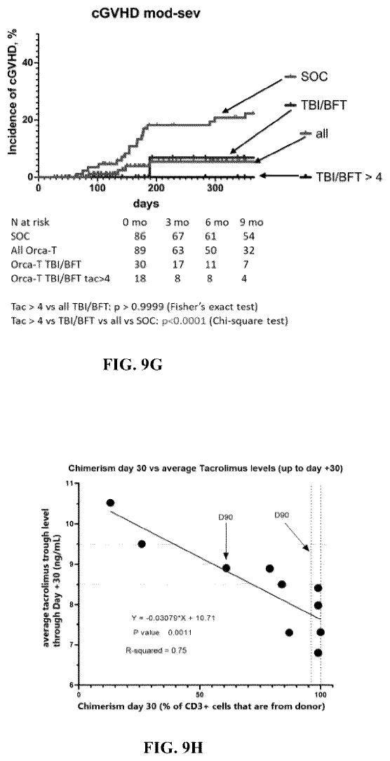

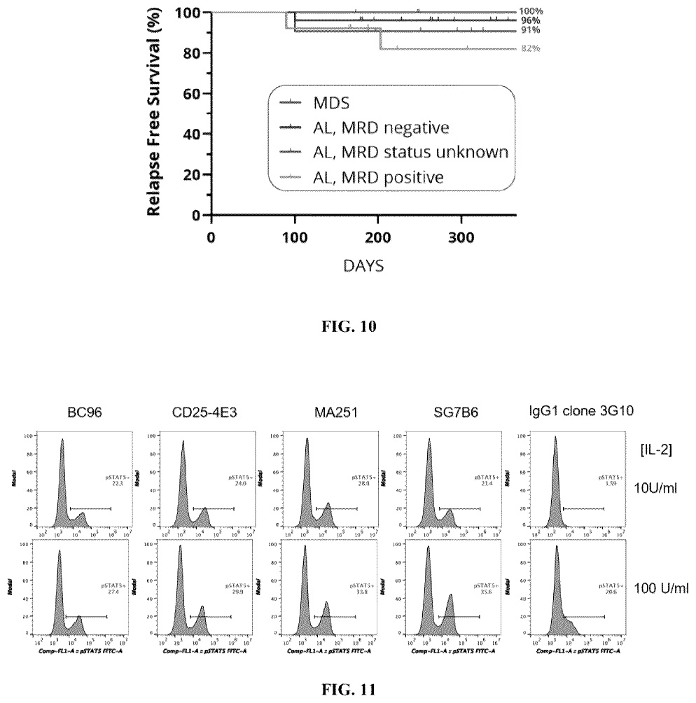

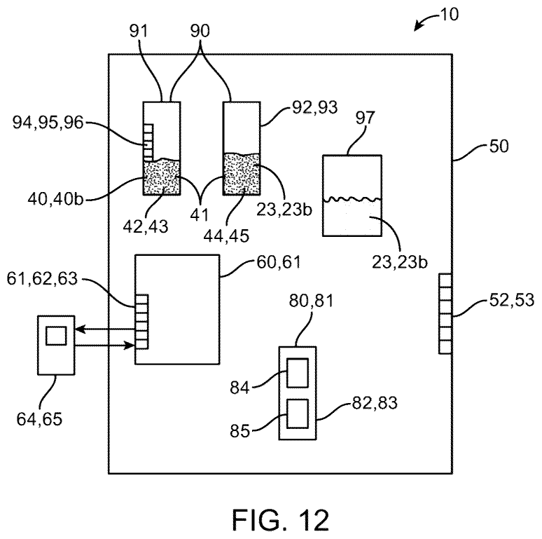

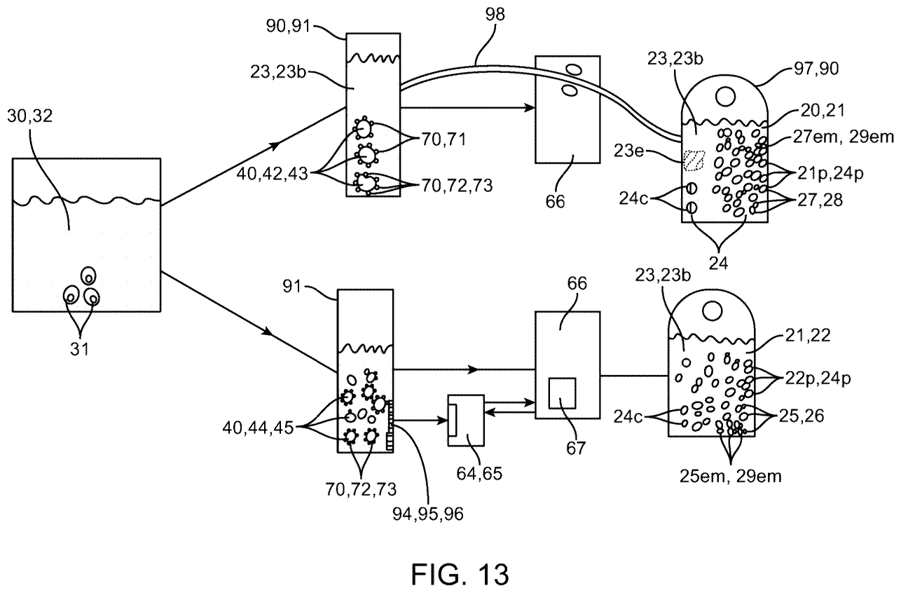

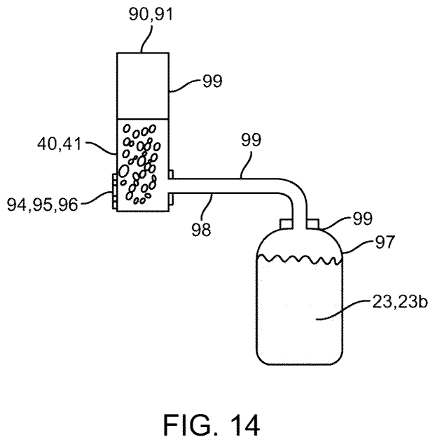

FIG. 3P shows flow cytometry data for B cell markers from a sample from a recipient of a composition of the disclosure compared to a healthy control. In all cases, the Y axis is for CD19+ staining. The left panels show gating of lymphocytes to identify B cells (CD19+) and T cells (CD3+). 13.4% of lymphocytes in the graft recipient were B cells, compared to 9.84% in the healthy control. The second from left panels show that 98.3-100% of cells gates as CD19+ were also CD20+. The panels second from the right show the fraction of B cells that are IgD+, which can be used to identify mature B cells. 92.1% of B cells in the graft recipient were IgD+, and 89.5% in the healthy control. The right-most panels show staining for CD27, which can be used to identify memory B cells, late plasmablasts, and plasma cells, for example. 43.6% of B cells in the graft recipient were CD27+, and 67.1% in the healthy control.

FIG. 4A shows the onset of grade ≥2 aGVHD in the study group (Orca-T) and the standard of care cohort through day +120 post-transplant. At nearly all timepoints, Orca-T data is below the standard of care data.

FIG. 4B shows the onset of grade ≥3 aGVHD in the study group (Orca T) and the standard of care cohort through day +120 post-transplant. At nearly all timepoints, Orca-T data is below the standard of care data.

FIG. 4C shows the onset of moderate to severe cGVHD in the study group (Orca-T) and the standard of care (SOC) cohort through day +365 post-transplant. At nearly all timepoints, Orca-T data is below the standard of care data.

FIG. 4D shows the non-relapse related mortality in the study group (Orca-T) and the standard of care (SOC) cohort through day +365 post-transplant. At nearly all timepoints, Orca-T data is below the standard of care data.

FIG. 4E shows relapse rates in the study group (Orca-T) and the standard of care cohort through day +365 post-transplant. At the final timepoint, Orca-T relapse rate is 16% and the standard of care relapse rate is 19%.

FIG. 4F shows GVHD and relapse-free survival rates in the study group (Orca-T) and the standard of care cohort through day +365 post-transplant. At nearly all timepoints, Orca-T data is above the standard of care data.

FIG. 4G shows cGVHD-free survival rates in the study group and the standard of care cohort through day +365 post-transplant. At nearly all timepoints, Orca-T data is above the standard of care data.

FIG. 4H shows overall survival rates in the study group and the standard of care cohort through day +365 post-transplant. At the final timepoint, Orca-T overall survival rate is 90% and the standard of care overall survival rate is 78%. FIG. 4I shows hospitalization days in a subset of the study group and the standard of care (SOC) cohort through day +365 post-transplant

FIG. 5 summarizes the disease status of a small subset of subjects in the study group before transplant and at day +90, +180, and +356 post-transplant. CR signifies complete remission, MRD signifies minimal residual disease.

FIGS. 6A-6F compare the aGVHD, cGVHD, relapse, relapse-free survival, GVHD and relapse free survival (GRFS) and overall survival rates in a subset of the patients in the study group that received different conditioning regimens.

FIGS. 7A-7H compare the aGVHD, cGVHD, non-relapse related mortality, relapse, relapse-free survival, GVHD and relapse free survival (GRFS) and overall survival rates in a subset of the patients in the study group that received different GVHD prophylactic agents.

FIGS. 8A-8C illustrate aGVHD and cGVHD rates in patients with different serum tacrolimus trough levels.

FIGS. 9A-9B compare the aGVHD and cGVHD levels in patients that had different serum tacrolimus levels.

FIGS. 9C-9D compare the aGVHD and cGVHD levels in patients that had different serum tacrolimus levels but were given the same conditioning regimen of busulfan and cyclophosphamide (Bu/Cy).

FIGS. 9E-9G compare the aGVHD and cGVHD levels in patients that had different serum tacrolimus levels but were given the same conditioning regimen of Total Body Irradiation (TBI)/Busulfan, Fludarabine, Thiotepa (TBI/BFT).

FIG. 9H shows the average trough tacrolimus level through day +30 post-transplant, plotted against the proportion of CD3+ cells of donor origin at day +30 (except that chimerism data is from day 90 where indicated by “D90”).

FIG. 10 illustrates relapse free survival in patients.

FIG. 11 illustrates IL2 stimulation of pSTAT5 signaling in Tregs using various clones of antibodies.

FIG. 12 is a lateral view illustrating an embodiment of a kit for the preparation of a therapeutic composition comprising cellular components for the treatment of a disease or condition such as cancer or an autoimmune disease.

FIG. 13 is a lateral view illustrating use of various components of the kit of FIG. 12 to prepare one or more therapeutic preparations comprising cellular components.

FIG. 14 is a lateral view illustrating an embodiment of container set comprising a column, blood bag and connecting tubing which can be included in embodiments of the kit.

FIG. 15 is a flow chart and schematic view illustrating an embodiment of a method of preparing a therapeutic composition comprising cellular components using embodiments of the therapeutic composition preparation kit.

### DETAILED DESCRIPTION

Embodiments of the disclosure provide compositions, systems, and methods for the treatment of diseases and conditions using various forms of cellular therapy such as hematopoietic stem cell transplantation. The diseases and conditions treated by various embodiments of the disclosure may include one or more of leukemia, lymphoma and other forms of stem cell-based cancer, graft versus host disease resulting from organ transplants (e.g., bone marrow transplant) and various autoimmune conditions. Embodiments of the disclosure also provide kits and methods for preparing and administering therapeutic compositions comprising one or more cells populations including, for example, populations of hematopoietic stem and progenitor cells (HSPC's); populations of regulatory T cells (Tregs), and/or population of conventional T cells (Tcons).

AlloHCT is the transplantation of multipotent hematopoietic stem and progenitor cells (HSPC's), usually derived from donor bone marrow, peripheral blood, or umbilical cord blood, into a recipient (i.e., a patient in need thereof). The recipient may be or may have been subjected to myeloablative conditioning, which kills hematopoietic cells, including tumor cells and host immune cells. The HSPC's transplanted into the recipient then reconstitutes the hematopoietic compartment. AlloHCT can be useful as a treatment for cancer due to the ability of donor T cells to exert anti-tumor effects, which is termed graft versus tumor (GVT). In patients with hematologic malignancies that are refractory to chemotherapy, alloHCT is associated with improved survival.

However, donor T cells can also attack non-tumor host cells, resulting in graft versus host disease (GVHD). GVHD is a major source of post-HCT complications and can be fatal. Management of GVHD can require immunosuppressive therapy or cytotoxic mediations, which can cause toxicity, increase susceptibility to infection, and/or blunt anti-tumor immunity. The early morbidity and mortality associated with acute graft versus host disease (aGVHD; which occurs within the first 100 days post-transplant) is a major factor limiting the success of alloHCT, as is the long-term morbidity associated with chronic GVHD (cGVHD; which occurs later than the first 100 days post-transplant). GVHD is a risk for both HLA-matched and HLA-mismatched transplantations. GVHD can occur even if the donor and recipient are HLA-matched, because the immune system can still recognize other differences between the donor tissues.

Both GVT and GVHD are largely mediated by conventional T cells (Tcons), which mount immune responses upon recognition of cognate antigen by T cell receptors. Depleting T cells from hematopoietic stem cell transplantation (HCT) grafts can reduce GVHD, but can also result in reduced GVT and increased likelihood of cancer relapse. Besides Tcons, Tregs are an additional subset of T cells; however, Tregs negatively regulate inflammation and promote immune tolerance. Tregs can prevent or reduce GVHD through their negative regulation of inflammation, including inflammation elicited by donor Tcons when they recognize recipient antigens.

Various embodiments of the disclosure provide methods for improved alloHCT. According to one or more embodiments, such methods may comprise administering to a subject certain cell components that comprise populations of cells, including a cell component comprising HSPC's, a cell component comprising Tregs, and a cell component comprising Tcons. Without wishing to be bound by theory, administering Tregs reduces the incidence and/or severity of GVHD, while administering Tcons enhances GVT. Various embodiments of the disclosure leverage these two effects. i.e., minimizing GVHD while enhancing GVT. In use, embodiments of the compositions, kits and methods disclosed herein, provide the benefit of retaining the graft-versus-tumor (GVT) effects of alloHCT administered to a subject having a cancer, while preventing or reducing graft versus host disease (GVHD) in the subject. In some embodiments, two or more populations of cells are administered at different times, for example, HSPC's and Tregs can be administered prior to Tcons to further reduce the incidence and severity of GVHD.

In some embodiments, the administering may enhance hematopoietic chimerism in the human subject. Following administration of transplant cells from a donor to a recipient, chimerism can be monitored in the recipient. Chimerism can refer to the mix of donor and host cells in an individual who has received an alloHCT. The risk of GVHD is markedly reduced in patients with mixed instead of complete chimerism, and achieving mixed chimerism is desirable for this reason. In addition, immunodeficiency and infection are more frequently observed in complete versus mixed chimerism. In some embodiments, the methods provided herein allow a human subject to achieve mixed chimerism.

Subjects who exhibit more than a 95% donor cells in a given cell lineage at any time post-transplantation can be referred to as having full donor chimerism. Subjects who exhibit greater than 1% but less than 95% donor DNA in such analysis can be referred to as having mixed chimerism. Subjects who exhibit mixed chimerism can be further classified according to the evolution of chimerism, where improving mixed chimerism can comprise a continuous increase in the proportion of donor cells over at least a 6-month period. Stable mixed chimerism can comprise fluctuations in the percentage of recipient cells over time, without complete loss of donor cells.

A determination of whether a subject is a full chimera, mixed chimera, or non-chimera can be made by an analysis of a hematopoietic cell sample from the graft recipient, e.g. peripheral blood or bone marrow. Analysis can be done by any convenient method of typing. In some embodiments, the degree of chimerism amongst all mononuclear cells, T cells, B cells, CD56+ NK cells, and CD15+ neutrophils is regularly monitored, using PCR with probes for microsatellite analysis. For example, commercial kits can be used to quantify donor and host genetic material extracted from cells based on polymorphisms in short terminal repeat lengths. Automated readers provide the percentage of donor type cells based on standard curves from artificial donor and host cell mixtures.

Many embodiments of the disclosure provide therapeutic compositions comprising one or more cell populations such as populations of hematopoietic stem progenitor cells (HSPC's) and regulatory T-cells (Treg). In these and related embodiments, the populations of cells may be processed to have a selected percentage of a cell type. For example the populations of cells comprise Treg cells may comprise at least about 50 percent (%) Tregs, more preferably at least about 80% and still more preferably at least about 90%.

Many embodiments of the disclosure provide compositions and methods for hematopoietic stem cell transplantation including compositions and methods for allogeneic hematopoietic stem cell transplantation (alloHCT). In some embodiments, the methods disclosed herein retain graft-versus-tumor (GVT) effects of alloHCT when a therapeutic composition is administered to a patient with cancer (e.g., leukemia or lymphoma or other stem cell malignancy), while reducing the incidence and severity of graft versus host disease (GVHD).

According to one embodiment, a therapeutic composition for hematopoietic cell transplantation to a human patient in need thereof comprises a first population of isolated CD45+ cells wherein at least a portion of the CD45+ cells have an antibody or other antigen binding agent bound to a marker on the cell surface which is used to separate CD34+ cells from a mixture of nucleated cells from a volume of blood from a human donor; and a second population of isolated CD45+ cells wherein at least about 50% of the isolated CD45+ cells are regulatory T (Treg) cells with higher amounts contemplated as well. Typically, the marker is a receptor such as a CD34⁺, CD+25 or CD4+; however other receptors known in the art also contemplated. In one or more embodiments, the mixture of nucleated cells comprises at least about 70% CD34+ cells with even higher levels contemplated including for example at least 90% CD45+ cells. Also, in one or more embodiments, the isolated CD45+ cells in the second population may comprise at least 70 percent Treg cells with even higher percentages contemplated, e.g., 80, 90, 95 percentages. Higher percentage of Treg cells in the second population provide the benefits of reduced incidence and severity of graft versus host diseases and associated its morbidity and mortality.

In one or more embodiments, the compositions and methods described herein can be used for treatment of one or more diseases or conditions including, for example, stem cell-based cancer (which also corresponds to various hematologic malignancies), graft versus host disease (GHVD) related to the treatment of the cancer, non-malignant hematologic disorders including sickle cell anemia and hemophilia or various autoimmune diseases such as multiple sclerosis, Crohn's disease or ankylosing spondylitis. In additional embodiments, the compositions and methods of the disclosure may also be used to as adjunct therapy in various organ, tissue, or cell transplants to reduce rejection of the transplant by the patient's immune system, including kidney, heart, lung and liver transplants and also transplants of specific cell types, including the mixed endocrine cell types making up the islets of Langerhans and various cells making up heart, lung and brain tissue including one more of myocytes, neurons and glial cells.

According to various embodiments, the first population of cells may have maximum thresholds on one or more non-CD-34+ cells in the populations. Such maximum thresholds may include for example, less than about 5% CD3⁺ cells, and less about 20% granulocytes with lower levels contemplated as well, for example, less than about 2% CD3⁺ cells, and less about 10% granulocytes. The first population may also have absolute limits on these and other cells on the basis of number of cells per kg patient weight, for example less than about 6.2×10⁵ granulocyte cells per kg patient weight (which may be the patient's actual weight or the patient's ideal weight), less than about 2×10⁵ monocyte cells per kg patient weight, and less than about 1.3×10⁵ B-cells and natural killer cells in combination per kg patient weight. In use, such upper limit thresholds on these or other cells allow amount of graft versus host disease (GVHD) and/or other adverse immune related responses associated with transplantation of HSPC's to a patient for the treatment of one or more conditions. In particular, such threshold they provide the benefits of reducing the incidence and/or severity of GVHD including GVHD associated morbidity and mortality. For example, they may reduce the severity of acute GVHD from grade 3 to grade 2 or lower from grade 2 to grade 1 or lower.

In some aspects, provided herein is a method of treating a population of human subjects in need thereof. The method may comprise administering to the human subjects at least two or more therapeutic compositions which may be produced using embodiments of the kits and methods descried herein, wherein the pharmaceutical compositions are selected from: a pharmaceutical composition comprising a population of hematopoietic stem and progenitor cells (HSPC's); a pharmaceutical composition may comprise a population of regulatory T cells (Tregs); and a pharmaceutical composition may comprise a population of conventional T cells (Tcons). According to various embodiments, one or more pharmaceutical compositions may collectively comprise a pharmaceutical dosing system and/or multicomponent pharmaceutical treatment. In some embodiments, each cell population comprise less than about 5 EU/ml endotoxins. In some embodiments, less than about 15% human subjects in a group of at least 100 human subjects administered the two or more pharmaceutical compositions develops a stage 2 or higher graft versus host disease (GVHD) response within 30 days after being administered the pharmaceutical composition may comprise the population of Tcons.

In some embodiments, administration of the cells populations into the patient may be done by infusing into the human subject the population of HSPC's, the population of Tregs, and the population of Tcons.

Embodiments of the disclosure provide a therapeutic kit comprising one or more therapeutic compositions described herein including for example compositions comprising cells populations (e.g., first and second cell populations such as HSPC's and Tregs) and instructions for use of the cell populations to treat a patient for one or more conditions such as various hematologic cancers. The instructions for use can include specific instructions for administering the cell populations to a patient including administration methods, dose (e.g., per kg patient weight) and dosing regimens including time sequences and rates of administration (e.g., infusion rate for IV infusion).

### I. Cell Components

Various embodiments of the disclosure provide compositions (e.g., therapeutic compositions) and methods for improving the outcomes associated with hematopoietic stem cell transplantation (HCT), for example, allogeneic hematopoietic stem cell transplantation (alloHCT) for the treatment of one or more conditions, such as various stem cell related cancers (e.g., leukemia and lymphoma). In many embodiments, such compositions may include and/or be associated with one or more cell components. Such cell-based compositions may be produced using one or more embodiments of the kits and production methods described herein including those for example which include CD+34 and CD+25 antibodies or other antigen binding agents. A cell component can comprise one or more populations of cells, for example, hematopoietic stem and progenitor cells (HSPC's), conventional T cells (Tcons), regulatory T cells (Tregs), invariant natural killer T cells (iNKTs), memory T cells (Tmems), and combinations thereof. For embodiments of therapeutic compositions including two population of CD45+ cells such as HSPC's and Tregs (in the first and second cell population) desirably at least 70 percent of the cells in the second population comprise Treg cells, with even higher percentages contemplated, e.g., 80, 90, 95 percentages. Higher percentage of Treg cells in the second population provide the benefits of reduced incidence and severity of graft versus host diseases and associated its morbidity and mortality.

In addition to the cells, and other components of therapeutic preparation, embodiments of the disclosure also provide parameters for cell components and methods of administering cell components that can contribute to successful clinical outcomes in alloHCT recipient subjects. Without wishing to be bound by any theory, parameters that can contribute to successful clinical outcomes in alloHCT recipient subjects include, without limitation, the particular cell populations administered, order and timing for the administration of different populations, purity standards for cell populations, methods for obtaining populations, methods of handling or storing populations (e.g., use of fresh versus frozen cell populations), dosages of populations administered, methods for obtaining populations, and combinations thereof.

HSPC's can have extensive self-renewal capacity, and an ability to differentiate into specialized cell types, for example, an ability to reconstitute all hematopoietic cell lineages. HSPC's can undergo asynchronous replication, where two daughter cells are produced with different phenotypes. HSPC's cells can exist in a mitotically quiescent form. HSPC's can be derived from bone marrow, peripheral blood, and/or umbilical cord blood.

Subsets of immune cells, such as conventional T cells (Tcons), regulatory T cells (Tregs), invariant natural killer T cells (iNKTs), and memory T cells (Tmems) can contribute to aspects of GVHD following alloHCT, and can also contribute to, for example, GVT immune responses, immune reconstitution, infection susceptibility, and patient survival.

GVHD can be mediated in large part by donor T cells, which can elicit inflammatory responses upon recognition of recipient antigens. T cell depletion (TCD) of cell components for transplantation to a subject can be undertaken to decrease the likelihood of acute and/or chronic GVHD. T cells can be depleted using methods including, but not limited to, physical adsorption of T cells to protein ligands such as lectins, immunodepleting with T cell specific antibodies, and immunoaffinity techniques (for example, use of T cell or lymphocyte-specific antibodies in immunoadsorption columns, magnetic activated cell sorting (MACS), or fluorescent activated cell sorting (FACS)). Applying TCD techniques to donor grafts can result in, for example, 10-fold to 10⁵-fold depletion of T cells, and reduced incidence of GVHD. However, TCD can also result in increased incidence of cancer relapse, as the lack of T cells can reduce a graft-versus-tumor (GVT) immune response. Additionally, TCD can result in impaired immune recovery, and increased susceptibility to infections.

Both GVT and GVHD can be largely mediated by conventional T cells (Tcons), which mount immune responses upon recognition of cognate antigen by T cell receptors (tumor antigens for GVT, non-tumor recipient antigens for GVHD). Tcons can, for example, contribute to GVT, GVHD, or a combination thereof. In some embodiments, administration of Tcons after administration of Tregs can be made so as to enhance GVT immunity, and/or reduce susceptibility to infection. The timing, dose and content of the Tcons can be adjusted so as to optimize GVT immunity and reduce infection risk

Tcons can broadly refer to all CD3+ T cells, cells expressing CD3 and CD4 or cells expressing CD3 and CD8, cells expressing medium to high levels of CD127, cells expressing CD3 and medium to high levels of CD127, cells expressing CD3, cells expressing medium to high levels of CD127, and cells expressing CD4 or CD8. In some embodiments, Tcons do not express Vα24Jα18 TCR. Tcons and regulatory T cells (“Tregs”) can be non-mutually-exclusive cell populations. In some embodiments, Tcons and Tregs are mutually exclusive cell populations.

Regulatory T cells (“Tregs”) are a specialized subpopulation of T cells that negatively regulate (e.g., suppress) activation of the immune system and thereby promote immune tolerance. Without wishing to be bound by theory, cell components of the disclosure comprising Tregs contribute to positive clinical outcomes by, for example, reducing the incidence and/or severity of GVHD in a transplant recipient subject, and/or improving immune reconstitution in a transplant recipient. Administering Tregs with HSPC's can, for example, facilitate retention of graft versus tumor (GVT) and reduced incidence and/or severity of GVHD. Without wishing to be bound by theory, administering Tregs can prevent or reduce GVHD, and administering Tcons can promote GVT effects, for example, relative to alternate hematopoietic stem cell transplantation (HCT) methods, wherein alternate HCT methods are distinct from the methods disclosed and/or claimed herein. In some embodiments, administering Tregs reduces the risk of developing GVHD, and administering Tcons promotes GVT effects relative to alternate alloHCT methods, wherein alternate HCT methods are distinct from the methods disclosed and/or claimed herein.

As used herein, an alternate composition lacks one or more cell components and/or prophylactic agents that are disclosed herein and/or recited in the claims. As examples, an alternate composition lacks one or more of a cell component comprising HSPC's, a cell component comprising Tregs, a cell component comprising Tcons, and a prophylactic agent.

There are a number of subsets of Tregs, for example, TCRαβp+CD4+ regulatory T cells, which include natural regulatory T cells (nTregs) and induced regulatory T cells (iTregs). nTregs can be T cells produced in the thymus and delivered to the periphery as a long-lived lineage of self-antigen-specific lymphocytes. iTregs can be recruited from circulating lymphocytes and acquire regulatory properties under particular conditions of stimulation in the periphery. nTregs and iTregs are CD4+CD25+; both can inhibit proliferation of CD4+CD25− T cells in a dose-dependent manner. In some embodiments, Tregs are anergic and do not proliferate upon TCR stimulation. In addition to being positive for CD4 and CD25, Tregs can be positive for the transcription factor FOXP3, an intracellular marker. Tregs can be identified or selected based on various marker expression profiles. Non-limiting examples of marker expression profiles that can be used to select Tregs include (1) CD4+CD25+CD127dim, (2) CD4+FOXP3+, (3) CD3+CD4+CD25+, (5) CD3+CD4+ CD25+ CD127dim, (6) CD3+ CD4+ CD25+ CD127dim FOXP3+, (7) CD3+FOXP3+, (8) CD3+CD4+FOXP3+, (9) CD3+CD4+CD25+FOXP3+, (10) CD3+CD25+FOXP3+, (11) CD3+CD25+CD127dim, (12) CD4+CD25+, (13) CD4+CD25+CD127dimFOXP3+, (14) FOXP3+, CD4+FOXP3+, (15) CD4+CD25+FOXP3+, (16) CD25+FOXP3+, and (17) CD25+CD127dim.

Selection based on certain expression profiles can be achieved based on extracellular markers and without requiring cell permeabilization, for example, selection based on CD4+CD25+CD127dim.

A cell component that comprises Tregs can, for example, reduce the incidence of graft rejection, reduce the incidence and/or severity of GVHD, promote hematopoietic reconstitution, promote immune reconstitution, promote mixed chimerism, or a combination thereof.

In various embodiments, a cell component of the disclosure can comprise invariant natural killer T cells (iNKTs). iNKTs are subclass of CD1d-restricted Natural Killer T (NKT) cells that express a highly conserved αβ-T cell receptor that comprises of Vα24Jα18 TCRα chain in humans (referred to herein as “Vα24Jα18+”). iNKT cells can be identified by binding with CD1d-multimers like that are loaded with α-galactosylceramide (GalCer), PBS-57, PBS-44 or other natural or synthetic glycolipids. Another method of identification is an antibody or combination of antibodies that specifically recognize the Vα24Jα18 region. Examples include a Vα24 antibody, a Jα18 antibody, or the monoclonal antibody clone 6B11 which binds specifically to a unique region of the Vα24Jα18 TCR and can be used to identify iNKT cells. iNKTs can be CD3+Vα24Jα18+.

In some embodiments, iNKTs can promote engraftment, promote GVT, reduce incidence and/or severity of GVHD, decrease susceptibility to cancer relapse, decrease susceptibility to infection, or a combination thereof. In some embodiments, iNKTs promote the activity of Tregs. In some embodiments, iNKTs promote the activity of HSPC's.

In various embodiments, a cell component of the disclosure can comprise memory T cells (Tmems). Tmems can refer to antigen-experienced T cells that express, for example, the phenotypic markers CD45RO, TCRα, TCRβ, CD3, CD4, CD95, and IL-2Rβ or the phenotypic markers CD45RO, TCRα, TCRβ, CD3, CD8, CD95, and IL-2Rβ. Tmems provide immunity and are capable of persisting for a long period of time in an inactive state. Tmems are able to rapidly acquire effector functions upon re-challenge with antigen. A population of Tmems can include any combination of the subclasses T central memory cells and T effector memory cells. In some embodiments, Tmems are CD3+CD45RA−CD45RO+. In the methods of the present disclosure, Tmems administered to a subject receiving alloHCT can, for example, promote GVT, reduce GVHD, decrease susceptibility to cancer relapse, decrease susceptibility to infection, or a combination thereof.

### A. Acquisition and Processing of Cells

In some embodiments, at least one mobilized peripheral blood donation is collected from a donor or at most two mobilized peripheral blood donations are collected from the donor.

In embodiments, at least one of the mobilized peripheral blood donations is processed and sorted to enrich CD34+ cells and Tregs. In some embodiments, the processing and sorting time of the one or more of the mobilized peripheral blood donations is less than about 35 hours, the processing and sorting time of the one or more of the mobilized peripheral blood donations is less than about 30 hours, the processing and sorting time of the one or more of the mobilized peripheral blood donations is less than about 25 hours, the processing and sorting time of the one or more of the mobilized peripheral blood donations is at most about 35 hours, and/or the processing and sorting time of the one or more of the mobilized peripheral blood donations is at most about 25 hours.

In various embodiments, the one or more of the mobilized peripheral blood donations is processed and sorted using one or more immune-separation particles (ISPs), e.g., ISPs comprise affinity reagents such as immuno-magnetic separation particles which may be antibodies each conjugated to an iron-containing particle. In some embodiments, the affinity reagents comprise a plurality of CD34-reagents (e.g., an anti-CD34 antibody) that binds to one or more CD34 receptors on a HSPC. In some cases, the ISPs may be antibodies.

In some cases, at least a portion of the plurality of ISPs are attached to CD34+ receptors on the HPSC's of the HSPC cell population; optionally, an average number of ISP's per HSPC in the HSPC cell population is less than about 20,000, an average number of ISP's per HSPC in the HSPC cell population is equal to or less than about 10,000, and/or an average number of ISP's per HSPC in the HSPC cell population is from about 1000 to about 20,000.

In some embodiments, an average number of ISP's per HSPC in the HSPC cell population may be about 1,500 to about 20,000. In some embodiments, an average number of ISP's per HSPC in the HSPC cell population may be at least about 1,500. In some embodiments, an average number of ISP's per HSPC in the HSPC cell population may be at most about 20,000. In some embodiments, an average number of ISP's per HSPC in the HSPC cell population may be about 1,500 to about 2,000, about 1,500 to about 5,000, about 1,500 to about 6,000, about 1,500 to about 10,000, about 1,500 to about 12,000, about 1,500 to about 15,000, about 1,500 to about 20,000, about 2,000 to about 5,000, about 2,000 to about 6,000, about 2,000 to about 10,000, about 2,000 to about 12,000, about 2,000 to about 15,000, about 2,000 to about 20,000, about 5,000 to about 6,000, about 5,000 to about 10,000, about 5,000 to about 12,000, about 5,000 to about 15,000, about 5,000 to about 20,000, about 6,000 to about 10,000, about 6,000 to about 12,000, about 6,000 to about 15,000, about 6,000 to about 20,000, about 10,000 to about 12,000, about 10,000 to about 15,000, about 10,000 to about 20,000, about 12,000 to about 15,000, about 12,000 to about 20,000, or about 15,000 to about 20,000. In some embodiments, an average number of ISP's per HSPC in the HSPC cell population may be about 1,500, about 2,000, about 5,000, about 6,000, about 10,000, about 12,000, about 15,000, or about 20,000. In some embodiments, an average number of ISP's per HSPC in the HSPC cell population may be at least 1,500, 2,000, 5,000, 6,000, 10,000, 12,000, 15,000, or 20,000. In some embodiments, an average number of ISP's per HSPC in the HSPC cell population may be at most 1,500, 2,000, 5,000, 6,000, 10,000, 12,000, 15,000, or 20,000.

In various embodiments, at least one of the cell populations have a plurality of immuno-separation particles (ISPs) attached to receptors on the cells of the cell population. In some cases, the plurality of ISPs are immuno-magnetic separation particles. In some embodiments, the plurality of ISPs comprise an antibody conjugated to an iron containing particle. In some cases, at least a portion of the plurality of ISPs are attached to CD34+ receptors on the HPSC's of the HSPC cell population; optionally, an average number of ISP's per HSPC in the HSPC cell population is less than about 6,000, an average number of ISP's per HSPC in the HSPC cell population is equal to or less than about 3,000, and/or an average number of ISP's per HSPC in the HSPC cell population is from about 1700 to about 3,000. In some cases, at least a portion of the plurality of ISPs are attached to CD25+ receptors on the cells of the Treg cell population; optionally, an average number of ISP's per T-reg cell in the Treg population is equal or less than about 1700 or an average number of ISPs per T-reg cell in the Treg population is from about 1400 to about 1700. In some cases, at least a portion of the plurality of ISPs are attached to CD3+ receptors on the cells of the heterogenous cell population; optionally, an average number of ISPs per cell in population of T heterogenous is less than about 1,000.

In some cases, at least a portion of the plurality of ISPs are attached to CD25+ receptors on the cells of the Treg cell population; optionally, an average number of ISP's per T-reg cell in the Treg population is equal or less than about 4000 or an average number of ISPs per T-reg cell in the Treg population is from about 1500 to about 2500. In some cases, at least a portion of the plurality of ISPs are attached to CD3+ receptors on the cells of the heterogenous cell population; optionally, an average number of ISPs per cell in population of T heterogenous is less than about 4,000.

In some embodiments, an average number of ISP's per Treg cells in the Treg cell population may be about 500 to about 4,000. In some embodiments, an average number of ISP's per Treg cells in the Treg cell population may be at least about 500. In some embodiments, an average number of ISP's per Treg cells in the Treg cell population may be at most about 4,000. In some embodiments, an average number of ISP's per Treg cells in the Treg cell population may be about 500 to about 1,000, about 500 to about 1,500, about 500 to about 2,000, about 500 to about 2,500, about 500 to about 3,000, about 500 to about 4,000, about 1,000 to about 1,500, about 1,000 to about 2,000, about 1,000 to about 2,500, about 1,000 to about 3,000, about 1,000 to about 4,000, about 1,500 to about 2,000, about 1,500 to about 2,500, about 1,500 to about 3,000, about 1,500 to about 4,000, about 2,000 to about 2,500, about 2,000 to about 3,000, about 2,000 to about 4,000, about 2,500 to about 3,000, about 2,500 to about 4,000, or about 3,000 to about 4,000. In some embodiments, an average number of ISP's per Treg cells in the Treg cell population may be about 500, about 1,000, about 1,500, about 2,000, about 2,500, about 3,000, or about 4,000. In some embodiments, an average number of ISP's per Treg cells in the Treg cell population may be at least 500, 1,000, 1,500, 2,000, 2,500, 3,000, or 4,000. In some embodiments, an average number of ISP's per Treg cells in the Treg cell population may be at most 500, 1,000, 1,500, 2,000, 2,500, 3,000, or 4,000.

In some embodiments, an average number of ISP's per Tcon cell in the Tcon cell population may be about 100 to about 1,000. In some embodiments, an average number of ISP's per Tcon cell in the Tcon cell population may be at least about 100. In some embodiments, an average number of ISP's per Tcon cell in the Tcon cell population may be at most about 1,000. In some embodiments, an average number of ISP's per Tcon cell in the Tcon cell population may be about 100 to about 200, about 100 to about 500, about 100 to about 1,000, about 200 to about 500, about 200 to about 1,000, or about 500 to about 1,000. In some embodiments, an average number of ISP's per Tcon cell in the Tcon cell population may be about 100, about 200, about 500, or about 1,000. In some embodiments, an average number of ISP's per Tcon cell in the Tcon cell population may be at least 100, 200, 500, or 1,000. In some embodiments, an average number of ISP's per Tcon cell in the Tcon cell population may be at most 100, 200, 500, or 1,000.

In various embodiments, cells of the mobilized peripheral blood donation are sorted such that the first population of CD45+ cells comprises at most about 10% granulocytes. In some cases, cells of the mobilized peripheral blood donation are sorted such that the first population of CD45+ cells comprises at most about 7% granulocytes.

In some embodiments, cells of the mobilized donor peripheral blood donation are sorted such that the first population of CD45+ cells comprises at most about 4% monocytes. In some cases, cells of the mobilized donor peripheral blood donation are sorted such that the first population of CD45+ cells comprises at least about 0.1% monocytes.

In embodiments, cells of the mobilized donor peripheral blood donation are sorted such that the population enriched for Tregs comprises at most about 10% CD25− cells.

A description will now be provided of the acquisition and processing of cells used in various embodiments of the disclosure. In one or more embodiments, the cellular components of therapeutic compositions described herein can be obtained from whole blood typically, from a donor. In many embodiments, such cellular components (e.g., HSPC's and Treg cells) can be obtained from a peripheral blood apheresis product using one or more embodiments of the kits described herein. Such blood product may include a mobilized peripheral blood apheresis product, e.g., mobilized by administration of one or more mobilizing agents such as GCSF, GM-CSF, mozobil, and combinations thereof, to a donor so as to cause HSPC's and other cells to be mobilized from the donor's bone marrow into their peripheral blood.

However, other embodiments of the disclosure, contemplate the use of non-mobilized blood including, for example, use of umbilical cord blood including that of the patient. Further, in additional or alternative embodiments, the blood product can be grown in vitro and/or may be derived from engineered blood cells including various nucleated cells such as stems cells and progenitor cells. Such engineered cells may be genetically engineered to have selected characteristics and may be grown in vitro or otherwise generated in a cell/bio incubator, bioreactor, cell culture dish or flask, cell culture well plate or other like device known in the cell culture arts

In various embodiments, a cell component can be obtained from at least one apheresis product, two apheresis products, three apheresis products, four apheresis products, five apheresis products, six apheresis products, or more. In some embodiments, a cell component of the disclosure is obtained from one apheresis product. In some embodiments, a cell component of the disclosure is obtained from two apheresis products. In some embodiments, a cell component of the disclosure is obtained from an apheresis product from one donor and an apheresis product from an at least second donor.

In various embodiments, one or more cell components and/cell populations described herein (e.g., HSPC and Treg cells) can be obtained from various organ, tissue/tissue sites or blood sources. For example, in one or more embodiments, such cellular components can be obtained from bone marrow, umbilical cord blood, peripheral blood, mobilized blood, mobilized peripheral blood, the thymus, lymph tissue or other tissue site in the body.

In many embodiments one or more of the cellular components described herein can be refined by selection from a population of cells, for example, peripheral blood or a peripheral blood apheresis product. Selection methods for cell populations may correspond to methods involving positive or negative selection of a cell population of interest or a combination of both. Selection methods for cell populations can comprise affinity reagents (also referred to as a selection reagent), including but not limited to an antibody, a full-length antibody, a fragment of an antibody, a naturally occurring antibody, a synthetic antibody, an engineered antibody, a full-length affibody, a fragment of an affibody, a full-length affilin, a fragment of an affilin, a full-length anticalin, a fragment of an anticalin, a full-length avimer, a fragment of an avimer, a full-length DARPin, a fragment of a DARPin, a full-length fynomer, a fragment of a fynomer, a full-length kunitz domain peptide, a fragment of a kunitz domain peptide, a full-length monobody, a fragment of a monobody, a peptide, or a polyaminoacid. In some embodiments, the affinity reagent is directly conjugated to a detection reagent and/or purification reagent. In some cases, the detection reagent and purification reagent may be the same. In other cases, the detection reagent and purification reagent may be different. For example, in some embodiments described herein, the detection reagent and/or purification reagent is fluorescent, magnetic, or the like one or more of which may be conjugated to an antibody or other antigen binding agent. In other embodiments, the detection reagent and/or purification reagent is only a magnetic particle (which may be conjugated to antigen binding agent) configured for use in column purification. Embodiments of kits described herein using antibodies or other antigen binding agent (which as described above fall in the category of affinity reagents) may include the same or different categories/types of affinity reagents. For example, in one or more embodiments, a kit for the preparation of cellular components administered to a patient may include magnetic particles conjugated to antibodies (or other antigen binding agent) for CD 34+ and CD 25+ cells (e.g., those cells possessing the respective marker) such as HSPC'S and Treg cells. In additional or related embodiments, a kit for the preparation of selected cell components described herein may include a fluorophore conjugated to an antibody (or other antigen binding agent) that binds to CD127+ cells such as Treg cells.

Affinity reagents can comprise immunoaffinity reagents, utilizing the binding specificity of antibodies or fragments or derivatives thereof to positively or negatively select for a cell population of interest. Cell selection methods which may be used by embodiments of the disclosure for generating a desired cell populations may include one or more of: i) an affinity agent and a column, such as magnetic activated cell sorting (MACS) with specific antibodies and microbeads; ii) fluorescent activated cell sorting (FACS), with cell populations sorted based on staining profiles with one or more fluorescently-conjugated antibodies; and iii) physical adsorption, for example, physical adsorption of T cells to protein ligands such as lectins. One or more of these selection methods and the associated materials (e.g., antibodies such as CD 34+ antibodies attached to a magnetic microbead) may be incorporated into various embodiments of the kits described here for isolating and processing selected cell population used in embodiment of the therapeutic compositions described herein.

Many embodiments of the disclosure contemplate the use of an antibody or other affinity reagent as a tool or means for generating the cell populations described herein by separating selected cells from cells in blood or blood product (e.g., that resulting from the processing of blood by apheresis or other method know in the hematologic arts) based on specificity and selectivity of the antibody for a particular cell type. Typically, HSPC's will typically be bound by CD34+ antibodies and the Tregs bound by CD25+ antibodies (both described herein) with antibodies to other cell markers also contemplated. In many embodiments, the antibody/affinity reagent for the first population of cells may correspond to an anti-CD34+ antibody/affinity reagent and the antibody/affinity reagent for the second population of cells may correspond to an anti-CD25⁺ antibody/affinity reagent. In additional embodiments, the antibody/affinity reagent for the second population may also correspond to an anti-CD117+ antibody/affinity reagent. The use of other antibodies or antigen binding agents to other antigens (e.g., receptors) unique to the desired cell type in each population is also contemplated.

In many embodiments, the antibody or other affinity reagents used to isolate cells in the first, second or other cell population is conjugated to a particle which allows for the separation of targeted cells (e.g., HSPC's, Tregs) from the blood and/or blood product and into the therapeutic composition. In various embodiments, the particle conjugated to the selected antibody or other affinity reagents (e.g., an anti CD34+ or CD25+ antibody) may be a nanoparticle and may correspond to one or more of a magnetic particle (i.e., capable of being attracted by a magnetic field) and/or a fluorophore. The magnetic particle may comprise one or more of iron, nickel or cobalt and combinations thereof. In particular embodiments, the particle may correspond to a nanoparticle including iron containing nanoparticles which may have a bead or other shape such as oval, rectangular. For ease of discussion, the particle will now be referred to as a bead. Typically, there is one bead or other particle attached to each antibody or other affinity reagent used for cell. As such, the number of the bead attached to each cell type (e.g., HSPC, Treg, etc.) may correspond in a one-to-one fashion to the number of antibodies attached to each cell type as described above and elsewhere herein. In alternative or additional embodiments, two, three, four or other number of beads may attached to each antibody or affinity reagent such that the number of beads attached to each cell may correspond to a multiple of the number of antibodies to each cell. Related to the determination to the number of beads or other particles to each cell, determinations can be made on the weight of the beads or other particles attached to each cell based on average bead weight which may be in the range of 10⁻⁷ to 10⁻¹⁰ gram range. Similar determinations can be made for the weight of the total number of antibodies attached to each cell, which may be in the range of 125 to 175 kilodaltons (kd) with a specific weight of about 150 kd.

In various embodiments, HSPC's can be obtained by harvesting cells from bone marrow or from peripheral blood, and in particular, peripheral blood of a donor who has received stem cell mobilizing agents as explained herein. Bone marrow can be aspirated from the posterior iliac crest or the anterior iliac crest while the donor is under either local or general anesthesia. HSPC's can be obtained by harvesting from peripheral blood, for example, by peripheral blood apheresis. The number of stem cells harvested can be increased by treating the donor with a mobilization agent, i.e., an agent that mobilizes stem cells from the bone marrow into peripheral blood. Non-limiting examples of mobilization agents include granulocyte colony-stimulating factor (G-CSF), granulocyte macrophage colony-stimulating factor (GM-CSF), mozobil, and combinations thereof. Techniques to mobilize stem cells into peripheral blood can comprise administering to a donor a mobilizing agent in a selected dose range, for example, 10 to 40 μ/kg/day of a mobilization agent. A mobilization agent can be administered to the donor in, for example, 1, 2, 3, 4, 5, 6, 7, 8, 9, or 10 doses. An apheresis product can be isolated from a donor about, for example, 1, 2, 3, 4, 5, 6, 7, 8, 9, 10, 11, 12, 13, 14, 15, 16, 17, 18, 19, 20, 21, 22, 23, 24, 26, 28, or 30 hour(s) after a dose of mobilization agent.

In many embodiments, the cell component of the therapeutic compositions described herein can comprise first and second populations of CD45+ cells. In many embodiments the first population of CD45+ cells comprise HSPC's. Such a population of HSPC's can be selected based on expression of a CD34+ marker present on or within the cell. In particular embodiments, CD34+ markers can be used to select HSPC's by isolating them from a mixture of nucleated cells from a blood donor. The mixture of nucleated cells may comprise specific amounts (e.g., percentages) of specific cells or cell types, for example at least about 70% CD34+ cells, less than about 5% CD3+ cells, and less than about 20% granulocytes. Lower and higher amounts of the enumerated cell types are also contemplated, including for example, less than about 10% granulocytes. Various immuno-separation methods known in the art can be used to isolate and generate a population of HSPC's or like cells using CD34+ markers. For example, a population of HSPC's can be isolated or otherwise selected using anti-CD34+ antibodies as part of a magnetic activated cell sorting (MACS) or fluorescent activated cell sorting (FACS) system. One or both of these methods may be used by embodiments for selecting and producing therapeutic compositions comprising CD34+ cells.

The number of HSPC's in a cell component or a population of cells can be determined by various methods know in the art, including for example, by quantifying CD34+ cells via flow cytometry. In some embodiments, dose calculations may be adjusted based on measures of cell viability measurements, e.g., viability determined via flow cytometry with propidium iodide or 7-AAD, or via trypan blue exclusion.

In one or more embodiments a second cell or other population of CD45+ cells one or more therapeutic compositions described herein may comprise a population of regulatory T-cells or Tregs. Desirably, at least at least 50 percent of the CD45+ cells in the second population are Treg cells with higher and lower percentages contemplated as well. According to various embodiments, a population of Tregs can be selected based on expression of one or more cell markers including CD3+, CD4+, CD25+, CD127+, FOXP3, and combinations thereof. In preferred embodiments selection is based on CD25+ and CD127+ markers. Embodiment of kits for generating a population Tregs may employ antibodies or other antigen binding or affinity reagents to one or more of these markers as a tool or means for selecting and/or refining Treg cells from an apheresis blood product or other blood product.

In various embodiments, cell selection methods which may be used by embodiments of the disclosure (including embodiments of kits described herein for generating populations of Treg cells may include one or more of: i) magnetic activated cell sorting (MACS) ii) using fluorescent activated cell sorting (FACS). In some embodiments a population of Tregs can be selected using multiple procedures, for example, multiple MACS selections, multiple FACS selections, or a combination of MACS and FACS selections. According to one embodiment, a first selection may be performed for expression of CD25, isolating CD25+ cells from a hematopoietic cell sample, for example with MACS. Then, a second selection may be performed by contacting the CD25+ cells with antibodies specific for CD4 and for CD127, where FACS is used to isolate cells that are CD4+CD127dim. Such approaches may be taken in one or more embodiments of the kits described herein for generating desired populations of Treg cells used in one or more embodiment of therapeutic compositions described herein.

According to various embodiments, a population of Tregs can be isolated from whole blood including for example peripheral blood apheresis product obtained from one or more donors. A population of Tregs can be isolated from a population of cells previously enriched and/or depleted for one or more other cell types, e.g., isolated from a population of cells depleted of CD34+ cells. In some embodiments, Tregs can be isolated from the flow-through fraction of a CD34+ MACS selection. Such approaches may be taken in one or more embodiments of the kits described herein for generating desired populations of Treg cells used in one or more embodiment of therapeutic compositions described herein.

The number of Tregs in a population of cells can be determined, for example, by flow cytometry, where Tregs can be identified as, for example, CD4+CD25+CD127dim or CD4+FOXP3+. Dose calculations can be adjusted based on measures of cell viability measurements, e.g., viability determined via flow cytometry with propidium iodide or 7-AAD, or via trypan blue exclusion.

In some embodiments, a donor cell sample may be enriched for Tregs. The Tregs may be enriched using an affinity reagent, such as an anti-human CD25 affinity reagent. In some cases, the CD25 affinity reagent may be any affinity reagent described herein such as an antibody. The amount of affinity reagent used to enrich the Treg may be less than the recommended amount as provided by the manufacturer. In some cases, the amount of affinity reagent used to enrich a Treg cell may occupy the CD25 polypeptides expressed on the surface of the Tregs in the donor cell sample. In some embodiments, an affinity reagent, such as an anti-human CD25 antibody may occupy (or be bound to) the CD25 polypeptides expressed on the surface of the Tregs. In some cases, if an affinity reagent is bound to the CD25 polypeptides expressed on the surface of the Treg, it may affect signaling of Tregs. In some cases, if Tregs have affinity reagents bound to the CD25 polypeptides expressed on the surface of the Tregs, IL2 signaling in the Tregs may be reduced. In some cases, if Tregs have affinity reagents bound to the CD25 polypeptides expressed on the surface of the Tregs, IL2 signaling and therefore phosphorylated STAT5 (pSTAT5) activation in the Tregs may be reduced. In some cases, the Treg enrichment uses an anti-human CD25 affinity reagent which may block IL2 signaling in Tregs. In some cases, the Treg enrichment uses an anti-human CD25 affinity reagent which does not block or does not reduce IL2 signaling in Tregs. In some cases, the Treg enrichment uses an anti-human CD25 affinity reagent which may block pSTAT5 activation in Tregs. In some cases, the Treg enrichment uses an anti-human CD25 affinity reagent which does not block or does not substantially reduce pSTAT5 activation in Tregs.

In some cases, the amount of an anti-human CD25 affinity reagent used to enrich Tregs may be such that the CD25 polypeptides expressed on the surface of the Tregs occupied by the affinity reagent may be 30% to 90%. In some cases, the amount of an anti-human CD25 affinity reagent used to enrich Tregs may be such that the CD25 polypeptides expressed on the surface of the Tregs occupied by the affinity reagent may be at least 30%. In some cases, the amount of an anti-human CD25 affinity reagent used to enrich Tregs may be such that the CD25 polypeptides expressed on the surface of the Tregs occupied by the affinity reagent may be at most 90%. In some cases, the amount of an anti-human CD25 affinity reagent used to enrich Tregs may be such that the CD25 polypeptides expressed on the surface of the Tregs occupied by the affinity reagent may be 30% to 35%, 30% to 40%, 30% to 50%, 30% to 60%, 30% to 65%, 30% to 70%, 30% to 75%, 30% to 80%, 30% to 85%, 30% to 90%, 35% to 40%, 35% to 50%, 35% to 60%, 35% to 65%, 35% to 70%, 35% to 75%, 35% to 80%, 35% to 85%, 35% to 90%, 40% to 50%, 40% to 60%, 40% to 65%, 40% to 70%, 40% to 75%, 40% to 80%, 40% to 85%, 40% to 90%, 50% to 60%, 50% to 65%, 50% to 70%, 50% to 75%, 50% to 80%, 50% to 85%, 50% to 90%, 60% to 65%, 60% to 70%, 60% to 75%, 60% to 80%, 60% to 85%, 60% to 90%, 65% to 70%, 65% to 75%, 65% to 80%, 65% to 85%, 65% to 90%, 70% to 75%, 70% to 80%, 70% to 85%, 70% to 90%, 75% to 80%, 75% to 85%, 75% to 90%, 80% to 85%, 80% to 90%, or 85% to 90%. In some cases, the amount of an anti-human CD25 affinity reagent used to enrich Tregs may be such that the CD25 polypeptides expressed on the surface of the Tregs occupied by the affinity reagent may be about 30%, 35%, 40%, 50%, 60%, 65%, 70%, 75%, 80%, 85%, or 90%. In some cases, the amount of an anti-human CD25 affinity reagent used to enrich Tregs may be such that the CD25 polypeptides expressed on the surface of the Tregs occupied by the affinity reagent may be at least 30%, 35%, 40%, 50%, 60%, 65%, 70%, 75%, 80%, or 85%. In some cases, the amount of an anti-human CD25 affinity reagent used to enrich Tregs may be such that the CD25 polypeptides expressed on the surface of the Tregs occupied by the affinity reagent may be at most 35%, 40%, 50%, 60%, 65%, 70%, 75%, 80%, 85%, or 90%.

In some cases, occupancy of the CD25 polypeptides expressed on the surface of the Tregs may be measured using an in vitro assay. An exemplary assay may comprise contacting Tregs with an affinity reagent may comprise: contacting a donor cell sample may comprise Tregs with an amount of an anti-human CD25 affinity reagent such that less than 85% of the CD25 polypeptides expressed on the surface of the Tregs in the donor cell sample are occupied (or bound to) by the affinity reagent according to an assay. The assay may comprise: (1) measuring the amount of background fluorescence from a first sample of a cell population, wherein the background fluorescence is emitted at a wavelength corresponding to a first fluorophore and wherein a Mean Fluorescence Intensity Zero (MFI-0) value is assigned the measured amount of background fluorescence; (2) contacting a second sample of a cell population with an anti-CD25 antibody which is conjugated to the first fluorophore, wherein the contacting occurs at a concentration, temperature, and time sufficient to achieve at least 90% saturation; (3) measuring the amount of fluorescence emitted from the cells of step (2), wherein a Mean Fluorescence Intensity Saturation (MFI-sat) value is assigned the measured amount of fluorescence; (4) contacting simultaneously a third sample of a cell population with the anti-CD25 antibody conjugated to the first fluorophore and an affinity reagent which lacks a fluorophore and under the contacting conditions used in step (2); (5) measuring the amount of fluorescence emitted from the cells of step (4), wherein a Mean Fluorescence Intensity X (MFI-x) value is assigned the measured amount of fluorescence; and (6) quantifying the percentage of receptor occupancy according to the following equation: % Receptor Occupancy=(MFI-x−MFI-0)/(MFI-sat−MFI-0)×100%.

In some embodiments, the Tregs are enriched from or isolated from the donor cell sample using the anti CD25 affinity reagents while still retaining IL2 stimulation of pSTAT5 activity. In some cases, Tregs isolated using an anti-human CD25 affinity reagent may exhibit 5% to 100% of the pSTAT5 activity in an in vitro assay as compared to Tregs that do not have CD25 polypeptides occupied by the affinity reagent. In some cases, Tregs isolated using an anti-human CD25 affinity reagent may exhibit at least 5% of the pSTAT5 activity in an in vitro assay as compared to Tregs that do not have CD25 polypeptides occupied by the affinity reagent. In some cases, Tregs isolated using an anti-human CD25 affinity reagent may exhibit at most 95% of the pSTAT5 activity in an in vitro assay as compared to Tregs that do not have CD25 polypeptides occupied by the affinity reagent. In some cases, Tregs isolated using an anti-human CD25 affinity reagent may exhibit 5% to 10%, 5% to 20%, 5% to 30%, 5% to 40%, 5% to 50%, 5% to 60%, 5% to 70%, 5% to 80%, 5% to 90%, 5% to 100%, 10% to 20%, 10% to 30%, 10% to 40%, 10% to 50%, 10% to 60%, 10% to 70%, 10% to 80%, 10% to 90%, 10% to 100%, 20% to 30%, 20% to 40%, 20% to 50%, 20% to 60%, 20% to 70%, 20% to 80%, 20% to 90%, 20% to 100%, 30% to 40%, 30% to 50%, 30% to 60%, 30% to 70%, 30% to 80%, 30% to 90%, 30% to 100%, 40% to 50%, 40% to 60%, 40% to 70%, 40% to 80%, 40% to 90%, 40% to 100%, 50% to 60%, 50% to 70%, 50% to 80%, 50% to 90%, 50% to 100%, 60% to 70%, 60% to 80%, 60% to 90%, 60% to 100%, 70% to 80%, 70% to 90%, 70% to 100%, 80% to 90%, 80% to 100%, or 90% to 100% of the pSTAT5 activity in an in vitro assay as compared to Tregs that do not have CD25 polypeptides occupied by the affinity reagent. In some cases, Tregs isolated using an anti-human CD25 affinity reagent may exhibit about 5%, 10%, 20%, 30%, 40%, 50%, 60%, 70%, 80%, 90%, or 100% of the pSTAT5 activity in an in vitro assay as compared to Tregs that do not have CD25 polypeptides occupied by the affinity reagent. In some cases, Tregs isolated using an anti-human CD25 affinity reagent may exhibit at least 5%, 10%, 20%, 30%, 40%, 50%, 60%, 70%, 80%, 90%, or 95% of the pSTAT5 activity in an in vitro assay as compared to Tregs that do not have CD25 polypeptides occupied by the affinity reagent. In some cases, Tregs isolated using an anti-human CD25 affinity reagent may exhibit at most 10%, 20%, 30%, 40%, 50%, 60%, 70%, 80%, 90%, or 95% of the pSTAT5 activity in an in vitro assay as compared to Tregs that do not have CD25 polypeptides occupied by the affinity reagent.

In some cases, pSTAT5 activity may be measured in a Treg cell with CD25 polypeptides occupied by an affinity reagent. In some cases, pSTAT5 activity may be measured in a Treg cell with CD25 polypeptides occupied using intracellular cytokine staining. An illustrative assay for such a detection may comprise: (1) obtaining a cell having been exposed to an affinity reagent; (2) contacting the cell with IL-2 at a concentration, temperature and time sufficient for the IL-2 to interact with its receptor on the cell and activate IL-2 signaling by the cell; (3) contacting the cell with each of: a first antibody conjugated with a first fluorophore and directed against CD3, a second antibody conjugated with a second fluorophore and directed against CD4, a third antibody conjugated with a third fluorophore and directed against CD25, a fourth antibody conjugated with a fourth fluorophore and directed against CD127, and a fifth antibody conjugated with a fifth fluorophore and directed against pSTAT5. In some cases, each of the first, second, third, fourth, and fifth fluorophore are different fluorophores; (4) subjecting the cell of step (3) to flow cytometry which may be gated for CD3+CD4+CD25+CD127− and collecting the CD3+CD4+CD25+CD127− cells; (5) detecting the amount of fluorescence from the fifth fluorophores and detecting the amount of florescence from the first, the second, and/or the third fluorophores in the collected cells of step (5); and (6) calculating the percentage of the collected cells which have fluorescence from the first, the second, and/or the third fluorophores and which have fluorescence from the fifth fluorophore, wherein this percentage represents the fraction of cells having receptors occupied by the affinity reagent.

In some embodiments, the amount of affinity reagents used to isolate the Tregs may be used in an amount wherein the Tregs exhibit pSTAT5 activity while maintaining a purity and yield of Tregs. In some cases, the Tregs isolated from a donor cell sample, such as a peripheral blood sample or a mobilized apheresis blood sample may have a certain amount of purity while exhibiting pSTAT5 activity and IL2 binding as described above. In some cases, purity of a population of Tregs isolated from a donor may be 20% to 95%. In some cases, purity of a population of Tregs isolated from a donor may be at least 20%. In some cases, purity of a population of Tregs isolated from a donor may be at most 95%. In some cases, purity of a population of Tregs isolated from a donor may be 20% to 30%, 20% to 40%, 20% to 50%, 20% to 60%, 20% to 70%, 20% to 80%, 20% to 90%, 20% to 95%, 30% to 40%, 30% to 50%, 30% to 60%, 30% to 70%, 30% to 80%, 30% to 90%, 30% to 95%, 40% to 50%, 40% to 60%, 40% to 70%, 40% to 80%, 40% to 90%, 40% to 95%, 50% to 60%, 50% to 70%, 50% to 80%, 50% to 90%, 50% to 95%, 60% to 70%, 60% to 80%, 60% to 90%, 60% to 95%, 70% to 80%, 70% to 90%, 70% to 95%, 80% to 90%, 80% to 95%, or 90% to 95%. In some cases, purity of a population of Tregs isolated from a donor may be about 20%, 30%, 40%, 50%, 60%, 70%, 80%, 90%, or 95%. In some cases, purity of a population of Tregs isolated from a donor may be at least 20%, 30%, 40%, 50%, 60%, 70%, 80%, 90%, or 95%. In some cases, purity of a population of Tregs isolated from a donor may be at most 20%, 30%, 40%, 50%, 60%, 70%, 80%, 90%, or 95%.

In some embodiments, a cell component can comprise a population of Tcons. In these and related embodiments, a population of Tcons can be sourced from peripheral blood. A population of Tcons can be sourced from a peripheral blood apheresis product.

In some embodiments, no selection steps are carried out, and a population of Tcons is sourced directly from an aliquot of peripheral blood or apheresis product. In some embodiments, a population of cells can be enriched for Tcons, for example, by sorting based on the expression of various markers using MACS, FACS, or a combination thereof. A population of Tcons can be enriched by sorting for CD3+ cells. A population of Tcons can be enriched by sorting for CD4+ and CD8+ cells. A population of Tcons can be enriched by negative selection, where non-Tcon cells are removed, for example, by MACS depletion of cells expressing CD34, CD19, CD25, or a combination thereof.

The number of Tcons present in a population can be quantified, for example, by quantifying CD3+ cells via flow cytometry. The number of CD3+ cells in an aliquot can be determined and a volume comprising an appropriate dose of CD3 cells administered to the recipient. Dose calculations can be adjusted based on measures of cell viability, e.g., viability determined via flow cytometry with propidium iodide or 7-AAD, or via trypan blue exclusion.

An apheresis product of the disclosure can be split into two portions, one portion used to provide Tcons cells and the other portion to isolate and purify HSPC's and Tregs. In alternate embodiments, CD34+ cells are isolated and purified from the apheresis product, creating a CD34-negative cell fraction from which the Treg are then isolated.

A cell component of the disclosure can comprise a population of iNKTs. A population of iNKTs can be sourced from peripheral blood. A population of iNKTs can be sourced from a peripheral blood apheresis product.

In various embodiments, a population of cells can be enriched for iNKTs, for example, by sorting based on the expression of various markers using MACS, FACS, or a combination thereof. A population of iNKTs can be enriched, for example, by sorting for CD3+Vα24Jα18+ cells.

The number of iNKTs present in a population can be quantified, for example, by quantifying CD3+Vα24Jα18+ cells via flow cytometry. The number of CD3+Vα24Jα18+ cells in an aliquot can be determined and a volume comprising an appropriate dose of iNKTs administered to the recipient. In some embodiments, dose calculations are adjusted based on measures of cell viability measurements, e.g., viability determined via flow cytometry with propidium iodide or 7-AAD, or via trypan blue exclusion.

A cell component of the disclosure can comprise a population of Tmems. A population of Tmems can be sourced from peripheral blood. A population of Tmems can be sourced from a peripheral blood apheresis product.

A population of cells can be enriched for Tmems, for example, by sorting based on the expression of various markers using MACS, FACS, or a combination thereof. A population of Tmems can be enriched, for example, by sorting for CD3+CD45RA−CD45RO+ cells.

The number of Tmems present in a population can be quantified, for example, by quantifying CD3+CD45RA−CD45RO+ cells via flow cytometry. The number of CD3+CD45RA−CD45RO+ cells in an aliquot can be determined and a volume comprising an appropriate dose of Tmems administered to the recipient. Dose calculations can be adjusted based on measures of cell viability measurements, e.g., viability determined via flow cytometry with propidium iodide or 7-AAD, or via trypan blue exclusion.

In various embodiments, a cell population or a cell component of the disclosure can be administered freshly after isolation, or after cryopreservation and subsequent thawing. Cells freshly isolated from a donor (“fresh cells”) can be administered to a recipient subject. Fresh cells can be stored in a buffer, for example, CliniMACS PBS-EDTA Buffer with 0.5% human serum albumin, or Plasma-Lyte-A, pH 7.4 supplemented with 2% human serum albumin. Fresh cells can be stored at a reduced temperature (e.g., 2-8° C.), and without being cryopreserved/frozen.

After acquiring a fresh population of cells from a donor, the fresh cells can be stored for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 37 hours, at least about 38 hours, at least about 39 hours, at least about 40 hours, at least about 44 hours, at least about 48 hours, at least about 50 hours, at least about 55 hours, at least about 60 hours, at least about 61 hours, at least about 62 hours, at least about 65 hours, at least about 70 hours, at least about 72 hours, at least about 80 hours, at least about 90 hours, at least about 96 hours, at least about 120 hours, at least about 150 hours, at least about 200 hours, at least about 300 hours, or more prior to administration to a subject.

After acquiring a fresh population of cells from a donor, the fresh cells can be stored for at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 12 hours, at most about 14 hours, at most about 16 hours, at most about 18 hours, at most about 20, at most 22 hours, at most about 24 hours, at most about 30 hours, at most about 36 hours, at most about 40 hours, at most about 48 hours, at most about 60 hours, at most about 70 hours, at most about 72 hours, at most about 80 hours, at most about 90 hours, at most about 96 hours, at most about 120 hours, at most about 150 hours, at most about 200 hours, or at most about 300 hours prior to administration to a subject.

Cells of the disclosure can be cryopreserved. In some embodiments, cryopreservation can be beneficial to the methods disclosed herein. For example, cryopreservation of Tcons prior to subsequent thawing and administering to a subject may reduce GVHD.

Cryopreservation can comprise addition of a preservative agent (e.g., DMSO), and gradual cooling of cells in a controlled-rate freezer to prevent or reduce osmotic cellular injury resulting from ice crystal formation. Cryopreservation can comprise commercial cryopreservation reagents and materials, for example, Cryobags and CryoStor® CS10.

Cryopreserved cells can be stored for periods of time ranging from hours to years at low temperatures. Cryopreserved cells can be stored at ultralow temperatures, for example, −50° C., −60° C., −70° C., −80° C., −90° C., −100° C., −110° C., −120° C., −130° C., −140° C., −150° C., −160° C., −170° C., −180° C., −190° C., −196° C., or less. Cryopreserved cells can be stored in storage devices comprising liquid nitrogen.

Cells can be cryopreserved before or after certain steps in the methods of the disclosure, for example, before or after sorting steps, before or after characterization steps, such as determining cell viability or the concentration of cells of a particular type.

In some embodiments, whole blood can be cryopreserved. Whole blood can be cryopreserved without sorting or characterization. Whole blood can be cryopreserved after sorting but without characterization. Whole blood can be cryopreserved after characterization but without sorting. Whole blood can be cryopreserved after characterization and sorting. Whole blood can be cryopreserved after quantifying a cell type of the disclosure Whole blood can be cryopreserved after quantifying conventional T cells (Tcons, e.g., CD3+ cells). Whole blood can be cryopreserved after quantifying viability of all cells or a population of cells of the disclosure (e.g., conventional T cells).

According to various embodiments, a peripheral blood apheresis product of the disclosure may be cryopreserved. A peripheral blood apheresis product can be cryopreserved without sorting or characterization. A peripheral blood apheresis product can be cryopreserved after sorting but without characterization. A peripheral blood apheresis product can be cryopreserved after characterization but without sorting. A peripheral blood apheresis product can be cryopreserved after characterization and sorting. A peripheral blood apheresis product can be cryopreserved after quantifying a cell type of the disclosure. A peripheral blood apheresis product can be cryopreserved after quantifying conventional T cells (Tcons, e.g., CD3+ cells). A peripheral blood apheresis product can be cryopreserved after quantifying viability of all cells or a population of cells of the disclosure (e.g., conventional T cells).

A population of cells sorted or selected from another population of cells can be cryopreserved, for example, a population of HSPC's, Tregs, Tcons, iNKTs, or Tmems can be cryopreserved.

A cell component of the disclosure can be cryopreserved for any amount of time. Cells of the disclosure may be cryopreserved for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 at least about 14 hours, at least about 16 hours, at least about 18 hours, at least about 20 hours, at least about 22 hours, at least about 24 hours, at least about 30 hours, at least about 36 hours at least about 48 hours, at least about 50 hours, at least about 55 hours, at least about 60 hours, at least about 61 hours, at least about 62 hours, at least about 65 hours, at least about 70 hours, at least about 72 hours, at least about 80 hours, at least about 90 hours, at least about 96 hours, at least about 120 hours, at least about 150 hours, at least about 200 hours, at least about 300 hours, or more prior to thawing and administration to a subject.

In some embodiments, a cell component of the disclosure is cryopreserved for at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 11 hours, at most about 12 at most about 14 hours, at most about 16 hours, at most about 18 hours, at most about 20 hours, at most about 22 hours, at most about 24 hours, at most about 30 hours, at most about 36 hours at most about 48 hours, at most about 50 hours, at most about 55 hours, at most about 60 hours, at most about 61 hours, at most about 62 hours, at most about 65 hours, at most about 70 hours, at most about 72 hours, at most about 80 hours, at most about 90 hours, at most about 96 hours, at most about 120 hours, at most about 150 hours, at most about 200 hours, or at most about 300 hours prior to thawing and administration to a subject.

In some embodiments, a cell component of the disclosure is cryopreserved for at least about 1 day, at least about 2 days, at least about 3 days, at least about 4 days, at least about 5 days, at least about 6 days, at least about 7 days, at least about 10 days, at least about 14 days, at least about 21 days, at least about 28 days, at least about 50 days, at least about 60 days, or at least about 96 days, or more prior to thawing and administration to a subject.

In some embodiments, a cell component of the disclosure is cryopreserved for at most about 1 day, at most about 2 days, at most about 3 days, at most about 4 days, at most about 5 days, at most about 6 days, at most about 7 days, at most about 10 days, at most about 14 days, at most about 21 days, at most about 28 days, at most about 50 days, at most about 60 days, or at most about 96 days prior to thawing and administration to a subject.

### B. Donors

A cell component can comprise cells that are from one or more donors that have each been Human leukocyte antigens (HLA)typed, for example, to determine a degree of HLA matching to a subject that will receive the cell component.

HLA, also broadly referred to as Major histocompatibility complex (MHC) antigens, can be protein molecules expressed on the surface of a cell that can confer an antigenic identity to that cell. HLA/MHC antigens are target molecules that can be recognized by T cells and natural killer (NK) cells as being derived from the same source of hematopoietic stem cells as the immune effector cells (“self”), or as being derived from another source of hematopoietic cells (“non-self”). HLA class I antigens (A, B, and C in humans) can be expressed by the vast majority of cells, while HLA class II antigens (DR, DP, and DQ in humans) can be expressed primarily on professional antigen presenting cells. Both HLA classes can be implicated in GVHD.

HLA antigens are encoded by highly polymorphic genes; a range of alleles exists for each HLA class I and II gene. Allelic gene products can differ in one or more amino acids in the a and/or f domain(s). Panels of specific antibodies or nucleic acid reagents can be used to determine HLA haplotypes of individuals, for example, using leukocytes that express class I and class II molecules. HLA alleles can be described at various levels of detail. Most designations begin with HLA- and the locus name, then * and some (even) number of digits specifying the allele. The first two digits can specify a group of alleles. The third through fourth digits, when present, can specify a synonymous allele. Digits five through six, when present, can denote any synonymous mutations within the coding frame of the gene. The seventh and eighth digits, when present, can distinguish mutations outside the coding region. Letters such as L, N, Q, or S may follow an allele's designation to specify an expression level or other non-genomic data known about it. Thus, a completely described allele may be up to 9 digits long, not including the HLA-prefix and locus notation.

The set of HLA alleles inherited from one parent forms a haplotype. HLA haploidentical can refer to a donor-recipient pair where one chromosome is matched at least at HLA-A; HLA-B, and HLA-DR between the donor and recipient. The haploidentical pair may or may not be matched at other alleles, e.g., other HLA genes on the other chromosome, or additional histocompatibility loci on either chromosome. Such donors can frequently occur in families, e.g. a parent can be haploidentical to a child; and siblings may be haploidentical.

A cell component can be from a donor that has been HLA-typed at any number of HLA alleles. A donor and a subject can be HLA matched, e.g., matched at all typed HLA alleles. A donor and a subject can be HLA mismatched, e.g., at least one HLA antigen can be mismatched between the donor and recipient.

In some embodiments, a donor and a subject can be HLA-typed at six alleles consisting of HLA-A, HLA-B, and HLA-DR alleles. The donor and subject can be matched at, for example 3/6 4/6, 5/6, or 6/6 of the alleles. In some embodiments, the donor and subject are matched at least at 5/6 alleles. In some embodiments, the donor and subject are matched at 6/6 alleles.

In some embodiments, a donor and a subject can be HLA-typed at eight alleles consisting of HLA-A, HLA-B, HLA-C, and HLA-DR alleles (e.g., HLA-DRB1 alleles). The donor and subject can be matched at, for example 4/8, 5/8, 6/8, 7/8, or 8/8 of the alleles. In some embodiments, the donor and subject are matched at least at 6/8 alleles. In some embodiments, the donor and subject are matched at least at 7/8 alleles. In some embodiments, the donor and subject are matched at 8/8 alleles.

In some embodiments, a donor and a subject can be HLA-typed at ten alleles consisting of HLA-A, HLA-B, HLA-C, and HLA-DR alleles (e.g., HLA-DRB1 alleles). The donor and subject can be matched at, for example 5/10, 6/10, 7/10, 8/10, 9/10, or 10/10 of the alleles. In some embodiments, the donor and subject are matched at least at 7/10 alleles. In some embodiments, the donor and subject are matched at least at 8/10 alleles. In some embodiments, the donor and subject are matched at least at 9/10 alleles. In some embodiments, the donor and subject are matched at 10/10 alleles.

A cell component can be generated from a matched sibling donor that is an 8/8 match for HLA-A, -B, -C, and -DRB1, all typed using DNA-based high-resolution methods. A cell component can be generated from a matched sibling donor that is an 7/8 match for HLA-A, -B, -C, and -DRB1, all typed using DNA-based high-resolution methods. A cell component can be generated from a matched sibling donor that is an 6/8 match for HLA-A, -B, -C, and -DRB1, all typed using DNA-based high-resolution methods. A cell component can be generated from a matched sibling donor that is an 10/10 match for HLA-A, -B, -C, and -DRB1, all typed using DNA-based high-resolution methods. A cell component can be generated from a matched sibling donor that is an 9/10 match for HLA-A, -B, -C, and -DRB1, all typed using DNA-based high-resolution methods. A cell component can be generated from a matched sibling donor that is an 8/10 match for HLA-A, -B, -C, and -DRB1, all typed using DNA-based high-resolution methods. A cell component can be generated from a matched sibling donor that is an 7/10 match for HLA-A, -B, -C, and -DRB1, all typed using DNA-based high-resolution methods.

A cell component can be generated from a matched unrelated donor that is a 8/8 match at HLA-A, -B, -C, and -DRB1, all typed using DNA-based high-resolution methods. A cell component can be generated from a matched unrelated donor that is a 7/8 match at HLA-A, -B, -C, and -DRB1, all typed using DNA-based high-resolution methods. A cell component can be generated from a matched unrelated donor that is a 6/8 match at HLA-A, -B, -C, and -DRB1, all typed using DNA-based high-resolution methods. A cell component can be generated from a matched unrelated donor that is a 10/10 match at HLA-A, -B, -C, and -DRB1, all typed using DNA-based high-resolution methods. A cell component can be generated from a matched unrelated donor that is a 9/10 match at HLA-A, -B, -C, and -DRB1, all typed using DNA-based high-resolution methods. A cell component can be generated from a matched unrelated donor that is a 8/10 match at HLA-A, -B, -C, and -DRB1, all typed using DNA-based high-resolution methods

A cell component can be derived from an allogeneic donor, i.e., a cell component taken from a different individual of the same species. An allogenic donor may be genetically related to the subject or unrelated to the subject. A cell component can be generated from a donor that is a first-degree blood relative of the subject. A cell component can be generated from a donor that is a second-degree blood relative of the subject. A cell component can be generated from a donor that is not related to the subject. A cell component can be generated from a donor that is HLA matched to a recipient subject. A cell component can be generated from a donor that is HLA mismatched to a recipient subject. A cell component can be generated from a donor that is haploidentical to a recipient subject. A cell component can be generated from a donor that is related to a recipient subject, for example, a parent, child, sibling, grandparent, grandchild, aunt, uncle, or cousin. A cell component can be generated from a donor that is at least 16 years old. A cell component can be generated from a donor that is at least 18 years old.

A cell component can be generated from a donor that meets eligibility criteria for donors of viable, leukocyte-rich cells or tissues as defined by relevant FDA Guidance for Industry. For example, a cell component can be generated from a donor that meets eligibility criteria outlined in any one or more of the following: Eligibility Determination for Donors of Human Cells, Tissues, and Cellular and Tissue-Based Products, 2007; Use of Donor Screening Tests to Test Donors of Human Cells, Tissues and Cellular and Tissue-Based Products for Infection with Treponema pallidum (Syphilis), 2015; Use of Nucleic Acid Tests to Reduce the Risk of Transmission of Hepatitis B Virus from Donors of Human Cells, Tissues, and Cellular and Tissue-Based Products, 2016; Use of Nucleic Acid Tests to Reduce the Risk of Transmission of West Nile Virus from Living Donors of Human Cells, Tissues, and Cellular and Tissue-Based Products (HCT/Ps), 2016; and Donor Screening Recommendations to Reduce the Risk of Transmission of Zika Virus by Human Cells, Tissues, and Cellular and Tissue-Based Products, 2018). The contents of each of which is incorporated herein by reference in its entirety. A cell component can be generated from a donor that meets any criteria for donation as specified by standard NMDP guidelines (NMDP donors).

A cell component can be generated from a donor that does not exhibit evidence of active infection. A cell component can be generated from a donor that is not seropositive for HIV-1 or -2, HTLV-1 or -2. A cell component can be generated from a donor that is not positive for anti-hepatitis C (HCV) antibody or HCV NAT. A cell component can be generated from a donor that tests negative for chronic HBV infection. A cell component can be generated from a donor that does not have high potential for Zika virus infection as defined as any of the following: (i) Medical diagnosis of Zika virus infection in the past 6 months; (ii) Residence in, or travel to, an area with active Zika virus transmission within the past 6 months; (iii) Unprotected sex within the past 6 months with a person who is known to have either of the risk factors (i) or (ii). A cell component can be generated from a donor that does not have signs or symptoms consistent with active Zika virus infection.

One or more cell components of the disclosure can be obtained from a single donor, for example, obtained from mobilized peripheral blood apheresis of a single donor. HSPC's, Tregs, Tcons, iNKTs, Tmems, or any combination thereof can be obtained from a single donor.

One or more cell populations of the disclosure can be obtained from one donor, and one or more additional cell populations of the disclosure can be obtained from a second donor. One cell population of the disclosure can be obtained from a single donor, and a second cell population of the disclosure can be obtained from multiple donors. Cell populations of the disclosure can be obtained from multiple donors, for example, obtained from mobilized peripheral blood apheresis of multiple donors. HSPC's can be obtained from multiple donors. Tregs can be obtained from multiple donors. Tcons can be obtained from multiple donors. iNKTs can be obtained from multiple donors. Tmems can be obtained from multiple donors.

### C. Doses of Cell Components

In various embodiments, the doses, characteristics and administration sequence of the various cell components/populations (e.g., HSPC's, Tregs, Tcons) of the therapeutic compositions described herein may be adjusted and/or otherwise selected for a given patient depending upon for example their age, weight, type and extent of their disease, any conditioning and/or immunosuppressive/GVHD agents administered prophylactically or other otherwise as well as other clinical considerations known to those skilled in the art. The dose and other characteristics of such cell components/populations of the therapeutic compositions can be adjusted using embodiment of the kits and associated therapeutic composition production methods described herein (e.g., magnetic or optical based cell separation using the antigen binding agents provided in the kit).

Doses of cell components administered to a subject may be based on the subject's body weight. In some cases, a subject's body weight may be used to determine a dose of one or more cell components to be administered to the subject. In some cases, a cell dose may be based on the ideal body weight of the subject instead of their actual weight, e.g., actual body weight. Ideal body weight may be a preferable method of dose calculation to avoid erroneous cell doses due to excess body fat and/or muscle mass. A subject's ideal body weight may be calculated using their height and sex. Other methods that calculate a subject's ideal body weight may be used. For instance, other methods which determine a subject's body fat percentage.

### HSPC's

According to one embodiment, a population of HSPC's in one more therapeutic compositions described herein may comprise more than about 1×10⁵ HSPC's per kilogram of ideal body weight of said human subject. In some embodiments, the population of HSPC's may comprise about 5×10⁵ to 2×10⁷ HSPC's per kilogram of ideal body weight the human subject.

A cell component that comprises a population of HSPC's can comprise at least about 1×10⁴, at least about 1×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.5×10⁶, at least about 7×10⁶, at least about 7.5×10⁶, at least about 8×10⁶, at least about 8.5×10⁶, at least about 9×10⁶, at least about 9.5×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1×10⁸, at least about 1.5×10⁸, at least about 2×10⁸, at least about 2.5×10⁸, at least about 3×108, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×109, or more HSPC's (e.g., CD34+ cells) per kg of recipient subject's actual body weight or ideal body weight.

A cell component that comprises a population of HSPC's can comprise at most about 1×10⁴, at most about 1×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, a most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, a most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.5×10⁶, at most about 7×10⁶, a most about 7.5×10⁶, at most about 8×10⁶, at most about 8.5×10⁶, at most about 9×10⁶, at most about 9.5×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1×10⁸, at most about 1.5×10⁸, at most about 2×10⁸, at most about 2.5×10, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×109 HSPC's (e.g., CD34+ cells) per kg of recipient subject's actual body weight or ideal body weight.

For example, a cell component that comprises a population of HSPC's can comprise 1×10⁴ to 1×10⁹, 1×10⁵ to 1×10⁸, 1×10⁵ to 2×10⁷, 5×10⁵ to 2×10⁷, 5×10⁵ to 1.5×10⁷, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ HSPC's (e.g., CD34+ cells) per kg of recipient subject's actual body weight or ideal body weight.

In various embodiments, one or more of the cell populations described herein may have selected amounts of antibodies (or other antigen binding agents) bound to at least a portion of the cells in the population, for example HSPC's, in the first population and Treg cells in the second cell populations. Typically, HSPC's will typically be bound by CD34+ antibodies and the T regs bound by CD25+ antibodies (both described herein) with antibodies to other cell markers also contemplated. The average amount of antibody (e.g., a CD25+ or CD4+ antibody) bound to at least a portion of the Tregs in the second population can range from about 500 to 10,000 per cell with specific embodiments of about 1000 to 10,000, 2500 to 10,000. Wider ranges and higher upper limits are also contemplated including for example about 1,000 to 20,000 antibodies per cell. In some embodiments, at least about 70% of the Treg cells in the second or other population may have bound antibody (or other antigen binding agent) and more preferably at least about 90% with higher amounts contemplated.

A population of HSPC's of the disclosure can have a defined level of purity for CD34+ cells. For example, a population of HSPC's of the disclosure can comprise at least about 5%, at least about at least about 10%, at least about at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or more CD34+ cells as a percentage of total cells, as a percentage of nucleated cells, or as a percentage of CD45+ cells.

A population of HSPC's of the disclosure can have a defined level of contaminating CD3+ cells. In some embodiments, at most about 1×10², at most about 2×10², at most about 3×10², at most about 4×10², at most about 5×10², at most about 6×10², at most about 7×10², at most about 8×10², at most about 9×10², at most about 1×10³, at most about 2×10³, at most about 3×10³, at most about 4×10³, at most about 5×10³, at most about 6×10³, at most about 7×10³, at most about 8×10³, at most about 9×10³, at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, or at most about 1×10⁵ CD3+ cells per kg of a recipient subject's body weight are present in a population of HSPC's of the disclosure or ideal body weight.

In some embodiments, a population of HSPC's of the disclosure comprises at most about 0.001%, at most about 0.002%, at most about 0.003%, at most about 0.004%, at most about 0.005%, at most about 0.006%, at most about 0.007%, at most about 0.008% 0.009%, at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, at most about 0.2%, at most about 0.3%, at most about 0.4%, at most about 0.5%, at most about 0.6%, at most about 0.7%, at most about 0.8%, at most about 0.9%, at most about 1%, at most about 1.1%, at most about 1.2%, at most about 1.3%, at most about 1.4%, at most about 1.5%, at most about 1.6%, at most about 1.7%, at most about 1.8%, at most about 1.9%, at most about 2%, at most about 2.1%, at most about 2.2%, at most about 2.3%, at most about 2.4%, at most about 2.5%, at most about 2.6%, at most about 2.7%, at most about 2.8%, at most about 2.9%, at most about 3%, at most about 3.1%, at most about 3.2%, at most about 3.3%, at most about 3.4%, at most about 3.5%, at most about 3.6%, at most about 3.7%, at most about 3.8%, at most about 3.9%, at most about 4%, at most about 5%, at most about 6%, at most about 7%, at most about 8%, at most about 9%, or at most about 10% CD3+ cells as a percentage of total cells, as a percentage of nucleated cells, or as a percentage of CD45+ cells.

In some embodiments, the population of HSPC's comprises less than 3 EU/ml endotoxins. In some embodiments, the population of HSPC's comprise less than 1 EU/ml endotoxins. A cell component that comprises a population of HSPC's can comprise 0.5 EU/ml endotoxins to 10 EU/ml endotoxins. A cell component that comprises a population of HSPC's can comprise at least 0.5 EU/ml endotoxins. A cell component that comprises a population of HSPC's can comprise at most 10 EU/ml endotoxins. A cell component that comprises a population of HSPC's can comprise 10 EU/ml endotoxins to 8 EU/ml endotoxins, 10 EU/ml endotoxins to 6 EU/ml endotoxins, 10 EU/ml endotoxins to 5 EU/ml endotoxins, 10 EU/ml endotoxins to 4 EU/ml endotoxins, 10 EU/ml endotoxins to 2 EU/ml endotoxins, 10 EU/ml endotoxins to 1 EU/ml endotoxins, 10 EU/ml endotoxins to 0.5 EU/ml endotoxins, 8 EU/ml endotoxins to 6 EU/ml endotoxins, 8 EU/ml endotoxins to 5 EU/ml endotoxins, 8 EU/ml endotoxins to 4 EU/ml endotoxins, 8 EU/ml endotoxins to 2 EU/ml endotoxins, 8 EU/ml endotoxins to 1 EU/ml endotoxins, 8 EU/ml endotoxins to 0.5 EU/ml endotoxins, 6 EU/ml endotoxins to 5 EU/ml endotoxins, 6 EU/ml endotoxins to 4 EU/ml endotoxins, 6 EU/ml endotoxins to 2 EU/ml endotoxins, 6 EU/ml endotoxins to 1 EU/ml endotoxins, 6 EU/ml endotoxins to 0.5 EU/ml endotoxins, 5 EU/ml endotoxins to 4 EU/ml endotoxins, 5 EU/ml endotoxins to 2 EU/ml endotoxins, 5 EU/ml endotoxins to 1 EU/ml endotoxins, 5 EU/ml endotoxins to 0.5 EU/ml endotoxins, 4 EU/ml endotoxins to 2 EU/ml endotoxins, 4 EU/ml endotoxins to 1 EU/ml endotoxins, 4 EU/ml endotoxins to 0.5 EU/ml endotoxins, 2 EU/ml endotoxins to 1 EU/ml endotoxins, 2 EU/ml endotoxins to 0.5 EU/ml endotoxins, or 1 EU/ml endotoxins to 0.5 EU/ml endotoxins. A cell component that comprises a population of HSPC's can comprise 10 EU/ml endotoxins, 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A cell component that comprises a population of HSPC's can comprise at least 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A cell component that comprises a population of HSPC's can comprise at most 10 EU/ml endotoxins, 8 EU/ml endotoxins, 6 EU/ml endotoxins, 5 EU/ml endotoxins, 4 EU/ml endotoxins, 2 EU/ml endotoxins or 1 EU/ml endotoxins.

According to one or more embodiments, a cell component that comprises a population of HSPC's can also comprise 0.5% w/w to 10% w/w unbound reagents. These unbound reagents may include any affinity reagents used for the sorting of HSPC's, for instance, antibodies, or purification particles or magnetic particles. A cell component that comprises a population of HSPC's can comprise at least 0.5% w/w unbound reagents. A cell component that comprises a population of HSPC's can comprise at most 10% w/w unbound reagents. A cell component that comprises a population of HSPC's can comprise 10% w/w to 8% w/w, 10% w/w to 6% w/w, 10% w/w to 5% w/w, 10% w/w to 4% w/w, 10% w/w to 2% w/w, 10% w/w to 1% w/w, 10% w/w to 0.5% w/w, 8% w/w to 6% w/w, 8% w/w to 5% w/w, 8% w/w to 4% w/w, 8% w/w to 2% w/w, 8% w/w to 1% w/w, 8% w/w to 0.5% w/w, 6% w/w to 5% w/w, 6% w/w to 4% w/w, 6% w/w to 2% w/w, 6% w/w to 1% w/w, 6% w/w to 0.5% w/w, 5% w/w to 4% w/w, 5% w/w to 2% w/w, 5% w/w to 1% w/w, 5% w/w to 0.5% w/w, 4% w/w to 2% w/w, 4% w/w to 1% w/w, 4% w/w to 0.5% w/w, 2% w/w to 1% w/w, 2% w/w to 0.5% w/w, or 1% w/w to 0.5% w/w unbound reagents. A cell component that comprises a population of HSPC's can comprise 10% w/w, 8% w/w, 6% w/w, 5% w/w, 4% w/w, 2% w/w, 1% w/w, or 0.5% w/w unbound reagents. A cell component that comprises a population of HSPC's can comprise at least 8% w/w, 6% w/w, 5% w/w, 4% w/w, 2% w/w, 1% w/w, or 0.5% w/w unbound reagents. A cell component that comprises a population of HSPC's can comprise at most 10% w/w, 8% w/w, 6% w/w, 5% w/w, 4% w/w, 2% w/w or 1% w/w unbound reagents.

A cell component that comprises a population of HSPC's can comprise 5×10³ to 90×10³ microbeads per cell. These microbeads may comprise microbeads used to purify the HSPC population, for instance, an anti-CD34 antibody comprising microbead used to sort the HSPC population. A cell component that comprises a population of HSPC's can comprise at least 5×10³ microbeads per cell. A cell component that comprises a population of HSPC's can comprise at most 90×10³ microbeads per cell. A cell component that comprises a population of HSPC's can comprise 90×10³ to 70×10³, 90×10³ to 50×10³, 90×10³ to 40×10³, 90×10³ to 30×10³, 90×10³ to 20×10³, 90×10³ to 10×10³, 90×10³ to 5×10³, 70×10³ to 50×10³, 70×10³ to 40×10³, 70×10³ to 30×10³, 70×10³ to 20×10³, 70×10³ to 10×10³, 70×10³ to 5×10³, 50×10³ to 40×10³, 50×10³ to 30×10³, 50×10³ to 20×10³, 50×10³ to 10×10³, 50×10³ to 5×10³, 40×10³ to 30×10³, 40×10³ to 20×10³, 40×10³ to 10×10³, 40×10³ to 5×10³, 30×10³ to 20×10³, 30×10³ to 10×10³, 30×10³ to 5×10³, 20×10³ to 10×10³, 20×10³ to 5×10³, or 10×10³ to 5×10³ microbeads per cell. A cell component that comprises a population of HSPC's can comprise 90×10³, 70×10³, 50×10³, 40×10³, 30×10³, 20×10³, 10×10³, or 5×10³ microbeads per cell. A cell component that comprises a population of HSPC's can comprise at least 70×10³, 50×10³, 40×10³, 30×10³, 20×10³, 10×10³, or 5×10³ microbeads per cell. A cell component that comprises a population of HSPC's can comprise at most 90×10³, 70×10³, 50×10³, 40×10³, 30×10³, 20×10³, or 10×10³ microbeads per cell. In some embodiments, the population of HSPC's may have less than 30,000 microbeads per cell. A microbead that is bound to a cell is bound via at least one antibody immobilized on the bead. In some cases, a microbead has one immobilized antibody. A microbead may have 100, 1,000, 10,000, 100,000, 1,000,000, 1,000,000,000 or more immobilized antibodies and any number of immobilized antibodies therebetween.

Embodiments of the disclosure provide therapeutic compositions for hematopoietic or other stem cell transplantation to a human or other patient in need thereof. According to one embodiment, a therapeutic composition for hematopoietic cell transplantation to a human patient in need thereof comprises a population of isolated hematopoietic stem and progenitor cells (HSPC) wherein at least a portion of the HSPC's have an antibody bound to a marker on the cell surface which is used to separate HSPC's from a mixture of nucleated cells from a mobilized blood donor, the antibody conjugated to a particle. Typically, the antibody is an anti-CD34⁺ antibody though others are also contemplated. In various embodiments the HSPC's can have an average number of conjugated antibodies attached to each cell in the range of about 500 to 10,000, with specific ranges of 1000, to 10,000, 2500 to 5000, 2500, to 7500, 2500 to 10,000 and 5000 to 10,000. Higher ranges are also contemplated for example 1000 to 20,000. Also in various embodiments at least about 70 percent (%) of HSPC's have bound antibody, more preferably at least about 80% and still more preferably at least about 90%. Typically the cell marks is a receptor such as CD34 receptor. Also the particle may correspond to a fluorophore particle such as those described herein or known in the art or a magnetic particle (that is one that is attracted by magnetic force) which may comprise one or more of iron, nickel, or cobalt. Various embodiments of cells produced using embodiments of the kits described herein may have selected amount of microbeads attached to each cells.

### Tregs

In various embodiments the population of Tregs cells in one or more therapeutic compositions described herein, may comprise more than about 1×10⁵ Tregs per kilogram of ideal body weight of said human subject. In some embodiments, the population of Tregs may comprise about 1×10⁵ to 1×10⁷ Tregs per kilogram of ideal body weight of said human subject. In other embodiments, the population Tregs may comprise about 5×10⁵ to 4×10⁶ Tregs per kilogram of ideal body weight of said human subject

According one or more embodiments, a cell component that comprises a population of Tregs can comprise at least about 1×10⁴, at least about 1×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.5×10⁶, at least about 7×10⁶, at least about 7.5×10⁶, at least about 8×10⁶, at least about 8.5×10⁶, at least about 9×10⁶, at least about 9.5×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1×10⁸, at least about 1.5×10⁸, at least about 2×10⁸, at least about 2.5×10⁸, at least about 3×10⁸, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more Tregs per kg of recipient subject's actual body weight or ideal body weight (e.g., where Tregs are CD4+CD25+CD127dim, CD3+CD4+CD25+, CD3+CD4+CD25+CD127dim, CD3+CD4+ CD25+ CD127dim FOXP3+, CD3+FOXP3+, CD3+CD4+FOXP3+, CD3+CD4+CD25+FOXP3+, CD3+CD25+FOXP3+, CD3+CD25+CD127dim, CD4+CD25+, CD4+CD25+CD127dimFOXP3+, FOXP3+, CD4+FOXP3+, CD4+CD25+FOXP3+, CD25+FOXP3+, or CD25+CD127dim).

A cell component that comprises a population of Tregs can comprise at most about 1×10⁴, at most about 1×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, a most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, a most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.5×10⁶, at most about 7×10⁶, a most about 7.5×10⁶, at most about 8×10⁶, at most about 8.5×10⁶, at most about 9×10⁶, at most about 9.5×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1×10⁸, at most about 1.5×10⁸, at most about 2×10⁸, at most about 2.5×10, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10, or at most about 1×10⁹ Tregs per kg of recipient subject's actual body weight or ideal body weight (e.g., where Tregs are CD4+CD25+CD127dim, CD3+CD4+CD25+, CD3+ CD4+ CD25+ CD127dim, CD3+CD4+ CD25+ CD127dim FOXP3+, CD3+FOXP3+, CD3+CD4+FOXP3+, CD3+CD4+CD25+FOXP3+, CD3+CD25+FOXP3+, CD3+CD25+CD127dim, CD4+CD25+, CD4+CD25+CD127dimFOXP3+, FOXP3+, CD4+FOXP3+, CD4+CD25+FOXP3+, CD25+FOXP3+, or CD25+CD127dim).

For example, a cell component that comprises a population of Tregs can comprise 1×10⁴ to 1×10⁹, 1×10⁵ to 1×10¹⁸, 1×10⁵ to 2×10⁷, 5×10⁵ to 2×10⁷, 5×10⁵ to 1.5×10⁷, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ Tregs per kg of recipient subject's actual body weight or ideal body weight (e.g., where Tregs are CD4+CD25+CD127dim, CD3+CD4+CD25+, CD3+CD4+CD25+CD127dim, CD3+ CD4+ CD25+ CD127dim FOXP3+, CD3+FOXP3+, CD3+CD4+FOXP3+, CD3+CD4+CD25+FOXP3+, CD3+CD25+FOXP3+, CD3+CD25+CD127dim, CD4+CD25+, CD4+CD25+CD127dimFOXP3+, FOXP3+, CD4+FOXP3+, CD4+CD25+FOXP3+, CD25+FOXP3+, or CD25+CD127dim).

A population of Tregs of the disclosure can have a defined level of purity for Treg cells. For example, a population of Tregs of the disclosure can comprise at least about 5%, at least about at least about 10%, at least about at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or more Treg cells as a percentage of total cells, as a percentage of nucleated cells, or as a percentage of CD45+ cells (e.g., where Tregs are CD4+CD25+CD127dim, CD3+CD4+CD25+, CD3+CD4+CD25+CD127dim, CD3+ CD4+ CD25+ CD127dim FOXP3+, CD3+FOXP3+, CD3+CD4+FOXP3+, CD3+CD4+CD25+FOXP3+, CD3+CD25+FOXP3+, CD3+CD25+CD127dim, CD4+CD25+, CD4+CD25+CD127dimFOXP3+, FOXP3+, CD4+FOXP3+, CD4+CD25+FOXP3+, CD25+FOXP3+, or CD25+CD127dim).

A population of Tregs of the disclosure can comprise 50% to 100%, 60% to 100%, 70% to 100%, 75% to 100%, 80% to 100%, 81% to 100%, 82% to 100%, 83% to 100%, 84% to 100%, 84% to 100%, 86% to 100%, 87% to 100%, 88% to 100%, 89% to 100%, 90% to 91%, 92% to 100%, 93% to 100%, 94% to 100%, 95% to 100%, 96% to 100%, 97% to 100%, 98% to 100%, 99% to 100%, 99.5% to 100%, 50% to 99%, 60% to 99%, 70% to 99%, 80% to 99%, 81% to 99%, 82% to 99%, 83% to 99%, 84% to 99%, 85% to 99%, 86% to 99%, 87% to 99%, 88% to 99%, 89% to 99%, 90% to 99%, 91% to 99%, 92% to 99%, 94% to 99%, 95% to 99%, 96% to 97%, 98% to 99%, 50% to 98%, 60% to 98%, 70% to 98%, 80% to 98%, 81% to 98%, 82% to 98%, 83% to 98%, 84% to 98%, 85% to 98%, 86% to 98%, 87% to 98%, 88% to 98%, 89% to 98%, 90% to 98%, 91% to 98%, 92% to 98%, 94% to 98%, 95% to 98%, 96% to 97%, 98% to 98%, 50% to 97%, 60% to 97%, 70% to 97%, 80% to 97%, 81% to 97%, 82% to 97%, 83% to 97%, 84% to 97%, 85% to 97%, 86% to 97%, 87% to 97%, 88% to 97%, 89% to 97%, 90% to 97%, 91% to 97%, 92% to 97%, 94% to 97%, 95% to 97%, 96% to 97%, 50% to 96%, 60% to 96%, 70% to 96%, 80% to 96%, 81% to 96%, 82% to 96%, 83% to 96%, 84% to 96%, 85% to 96%, 86% to 96%, 87% to 96%, 88% to 96%, 89% to 96%, 90% to 96%, 91% to 96%, 92% to 96%, 94% to 96%, 95% to 96%, 50% to 95%, 60% to 95%, 70% to 95%, 80% to 95%, 81% to 95%, 82% to 95%, 83% to 95%, 84% to 95%, 85% to 95%, 86% to 95%, 87% to 95%, 88% to 95%, 89% to 95%, 90% to 95%, 91% to 95%, 92% to 95%, or 94% to 95% Tregs as a percentage of total cells, nucleated cells, or CD45+ cells (e.g., where the Tregs are CD4+CD25+CD127dim, CD3+CD4+CD25+, CD3+ CD4+ CD25+ CD127dim, CD3+CD4+CD25+CD127dim FOXP3+, CD3+FOXP3+, CD3+CD4+FOXP3+, CD3+CD4+CD25+FOXP3+, CD3+CD25+FOXP3+, CD3+CD25+CD127dim, CD4+CD25+, CD4+CD25+CD127dimFOXP3+, FOXP3+, CD4+FOXP3+, CD4+CD25+FOXP3+, CD25+FOXP3+, or CD25+CD127dim).

A population of Tregs of the disclosure can have a defined level of contaminating non-Treg cells. In some embodiments, at most about 1×10², at most about 2×10², at most about 3×10², at most about 4×10², at most about 5×10², at most about 6×10², at most about 7×10², at most about 8×10², at most about 9×10², at most about 1×10³, at most about 2×10³, at most about 3×10³, at most about 4×10³, at most about 5×10³, at most about 6×10³, at most about 7×10³, at most about 8×10³, at most about 9×10³, at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, or at most about 1×10⁵ non-Treg cells per kg of body weight or ideal body weight are present in a population of Tregs of the disclosure, where non-Treg cells are FOXP3− or CD127+/bright.

In some embodiments, a population of Tregs of the disclosure comprises at most about 0.001%, at most about 0.002%, at most about 0.003%, at most about 0.004%, at most about 0.005%, at most about 0.006%, at most about 0.007%, at most about 0.008% 0.009%, at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, at most about 0.2%, at most about 0.3%, at most about 0.4%, at most about 0.5%, at most about 0.6%, at most about 0.7%, at most about 0.8%, at most about 0.9%, at most about 1%, at most about 1.1%, at most about 1.2%, at most about 1.3%, at most about 1.4%, at most about 1.5%, at most about 1.6%, at most about 1.7%, at most about 1.8%, at most about 1.9%, at most about 2%, at most about 2.1%, at most about 2.2%, at most about 2.3%, at most about 2.4%, at most about 2.5%, at most about 2.6%, at most about 2.7%, at most about 2.8%, at most about 2.9%, at most about 3%, at most about 3.1%, at most about 3.2%, at most about 3.3%, at most about 3.4%, at most about 3.5%, at most about 3.6%, at most about 3.7%, at most about 3.8%, at most about 3.9%, at most about 4%, at most about 5%, at most about 6%, at most about 7%, at most about 8%, at most about 9%, or at most about 10% non-Treg cells, where non-Treg cells are FOXP3− or CD127+/bright.

In some embodiments, the population of Tregs comprise less than 3 EU/ml endotoxins. In some embodiments, the population of Tregs comprises less than 1 EU/ml endotoxins. A cell component that comprises a population of Tregs can comprise 0.5 EU/ml endotoxins to 10 EU/ml endotoxins. A cell component that comprises a population of Tregs can comprise at least 0.5 EU/ml endotoxins. A cell component that comprises a population of Tregs can comprise at most 10 EU/ml endotoxins. A cell component that comprises a population of Tregs can comprise 10 EU/ml endotoxins to 8 EU/ml endotoxins, 10 EU/ml endotoxins to 6 EU/ml endotoxins, 10 EU/ml endotoxins to 5 EU/ml endotoxins, 10 EU/ml endotoxins to 4 EU/ml endotoxins, 10 EU/ml endotoxins to 2 EU/ml endotoxins, 10 EU/ml endotoxins to 1 EU/ml endotoxins, 10 EU/ml endotoxins to 0.5 EU/ml endotoxins, 8 EU/ml endotoxins to 6 EU/ml endotoxins, 8 EU/ml endotoxins to 5 EU/ml endotoxins, 8 EU/ml endotoxins to 4 EU/ml endotoxins, 8 EU/ml endotoxins to 2 EU/ml endotoxins, 8 EU/ml endotoxins to 1 EU/ml endotoxins, 8 EU/ml endotoxins to 0.5 EU/ml endotoxins, 6 EU/ml endotoxins to 5 EU/ml endotoxins, 6 EU/ml endotoxins to 4 EU/ml endotoxins, 6 EU/ml endotoxins to 2 EU/ml endotoxins, 6 EU/ml endotoxins to 1 EU/ml endotoxins, 6 EU/ml endotoxins to 0.5 EU/ml endotoxins, 5 EU/ml endotoxins to 4 EU/ml endotoxins, 5 EU/ml endotoxins to 2 EU/ml endotoxins, 5 EU/ml endotoxins to 1 EU/ml endotoxins, 5 EU/ml endotoxins to 0.5 EU/ml endotoxins, 4 EU/ml endotoxins to 2 EU/ml endotoxins, 4 EU/ml endotoxins to 1 EU/ml endotoxins, 4 EU/ml endotoxins to 0.5 EU/ml endotoxins, 2 EU/ml endotoxins to 1 EU/ml endotoxins, 2 EU/ml endotoxins to 0.5 EU/ml endotoxins, or 1 EU/ml endotoxins to 0.5 EU/ml endotoxins. A cell component that comprises a population of Tregs can comprise 10 EU/ml endotoxins, 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A cell component that comprises a population of Tregs can comprise at least 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A cell component that comprises a population of Tregs can comprise at most 10 EU/ml endotoxins, 8 EU/ml endotoxins, 6 EU/ml endotoxins, 5 EU/ml endotoxins, 4 EU/ml endotoxins, 2 EU/ml endotoxins or 1 EU/ml endotoxins.

A cell component that comprises a population of Tregs can comprise 0.5% w/w to 10% w/w unbound reagents. These unbound reagents may include any affinity reagents used for the sorting of Tregs, for instance, antibodies, or purification particles or magnetic particles. A cell component that comprises a population of Tregs can comprise at least 0.5% w/w unbound reagents. A cell component that comprises a population of Tregs can comprise at most 10% w/w unbound reagents. A cell component that comprises a population of Tregs can comprise 10% w/w to 8% w/w, 10% w/w to 6% w/w, 10% w/w to 5% w/w, 10% w/w to 4% w/w, 10% w/w to 2% w/w, 10% w/w to 1% w/w, 10% w/w to 0.5% w/w, 8% w/w to 6% w/w, 8% w/w to 5% w/w, 8% w/w to 4% w/w, 8% w/w to 2% w/w, 8% w/w to 1% w/w, 8% w/w to 0.5% w/w, 6% w/w to 5% w/w, 6% w/w to 4% w/w, 6% w/w to 2% w/w, 6% w/w to 1% w/w, 6% w/w to 0.5% w/w, 5% w/w to 4% w/w, 5% w/w to 2% w/w, 5% w/w to 1% w/w, 5% w/w to 0.5% w/w, 4% w/w to 2% w/w, 4% w/w to 1% w/w, 4% w/w to 0.5% w/w, 2% w/w to 1% w/w, 2% w/w to 0.5% w/w, or 1% w/w to 0.5% w/w unbound reagents. A cell component that comprises a population of Tregs can comprise 10% w/w, 8% w/w, 6% w/w, 5% w/w, 4% w/w, 2% w/w, 1% w/w, or 0.5% w/w unbound reagents. A cell component that comprises a population of Tregs can comprise at least 8% w/w, 6% w/w, 5% w/w, 4% w/w, 2% w/w, 1% w/w, or 0.5% w/w unbound reagents. A cell component that comprises a population of Tregs can comprise at most 10% w/w, 8% w/w, 6% w/w, 5% w/w, 4% w/w, 2% w/w or 1% w/w unbound reagents.

A cell component that comprises a population of Tregs can comprise 1×10³ to 50×10³ microbeads per cell. These microbeads may comprise microbeads used to purify the Treg population, for instance, an anti-CD25 antibody comprising microbead used to sort the Treg population, or an anti-CD4 antibody comprising microbead used to sort the Treg population, and/or an anti-CD127 antibody comprising microbead used to sort the Treg population. A cell component that comprises a population of Tregs can comprise at least 1×10³ microbeads per cell. A cell component that comprises a population of Tregs can comprise at most 50×10³ microbeads per cell. A cell component that comprises a population of Tregs can comprise 50×10³ to 40×10³, 50×10³ to 30×10³, 50×10³ to 20×10³, 50×10³ to 10×10³, 50×10³ to 5×10³, 50×10³ to 4×10³, 50×10³ to 2×10³, 50×10³ to 1×10³, 40×10³ to 30×10³, 40×10³ to 20×10³, 40×10³ to 10×10³, 40×10³ to 5×10³, 40×10³ to 4×10³, 40×10³ to 2×10³, 40×10³ to 1×10³, 30×10³ to 20×10³, 30×10³ to 10×10³, 30×10³ to 5×10³, 30×10³ to 4×10³, 30×10³ to 2×10³, 30×10³ to 1×10³, 20×10³ to 10×10³, 20×10³ to 5×10³, 20×10³ to 4×10³, 20×10³ to 2×10³, 20×10³ to 1×10³, 10×10³ to 5×10³, 10×10³ to 4×10³, 10×10³ to 2×10³, 10×10³ to 1×10³, 5×10³ to 4×10³, 5×10³ to 2×10³, 5×10³ to 1×10³, 4×10³ to 2×10³, 4×10³ to 1×10³, or 2×10³ to 1×10³ microbeads per cell. A cell component that comprises a population of Tregs can comprise 50×10³, 40×10³, 30×10³, 20×10³, 10×10³, 5×10³, 4×10³, 2×10³, or 1×10³ microbeads per cell. Various embodiments of cells produced using embodiments of the kits described herein may have selected amount of microbeads attached to each cells. For example, a population of Treg cells produced using embodiments of the kits and methods described herein may have less than about 30,000 microbeads per cell. A microbead that is bound to a cell is bound via at least one antibody immobilized on the bead. In some cases, a microbead has one immobilized antibody. A microbead may have 100, 1,000, 10,000, 100,000, 1,000,000, 1,000,000,000 or more immobilized antibodies and any number of immobilized antibodies therebetween.

The average amount of antibody (e.g., a CD25+ or CD4+ antibody) bound to at least a portion of the Tregs can range from about 500 to 10,000 per cell with specific embodiments of about 1000 to 10,000, 2500 to 10,000. Wider ranges and higher upper limits are also contemplated including for example about 1,000 to 20,000 antibodies per cell. In some embodiments, at least about 70% of the Treg cells in the second or other population may have bound antibody (or other antigen binding agent) and more preferably at least about 90% with higher amounts contemplated.

In another aspect, embodiments of the disclosure provide therapeutic compositions for hematopoietic or other stem cell transplantation to a human or other patient in need thereof. According to one embodiment, a therapeutic composition for hematopoietic cell transplantation to a human patient in need thereof comprises a population of isolated T cells wherein at least a portion of the T cells have an anti-CD4+ antibody bound to a marker on the cell surface which is used to separate T cells from a mixture of nucleated cells from a mobilized blood donor. Typically, the T-cells is a regulatory T-cell or Treg. The average amount of anti CD4+ or other antibody bound to at least a portion of the Tregs in can range from about 1000 to 400,000 antibodies per cell with a specific embodiment of about 50,000 to 400,000. Wider ranges and higher upper limits are also contemplated. In some embodiments, at least about 70% of the Treg cells may have bound antibody (or other antigen binding agent) and more preferably at least about 90% with higher amounts contemplated. Also, typically the marker is receptor in particular a CD+4 receptor. According to another embodiment, the therapeutic composition for hematopoietic cell transplantation to a human patient in need thereof comprises a population of isolated T regulatory (Treg) cells wherein at least a portion of the Treg cells have a particle bound to a receptor on the cell surface which is used to separate Treg cells from a mixture of nucleated cells from a mobilized blood donor, the antibody conjugated to a particle. The particle may be attached to the Treg cells by one or more linking molecules such as antibody other antigen or affinity binding molecules. In some embodiments, the particle can be attached directly to the cells by means polar, Van Der Waals or other chemical or physical forces known in the art. The particle may correspond to a fluorophore particle such as those described herein or known in the art or a magnetic particle (that is one that is attracted by magnetic force) which may comprise one or more of iron, nickel, or cobalt.

### Tcons

In some embodiments, the population of Tcons may comprise fewer than 1×10⁷ Tcons per kilogram of ideal body weight of said human subject. In some embodiments, the population of Tcons may comprise 1×10⁵ to 1×10⁷ Tcons per kilogram of ideal body weight of said human subject. In some embodiments, the population of Tcons may comprise 5×10⁵ to 4×10⁶ Tcons per kilogram of ideal body weight of said human subject.

A cell component that comprises a population of Tcons can comprise at least about 1×10⁴, at least about 1×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.5×10⁶, at least about 7×10⁶, at least about 7.5×10⁶, at least about 8×10⁶, at least about 8.5×10⁶, at least about 9×10⁶, at least about 9.5×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1×10⁸, at least about 1.5×10⁸, at least about 2×10⁸, at least about 2.5×10⁸, at least about 3×108, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more Tcons per kg of recipient subject's actual body weight or ideal body weight (e.g., where Tcons are CD3+ or CD3+ CD127+/bright).

A cell component that comprises a population of Tcons can comprise at most about 1×10⁴, at most about 1×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, a most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, a most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.5×10⁶, at most about 7×10⁶, a most about 7.5×10⁶, at most about 8×10⁶, at most about 8.5×10⁶, at most about 9×10⁶, at most about 9.5×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1×10⁸, at most about 1.5×10⁸, at most about 2×10⁸, at most about 2.5×10, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ Tcons per kg of recipient subject's actual body weight or ideal body weight (e.g., where Tcons are CD3+ or CD3+CD127+/bright).

For example, a cell component that comprises a population of Tcons can comprise 1×10⁴ to 1×10⁹, 1×10⁵ to 1×10¹⁸, 1×10⁵ to 2×10⁷, 5×10⁵ to 2×10⁷, 5×10⁵ to 1.5×10⁷, 5×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ Tcons per kg of recipient subject's actual body weight or ideal body weight (e.g., where Tcons are CD3+ or CD3+CD127+/bright).

A population of Tcons of the disclosure can have a defined level of purity for Tcon cells. For example, a population of Tcons of the disclosure can comprise at least about 5%, at least about at least about 10%, at least about at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or more Tcon cells as a percentage of total cells, as a percentage of nucleated cells, or as a percentage of CD45+ cells (e.g., where Tcons are CD3+ or CD3+ CD127+/bright).

A population of Tcons of the disclosure can comprise 50% to 100%, 60% to 100%, 70% to 100%, 75% to 100%, 80% to 100%, 81% to 100%, 82% to 100%, 83% to 100%, 84% to 100%, 84% to 100%, 86% to 100%, 87% to 100%, 88% to 100%, 89% to 100%, 90% to 91%, 92% to 100%, 93% to 100%, 94% to 100%, 95% to 100%, 96% to 100%, 97% to 100%, 98% to 100%, 99% to 100%, 99.5% to 100%, 50% to 99%, 60% to 99%, 70% to 99%, 80% to 99%, 81% to 99%, 82% to 99%, 83% to 99%, 84% to 99%, 85% to 99%, 86% to 99%, 87% to 99%, 88% to 99%, 89% to 99%, 90% to 99%, 91% to 99%, 92% to 99%, 94% to 99%, 95% to 99%, 96% to 97%, 98% to 99%, 50% to 98%, 60% to 98%, 70% to 98%, 80% to 98%, 81% to 98%, 82% to 98%, 83% to 98%, 84% to 98%, 85% to 98%, 86% to 98%, 87% to 98%, 88% to 98%, 89% to 98%, 90% to 98%, 91% to 98%, 92% to 98%, 94% to 98%, 95% to 98%, 96% to 97%, 98% to 98%, 50% to 97%, 60% to 97%, 70% to 97%, 80% to 97%, 81% to 97%, 82% to 97%, 83% to 97%, 84% to 97%, 85% to 97%, 86% to 97%, 87% to 97%, 88% to 97%, 89% to 97%, 90% to 97%, 91% to 97%, 92% to 97%, 94% to 97%, 95% to 97%, 96% to 97%, 50% to 96%, 60% to 96%, 70% to 96%, 80% to 96%, 81% to 96%, 82% to 96%, 83% to 96%, 84% to 96%, 85% to 96%, 86% to 96%, 87% to 96%, 88% to 96%, 89% to 96%, 90% to 96%, 91% to 96%, 92% to 96%, 94% to 96%, 95% to 96%, 50% to 95%, 60% to 95%, 70% to 95%, 80% to 95%, 81% to 95%, 82% to 95%, 83% to 95%, 84% to 95%, 85% to 95%, 86% to 95%, 87% to 95%, 88% to 95%, 89% to 95%, 90% to 95%, 91% to 95%, 92% to 95%, or 94% to 95%, Tcons as a percentage of total cells, nucleated cells, or CD45+ cells (e.g., where Tcons are CD3+ or CD3+ CD127+/bright).

A population of Tcons of the disclosure can have a defined level of contaminating non-Tcon cells. In some embodiments, at most about 1×10², at most about 2×10², at most about 3×10², at most about 4×10², at most about 5×10², at most about 6×10², at most about 7×10², at most about 8×10², at most about 9×10², at most about 1×10³, at most about 2×10³, at most about 3×10³, at most about 4×10³, at most about 5×10³, at most about 6×10³, at most about 7×10³, at most about 8×10³, at most about 9×10³, at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, or at most about 1×10⁵ non-Tcon cells per kg of recipient subject's actual body weight or ideal body weight are present in a population of Tcons of the disclosure, (e.g., where non-Tcon cells are CD3− or CD3+CD127dim).

In some embodiments, a population of Tcons of the disclosure comprises at most about 0.001%, at most about 0.002%, at most about 0.003%, at most about 0.004%, at most about 0.005%, at most about 0.006%, at most about 0.007%, at most about 0.008% 0.009%, at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, at most about 0.2%, at most about 0.3%, at most about 0.4%, at most about 0.5%, at most about 0.6%, at most about 0.7%, at most about 0.8%, at most about 0.9%, at most about 1%, at most about 1.1%, at most about 1.2%, at most about 1.3%, at most about 1.4%, at most about 1.5%, at most about 1.6%, at most about 1.7%, at most about 1.8%, at most about 1.9%, at most about 2%, at most about 2.1%, at most about 2.2%, at most about 2.3%, at most about 2.4%, at most about 2.5%, at most about 2.6%, at most about 2.7%, at most about 2.8%, at most about 2.9%, at most about 3%, at most about 3.1%, at most about 3.2%, at most about 3.3%, at most about 3.4%, at most about 3.5%, at most about 3.6%, at most about 3.7%, at most about 3.8%, at most about 3.9%, at most about 4%, at most about 5%, at most about 6%, at most about 7%, at most about 8%, at most about 9%, or at most about 10% non-Tcon cells, (e.g., where non-Tcon cells are CD3− or CD3+CD127dim).

A cell component that comprises a population of Tcons can comprise 0.5 EU/ml endotoxins to 10 EU/ml endotoxins. A cell component that comprises a population of Tcons can comprise at least 0.5 EU/ml endotoxins. A cell component that comprises a population of Tcons can comprise at most 10 EU/ml endotoxins. A cell component that comprises a population of Tcons can comprise 10 EU/ml endotoxins to 8 EU/ml endotoxins, 10 EU/ml endotoxins to 6 EU/ml endotoxins, 10 EU/ml endotoxins to 5 EU/ml endotoxins, 10 EU/ml endotoxins to 4 EU/ml endotoxins, 10 EU/ml endotoxins to 2 EU/ml endotoxins, 10 EU/ml endotoxins to 1 EU/ml endotoxins, 10 EU/ml endotoxins to 0.5 EU/ml endotoxins, 8 EU/ml endotoxins to 6 EU/ml endotoxins, 8 EU/ml endotoxins to 5 EU/ml endotoxins, 8 EU/ml endotoxins to 4 EU/ml endotoxins, 8 EU/ml endotoxins to 2 EU/ml endotoxins, 8 EU/ml endotoxins to 1 EU/ml endotoxins, 8 EU/ml endotoxins to 0.5 EU/ml endotoxins, 6 EU/ml endotoxins to 5 EU/ml endotoxins, 6 EU/ml endotoxins to 4 EU/ml endotoxins, 6 EU/ml endotoxins to 2 EU/ml endotoxins, 6 EU/ml endotoxins to 1 EU/ml endotoxins, 6 EU/ml endotoxins to 0.5 EU/ml endotoxins, 5 EU/ml endotoxins to 4 EU/ml endotoxins, 5 EU/ml endotoxins to 2 EU/ml endotoxins, 5 EU/ml endotoxins to 1 EU/ml endotoxins, 5 EU/ml endotoxins to 0.5 EU/ml endotoxins, 4 EU/ml endotoxins to 2 EU/ml endotoxins, 4 EU/ml endotoxins to 1 EU/ml endotoxins, 4 EU/ml endotoxins to 0.5 EU/ml endotoxins, 2 EU/ml endotoxins to 1 EU/ml endotoxins, 2 EU/ml endotoxins to 0.5 EU/ml endotoxins, or 1 EU/ml endotoxins to 0.5 EU/ml endotoxins. A cell component that comprises a population of Tcons can comprise about 10 EU/ml endotoxins, 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A cell component that comprises a population of Tcons can comprise at least 8 EU/ml endotoxins, 6 EU/ml endotoxins, 5 EU/ml endotoxins, 4 EU/ml endotoxins, 2 EU/ml endotoxins, 1 EU/ml endotoxins, or 0.5 EU/ml endotoxins. A cell component that comprises a population of Tcons can comprise at most 10 EU/ml endotoxins, 8 EU/ml endotoxins, 6 EU/ml endotoxins, 5 EU/ml endotoxins, 4 EU/ml endotoxins, 2 EU/ml endotoxins or 1 EU/ml endotoxins. In some embodiments, the population of Tcons may comprise less than 3 EU/ml endotoxins. In some embodiments, the population of Tcons comprises less than 1 EU/ml endotoxins.

A cell component that comprises a population of Tcons can comprise less than 0.1% w/w to 3% w/w unbound reagents. These unbound reagents may include any affinity reagents used for the sorting of Tcons or other cell populations, for instance, antibodies, or purification particles or magnetic particles. A cell component that comprises a population of Tcons can comprise less than about 0.1% w/w unbound reagents. A cell component that comprises a population of Tcons can comprise less than 3% w/w to 2% w/w, 3% w/w to 1% w/w, 3% w/w to 0.5% w/w, 3% w/w to 0.25% w/w, 3% w/w to 0.1% w/w, 2% w/w to 1% w/w, 2% w/w to 0.5% w/w, 2% w/w to 0.25% w/w, 2% w/w to 0.1% w/w, 1% w/w to 0.5% w/w, 1% w/w to 0.25% w/w, 1% w/w to 0.1% w/w, 0.5% w/w to 0.25% w/w, 0.5% w/w to 0.1% w/w, or 0.25% w/w to 0.1% w/w unbound reagents. A cell component that comprises a population of Tcons can comprise less than about 3% w/w, 2% w/w, 1% w/w, 0.5% w/w, 0.25% w/w, or 0.1% w/w unbound reagents.

A cell component that comprises a population of Tcons can comprise less than 50 to 2,000 microbeads per cell. These microbeads may comprise microbeads used to purify the Tcon population or other cell populations, for instance, a CD25 microbead, or a CD4 microbead, or a CD127 microbead, or a CD34 microbead used to sort a cell population. A cell component that comprises a population of Tcons can comprise less than 2,000 microbeads per cell. A cell component that comprises a population of Tcons can comprise less than 2,000 to 1,000, 2,000 to 700, 2,000 to 500, 2,000 to 300, 2,000 to 100, 2,000 to 50, 1,000 to 700, 1,000 to 500, 1,000 to 300, 1,000 to 100, 1,000 to 50, 700 to 500, 700 to 300, 700 to 100, 700 to 50, 500 to 300, 500 to 100, 500 to 50, 300 to 100, 300 to 50, or 100 to 50 microbeads per cell. A cell component that comprises a population of Tcons can comprise about 2,000, 1,000, 700, 500, 300, 100, or 50 microbeads per cell. Various embodiments of cells produced using embodiments of the kits described herein may have selected amount of microbeads attached to each cells. For example, a population of Tcons produced using embodiments of the kits and methods described herein may have less than about 30,000 microbeads per cell or less than 20,000 microbeads per cell or less than 5,000 microbeads per cell. A microbead that is bound to a cell is bound via at least one antibody immobilized on the bead. In some cases, a microbead has one immobilized antibody. A microbead may have 100, 1,000, 10,000, 100,000, 1,000,000, 1,000,000,000 or more immobilized antibodies and any number of immobilized antibodies therebetween. A cell component that comprises a population of Tcons can comprise no microbeads per cell.

In the methods of the disclosure, the ratio of Tcons:Tregs administered to a subject can be, for example, about 1:100, 1:50, 1:25, 1:20, 1:15, 1:10, 1:9, 1:8, 1:7, 1:6, 1:5, 1:4, 1:3, 1:2.5, 1:2, 1.5:2, 1:1.5, 1:1, 1.5:1, 2:1, 2:1.5, 2.5:1, 3:1, 4:1, 5:1, 6:1, 7:1, 8:1, 9:1, 10:1, 15:1, 20:1, 25:1, 50:1, or 100:1.

In some embodiments, the population of Tcons has been cryopreserved prior to the administering of the population Tcons. Tcons can be cryopreserved for any amount of time. Tcons may be cryopreserved for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 at least about 14 hours, at least about 16 hours, at least about 18 hours, at least about 20 hours, at least about 22 hours, at least about 24 hours, at least about 30 hours, at least about 36 hours at least about 48 hours, at least about 50 hours, at least about 55 hours, at least about 60 hours, at least about 61 hours, at least about 62 hours, at least about 65 hours, at least about 70 hours, at least about 72 hours, at least about 80 hours, at least about 90 hours, at least about 96 hours, at least about 120 hours, at least about 150 hours, at least about 200 hours, at least about 300 hours, or more prior to thawing and administration to a subject.

In some embodiments, Tcons are cryopreserved for at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 11 hours, at most about 12 at most about 14 hours, at most about 16 hours, at most about 18 hours, at most about 20 hours, at most about 22 hours, at most about 24 hours, at most about 30 hours, at most about 36 hours at most about 48 hours, at most about 50 hours, at most about 55 hours, at most about 60 hours, at most about 61 hours, at most about 62 hours, at most about 65 hours, at most about 70 hours, at most about 72 hours, at most about 80 hours, at most about 90 hours, at most about 96 hours, at most about 120 hours, at most about 150 hours, at most about 200 hours, or at most about 300 hours prior to thawing and administration to a subject.

In some embodiments, Tcons are cryopreserved for at least about 1 day, at least about 2 days, at least about 3 days, at least about 4 days, at least about 5 days, at least about 6 days, at least about 7 days, at least about 10 days, at least about 14 days, at least about 21 days, at least about 28 days, at least about 50 days, at least about 60 days, or at least about 96 days, or more prior to thawing and administration to a subject.

In some embodiments, Tcons are cryopreserved for at most about 1 day, at most about 2 days, at most about 3 days, at most about 4 days, at most about 5 days, at most about 6 days, at most about 7 days, at most about 10 days, at most about 14 days, at most about 21 days, at most about 28 days, at most about 50 days, at most about 60 days, or at most about 96 days prior to thawing and administration to a subject.

iNKTs

In some embodiments, the method may comprise administering to a human subject a population of invariant natural killer T cells (iNKTs). In some embodiments, the iNKTs are CD3+ Vα24Jα18+. In some embodiments, the population of iNKTs may comprise more than 5×10² iNKTs per kilogram of ideal body weight of the human subject. In some embodiments, the population of iNKTs may comprise 5×10² to 1×10⁷ iNKTs per kilogram of ideal body weight of the human subject.

A cell component that comprises a population of iNKTs can comprise at least about 1×10⁴, at least about 1×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.5×10⁶, at least about 7×10⁶, at least about 7.5×10⁶, at least about 8×10⁶, at least about 8.5×10⁶, at least about 9×10⁶, at least about 9.5×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1×10⁸, at least about 1.5×10⁸, at least about 2×10⁸, at least about 2.5×10⁸, at least about 3×108, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more iNKTs per kg of recipient subject's actual body weight or ideal body weight (e.g., where iNKTs are CD3+ Vα24Jα18+).

A cell component that comprises a population of iNKTs can comprise at most about 1×10⁴, at most about 1×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, a most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, a most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.5×10⁶, at most about 7×10⁶, at most about 7.5×10⁶, at most about 8×10⁶, at most about 8.5×10⁶, at most about 9×10⁶, at most about 9.5×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1×10⁸, at most about 1.5×10⁸, at most about 2×10⁸, at most about 2.5×10⁸, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ iNKTs per kg of recipient subject's actual body weight or ideal body weight (e.g., where iNKTs are CD3+ Vα24Jα18+).

For example, a cell component that comprises a population of iNKTs can comprise 1×10⁴ to 1×10⁹, 1×10⁵ to 1×10¹, 1×10⁵ to 2×10⁷, 5×10⁵ to 2×10⁷, 5×10⁵ to 1.5×10⁷, 1×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ iNKTs per kg of recipient subject's actual body weight or ideal body weight (e.g., where iNKTs are CD3+ Vα24Jα18+).

A population of iNKTs of the disclosure can have a defined level of purity for iNKT cells. For example, a population of iNKTs of the disclosure can comprise at least about 5%, at least about at least about 10%, at least about at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or more iNKT cells as a percentage of total cells, as a percentage of nucleated cells, or as a percentage of CD45+ cells (e.g., where iNKTs are CD3+ Vα24Jα18+).

A population of iNKTs of the disclosure can comprise 50% to 100%, 60% to 100%, 70% to 100%, 75% to 100%, 80% to 100%, 81% to 100%, 82% to 100%, 83% to 100%, 84% to 100%, 84% to 100%, 86% to 100%, 87% to 100%, 88% to 100%, 89% to 100%, 90% to 91%, 92% to 100%, 93% to 100%, 94% to 100%, 95% to 100%, 96% to 100%, 97% to 100%, 98% to 100%, 99% to 100%, 99.5% to 100%, 50% to 99%, 60% to 99%, 70% to 99%, 80% to 99%, 81% to 99%, 82% to 99%, 83% to 99%, 84% to 99%, 85% to 99%, 86% to 99%, 87% to 99%, 88% to 99%, 89% to 99%, 90% to 99%, 91% to 99%, 92% to 99%, 94% to 99%, 95% to 99%, 96% to 97%, 98% to 99%, 50% to 98%, 60% to 98%, 70% to 98%, 80% to 98%, 81% to 98%, 82% to 98%, 83% to 98%, 84% to 98%, 85% to 98%, 86% to 98%, 87% to 98%, 88% to 98%, 89% to 98%, 90% to 98%, 91% to 98%, 92% to 98%, 94% to 98%, 95% to 98%, 96% to 97%, 98% to 98%, 50% to 97%, 60% to 97%, 70% to 97%, 80% to 97%, 81% to 97%, 82% to 97%, 83% to 97%, 84% to 97%, 85% to 97%, 86% to 97%, 87% to 97%, 88% to 97%, 89% to 97%, 90% to 97%, 91% to 97%, 92% to 97%, 94% to 97%, 95% to 97%, 96% to 97%, 50% to 96%, 60% to 96%, 70% to 96%, 80% to 96%, 81% to 96%, 82% to 96%, 83% to 96%, 84% to 96%, 85% to 96%, 86% to 96%, 87% to 96%, 88% to 96%, 89% to 96%, 90% to 96%, 91% to 96%, 92% to 96%, 94% to 96%, 95% to 96%, 50% to 95%, 60% to 95%, 70% to 95%, 80% to 95%, 81% to 95%, 82% to 95%, 83% to 95%, 84% to 95%, 85% to 95%, 86% to 95%, 87% to 95%, 88% to 95%, 89% to 95%, 90% to 95%, 91% to 95%, 92% to 95%, or 94% to 95%, iNKTs as a percentage of total cells, nucleated cells, or CD45+ cells (e.g., where iNKTs are CD3+ Vα24Jα18+).

A population of iNKTs of the disclosure can have a defined level of contaminating non-iNKT cells. In some embodiments, at most about 1×10², at most about 2×10², at most about 3×10², at most about 4×10², at most about 5×10², at most about 6×10², at most about 7×10², at most about 8×10², at most about 9×10², at most about 1×10³, at most about 2×10³, at most about 3×10³, at most about 4×10³, at most about 5×10³, at most about 6×10³, at most about 7×10³, at most about 8×10³, at most about 9×10³, at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, or at most about 1×10⁵ non-iNKT cells per kg of recipient subject's actual body weight or ideal body weight are present in a population of iNKTs of the disclosure, (e.g., where non-iNKT cells are Vα24Jα18−).

In some embodiments, a population of iNKTs of the disclosure comprises at most about 0.001%, at most about 0.002%, at most about 0.003%, at most about 0.004%, at most about 0.005%, at most about 0.006%, at most about 0.007%, at most about 0.008% 0.009%, at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, at most about 0.2%, at most about 0.3%, at most about 0.4%, at most about 0.5%, at most about 0.6%, at most about 0.7%, at most about 0.8%, at most about 0.9%, at most about 1%, at most about 1.1%, at most about 1.2%, at most about 1.3%, at most about 1.4%, at most about 1.5%, at most about 1.6%, at most about 1.7%, at most about 1.8%, at most about 1.9%, at most about 2%, at most about 2.1%, at most about 2.2%, at most about 2.3%, at most about 2.4%, at most about 2.5%, at most about 2.6%, at most about 2.7%, at most about 2.8%, at most about 2.9%, at most about 3%, at most about 3.1%, at most about 3.2%, at most about 3.3%, at most about 3.4%, at most about 3.5%, at most about 3.6%, at most about 3.7%, at most about 3.8%, at most about 3.9%, at most about 4%, at most about 5%, at most about 6%, at most about 7%, at most about 8%, at most about 9%, or at most about 10% non-iNKT cells, (e.g., where non-iNKT cells are Vα24Jα18−).

### Tmems

In various embodiments, the method of treating a patient for various conditions (e.g., cancer or autoimmune disease) may comprise administering to the human subject a population of memory T cells (Tmems). The Tmems may correspond to CD3+ CD45RA− CD45RO+ cells. The number of Tmems in the Tmem population may be in the range of about 3×10⁵ to 1×10⁹ and in specific embodiments may be 3×10⁵, 1×10⁶, 1×10⁷ and 1×10⁸ Tmems per kilogram of ideal body weight of the human subject.

A cell component that comprises a population of Tmems can comprise at least about 1×10⁴, at least about 1×10⁵, at least about 5×10⁵, at least about 6×10⁵, at least about 7×10⁵, at least about 8×10⁵, at least about 9×10⁵, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5×10⁶, at least about 5.1×10⁶, at least about 5.2×10⁶, at least about 5.3×10⁶, at least about 5.4×10⁶, at least about 5.5×10⁶, at least about 5.6×10⁶, at least about 5.7×10⁶, at least about 5.8×10⁶, at least about 5.9×10⁶, at least about 6×10⁶, at least about 6.5×10⁶, at least about 7×10⁶, at least about 7.5×10⁶, at least about 8×10⁶, at least about 8.5×10⁶, at least about 9×10⁶, at least about 9.5×10⁶, at least about 1×10⁷, at least about 1.5×10⁷, at least about 2×10⁷, at least about 2.5×10⁷, at least about 3×10⁷, at least about 3.5×10⁷, at least about 4×10⁷, at least about 4.5×10⁷, at least about 5×10⁷, at least about 5.5×10⁷, at least about 6×10⁷, at least about 6.5×10⁷, at least about 7×10⁷, at least about 7.5×10⁷, at least about 8×10⁷, at least about 8.5×10⁷, at least about 9×10⁷, at least about 9.5×10⁷, at least about 1×10⁸, at least about 1×10⁸, at least about 1.5×10⁸, at least about 2×10, at least about 2.5×10⁸, at least about 3×108, at least about 3.5×10⁸, at least about 4×10⁷, at least about 4.5×10⁸, at least about 5×10⁸, at least about 5.5×10⁸, at least about 6×10⁸, at least about 6.5×10⁸, at least about 7×10⁸, at least about 7.5×10⁸, at least about 8×10⁸, at least about 8.5×10⁸, at least about 9×10⁸, at least about 9.5×10⁸, at least about 1×10⁹, or more Tmems per kg of recipient subject's actual body weight or ideal body weight (e.g., where Tmems are CD3+CD45RA-CD45RO+).

A cell component that comprises a population of Tmems can comprise at most about 1×10⁴, at most about 1×10⁵, at most about 5×10⁵, at most about 6×10⁵, at most about 7×10⁵, at most about 8×10⁵, at most about 9×10⁵, at most about 1×10⁶, at most about 1.1×10⁶, at most about 1.2×10⁶, at most about 1.3×10⁶, at most about 1.4×10⁶, at most about 1.5×10⁶, at most about 1.6×10⁶, at most about 1.7×10⁶, at most about 1.8×10⁶, at most about 1.9×10⁶, at most about 2×10⁶, at most about 2.1×10⁶, at most about 2.2×10⁶, at most about 2.3×10⁶, at most about 2.4×10⁶, at most about 2.5×10⁶, at most about 2.6×10⁶, at most about 2.7×10⁶, at most about 2.8×10⁶, at most about 2.9×10⁶, at most about 3×10⁶, at most about 3.1×10⁶, at most about 3.2×10⁶, at most about 3.3×10⁶, at most about 3.4×10⁶, at most about 3.5×10⁶, at most about 3.6×10⁶, at most about 3.7×10⁶, at most about 3.8×10⁶, at most about 3.9×10⁶, at most about 4×10⁶, at most about 4.1×10⁶, at most about 4.2×10⁶, at most about 4.3×10⁶, at most about 4.4×10⁶, a most about 4.5×10⁶, at most about 4.6×10⁶, at most about 4.7×10⁶, at most about 4.8×10⁶, at most about 4.9×10⁶, at most about 5×10⁶, at most about 5.1×10⁶, at most about 5.2×10⁶, at most about 5.3×10⁶, a most about 5.4×10⁶, at most about 5.5×10⁶, at most about 5.6×10⁶, at most about 5.7×10⁶, at most about 5.8×10⁶, at most about 5.9×10⁶, at most about 6×10⁶, at most about 6.5×10⁶, at most about 7×10⁶, a most about 7.5×10⁶, at most about 8×10⁶, at most about 8.5×10⁶, at most about 9×10⁶, at most about 9.5×10⁶, at most about 1×10⁷, at most about 1.5×10⁷, at most about 2×10⁷, at most about 2.5×10⁷, at most about 3×10⁷, at most about 3.5×10⁷, at most about 4×10⁷, at most about 4.5×10⁷, at most about 5×10⁷, at most about 5.5×10⁷, at most about 6×10⁷, at most about 6.5×10⁷, at most about 7×10⁷, at most about 7.5×10⁷, at most about 8×10⁷, at most about 8.5×10⁷, at most about 9×10⁷, at most about 9.5×10⁷, at most about 1×10⁸, at most about 1×10⁸, at most about 1.5×10⁸, at most about 2×10⁸, at most about 2.5×10⁸, at most about 3×10⁸, at most about 3.5×10⁸, at most about 4×10⁷, at most about 4.5×10⁸, at most about 5×10⁸, at most about 5.5×10⁸, at most about 6×10⁸, at most about 6.5×10⁸, at most about 7×10⁸, at most about 7.5×10⁸, at most about 8×10⁸, at most about 8.5×10⁸, at most about 9×10⁸, at most about 9.5×10⁸, or at most about 1×10⁹ Tmems per kg of recipient subject's actual body weight or ideal body weight (e.g., where Tmems are CD3+CD45RA−CD45RO+).

For example, a cell component that comprises a population of Tmems can comprise 1×10⁴ to 1 ×10⁹, 1×10⁵ to 1×10⁵, 1×10⁵ to 2×10⁷, 5×10⁵ to 2×10⁷, 5×10⁵ to 1.5×10⁷, 1×10⁵ to 1×10⁷, 5×10⁵ to 9×10⁶, 5×10⁵ to 8×10⁶, 5×10⁵ to 7×10⁶, 5×10⁵ to 6×10⁶, 5×10⁵ to 5×10⁶, 5×10⁵ to 4×10⁶, 5×10⁵ to 3×10⁶, 5×10⁵ to 2×10⁶, 5×10⁵ to 1×10⁶, 1×10⁶ to 1.5×10⁷, 1×10⁶ to 1×10⁷, 1×10⁶ to 9×10⁶, 1×10⁶ to 8×10⁶, 1×10⁶ to 7×10⁶, 1×10⁶ to 6×10⁶, 1×10⁶ to 5×10⁶, 1×10⁶ to 4×10⁶, 1×10⁶ to 3×10⁶, 1×10⁶ to 2×10⁶, 1.5×10⁶ to 1.5×10⁷, 1.5×10⁶ to 1×10⁷, 1.5×10⁶ to 9×10⁶, 1.5×10⁶ to 8×10⁶, 1.5×10⁶ to 7×10⁶, 1.5×10⁶ to 6×10⁶, 1.5×10⁶ to 5×10⁶, 1.5×10⁶ to 4×10⁶, 1.5×10⁶ to 3×10⁶, 1.5×10⁶ to 2×10⁶, 2×10⁶ to 1.5×10⁷, 2×10⁶ to 1×10⁷, 2×10⁶ to 9×10⁶, 2×10⁶ to 8×10⁶, 2×10⁶ to 7×10⁶, 2×10⁶ to 6×10⁶, 2×10⁶ to 5×10⁶, 2×10⁶ to 4×10⁶, 2×10⁶ to 3×10⁶, 2.5×10⁶ to 1.5×10⁷, 2.5×10⁶ to 1×10⁷, 2.5×10⁶ to 9×10⁶, 2.5×10⁶ to 8×10⁶, 2.5×10⁶ to 7×10⁶, 2.5×10⁶ to 6×10⁶, 2.5×10⁶ to 5×10⁶, 2.5×10⁶ to 4×10⁶, or 2.5×10⁶ to 3×10⁶ Tmems per kg of recipient subject's actual body weight or ideal body weight (e.g., where Tmems are CD3+CD45RA−CD45RO+).

A population of Tmems of the disclosure can have a defined level of purity for Tmem cells. For example, a population of Tmems of the disclosure can comprise at least about 5%, at least about at least about 10%, at least about at least about 15%, at least about 20%, at least about 25%, at least about 30%, at least about 35%, at least about 40%, at least about 45%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5%, or more Tmem cells as a percentage of total cells, as a percentage of nucleated cells, or as a percentage of CD45+ cells (e.g., where Tmems are CD3+CD45RA−CD45RO+).

A population of Tmems of the disclosure can comprise 50% to 100%, 60% to 100%, 70% to 100%, 75% to 100%, 80% to 100%, 81% to 100%, 82% to 100%, 83% to 100%, 84% to 100%, 84% to 100%, 86% to 100%, 87% to 100%, 88% to 100%, 89% to 100%, 90% to 91%, 92% to 100%, 93% to 100%, 94% to 100%, 95% to 100%, 96% to 100%, 97% to 100%, 98% to 100%, 99% to 100%, 99.5% to 100%, 50% to 99%, 60% to 99%, 70% to 99%, 80% to 99%, 81% to 99%, 82% to 99%, 83% to 99%, 84% to 99%, 85% to 99%, 86% to 99%, 87% to 99%, 88% to 99%, 89% to 99%, 90% to 99%, 91% to 99%, 92% to 99%, 94% to 99%, 95% to 99%, 96% to 97%, 98% to 99%, 50% to 98%, 60% to 98%, 70% to 98%, 80% to 98%, 81% to 98%, 82% to 98%, 83% to 98%, 84% to 98%, 85% to 98%, 86% to 98%, 87% to 98%, 88% to 98%, 89% to 98%, 90% to 98%, 91% to 98%, 92% to 98%, 94% to 98%, 95% to 98%, 96% to 97%, 98% to 98%, 50% to 97%, 60% to 97%, 70% to 97%, 80% to 97%, 81% to 97%, 82% to 97%, 83% to 97%, 84% to 97%, 85% to 97%, 86% to 97%, 87% to 97%, 88% to 97%, 89% to 97%, 90% to 97%, 91% to 97%, 92% to 97%, 94% to 97%, 95% to 97%, 96% to 97%, 50% to 96%, 60% to 96%, 70% to 96%, 80% to 96%, 81% to 96%, 82% to 96%, 83% to 96%, 84% to 96%, 85% to 96%, 86% to 96%, 87% to 96%, 88% to 96%, 89% to 96%, 90% to 96%, 91% to 96%, 92% to 96%, 94% to 96%, 95% to 96%, 50% to 95%, 60% to 95%, 70% to 95%, 80% to 95%, 81% to 95%, 82% to 95%, 83% to 95%, 84% to 95%, 85% to 95%, 86% to 95%, 87% to 95%, 88% to 95%, 89% to 95%, 90% to 95%, 91% to 95%, 92% to 95%, or 94% to 95%, Tmems as a percentage of total cells, nucleated cells, or CD45+ cells (e.g., where Tmems are CD3+CD45RA−CD45RO+).

A population of Tmems of the disclosure can have a defined level of contaminating non-Tmem cells. In some embodiments, at most about 1×10², at most about 2×10², at most about 3×10², at most about 4×10², at most about 5×10², at most about 6×10², at most about 7×10², at most about 8×10², at most about 9×10², at most about 1×10³, at most about 2×10³, at most about 3×10³, at most about 4×10³, at most about 5×10³, at most about 6×10³, at most about 7×10³, at most about 8×10³, at most about 9×10³, at most about 1×10⁴, at most about 2×10⁴, at most about 3×10⁴, at most about 4×10⁴, at most about 5×10⁴, at most about 6×10⁴, at most about 7×10⁴, at most about 8×10⁴, at most about 9×10⁴, or at most about 1×10⁵ non-Tmem cells per kg of recipient subject's actual body weight or ideal body weight are present in a population of Tmems of the disclosure, (e.g., where the non-Tmem cells are CD45RO−).

In some embodiments, a population of Tmems of the disclosure comprises at most about 0.001%, at most about 0.002%, at most about 0.003%, at most about 0.004%, at most about 0.005%, at most about 0.006%, at most about 0.007%, at most about 0.008% 0.009%, at most about 0.01%, at most about 0.02%, at most about 0.03%, at most about 0.04%, at most about 0.05%, at most about 0.06%, at most about 0.07%, at most about 0.08%, at most about 0.09%, at most about 0.1%, at most about 0.2%, at most about 0.3%, at most about 0.4%, at most about 0.5%, at most about 0.6%, at most about 0.7%, at most about 0.8%, at most about 0.9%, at most about 1%, at most about 1.1%, at most about 1.2%, at most about 1.3%, at most about 1.4%, at most about 1.5%, at most about 1.6%, at most about 1.7%, at most about 1.8%, at most about 1.9%, at most about 2%, at most about 2.1%, at most about 2.2%, at most about 2.3%, at most about 2.4%, at most about 2.5%, at most about 2.6%, at most about 2.7%, at most about 2.8%, at most about 2.9%, at most about 3%, at most about 3.1%, at most about 3.2%, at most about 3.3%, at most about 3.4%, at most about 3.5%, at most about 3.6%, at most about 3.7%, at most about 3.8%, at most about 3.9%, at most about 4%, at most about 5%, at most about 6%, at most about 7%, at most about 8%, at most about 9%, or at most about 10% non-Tmem cells, (e.g., where the non-Tmem cells are CD45RO−).

### Heterogenous Cell Component

According to some embodiments, a method of transplanting T-cell population into a human may comprise: administering a heterogenous cell population comprising lymphocytes, granulocytes and monocytes, wherein at least 30% of said lymphocytes comprises conventional T cells (Tcons) and administering a population of regulatory T cells (Tregs). In some cases, the heterogenous cell component and/or the population of Tregs comprise less than 5 EU/ml endotoxins.

In an exemplary embodiment, the method may comprise administering to a patient in need thereof, a population of hematopoietic stem and progenitor cells (HSPC's), administering a heterogenous cell population comprising lymphocytes, granulocytes and monocytes, wherein at least 30% of said lymphocytes comprises conventional T cells (Tcons), and administering a population of regulatory T cells (Tregs). In various embodiments, the populations of Tregs and HPSPC's may have varying amounts of micro-beads attached to each cell. For example, in some embodiments, the population of Tregs comprises less than 30,000 microbeads per cell and the heterogenous cell population comprises less than 1,000 microbeads per cell. In some embodiments, the population of HSPC's comprises less than 20,000 microbeads per cell.

In some embodiments, a heterogenous cell component may be administered to a subject. A heterogenous cell component may comprise many different cell types found in the peripheral blood of a human donor. A heterogenous cell component may comprise granulocytes, monocytes and lymphocytes. A heterogenous cell component may comprise T cells (such as Tcons, Tregs, Tmems, naïve T cells, CD4+ T cells, NK-T cells), B cells, NK cells, HSPC's, dendritic cells (such as plasmacytoid dendritic cells and myeloid dendritic cells) and other cell populations found in peripheral blood. A heterogenous cell component may be administered to a subject in addition to the other populations described herein. For instance, a heterogenous cell population may be administered with HSPC's as described herein. In some cases, a heterogenous cell population may be administered with HSPC's and Tregs as described herein. In some cases, a heterogenous cell population may be administered with Tregs as described herein. In some cases, a heterogenous cell population may be administered with Tcons as described herein. In some cases, a heterogenous cell population may be administered instead of the Tcon population as described herein.

In some embodiments, a heterogenous cell component administered to a subject may comprise a combination of granulocytes and monocytes. A combination of granulocytes and monocytes may comprise from 30% to 80% of the heterogenous cell component. At least 30% of the heterogenous cell component may comprise a combination of granulocytes and monocytes. At most 80% of the heterogenous cell component may comprise a combination of granulocytes and monocytes. In some cases, 30% to 40%, 30% to 50%, 30% to 60%, 30% to 70%, 30% to 80%, 40% to 50%, 40% to 60%, 40% to 70%, 40% to 80%, 50% to 60%, 50% to 70%, 50% to 80%, 60% to 70%, 60% to 80%, or 70% to 80% of the heterogenous cell component may comprise a combination of granulocytes and monocytes. In some cases, 30%, 40%, 50%, 60%, 70%, or 80% of the heterogenous cell component may comprise a combination of granulocytes and monocytes. In some cases, at least 30%, 40%, 50%, 60% or 70% of the heterogenous cell component may comprise a combination of granulocytes and monocytes. In some cases, at most 40%, 50%, 60%, 70%, or 80% of the heterogenous cell component may comprise a combination of granulocytes and monocytes.

In some embodiments, a heterogenous cell component administered to the subject may comprise lymphocytes. Lymphocytes comprise CD45+ cells. Lymphocytes may comprise from 8% to 50% of the heterogenous cell component. In some cases, at least 8% of the heterogenous cell component may comprise lymphocytes. In some cases, at most 50% of the heterogenous cell component may comprise lymphocytes. In some cases, 8% to 10%, 8% to 20%, 8% to 25%, 8% to 30%, 8% to 40%, 8% to 45%, 8% to 50%, 10% to 20%, 10% to 25%, 10% to 30%, 10% to 40%, 10% to 45%, 10% to 50%, 20% to 25%, 20% to 30%, 20% to 40%, 20% to 45%, 20% to 50%, 25% to 30%, 25% to 40%, 25% to 45%, 25% to 50%, 30% to 40%, 30% to 45%, 30% to 50%, 40% to 45%, 40% to 50%, or 45% to 50% of the heterogenous cell component may comprise lymphocytes. In some cases, 8%, 10%, 20%, 25%, 30%, 40%, 45%, or 50% of the heterogenous cell component may comprise lymphocytes. In some cases, at least 8%, 10%, 20%, 25%, 30%, 40% or 45% of the heterogenous cell component may comprise lymphocytes. In some cases, at most 10%, 20%, 25%, 30%, 40%, 45%, or 50% of the heterogenous cell component may comprise lymphocytes.

In some embodiments, lymphocytes in the heterogenous cell component may comprise Tcons. Tcons may comprise from 40% to 85% of the lymphocyte subset of the heterogenous cell component. In some cases, at least 40% of the lymphocyte subset may comprise Tcons. In some cases, at most 85% of the lymphocyte subset may comprise Tcons. In some cases, 40% to 50%, 40% to 60%, 40% to 65%, 40% to 70%, 40% to 75%, 40% to 80%, 40% to 85%, 50% to 60%, 50% to 65%, 50% to 70%, 50% to 75%, 50% to 80%, 50% to 85%, 60% to 65%, 60% to 70%, 60% to 75%, 60% to 80%, 60% to 85%, 65% to 70%, 65% to 75%, 65% to 80%, 65% to 85%, 70% to 75%, 70% to 80%, 70% to 85%, 75% to 80%, 75% to 85%, or 80% to 85% of the lymphocyte subset may comprise Tcons. In some cases, 40%, 50%, 60%, 65%, 70%, 75%, 80%, or 85% of the lymphocyte subset may comprise Tcons. In some cases, at least 40%, 50%, 60%, 65%, 70%, 75% or 80% of the lymphocyte subset may comprise Tcons. In some cases, at most 50%, 60%, 65%, 70%, 75%, 80%, or 85% of the lymphocyte subset may comprise Tcons.

In some embodiments, CD3+ lymphocytes in the heterogenous cell component may comprise CD4+ T cells. In some cases, 30% to 70% of the CD3+ lymphocyte subset may comprise CD4+ T cells. In some cases, at least 30% of the CD3+ lymphocyte subset may comprise CD4+ T cells. In some cases, at most 70% of the CD3+ lymphocyte subset may comprise CD4+ T cells. In some cases, 30% to 40%, 30% to 50%, 30% to 60%, 30% to 70%, 40% to 50%, 40% to 60%, 40% to 70%, 50% to 60%, 50% to 70%, or 60% to 70% of the CD3+ lymphocyte subset may comprise CD4+ T cells. In some cases, 30%, 40%, 50%, 60%, or 70% of the CD3+ lymphocyte subset may comprise CD4+ T cells. In some cases, at least 30%, 40%, 50% or 60% of the CD3+ lymphocyte subset may comprise CD4+ T cells. In some cases, at most 40%, 50%, 60%, or 70% of the CD3+ lymphocyte subset may comprise CD4+ T cells.

In some embodiments, CD3+ lymphocytes in the heterogenous cell component may comprise CD8+ T cells. In some cases, 20% to 65% of the CD3+ lymphocyte subset may comprise CD8+ T cells. In some cases, at least 20% of the CD3+ lymphocyte subset may comprise CD8+ T cells. In some cases, at most 65% of the CD3+ lymphocyte subset may comprise CD8+ T cells. In some cases, 20% to 30%, 20% to 40%, 20% to 50%, 20% to 60%, 20% to 65%, 30% to 40%, 30% to 50%, 30% to 60%, 30% to 65%, 40% to 50%, 40% to 60%, 40% to 65%, 50% to 60%, 50% to 65%, or 60% to 65% of the CD3+ lymphocyte subset may comprise CD8+ T cells. In some cases, 20%, 30%, 40%, 50%, 60%, or 65% of the CD3+ lymphocyte subset may comprise CD8+ T cells. In some cases, at least 20%, 30%, 40%, 50% or 60% of the CD3+ lymphocyte subset may comprise CD8+ T cells. In some cases, at most 30%, 40%, 50%, 60%, or 65% of the CD3+ lymphocyte subset may comprise CD8+ T cells.

In some embodiments, lymphocytes in the heterogenous cell component may comprise B cells. In some cases, 4% to 35% of the lymphocyte subset may comprise B cells. In some cases, at least 4% of the lymphocyte subset may comprise B cells. In some cases, at most 35% of the lymphocyte subset may comprise B cells. In some cases, 4% to 5%, 4% to 10%, 4% to 20%, 4% to 30%, 4% to 35%, 5% to 10%, 5% to 20%, 5% to 30%, 5% to 35%, 10% to 20%, 10% to 30%, 10% to 35%, 20% to 30%, 20% to 35%, or 30% to 35% of the lymphocyte subset may comprise B cells. In some cases, 4%, 5%, 10%, 20%, 30%, or 35% of the lymphocyte subset may comprise B cells. In some cases, at least 4%, 5%, 10%, 20% or 30% of the lymphocyte subset may comprise B cells. In some cases, at most 5%, 10%, 20%, 30%, or 35% of the lymphocyte subset may comprise B cells. B cells may be CD45+CD19+ or CD45+CD19+CD3− cells.

In some embodiments, lymphocytes in the heterogenous cell component may comprise NK cells. In some cases, 4% to 35% of the lymphocyte subset may comprise NK cells. In some cases, at least 4% of the lymphocyte subset may comprise NK cells. In some cases, at most 35% of the lymphocyte subset may comprise NK cells. In some cases, 4% to 5%, 4% to 10%, 4% to 20%, 4% to 30%, 4% to 35%, 5% to 10%, 5% to 20%, 5% to 30%, 5% to 35%, 10% to 20%, 10% to 30%, 10% to 35%, 20% to 30%, 20% to 35%, or 30% to 35% of the lymphocyte subset may comprise NK cells. In some cases, 4%, 5%, 10%, 20%, 30%, or 35% of the lymphocyte subset may comprise NK cells. In some cases, at least 4%, 5%, 10%, 20% or 30% of the lymphocyte subset may comprise NK cells. In some cases, at most 5%, 10%, 20%, 30%, or 35% of the lymphocyte subset may comprise NK cells. NK cells may be CD45+CD56+ or CD45+CD56+CD3− cells.

In some embodiments, CD3+ lymphocytes in the heterogenous cell component may comprise NK-T cells. In some cases, 3% to 30% of the CD3+ lymphocyte subset may comprise NK-T cells. In some cases, at least 4% of the CD3+ lymphocyte subset may comprise NK-T cells. In some cases, at most 35% of the CD3+ lymphocyte subset may comprise NK-T cells. In some cases, 3% to 5%, 3% to 10%, 3% to 20%, 3% to 30%, 5% to 10%, 5% to 20%, 5% to 30%, 10% to 20%, 10% to 30%, 20% to 30%, of the CD3+ lymphocyte subset may comprise NK-T cells. In some cases, 3%, 5%, 10%, 20% or 30% of the CD3+ lymphocyte subset may comprise NK-T cells. In some cases, at least 3%, 5%, 10% or 20% of the CD3+ lymphocyte subset may comprise NK-T cells. In some cases, at most 10%, 20% or 30% of the CD3+ lymphocyte subset may comprise NK-T cells. NK-T cells may be CD45+CD56+ or CD45+CD56+CD3+ cells.

In some embodiments, lymphocytes in the heterogenous cell component may comprise CD34+ cells. In some cases, 0.1% to 2% of the lymphocyte subset may comprise CD34+ cells. In some cases, at least 0.1% of the lymphocyte subset may comprise CD34+ cells. In some cases, at most 2% of the lymphocyte subset may comprise CD34+ cells. In some cases, 0.1% to 0.5%, 0.1% to 1%, 0.1% to 1.5%, 0.1% to 2%, 0.5% to 1%, 0.5% to 1.5%, 0.5% to 2%, 1% to 1.5%, 1% to 2%, or 1.5% to 2% of the lymphocyte subset may comprise CD34+ cells. In some cases, 0.1%, 0.5%, 1%, 1.5%, or 2% of the lymphocyte subset may comprise CD34+ cells. In some cases, at least 0.1%, 0.5%, 1% or 1.5% of the lymphocyte subset may comprise CD34+ cells. In some cases, at most 0.5%, 1%, 1.5%, or 2% of the lymphocyte subset may comprise CD34+ cells.

### D. Sequence and Timing of Administration of Cell Components

Disclosed herein are methods for enhanced allogeneic hematopoietic stem cell transplantation, comprising administering to a subject cell components that comprise populations of cells.

In some embodiments, cell components that comprise population of hematopoietic stem and progenitor cells (HSPC's), a population of cells comprising regulatory T cells (Tregs), and a population of conventional T cells (Tcons) are administered to a subject.

The population of HSPC's and the population of Tregs can be administered at the same or similar times, or at different times. In some embodiments, the population of HSPC's and the population of Tregs are administered on the same day.

In still another aspect, embodiments of the disclosure provide methods of treatments of a patient for one or more conditions using hematopoietic cell transplantation, where the method comprises administering to the patient a therapeutic composition comprising one or more cell populations described herein, e.g., a first populations of HSPC's or other CD34+ cell and a second population of T regs. The one or more conditions to be treated may include hematologic malignancy, non-malignant hematologic conditions and various autoimmune diseases. The doses of cell populations (e.g., total cells and number of cells per kg patient weight) can be adjusted for each patient and their particular condition. In specific embodiments, the dose can be adjusted by patient weight including for example kg patient weight or kg ideal patient weight the latter term known in the clinical and pharmaceutical arts. The cell populations making up a given therapeutic composition can be administered in selected time sequences e.g., concurrently or sequentially with one following another immediately or after selected intervals or after a particular event occurs or is achieved, e.g., patient hematocrit or blood cell count achieves a certain level. The time intervals may be selected so as to achieve or optimize a desired clinical outcome. In particular embodiments, the selected cell populations can be administered in a time sequence relative to the administration of HSPC's or other stem cell transplant. For example, in one or more embodiments, the Treg cells can be administered immediately or soon after the administration of the HSPC's. Further, populations of Tcons described herein can be administered one to two days after the Tregs. In use, these and related embodiments, prevent or minimize the patient's immune system from attacking the transplanted HSPC (or other organ) and the Tcons from attacking the patient's own tissue, resulting in a condition known as graft versus host disease (GVHD). This in turn, allows for reduced or minimal amounts of immunosuppressive agents to be given to the patient pre or post-transplant resulting in a reduced incidence or risk of relapse of their cancer and reduced incidence or risk of infection from an immune-suppressive drug regimen. The combination of these effects provides the benefit of improved clinical outcomes for the patient including one or more of a reduction in the occurrence or severity of GVHD (both acute and chronic forms), reduction in the occurrence or severity of cancer relapse and increased overall survival.

In some aspects, the invention provides a method of transplanting a conventional T cell (Tcon) population into a human subject without eliciting a stage 2 or higher graft versus host disease (GVHD) response up to 100 days after transplantation. The method may comprise: administering a population of conventional T cells (Tcons) and administering a population of regulatory T cells (Tregs). The population of Tcons may be administered at least 12 hours after the population of Tregs may be administered. The population of Tcons and the population of Tregs may comprise less than 5 EU/ml endotoxins.

In some aspects, the invention provides methods of transplanting a conventional T cell (Tcon) population into a human subject without eliciting a stage 2 or higher graft versus host disease (GVHD) response up to 100 days after transplanting. The method may comprise: administering a population of conventional T cells (Tcons) and administering a population of regulatory T cells (Tregs). The population of Tcons may be administered at least 12 hours after the population of Tregs may be administered. The population of Tcons and/or the population of Tregs may comprise less than 30,000 microbeads per cell.

In some embodiments, the population of Tcons may be administered at least 12 hours after the population of HSPC's. In some embodiments, the population of Tcons may be administered 24 to 96 hours after the population of HSPC's. In some embodiments, the population of Tcons may be administered 36 to 60 hours after the population of HSPC's. In some embodiments, the population of Tcons may be administered at least 12 hours after the population of cells may comprise Tregs. In some embodiments, the population of Tcons may be administered 24 to 96 hours after the population of cells may comprise Tregs. In some embodiments, the population of Tcons may be administered 36 to 60 hours after the population of cells may comprise Tregs

The population of HSPC's and the population of Tregs can administered at most about 1, 2, 3, 4,5, 6,7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours apart.

The population of Tcons can be administered to the subject after the population of HSPC's.

The population of Tcons can be administered to the subject at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73,74, 75, 76, 77, 78, 79, 80, 81, 82, 83, 84, 85, 86, 87, 88, 89, 90,91, 92, 93, 94, 95, or 96 hours after the population of HSPC's.

In some embodiments, the population of Tcons is administered to the subject at most about 6, 7, 8,9, 10, 11, 12, 13, 14, 15, 16, 17, 18, 19, 20, 21, 22,23, 24, 25, 26, 27,28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, or 120 hours after the population of HSPC's.

The population of Tcons can be administered to the subject, for example, between about 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the population of HSPC's.

The population of Tcons can be administered to the subject after the population of Tregs.

The population Tcons can be administered to the subject greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of Tregs.

In some embodiments, the population of Tcons is administered to the subject at most about 6, 7, 8,9, 10, 11, 12, 13, 14, 15, 16, 17, 18, 19, 20, 21, 22,23, 24, 25, 26, 27,28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of Tregs.

The population of Tcons can be administered to the subject, for example, between about 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the population of Tregs.

The population of Tcons can be administered to the subject after the population of HSPC's and the population of Tregs.

The population Tcons can administered to the subject, for example, greater than at least about 6, 7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of HSPC's and the population of Tregs.

In some embodiments, the population of Tcons is administered to the subject at most about 6, 7, 8,9, 10, 11, 12, 13, 14, 15, 16, 17, 18, 19, 20, 21, 22,23, 24, 25, 26, 27,28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of HSPC's and the population of Tregs.

The population of Tcons can be administered to the subject, for example, between about 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the population of HSPC's and the population of Tregs.

In some embodiments, a population of hematopoietic stem and progenitor cells (HSPC's), a population of cells comprising regulatory T cells (Tregs), a population of conventional T cells (Tcons), and a population of invariant natural killer T cells (iNKTs) are administered to a subject.

The population of iNKTs can be administered to the subject at the same time or at a similar time as the population of HSPC's. In some embodiments, the population of iNKTs is administered to the subject after the population of HSPC's.

The population of iNKTs can be administered to the subject greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of HSPC's.

In some embodiments, the population of iNKTs is administered to the subject at most about 6, 7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of HSPC's.

The population of iNKTs can be administered to the subject, for example, between about 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the population of HSPC's.

The population of iNKTs can be administered to the subject at the same time or at a similar time as the population of Tregs. In some embodiments, the population of iNKTs is administered to the subject after the population of Tregs.

A population of iNKTs can be administered to the subject greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of Tregs.

In some embodiments, the population of iNKTs is administered to the subject at most about 6, 7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of Tregs.

The population of iNKTs can be administered to the subject, for example, between about 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the population of Tregs.

In some embodiments, a population of hematopoietic stem and progenitor cells (HSPC's), a population of cells comprising regulatory T cells (Tregs), a population of conventional T cells (Tcons), and a population of memory T cells (Tmems) are administered to a subject.

A population of Tmems can be administered to the subject at the same time or at a similar time as the population of HSPC's. In some embodiments, the population of Tmems is administered to the subject after the population of HSPC's.

The population of Tmems can be administered to the subject greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of HSPC's.

In some embodiments, the population of Tmems is administered to the subject at most about 6, 7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of HSPC's.

The population of Tmems can be administered to the subject, for example, between about 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the population of HSPC's.

The population of Tmems can be administered to the subject at the same time or at a similar time as the population of Tregs. In some embodiments, the population of Tmems is administered to the subject after the population of Tregs.

The population of Tmems can be administered to the subject greater than at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of Tregs.

In some embodiments, the population of Tmems is administered to the subject at most about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours after the population of Tregs.

The population of Tmems can be administered to the subject, for example, between about 6-96, 12-84, 12-72, 12-66, 12-60, 12-54, 12-48, 12-42, 12-36, 12-30, 12-24, 12-18, 18-72, 18-66, 18-60, 18-54, 18-48, 18-42, 18-36, 18-30, 18-24, 24-72, 24-66, 24-60, 24-54, 24-48, 24-42, 24-36, 24-30, 30-72, 30-66, 30-60, 30-54, 30-48, 30-42, 30-36, 36-72, 36-66, 36-60, 36-54, 36-48, 36-42, 42-72, 42-66, 42-60, 42-54, 42-48, 48-72, 48-66, 48-60, 48-54, 54-72, 54-66, 54-60, 60-72, 60-66, or 66-72 hours after the population of Tregs.

### II. Subjects

Provided herein are compositions for administration to a recipient subject having a cancer, and methods of administering the same. The compositions and methods can be useful for treating or reducing cancer in the subject. In some embodiments, a population of conventional T cells (Tcons) is administered to the subject in order to elicit graft-versus-tumor (GVT) immune responses and with reduced graft versus host disease (GVHD).

In some embodiments, a subject is at least 13, at least 14, at least 15, at least 16, at least 17, at least 18, at least 19, at least 20, at least 21, at least 22, at least 23, at least 24, or at least 25 years of age. In some embodiments, a subject is at least 18 years of age. In some embodiments, a subject is at least 16 years of age. In some embodiments, a subject is at least 13 years of age.

In some embodiments, a subject is at most 50, at most 55, at most 60, at most 65, at most 70, at most 75, or at most 80 years of age. In some embodiments, a subject is at most 65 years of age. In some embodiments, a subject is at most 70 years of age.

### A. Conditions

Another aspect provides a method of treating a human subject diagnosed with a hematologic malignancy. The method comprises administering to the human subject a solution comprising the first population of CD45+ cells, a solution comprising the population of cells enriched for regulatory T cells (Tregs), a solution comprising the second population of CD45+ cells, and a solution comprising one or more doses of the GVHD prophylactic agent (e.g., tacrolimus). In this aspect, the solution comprising the first population of CD45+ cells, the solution comprising the population of cells enriched for regulatory Tregs, the solution comprising the second population of CD45+ cells, and the solution comprising one or more doses of the GVHD prophylactic agent are as defined according to any herein disclosed multi-component pharmaceutical treatment.

A further aspect provides a method of transplanting a conventional T cell (Tcons) population as a part of a treatment regimen for a hematologic malignancy in which the method reduces a risk and/or severity of an adverse event associated with the treatment regimen. The method comprises administering to the patient a population of regulatory T cells (Tregs) comprising Tregs and a liquid suspending the Tregs; administering to the patient a heterogenous cell population comprising lymphocytes, granulocytes, monocytes and a liquid suspending said cells. In this aspect, at least about 30% of said lymphocytes comprise Tcons. and after administration of the cell populations, the patient has a reduced risk and/or severity of the adverse event as compared to hematologic malignancy patients who received Tcons but did not receive Tregs.

A yet further aspect provides a method of transplanting cell populations into a human patient as a part of a treatment regimen for a hematologic malignancy in which the method reduces a risk and/or severity of an adverse event associated with the treatment regimen. The method comprises providing a population of hematopoietic stem and progenitor cells (HSPCs) to be administered to the patient; the population of HSPCs comprising HSPCs and a liquid suspending the HSPCs; providing a population of regulatory T cells (Tregs) to be administered to the patient, the population of Tregs comprising Tregs and a liquid suspending the Tregs; and providing a heterogenous cell population to be administered to the patient, the heterogenous cell population comprising lymphocytes, granulocytes, monocytes and a liquid suspending said cells. In this aspect, at least about 30% of said lymphocyte comprise conventional T cells (Tcons) and after administration of the cell populations, the patient has a reduced risk and/or severity of the adverse event as compared to hematologic malignancy patients who received a Tcon cell population but did not receive a T-reg cell population.

Another aspect provides a method of transplanting cell populations into a human patient as a part of a treatment regimen for a hematologic malignancy. The method comprises administering to the patient a population of hematopoietic stem and progenitor cells (HSPCs; the population of HSPCs comprising HSPCs and a liquid suspending the HSPCs; administering to the patient a population of regulatory T cells (Tregs) to be administered to the patient, the population of Tregs comprising Tregs and a liquid suspending the Tregs; and administering to the patient a heterogenous cell population to be administered to the patient, the heterogenous cell population comprising lymphocytes, granulocytes, monocytes and a liquid suspending said cells, wherein at least about 30% of said lymphocyte comprise conventional T cells (Tcons); and administering to the patient over a period of time up to about 180 days a single graft versus host disease (GVHD) prophylactic agent (GVHDPA) comprising tacrolimus (tacrolimus GHVDPA), wherein the tacrolimus GHVDPA is administered to maintain a concentration of tacrolimus in the patient's blood above a threshold level during the period of time; and wherein a risk and/or severity of GHVD associated with the treatment regimen for the hematologic malignancy is significantly reduced.

The methods of the disclosure can be used for treating a subject (e.g., a human subject) with a cancer. In some embodiments, the subject has been treated for cancer, e.g. by treatment with a chemotherapeutic drug or with radiation. In some embodiments, the human subject may be treated for cancer prior to the administration of a cell population. The methods of the disclosure can be useful for treating a hematologic malignancy, for example, leukemia or lymphoma. Examples of hematologic malignancies that can be treated by the methods of the disclosure include, but are not limited to, acute lymphocytic leukemia (ALL), acute myelogenous leukemia (AML), chronic myelogenous leukemia (CML), multiple myeloma, and lymphomas such as Hodgkin and non-Hodgkin lymphomas. A cancer can be a solid tumor. In some embodiments, the cancer is a primary or metastatic tumor.

The types of cancer that can be treated using the methods of the present disclosure include but are not limited to leukemia, lymphoma, adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and pharyngeal cancer, acute lymphocytic leukemia, acute myeloid leukemia, children's leukemia, chronic lymphocytic leukemia, chronic myeloid leukemia, liver cancer, lung cancer, lung carcinoid tumors, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, nonmelanoma skin cancers, stomach cancer, testicular cancer; thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, and Waldenstrom's macroglobulinemia.

Patients with high-risk hematologic malignancies are rarely cured with standard chemotherapy. High-risk malignancies include, for example, leukemia or lymphoma that has progressed beyond first remission, or leukemia or lymphoma with refractory relapse.

A subject that receives a composition of the disclosure can have, for example, acute myeloid leukemia, acute lymphoid leukemia, mixed phenotype leukemia, myelofibrosis, high-risk myelodysplastic syndrome, very high-risk myelodysplastic syndrome, myelofibrosis (MF) that is eligible for transplant per National Comprehensive Cancer Network Guidelines, intermediate-2- or high-risk MF according to the IPSS, DIPSS or DIPSS-plus scoring systems, intermediate-1-risk MF associated with high-risk features such as high symptoms burden, low platelet counts, or complex cytogenetics, primary myelofibrosis, myelofibrosis evolved from another myeloproliferative neoplasm, myelodysplastic syndrome, non-Hodgkin lymphoma, a non-malignant indication for allogeneic hematopoietic stem cell transplantation (alloHCT) such as sickle cell anemia.

In various embodiments, embodiments of the therapeutic compositions can be administered to patient who have the following diseases or conditions. In some embodiments, a subject has acute myeloid leukemia. In some embodiments, a subject has acute lymphoid leukemia. In some embodiments, a subject has mixed phenotype leukemia. In some embodiments, a subject has high-risk myelodysplastic syndrome. In some embodiments, a subject has very high-risk myelodysplastic syndrome. In some embodiments, a subject has myelofibrosis (MF) that is eligible for transplant per National Comprehensive Cancer Network Guidelines. In some embodiments, a subject has intermediate-2- or high-risk myelofibrosis according to the IPSS, DIPSS or DIPSS-plus scoring systems. In some embodiments, a subject has intermediate-1-risk myelofibrosis associated with high-risk features such as high symptoms burden, low platelet counts, or complex cytogenetics. In some embodiments, a subject has primary myelofibrosis. In some embodiments, a subject has myelofibrosis. In some embodiments, a subject has myelofibrosis evolved from another myeloproliferative neoplasm. In some embodiments, a subject has myelodysplastic syndrome. In some embodiments, a subject has non-Hodgkin lymphoma. In some embodiments, a subject has a non-malignant indication for alloHCT.

In some embodiments, the administering enhances cancer remission in the human subject as compared to the human subject prior to the administering. In various embodiments, embodiments of the therapeutic compositions can be administered to patient who are in the following states of remission and/or disease. A subject can be in complete remission (CR). A subject can be in complete remission with incomplete hematologic recovery (CRi), e.g., without the presence of known minimal residual disease. A subject can have minimal residual disease. A subject can have no evidence of minimal residual disease. A subject can have active disease. A subject can have a leukemia (e.g., acute myeloid, acute lymphoid, or mixed phenotype) that is not in morphologic CR with bone marrow infiltration by leukemic blasts of ≤10%. A subject can have a leukemia (e.g., acute myeloid, acute lymphoid, or mixed phenotype) that is in morphologic CR with evidence of minimal residual positivity by either multiparameter flow cytometric analysis or by a nucleic acid-based technique.

Complete remission (CR) for acute myeloid, lymphoid or mixed phenotype leukemia can be indicated by meeting all of the following criteria: (i) Bone marrow blasts≤5%; (ii) Absence of circulating blasts and blasts with Auer rods; (ii) Absence of extramedullary disease 4. ANC ≥1.0×10⁹/L (1,000/μL); (iii) Platelet count ≥100×10⁹/L (100,000/μL); and (iv) Independence of red cell transfusions. Complete Response with Incomplete Hematologic Recovery (CRi) can be indicated by meeting all the CR criteria except for residual neutropenia (<1.0×10⁹/L) or thrombocytopenia (<100×10⁹/L).

### B. Sensitivities

In some embodiments, a subject does not have a known allergy or hypersensitivity to, or intolerance of, tacrolimus. In some embodiments, a subject does not have a known allergy or hypersensitivity to, or intolerance of, sirolimus.

In some embodiments, subjects are not sensitive to iron dextran (e.g., subjects with sensitivity to iron dextran are not eligible to receive a composition of the disclosure. In some cases, this may be because of the magnetic beads used in some embodiments to isolate, deplete, and/or purify cell types).

In some embodiments, subjects are not sensitive to products derived from cyanine dyes (e.g., subjects with sensitivity to products derived from cyanine dyes are not eligible to receive a composition of the disclosure).

In some embodiments, subjects are not sensitive to proteins products derived from murine sources (e.g., subjects with sensitivity to proteins products derived from murine sources are not eligible to receive a composition of the disclosure).

In some embodiments, subjects are not sensitive to proteins products derived from bovine sources (e.g., subjects with sensitivity to proteins products derived from bovine sources are not eligible to receive a composition of the disclosure).

In some embodiments, subjects are not sensitive to proteins products derived from algal sources (e.g., subjects with sensitivity to proteins products derived from algal sources are not eligible to receive a composition of the disclosure).

In some embodiments, subjects are not sensitive to proteins products derived from Streptomyces avidinii (e.g., subjects with sensitivity to proteins products derived from Streptomyces avidinii are not eligible to receive a composition of the disclosure).

### C. Organ Function and Biomarkers

A subject can have an estimated glomerular filtration rate (eGFR) >30 mL/minute. A subject can have an estimated glomerular filtration rate (eGFR) >40 mL/minute. A subject can have an eGCF of >50 mL/minute. A subject can have an estimated glomerular filtration rate (eGFR) >60 mL/minute.

A subject can have a cardiac ejection fraction at rest ≥45%, or shortening fraction of ≥27% by echocardiogram or radionuclide scan (MUGA).

A subject can have a diffusing capacity of the lung for carbon monoxide (DLCO) (adjusted for hemoglobin) of ≥50%.

A subject can have a negative serum or urine beta-HCG test, e.g., in females of childbearing potential within 3 weeks of registration.

A subject can have total bilirubin <2 times upper limit of normal (ULN).

A subject can have Gilbert's syndrome, wherein hemolysis has been excluded.

A subject can have an ALT reading within 3 times upper limit of normal (ULN). A subject can have an AST reading within 3 times upper limit of normal (ULN).

### D. Additional Therapies and Other Subject Characteristics

In some embodiments, a subject has not received a prior alloHCT. In some embodiments, a subject is not a candidate for autologous transplant. In some embodiments, a subject is not receiving corticosteroids or other immunosuppressive therapy. In some embodiments, a subject is receiving topical corticosteroids or oral systemic corticosteroid doses less than or equal to 10 mg/day. In some embodiments, a subject does not receive donor lymphocyte infusion (DLI). In some embodiments, a subject does not receive a T cell depleting pharmaceutical, e.g., post-transplant cyclophosphamide (Cy), peri-transplant anti-thymocyte globulin (ATG), or alemtuzumab. In some embodiments, a subject that has previously been exposed to a T cell-depleting agent has a 5 half-life washout of the agent prior to planned transplant day 0 (day of infusion of the cell components of the graft). In some embodiments, a subject is not positive for anti-donor HLA antibodies against a mismatched allele in the selected donor as determined by either: (a) a positive crossmatch test of any titer; or (b) the presence of anti-donor HLA antibody to any HLA locus. In some embodiments, the subject has a Karnofsky performance score ≥70%. In some embodiments, a subject does not have a hematopoietic cell transplantation-specific Comorbidity Index (HCT-CI) of >4. In some embodiments, a subject does not have an uncontrolled bacterial, viral or fungal infection. In some embodiments, a subject is not taking antimicrobial therapy and with progression or no clinical improvement in infection. In some embodiments, a subject is not seropositive for HIV-1 or -2, HTLV-1 or -2, Hepatitis B sAg, or Hepatitis C antibody. In some embodiments, a subject does not have an uncontrolled autoimmune disease that requires active immunosuppressive treatment. In some embodiments, a subject does has not had concurrent malignancies or active disease within 1 year, for example, excluding non-melanoma skin cancers that have been curatively resected. In some embodiments, a subject does not exhibit psychosocial circumstances that preclude the patient being able to go through transplant or participate responsibly in follow up care. In some embodiments, a subject is not pregnant or breastfeeding. In some embodiments, a subject does not have a serious medical condition or abnormality in clinical laboratory tests that, in the medical professional's judgment, precludes the subject's safety upon receipt of a composition of the disclosure. In some embodiments, a subject is eligible for myeloablative alloHCT.

In some embodiments, a subject receives a prophylactic agent to reduce the risk of bacterial, fungal, and/or viral infection, e.g., during the peri-transplant period.

In some embodiments, a subject receives a supportive therapy for alloHCT-related toxicity. In some embodiments, a subject does not receive a supportive therapy for alloHCT-related toxicity. In some embodiments, a subject receives a growth factor. In some embodiments, a subject does not receive a growth factor. In some embodiments, a subject receives intravenous immunoglobulin. In some embodiments, a subject does not receive intravenous immunoglobulin. In some embodiments, a subject receives an analgesic. In some embodiments, a subject does not receive an analgesic. In some embodiments, a subject receives an anti-emetic. In some embodiments, a subject does not receive an anti-emetic. In some embodiments, a subject receives electrolyte replacement. In some embodiments, a subject does not receive electrolyte replacement. In some embodiments, a subject receives a tyrosine kinase inhibitor (e.g., a FLT3 inhibitor). In some embodiments, a subject does not receive a tyrosine kinase inhibitor (e.g., a FLT3 inhibitor). In some embodiments, a subject receives prednisone or an equivalent thereof, e.g., at a dose of ≤10 mg/day. In some embodiments, a subject does not receive prednisone or an equivalent thereof. In some embodiments, a subject receives corticosteroid treatment to manage GVHD. In some embodiments, a subject does not receive corticosteroid treatment to manage GVHD. In some embodiments, a subject receives high-dose corticosteroid treatment to manage GVHD. In some embodiments, a subject does not receive high-dose corticosteroid treatment to manage GVHD. In some embodiments, a subject receives corticosteroid treatment to manage, for example, adrenal insufficiency, hypersensitivity reactions, or other non-cancer-related symptoms including premedication for known hypersensitivity reactions to contrast for scans. In some embodiments, a subject does not receive corticosteroid treatment. In some embodiments, a subject receives an immunosuppressive medication. In some embodiments, a subject does not receive an immunosuppressive medication. In some embodiments, a subject receives a donor lymphocyte infusion. In some embodiments, a subject does not receive a donor lymphocyte infusion.

### E. Conditioning Regimen

Conditioning regimens can be used as part of an alloHCT regimen of the disclosure. Chemotherapy and/or irradiation given soon before a transplant is called a conditioning regimen. Conditioning regimens can help eradicate a patient's disease prior to the infusion of HSPCs, suppress immune reactions, and allow a donor HSPCs to reconstitute the vacant hematopoietic compartment that results from the conditioning regimen. In some embodiments of the methods of the disclosure, a subject can be treated with myeloablative conditioning prior to infusion of cell populations described herein. In some embodiments of the methods of the disclosure, a subject can be treated with myeloreductive conditioning prior to infusion of cell populations described herein. In some embodiments of the methods of the disclosure, a subject can be treated with a reduced intensity myeloablative conditioning prior to infusion of cell populations described herein. In some embodiments of the methods of the disclosure, a subject can be treated with non-myeloablative conditioning prior to administering a cell population or cell populations described herein.

As used herein, the term conditioning regimen and the like applies to myeloablative conditioning, myeloreductive conditioning, reduced intensity myeloablative therapy/conditioning, and/or non-myeloablative conditioning. As used herein, the term myeloablative therapy/conditioning also includes myeloreductive conditioning and reduced intensity myeloablative conditioning.

In aspects and embodiments, a treatment and/or method further comprises a conditioning regimen, wherein the conditioning regimen is administered before administration of one of (a) a solution comprising a first population of CD45+ cells comprising hematopoietic stem and progenitor cells (HSPCs) and granulocytes wherein at most about 10% of the first population of CD45+ cells comprise granulocytes; (b) a solution comprising a population of cells enriched for regulatory T cells (Tregs); and (c) a solution comprising a second population of CD45+ cells wherein the second population of CD45+ cells comprise at least about 20% CD3+ conventional T cells (Tcons), at least about 10% monocytes, and at least about 10% granulocytes; and (d) a solution comprising one or more doses of a graft vs host disease (GVHD) prophylactic agent. In embodiments, the conditioning regimen is a myeloablative conditioning regimen. In some cases, the conditioning regimen comprises at least three conditioning reagents, wherein at least one conditioning reagent is thiotepa. In various embodiments, the myeloablative conditioning regimen comprises at least one dose of thiotepa, e.g., at least about 5 milligrams thiotepa per kilogram of the human subject's actual or ideal body weight or at least about 10 milligrams thiotepa per kilogram of the human subject's actual or ideal body weight. In some embodiments, the conditioning regimen comprises one or more doses of busulfan, fludarabine and thiotepa. In embodiments, the one or more doses comprises from about 5 to about 12 mg of thiotepa per kg human subject's actual or ideal body weight, from about 7 to about 11 mg of busulfan per kg human subject actual or ideal body weight, and from about 100 to about 200 mg of fludarabine per meter² body surface area respectively.

In various embodiments, the method further comprises administering a myeloablative conditioning regimen to the patient prior to the administration of any cell population, the conditioning regimen comprising administration of at least one conditioning agent to the patient.

In some embodiments, the patient does not receive any irradiation as part of the myeloablative conditioning regimen.

In embodiments, the at least one conditioning agent is administered from about two to about ten days prior to the administration of any of the cell populations. In some cases, the at least one conditioning agent is administered about five days prior to the administration of any of the cell populations.

In various embodiments, the human subject has undergone myeloablative conditioning regimen before administration of any cell populations and the adverse event is associated with the myeloablative conditioning.

In some embodiments, the at least one conditioning agent comprises thiotepa. In some cases, a dose of thiotepa administered to the patient is in a range of from about 5 to about 10 mg per kilogram of actual or ideal body weight.

In embodiments, the at least one conditioning agent comprises busulfan and fludarabine. In some cases, doses of thiotepa, busulfan, and fludarabine administered to the patient comprise about 10 mg per kilogram of the patient's actual or ideal body weight, about 9.6 mg per kilogram of the patient's actual or ideal body weight, and about 150 mg per meter² body surface area respectively.

In some embodiments, the subject has been conditioned with radiation, chemotherapy, recombinant proteins, antibodies, or toxin-conjugated antibodies, or any combination thereof prior to administering a cell population or cell populations described herein. In some embodiments, the subject is conditioned for cellular graft therapy by first treating the subject with myeloablative therapy. Exemplary myeloablative therapies include chemotherapy or radiotherapy. Myleoablative therapies are thought to provide therapeutic benefit by debulking a tumor and/or reducing the number of cancer cells. Myeloablative regimens eradicate a sufficient number of HSCs that the patient would otherwise increase the chances of a patient developing GVHD. When HSPCs are subsequently administered to the myeloablated subject, the donor cells can further attack the cancer and/or and reconstitute the blood and the immune system of the subject.

In some embodiments, the myeloablative therapy comprises administration of thiotepa (TTP), busulfan, cyclophosphamide, Total Body Irradiation (TBI), fludarabine, etoposide, or any combination thereof. In some embodiments, the myeloablative therapy comprises administration an anti-cKIT antibody. In some embodiments, the myeloablative therapy comprises administration an antibody drug conjugate. The antibody drug conjugate can be, for example, anti-CD45-saporin or anti-cKit-saporin therapeutic antibodies. In some embodiments, the myeloablative therapy is a reduced intensity conditioning therapy. Exemplary conditioning regimens are described in Table 15.

A conditioning regimen of this disclosure may comprise one or more doses of busulfan. A conditioning regimen of this disclosure may comprise fludarabine. A conditioning regimen of this disclosure may comprise one or more doses of Cyclophosphamide. A conditioning regimen of this disclosure may comprise one or more doses of Melphalan. A conditioning regimen of this disclosure may comprise one or more doses of Etoposide.

The methods of the disclosure can comprise administration of a combination of conditioning reagents prior to the administration of the cells. A conditioning regimen as described herein may comprise administering 1, 2, 3 or 4 different conditioning reagents. The conditioning reagents used herein may be alkylating agents. The conditioning reagents used herein may be myeloablative. The conditioning reagents used herein may be non-myeloablative. The conditioning reagents used herein may be myeloreductive. The conditioning reagents used herein may be a form of chemotherapy.

The conditioning regimen described herein may comprise administration of an alkylating agent such as thiotepa (TTP). A conditioning regimen of this disclosure comprising TTP may comprise at least one more conditioning reagent. The conditioning reagents administered to a subject in addition to TTP may comprise one or more reagents selected from busulfan, dimethyl myleran, prednisone, methyl prednisolone, azathioprine, cyclophosphamide, cyclosparine, monoclonal antibodies against T cells, antilymphocyte globulin and anti-thymocyte globulin, fludarabine, etoposide, radiation, total body irradiation (TBI). Aspects and embodiments include any combination of TTP with the one or more conditioning reagents. In some embodiments, a subject is administered a conditioning regimen comprising thiotepa, busulfan, and fludarabine. In some embodiments, a subject is administered a conditioning regimen comprising thiotepa, fludarabine, and TBI (e.g., HFTBI).

The conditioning regimen described herein may comprise administration of an alkylating agent such as TTP. In some cases, a conditioning regimen of the disclosure may comprise TTP administration on more than one day. A conditioning regimen of the disclosure may comprise administering 2 mg/kg to 14 mg/kg TTP to a subject. A conditioning regimen of this disclosure may comprise administering at least 3 mg/kg TTP to a subject. A conditioning regimen of this disclosure may comprise administering at most 14 mg/kg TTP to a subject. A conditioning regimen of this disclosure may comprise administering 2 mg/kg to 5 mg/kg, 2 mg/kg to 6 mg/kg, 2 mg/kg to 8 mg/kg, 2 mg/kg to 10 mg/kg, 2 mg/kg to 12 mg/kg, 2 mg/kg to 14 mg/kg, 5 mg/kg to 6 mg/kg, 5 mg/kg to 8 mg/kg, 5 mg/kg to 10 mg/kg, 5 mg/kg to 12 mg/kg, 5 mg/kg to 14 mg/kg, 6 mg/kg to 8 mg/kg, 6 mg/kg to 10 mg/kg, 6 mg/kg to 12 mg/kg, 6 mg/kg to 14 mg/kg, 8 mg/kg to 10 mg/kg, 8 mg/kg to 12 mg/kg, 8 mg/kg to 14 mg/kg, 10 mg/kg to 12 mg/kg, 10 mg/kg to 14 mg/kg, or 12 mg/kg to 14 mg/kg TTP to a subject. A conditioning regimen of this disclosure may comprise administering 2 mg/kg, 3 mg/kg, 4 mg/kg, 5 mg/kg, 6 mg/kg, 8 mg/kg, 10 mg/kg, 12 mg/kg, or 14 mg/kg TTP to a subject. A conditioning regimen of this disclosure may comprise administering at most 2 mg/kg, 3 mg/kg, 4 mg/kg, 5 mg/kg, 6 mg/kg, 8 mg/kg, 10 mg/kg or 12 mg/kg TTP to a subject. A conditioning regimen of this disclosure may comprise administering at least 2 mg/kg, 3 mg/kg, 4 mg/kg, 5 mg/kg, 6 mg/kg, 8 mg/kg, 10 mg/kg, 12 mg/kg, or 14 mg/kg TTP to a subject.

As used herein, a recited dose, e.g., # mg/kg, may be relative to a subject's actual body weight (in kg) or relative to the subject's ideal body weight (in kg). Or the recited dose may be relative to a subject's adjusted body weight (ABW) if the subject's actual body weight is greater than 120% of the ideal body weight (IBW).

A subject administered one or more cell populations described herein may be administered one or more doses of TTP prior to the cell transplant. A subject receiving one or more cell populations described herein may be administered 1, 2, 3, 4, 5, 6, 7, 8, 9 or 10 doses of TTP prior to the cell transplant. In some cases, each dose of TTP has the same concentration. In some cases, one or more doses of TTP have different concentrations. A subject may be administered 1 mg/kg to 10 mg/kg TTP in a single dose. A subject may be administered at least 1 mg/kg TTP in a single dose. A subject may be administered at most 10 mg/kg TTP in a single dose. A subject may be administered 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 1 mg/kg to 10 mg/kg, 2 mg/kg to 3 mg/kg, 2 mg/kg to 4 mg/kg, 2 mg/kg to 5 mg/kg, 2 mg/kg to 6 mg/kg, 2 mg/kg to 7 mg/kg, 2 mg/kg to 8 mg/kg, 2 mg/kg to 9 mg/kg, 2 mg/kg to 10 mg/kg, 3 mg/kg to 4 mg/kg, 3 mg/kg to 5 mg/kg, 3 mg/kg to 6 mg/kg, 3 mg/kg to 7 mg/kg, 3 mg/kg to 8 mg/kg, 3 mg/kg to 9 mg/kg, 3 mg/kg to 10 mg/kg, 4 mg/kg to 5 mg/kg, 4 mg/kg to 6 mg/kg, 4 mg/kg to 7 mg/kg, 4 mg/kg to 8 mg/kg, 4 mg/kg to 9 mg/kg, 4 mg/kg to 10 mg/kg, 5 mg/kg to 6 mg/kg, 5 mg/kg to 7 mg/kg, 5 mg/kg to 8 mg/kg, 5 mg/kg to 9 mg/kg, 5 mg/kg to 10 mg/kg, 6 mg/kg to 7 mg/kg, 6 mg/kg to 8 mg/kg, 6 mg/kg to 9 mg/kg, 6 mg/kg to 10 mg/kg, 7 mg/kg to 8 mg/kg, 7 mg/kg to 9 mg/kg, 7 mg/kg to 10 mg/kg, 8 mg/kg to 9 mg/kg, 8 mg/kg to 10 mg/kg, or 9 mg/kg to 10 mg/kg TTP in a single dose. A subject may be administered 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg TTP in a single dose. A subject may be administered at most 1 mg/kg, 2 mg/kg, 3 mg/kg, 4 mg/kg, 5 mg/kg, 6 mg/kg, 7 mg/kg, 8 mg/kg, 9 mg/kg or 10 mg/kg TTP in a single dose. A subject may be administered at least 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg TTP in a single dose.

The methods of the disclosure can comprise administration of a combination of conditioning reagents prior to the administration of the cells. A conditioning regimen as described herein may comprise administering 1, 2, 3 or 4 different conditioning reagents. The conditioning reagents used herein may be alkylating agents. The conditioning reagents used herein may be myeloablative. The conditioning reagents used herein may be non-myeloablative. The conditioning reagents used herein may be myeloreductive. The conditioning reagents used herein may be a form of chemotherapy.

A conditioning regimen of this disclosure may comprise one or more doses of busulfan. One or more doses of busulfan may be administered to a subject before the administration of one or more doses of another conditioning reagent such as TTP. One or more doses of busulfan may be administered to a subject after the administration of one or more doses of another conditioning reagent such as TTP. One or more doses of busulfan may be administered to a subject along with the administration of one or more doses of another conditioning reagent such as TTP.

A conditioning regimen of this disclosure may comprise administering about 6 mg/kg to about 12 mg/kg busulfan to a subject. A conditioning regimen of this disclosure may comprise administering at least about 6 mg/kg busulfan to a subject. A conditioning regimen of this disclosure may comprise administering at most about 12 mg/kg busulfan to a subject. A conditioning regimen of this disclosure may comprise administering about 6 mg/kg to about 7 mg/kg, about 6 mg/kg to about 8 mg/kg, about 6 mg/kg to about 9 mg/kg, about 6 mg/kg to about 10 mg/kg, about 6 mg/kg to about 11 mg/kg, about 6 mg/kg to about 12 mg/kg, about 7 mg/kg to about 8 mg/kg, about 7 mg/kg to about 9 mg/kg, about 7 mg/kg to about 10 mg/kg, about 7 mg/kg to about 11 mg/kg, about 7 mg/kg to about 12 mg/kg, about 8 mg/kg to about 9 mg/kg, about 8 mg/kg to about 10 mg/kg, about 8 mg/kg to about 11 mg/kg, about 8 mg/kg to about 12 mg/kg, about 9 mg/kg to about 10 mg/kg, about 9 mg/kg to about 11 mg/kg, about 9 mg/kg to about 12 mg/kg, about 10 mg/kg to about 11 mg/kg, about 10 mg/kg to about 12 mg/kg, or about 11 mg/kg to about 12 mg/kg busulfan to a subject. A conditioning regimen of this disclosure may comprise administering 6 mg/kg, 7 mg/kg, 8 mg/kg, 9 mg/kg, 10 mg/kg, 11 mg/kg, or 12 mg/kg busulfan to a subject. A conditioning regimen of this disclosure may comprise administering at least 6 mg/kg, 7 mg/kg, 8 mg/kg, 9 mg/kg, 10 mg/kg or 11 mg/kg busulfan to a subject. A conditioning regimen of this disclosure may comprise administering at most 7 mg/kg, 8 mg/kg, 9 mg/kg, 10 mg/kg, 11 mg/kg, or 12 mg/kg busulfan to a subject.

A subject receiving one or more cell populations described herein may be administered one or more doses of busulfan prior to the cell transplant. A subject receiving one or more cell components described herein may be administered 1, 2, 3, 4, 5, 6, 7, 8, 9 or 10 doses of busulfan prior to the cell transplant. In some cases, each dose of busulfan has the same concentration. In some cases, one or more doses of busulfan have different concentrations. A subject may be administered 1 mg/kg to 10 mg/kg busulfan in a single dose. A subject may be administered at least 1 mg/kg busulfan in a single dose. A subject may be administered at least 2 mg/kg busulfan in a single dose. A subject may be administered at least 3 mg/kg busulfan in a single dose.

A conditioning regimen of this disclosure may comprise one or more doses of fludarabine. One or more doses of fludarabine may be administered to a subject before the administration of one or more doses of another conditioning reagent such as TTP. One or more doses of fludarabine may be administered to a subject after the administration of one or more doses of another conditioning reagent such as TTP. One or more doses of fludarabine may be administered to a subject along with the administration of one or more doses of another conditioning reagent such as TTP.

A conditioning regimen of this disclosure may comprise administering 20 mg/m² to 180 mg/m² fludarabine to a subject based on the surface area of the subject. A conditioning regimen of this disclosure may comprise administering at least 20 mg/m² fludarabine to a subject. A conditioning regimen of this disclosure may comprise administering at most 180 mg/m² fludarabine to a subject. A conditioning regimen of this disclosure may comprise administering 20 mg/m² to 30 mg/m², 20 mg/m² to 40 mg/m², 20 mg/m² to 50 mg/m², 20 mg/m² to 60 mg/m², 20 mg/m² to 80 mg/m², 20 mg/m² to 100 mg/m², 20 mg/m² to 120 mg/m², 20 mg/m² to 150 mg/m², 20 mg/m² to 180 mg/m², 30 mg/m² to 40 mg/m², 30 mg/m² to 50 mg/m², 30 mg/m² to 60 mg/m², 30 mg/m² to 80 mg/m², 30 mg/m² to 100 mg/m², 30 mg/m² to 120 mg/m², 30 mg/m² to 150 mg/m², 30 mg/m² to 180 mg/m², 40 mg/m² to 50 mg/m², 40 mg/m² to 60 mg/m², 40 mg/m² to 80 mg/m², 40 mg/m² to 100 mg/m², 40 mg/m² to 120 mg/m², 40 mg/m² to 150 mg/m², 40 mg/m² to 180 mg/m², 50 mg/m² to 60 mg/m², 50 mg/m² to 80 mg/m², 50 mg/m² to 100 mg/m², 50 mg/m² to 120 mg/m², 50 mg/m² to 150 mg/m², 50 mg/m² to 180 mg/m², 60 mg/m² to 80 mg/m², 60 mg/m² to 100 mg/m², 60 mg/m² to 120 mg/m², 60 mg/m² to 150 mg/m², 60 mg/m² to 180 mg/m², 80 mg/m² to 100 mg/m², 80 mg/m² to 120 mg/m², 80 mg/m² to 150 mg/m², 80 mg/m² to 180 mg/m², 100 mg/m² to 120 mg/m², 100 mg/m² to 150 mg/m², 100 mg/m² to 180 mg/m², 120 mg/m² to 150 mg/m², 120 mg/m² to 180 mg/m², or 150 mg/m² to 180 mg/m² fludarabine to a subject. A conditioning regimen of this disclosure may comprise administering 20 mg/m², 30 mg/m², 40 mg/m², 50 mg/m², 60 mg/m², 80 mg/m², 100 mg/m², 120 mg/m², 150 mg/m², or 180 mg/m² fludarabine to a subject. A conditioning regimen of this disclosure may comprise administering at least 20 mg/m², 30 mg/m², 40 mg/m², 50 mg/m², 60 mg/m², 80 mg/m², 100 mg/m², 120 mg/m² or 150 mg/m² fludarabine to a subject. A conditioning regimen of this disclosure may comprise administering at most 30 mg/m², 40 mg/m², 50 mg/m², 60 mg/m², 80 mg/m², 100 mg/m², 120 mg/m², 150 mg/m², or 180 mg/m² fludarabine to a subject.

A subject receiving one or more cell components described herein may be administered one or more doses of fludarabine prior to the cell transplant. A subject receiving one or more cell components described herein may be administered 1, 2, 3, 4, 5, 6, 7, 8, 9 or 10 doses of fludarabine prior to the cell transplant. In some cases, each dose of fludarabine has the same concentration. In some cases, one or more doses of fludarabine have different concentrations. A subject may be administered 20 to 60 mg/m² dose of fludarabine in a single dose. A subject may be administered at least 30 mg/m² fludarabine in a single dose. A subject may be administered at least 40 mg/m² fludarabine in a single dose. A subject may be administered at least 50 mg/m² fludarabine in a single dose.

A conditioning regimen of this disclosure may comprise administering 20 mg/m² to 180 mg/m² melphalan to a subject based on the surface area of the subject. A conditioning regimen of this disclosure may comprise administering at least 20 mg/m² melphalan to a subject. A conditioning regimen of this disclosure may comprise administering at most 180 mg/m² melphalan to a subject. A conditioning regimen of this disclosure may comprise administering 20 mg/m² to 30 mg/m², 20 mg/m² to 40 mg/m², 20 mg/m² to 50 mg/m², 20 mg/m² to 60 mg/m², 20 mg/m² to 80 mg/m², 20 mg/m² to 100 mg/m², 20 mg/m² to 120 mg/m², 20 mg/m² to 150 mg/m², 20 mg/m² to 180 mg/m², 30 mg/m² to 40 mg/m², 30 mg/m² to 50 mg/m², 30 mg/m² to 60 mg/m², 30 mg/m² to 80 mg/m², 30 mg/m² to 100 mg/m², 30 mg/m² to 120 mg/m², 30 mg/m² to 150 mg/m², 30 mg/m² to 180 mg/m², 40 mg/m² to 50 mg/m², 40 mg/m² to 60 mg/m², 40 mg/m² to 80 mg/m², 40 mg/m² to 100 mg/m², 40 mg/m² to 120 mg/m², 40 mg/m² to 150 mg/m², 40 mg/m² to 180 mg/m², 50 mg/m² to 60 mg/m², 50 mg/m² to 80 mg/m², 50 mg/m² to 100 mg/m², 50 mg/m² to 120 mg/m², 50 mg/m² to 150 mg/m², 50 mg/m² to 180 mg/m², 60 mg/m² to 80 mg/m², 60 mg/m² to 100 mg/m², 60 mg/m² to 120 mg/m², 60 mg/m² to 150 mg/m², 60 mg/m² to 180 mg/m², 80 mg/m² to 100 mg/m², 80 mg/m² to 120 mg/m², 80 mg/m² to 150 mg/m², 80 mg/m² to 180 mg/m², 100 mg/m² to 120 mg/m², 100 mg/m² to 150 mg/m², 100 mg/m² to 180 mg/m², 120 mg/m² to 150 mg/m², 120 mg/m² to 180 mg/m², or 150 mg/m² to 180 mg/m² melphalan to a subject. A conditioning regimen of this disclosure may comprise administering 20 mg/m², 30 mg/m², 40 mg/m², 50 mg/m², 60 mg/m², 80 mg/m², 100 mg/m², 120 mg/m², 150 mg/m², or 180 mg/m² melphalan to a subject. A conditioning regimen of this disclosure may comprise administering at least 20 mg/m², 30 mg/m², 40 mg/m², 50 mg/m², 60 mg/m², 80 mg/m², 100 mg/m², 120 mg/m² or 150 mg/m² melphalan to a subject. A conditioning regimen of this disclosure may comprise administering at most 30 mg/m², 40 mg/m², 50 mg/m², 60 mg/m², 80 mg/m², 100 mg/m², 120 mg/m², 150 mg/m², or 180 mg/m² melphalan to a subject.

A subject receiving one or more cell components described herein may be administered one or more doses of melphalan prior to the cell transplant. A subject receiving one or more cell components described herein may be administered 1, 2, 3, 4, 5, 6, 7, 8, 9 or 10 doses of melphalan prior to the cell transplant. In some cases, each dose of melphalan has the same concentration. In some cases, one or more doses of melphalan have different concentrations.

A conditioning regimen of this disclosure may comprise administering 100 mg/kg to 140 mg/kg cyclophosphamide. A conditioning regimen of this disclosure may comprise administering at least 100 mg/kg cyclophosphamide. A conditioning regimen of this disclosure may comprise administering at most 140 mg/kg cyclophosphamide. A conditioning regimen of this disclosure may comprise administering 100 mg/kg to 110 mg/kg, 100 mg/kg to 120 mg/kg, 100 mg/kg to 130 mg/kg, 100 mg/kg to 140 mg/kg, 110 mg/kg to 120 mg/kg, 110 mg/kg to 130 mg/kg, 110 mg/kg to 140 mg/kg, 120 mg/kg to 130 mg/kg, 120 mg/kg to 140 mg/kg, or 130 mg/kg to 140 mg/kg cyclophosphamide. A conditioning regimen of this disclosure may comprise administering about 100 mg/kg, 110 mg/kg, 120 mg/kg, 130 mg/kg, or 140 mg/kg cyclophosphamide. A conditioning regimen of this disclosure may comprise administering at least 100 mg/kg, 110 mg/kg, 120 mg/kg or 130 mg/kg cyclophosphamide. A conditioning regimen of this disclosure may comprise administering at most 110 mg/kg, 120 mg/kg, 130 mg/kg, or 140 mg/kg cyclophosphamide.

A subject receiving one or more cell components described herein may be administered one or more doses of cyclophosphamide prior to the cell transplant. A subject receiving one or more cell components described herein may be administered 1, 2, 3, 4, 5, 6, 7, 8, 9 or 10 doses of cyclophosphamide prior to the cell transplant. In some cases, each dose of cyclophosphamide has the same concentration. In some cases, one or more doses of cyclophosphamide have different concentrations.

A conditioning regimen of this disclosure may comprise administering 40 mg/kg to 80 mg/kg etoposide. A conditioning regimen of this disclosure may comprise administering at least 40 mg/kg etoposide. A conditioning regimen of this disclosure may comprise administering at most 80 mg/kg etoposide. A conditioning regimen of this disclosure may comprise administering 40 mg/kg to 50 mg/kg, 40 mg/kg to 60 mg/kg, 40 mg/kg to 70 mg/kg, 40 mg/kg to 80 mg/kg, 50 mg/kg to 60 mg/kg, 50 mg/kg to 70 mg/kg, 50 mg/kg to 80 mg/kg, 60 mg/kg to 70 mg/kg, 60 mg/kg to 80 mg/kg, or 70 mg/kg to 80 mg/kg etoposide. A conditioning regimen of this disclosure may comprise administering about 40 mg/kg, 50 mg/kg, 60 mg/kg, 70 mg/kg, or 80 mg/kg etoposide. A conditioning regimen of this disclosure may comprise administering at least 40 mg/kg, 50 mg/kg, 60 mg/kg or 70 mg/kg etoposide. A conditioning regimen of this disclosure may comprise administering at most 50 mg/kg, 60 mg/kg, 70 mg/kg, or 80 mg/kg etoposide.

A subject receiving one or more cell components described herein may be administered one or more doses of etoposide prior to the cell transplant. A subject receiving one or more cell components described herein may be administered 1, 2, 3, 4, 5, 6, 7, 8, 9 or 10 doses of etoposide prior to the cell transplant. In some cases, each dose of etoposide has the same concentration. In some cases, one or more doses of etoposide have different concentrations.

A conditioning regimen of this disclosure comprising TTP may comprise one or more doses of total body irradiation (TBI) such as hyperfractionated TBI (HFTBI). One or more doses of HFTBI may be administered to a subject before the administration of one or more doses of another conditioning reagent such as TTP. One or more doses of HFTBI may be administered to a subject after the administration of one or more doses of another conditioning reagent such as TTP. One or more doses of HFTBI may be administered to a subject along with the administration of one or more doses of another conditioning reagent such as TTP.

A conditioning regimen of this disclosure may comprise administering 800 cGy to 1,500 cGy HFTBI to a subject. A conditioning regimen of this disclosure may comprise administering at least 800 cGy HFTBI to a subject. A conditioning regimen of this disclosure may comprise administering at most 1,500 cGy HFTBI to a subject. A conditioning regimen of this disclosure may comprise administering 800 cGy to 900 cGy, 800 cGy to 1,000 cGy, 800 cGy to 1,100 cGy, 800 cGy to 1,200 cGy, 800 cGy to 1,300 cGy, 800 cGy to 1,375 cGy, 800 cGy to 1,400 cGy, 800 cGy to 1,500 cGy, 900 cGy to 1,000 cGy, 900 cGy to 1,100 cGy, 900 cGy to 1,200 cGy, 900 cGy to 1,300 cGy, 900 cGy to 1,375 cGy, 900 cGy to 1,400 cGy, 900 cGy to 1,500 cGy, 1,000 cGy to 1,100 cGy, 1,000 cGy to 1,200 cGy, 1,000 cGy to 1,300 cGy, 1,000 cGy to 1,375 cGy, 1,000 cGy to 1,400 cGy, 1,000 cGy to 1,500 cGy, 1,100 cGy to 1,200 cGy, 1,100 cGy to 1,300 cGy, 1,100 cGy to 1,375 cGy, 1,100 cGy to 1,400 cGy, 1,100 cGy to 1,500 cGy, 1,200 cGy to 1,300 cGy, 1,200 cGy to 1,375 cGy, 1,200 cGy to 1,400 cGy, 1,200 cGy to 1,500 cGy, 1,300 cGy to 1,375 cGy, 1,300 cGy to 1,400 cGy, 1,300 cGy to 1,500 cGy, 1,375 cGy to 1,400 cGy, 1,375 cGy to 1,500 cGy, or 1,400 cGy to 1,500 cGy HFTBI to a subject. A conditioning regimen of this disclosure may comprise administering about 800 cGy, 900 cGy, 1,000 cGy, 1,100 cGy, 1,200 cGy, 1,300 cGy, 1,375 cGy, 1,400 cGy, or 1,500 cGy HFTBI to a subject. A conditioning regimen of this disclosure may comprise administering at least 800 cGy, 900 cGy, 1,000 cGy, 1,100 cGy, 1,200 cGy, 1,300 cGy, 1,375 cGy or 1,400 cGy HFTBI to a subject. A conditioning regimen of this disclosure may comprise administering at most 900 cGy, 1,000 cGy, 1,100 cGy, 1,200 cGy, 1,300 cGy, 1,375 cGy, 1,400 cGy, or 1,500 cGy HFTBI to a subject.

A subject receiving one or more cell components described herein may be administered one or more doses of HFTBI prior to the cell transplant. A subject receiving one or more cell components described herein may be administered 1, 2, 3, 4, 5, 6, 7, 8, 9 or 10 doses of HFTBI prior to the cell transplant. In some cases, each dose of HFTBI has the same concentration. In some cases, one or more doses of HFTBI have different concentrations. A subject may be administered a 75 to 150 cGys of HFTBI in a single dose. A subject may be administered at least 75 cGys HFTBI in a single dose. A subject may be administered at least 100 cGys HFTBI in a single dose. A subject may be administered at least 125 cGys HFTBI in a single dose.

Exemplary Conditioning Regimen 1: In some embodiments, a subject is administered a conditioning regimen comprising thiotepa, busulfan, and fludarabine. In some embodiments, a subject is administered thiotepa at 5 mg/kg actual body weight or ideal body weight. In some embodiments, a subject is administered thiotepa at about 5 mg/kg for two days (e.g., consecutive days). In some embodiments, a subject is administered busulfan at about 3.2 mg/kg actual body weight or ideal body weight. In some embodiments, a subject is administered busulfan at about 3.2 mg/kg daily for three days (e.g., consecutive days). In some embodiments, a subject is administered fludarabine at about 50 mg/m². (meters squared−body surface area). In some embodiments, a subject is administered fludarabine at about 50 mg/m² for three days (e.g., consecutive days). In some embodiments, a subject is administered thiotepa at 5 mg/kg actual body weight or ideal body weight, is administered busulfan at about 3.2 mg/kg actual body weight or ideal body weight, and is administered fludarabine at about 50 mg/m². (meters squared−body surface area). In some embodiments, a subject is administered thiotepa at about 5 mg/kg for two days (e.g., consecutive days), is administered busulfan at about 3.2 mg/kg daily for three days (e.g., consecutive days), and is administered fludarabine at about 50 mg/m² for three days (e.g., consecutive days). In some embodiments, a subject is administered thiotepa at about 5 mg/kg on days −7 and −6, is administered busulfan at about 3.2 mg/kg daily on days −5 to −3, and is administered fludarabine at about 50 mg/m² on days −5 to −3.

Exemplary Conditioning Regimen 2: In some embodiments, a subject is administered a conditioning regimen comprising thiotepa, fludarabine, and TBI (e.g., HFTBI). In some embodiments, a subject is administered thiotepa at 5 mg/kg actual body weight or ideal body weight. In some embodiments, a subject is administered thiotepa at about 5 mg/kg for two days (e.g., consecutive days). In some embodiments, a subject is administered fludarabine at about 25 mg/m². (meters squared−body surface area). In some embodiments, a subject is administered fludarabine at about 25 mg/m² for three days (e.g., consecutive days). In some embodiments, a subject is administered HFTBI of 125 cGy (centigray). In some embodiments, a subject is administered HFTBI in 11 fractions of 125 cGy. In some embodiments, a subject is administered HFTBI in 11 fractions of 125 cGy over 4 days. In some embodiments, a subject is administered thiotepa at 5 mg/kg actual body weight or ideal body weight, is administered fludarabine at about 25 mg/m². (meters squared−body surface area), and is administered HFTBI of 125 cGy. In some embodiments, a subject is administered thiotepa at about 5 mg/kg for two days (e.g., consecutive days), is administered fludarabine at about 50 mg/m² for three days (e.g., consecutive days), and is administered HFTBI in 11 fractions of 125 cGy. In some embodiments, a subject is administered thiotepa at about 5 mg/kg on days −7 and −6, is administered fludarabine at about 50 mg/m² on days −5 to −3, and is administered HFTBI in 11 fractions of 125 cGy over 4 days.

A subject may receive the one or more cell populations described herein 1 day after completing a conditioning regimen. A subject may receive the one or more cell components described herein 2, 3, 4, 5, 6, 7, 8, 9, 10 days after completing a conditioning regimen. A subject may receive the one or more cell components described herein 1 day after receiving a final dose of a conditioning reagent such as TTP. A subject may receive the one or more cell components described herein 2, 3, 4, 5, 6, 7, 8, 9 or 10 days after receiving a final dose of a conditioning reagent such as TTP. A subject may receive the one or more cell components described herein 1 day after receiving a first dose of a conditioning reagent such as TTP. A subject may receive the one or more cell components described herein 2, 3, 4, 5, 6, 7, 8, 9 or 10 days after receiving a first dose of a conditioning reagent such as TTP.

### III. Graft Versus Host Disease (Gvhd)

Graft versus host disease (GVHD) is a significant cause of morbidity and mortality in hematopoietic stem cell transplantation (HCT) recipients. The present disclosure provides compositions and methods that reduce the incidence of GVHD, reduce the severity of GVHD, reduce the relative risk of GVHD, prevent GVHD, or a combination thereof in alloHCT recipients.

Graft-versus-host disease (GVHD) is an inflammatory disease that can occur in the allogeneic transplant setting. GVHD involves donor cells (graft) attacking recipient cells (host). GVHD can be life-threatening and can involve, for example, the skin, the intestines, and/or the liver. The morbidity and mortality associated GVHD can be a major factor limiting the success of alloHCT. GVHD can occur despite use of an HLA-matched sibling donor, and the use of various GVHD prophylactic/immunosuppressive agents, for example, use of two or more of tacrolimus, sirolimus, cyclosporine, methotrexate, mycophenolate, anti-thymocyte globulin and corticosteroids.

### A. GVHD Classification and Grading

GVHD can be classified into acute GVHD (aGVHD) and chronic GVHD (cGVHD). In some embodiments, GVHD that occurs within the first 100 days post-transplant can be referred to as acute GVHD (aGVHD), and GVHD after the first 100 days can be referred to as chronic GVHD (cGVHD). cGVHD is a major source of late treatment-related complications and can be life-threatening. In addition to inflammation, cGVHD can lead to the development of fibrosis, which can result in functional disability.

aGVHD and cGVHD can be graded using a system that first evaluates GVHD stages for the skin, liver, and gut, and then combines scoring from the organ staging to determine an overall GVHD grade. An example of a GVHD staging criteria that can be used for individual organs are provided in TABLE 1:

TABLE 1

| Organ | Stage | Description |
|---|---|---|
| Skin | 0 | No active (erythematous) GVHD rash |
| | 1 | Maculo-papular eruption over <25% of body area |
| | 2 | Maculo-papular eruption over >25 to 50% of body area |
| | 3 | Generalized erythroderma/maculo-papular eruption over >50% of body area |
| | 4 | Generalized erythroderma/maculo-papular eruption over >50% of body area |
| | | plus bullous formation and desquamation over >5% of body area |
| Liver | 0 | Bilirubin <2 mg/dL |
| | 1 | Bilirubin 2.0 to 3.0 mg/dL; |
| | | serum glutamic oxaloacetic transaminase (SGOT) SGOT 150 to 750IU |
| | 2 | Bilirubin 3.1 to 6.0 mg/dL |
| | 3 | Bilirubin 6.1 to 15.0 mg/dL |
| | 4 | Bilirubin >15.0 mg/dL |
| Gut | 0 | <500 mL/day or <3 episodes/day |
| | 1 | Diarrhea >30 mL/kg or >500 mL/day |
| | 2 | Diarrhea >60 mL/kg or >1000 mL/day |
| | 3 | Diarrhea >90 mL/kg or >1500 mL/day |
| | 4 | Diarrhea >90 mL/kg or >2000 mL/day; or severe abdominal pain with or |
| | | without ileus |

TABLE 2 provides one set of criteria for assessing overall GVHD grade.

TABLE 2

| | | | | | ECOG |
|---|---|---|---|---|---|
| Grade | Skin | Liver | | Gut | performance |
| 1 | 1 to 2 | 0 | | 0 | 0 |
| 2 | 1 to 3 | 1 | and/or | 1 | 0-1 |
| 3 | 2 to 3 | 2 to 4 | and/or | 2 to 3 | 2-3 |
| 4 | 2 to 4 | 2 to 4 | and/or | 2 to 3 | 3-4 |

aGVHD grade can also be determined based on most severe target organ involvement as defined in the MAGIC standardization criteria described by Harris et al., “International, multicenter standardization of acute graft-versus-host disease clinical data collection: a report from the Mount Sinai Acute GVHD International Consortium. Biology of Blood and Marrow Transplantation 22.1(2016): 4-10, the contents of which is incorporated herein by reference in its entirety. For example, aGVHD organ staging can be evaluated according to TABLE 3, and overall aGVHD grade can be assessed as follows:

Grade 0: No Stage 1-4 of any organ.

Grade I: Stage 1-2 skin without liver, upper GI, or lower GI involvement.

Grade II: Stage 3 rash and/or Stage 1 liver and/or Stage 1 upper GI and/or Stage 1 lower GI.

Grade III: Stage 2-3 liver and/or Stage 2-3 lower GI, with Stage 0-3 skin and/or Stage 0-1 upper GI. Grade IV: Stage 4 skin, liver, or lower GI involvement, with Stage 0-1 upper GI.

TABLE 3

| | Skin (Active | Liver | | | |
|---|---|---|---|---|---|
| Stage | Erythema Only) | (Bilirubin) | | Upper GI | Lower GI (stool output/day) |
| 0 | No active | <2 | mg/dL | No or intermittent | Adult: <500 mL/day |
| | (erythematous) | | | nausea, vomiting or | or <3 episodes/day |
| | GvHD rash | | | anorexia | Child: <10 mL/kg/day |
| | | | | | or <4 episodes/day |
| 1 | Maculopapular | 2-3 | mg/dL | Persistent nausea, | Adult: 500-999 mL/day |
| | rash <25% BSA | | | vomiting or | or 3-4 episodes/day |
| | | | | anorexia | Child: 10-19.9 mL/kg/day |
| | | | | | or 5-7 episodes/day |
| 2 | Maculopapular rash | 3.1-6 | mg/dL | | Adult: 1000-1500 mL/day |
| | 25-50% BSA | | | | or 57 episodes/day |
| | | | | | Child: 20-30 ml/kg/day |
| | | | | | or 7-10 episodes/day |
| 3 | Maculopapular | 6.1-15 | mg/dL | | Adult: >1500 mL/day |
| | rash >50% BSA | | | | or >7 episodes/day |
| | | | | | Child: >30 mL/kg/day |
| | | | | | or >10 episodes/day |
| 4 | Generalized | >15 | mg/dL | | Severe abdominal pain with or |

| | |
|---|---|
| erythroderma | without ileus or grossly bloody |
| (>50% BSA) plus | stool (regardless of stool volume) |
| bullous formation | |
| and desquamation | |
| >5% BSA | |

cGVHD can also be assessed by the method described by Jagasia et al., “National Institutes of Health consensus development project on criteria for clinical trials in chronic graft-versus-host disease: I. The 2014 diagnosis and staging working group report.” Biology of Blood and Marrow Transplantation 21.3 (2015): 389-401, the contents of which is incorporated herein by reference in its entirety. To establish a diagnosis of cGVHD, these criteria can require, for example, at least one diagnostic manifestation of chronic GVHD or at least one distinctive manifestation plus a pertinent biopsy, laboratory or other test (e.g. PFTs, Schirmer's test), evaluation by a specialist (ophthalmologist, gynecologist) or radiographic imaging showing chronic GVHD in the same or another organ.

Organ systems can be scored as described in Jagasia et al., and Mild cGVHD can be present when one or two organs are involved with no more than score 1, plus a lung score of zero; moderate cGVHD can be present when three or more organs are involved with no more than score 1, or when at least one non-lung organ has a score of 2, or when the lungs have a score of 1; and severe cGVHD can be present when at least one organ has a score of 4, or the lungs have a score of 2 or 3.

TABLE 4 provides diagnostic and distinctive manifestations of cGVHD. infection, drug effect, malignancy, or other causes are excluded for distinctive manifestations. Bronchiolitis obliterans syndrome can be diagnostic for lung chronic GVHD only if distinctive sign or symptom present in another organ. Diagnosis of chronic GVHD based on myositis or polymyositis can require a biopsy.

TABLE 4

| Organ or | Diagnostic (Sufficient to Establish | Distinctive¹ (Seen in chronic GVHD, but |
|---|---|---|
| Site | the Diagnosis of chronic GVHD) | Insufficient Alone to Establish a Diagnosis) |
| Skin | Poikiloderma Lichen planus-like | Depigmentation Papulosquamous |
| | features | lesions |
| | Sclerotic features Morphea-like | |
| | features | |
| | Lichen sclerosus-like features | |
| Nails | | Dystrophy |
| | | Longitudinal ridging, splitting or brittle features |
| | | Onycholysis |
| | | Pterygium unguis |
| | | Nail loss (usually symmetric, affects most nails) |
| Scalp and | | New onset of scarring or nonscarring scalp |
| body hair | | alopecia (after recovery from chemoradiotherapy) |
| | | Loss of body hair |
| Mouth | Lichen planus-like changes | Xerostomia Mucoceles Mucosal |
| | | atrophy Ulcers |
| | | Pseudomembranes |
| Eyes | | New onset dry, gritty, or painful eyes |
| | | Cicatricial conjunctivitis |
| | | Keratoconjunctivitis sicca |
| | | Confluent areas of punctate keratopathy |
| Genitalia | Lichen planus-like features Lichen | Erosions Fissures |
| | sclerosus-like features | |
| Females | Vaginal scarring or clitoral/labial | Ulcers |
| | agglutination | |
| Males | Phimosis or urethral/meatus scarring or | |
| | stenosis | |
| GI Tract | Esophageal web Strictures or stenosis | |
| | in the upper to | |
| | mid third of the esophagus | |
| Lung | Bronchiolitis obliterans diagnosed | Air trapping and bronchiectasis on chest CT |
| | with lung biopsy | |
| | Bronchiolitis obliterans syndrome² | |
| Muscles, | Joint stiffness or contractures | Myositis or polymyositis³ |
| fascia, joints | secondary to fasciitis or sclerosis | |

In some embodiments, GVHD severity can be graded using the Glucksberg grade (I-IV) or the International Bone Marrow Transplant Registry (IBMTR) grading system (A-D). The severity of acute GVHD can be determined by an assessment of the degree of involvement of the skin, liver, and gastrointestinal tract. The stages of individual organ involvement are combined with (Glucksberg) or without (IBMTR) the patient's performance status to produce an overall grade.

### B. GVHD Prophylaxis and Treatment

Immunosuppressive agents can be used to reduce the likelihood of GVHD (GVHD prophylactic agents), or to treat GVHD once it occurs (GVHD therapeutic agents). However, in some cases, the use of GVHD prophylactic agents, GVHD therapeutic agents, or both can be insufficient to effectively prevent or treat GVHD. For example, the incidence of GVHD in graft recipients can be high despite use of use of tacrolimus, sirolimus, cyclosporine, methotrexate, mycophenolate, anti-thymocyte globulin, corticosteroids, or a combination thereof (e.g., two or more of the agents).

Administering multiple GVHD prophylactic and/or therapeutic agents, high doses of GVHD prophylactic and/or therapeutic agents, or both can fail to effectively treat GVHD in many alloHCT settings or can result in increased susceptibility to infection and decreased graft versus tumor therapeutic effects.

Non-limiting examples of GVHD prophylactic and/or GVHD therapeutic agents that can be used include calcineurin inhibitors (e.g., tacrolimus, cyclosporine A), sirolimus, monoclonal antibodies, methotrexate, mycophenolate, anti-thymocyte globulin, corticosteroids, azathioprine, and mycophenolate mofetil. Monoclonal antibodies useful as immunosuppressive agents include, for example, antagonist antibodies, (e.g., antibodies that antagonize IL-2R such as basiliximab and daclizumab), and antibodies that deplete an immune cell population by antibody dependent cellular cytotoxicity (e.g., anti-CD3 antibodies for T cell depletion such as muromonab-CD3).

Compositions and methods described herein may comprise administering one or more GVHD prophylactic agents to an alloHCT recipient. GVHD prophylaxis in such cases should be considered different from GVHD treatment such that the GVHD prophylactic agent will be administered to the alloHCT recipient before an incidence of GVHD is assessed. In some cases, an alloHCT recipient may be administered one or more GVHD prophylactic agents but not a GVHD therapeutic agent. In some cases, a alloHCT recipient does not require treatment for GVHD and/or does not receive treatment for GVHD.

Compositions and methods disclosed herein can produce one or more of the following benefits: reduce the incidence of GVHD, reduce the severity of GVHD, reduce the relative risk of GVHD, prevent GVHD, or a combination thereof in alloHCT recipients. In some embodiments, such benefits are achieved despite administering no GVHD prophylactic agents as disclosed herein. In some embodiments, such benefits are achieved despite administering a reduced number of GVHD prophylactic agents (e.g., a single GVHD prophylactic agents), a low dose of GVHD prophylactic agent(s), or a combination thereof as disclosed herein. In some embodiments, 1 GVHD prophylactic agent is administered to a subject. In some embodiments, no more than GVHD prophylactic agent is administered to a subject. In some embodiments, 2 GVHD prophylactic agents are administered to a subject. In some embodiments, no more than 2 GVHD prophylactic agents are administered to a subject.

In some embodiments, one or more GVHD prophylactic agents may be administered to an alloHCT recipient for a duration of 2 days, 3 days, 4 days, 5 days, 6 days, 7 days, 2 weeks, 3 weeks, 4 weeks, 2 months, 3 months, 4 months, 5 months, 6 months, 7 months, 8 months, 9 months, 10 months, 11 months, 12 months, 18 months, 24 months, 36 months post-transplant of one or more cell populations. One or more GVHD prophylactic agents may be administered to an alloHCT recipient starting the day of transplant of one or more cell populations. For instance, a GVHD prophylactic regimen may begin the day an HSPC cell component and/or a Treg cell component is administered to the recipient. Alternatively, a GVHD prophylactic regimen may begin the day a Tcon cell component is administered to the patient.

In some embodiments, tacrolimus is not administered to a subject. In some embodiments, sirolimus is not administered to a subject. In some embodiments, cyclosporine is not administered to a subject. In some embodiments, methotrexate is not administered to a subject. In some embodiments, mycophenolate is not administered to a subject. In some embodiments, anti-thymocyte globulin is not administered to a subject. In some embodiments, corticosteroids are not administered to a subject.

Aspects and embodiments herein provide a method of transplanting cell populations into a human patient as a part of a treatment regimen for a hematologic malignancy. The method comprises administering to the patient a population of hematopoietic stem and progenitor cells (HSPCs; the population of HSPCs comprising HSPCs and a liquid suspending the HSPCs; administering to the patient a population of regulatory T cells (Tregs) to be administered to the patient, the population of Tregs comprising Tregs and a liquid suspending the Tregs; and administering to the patient a heterogenous cell population to be administered to the patient, the heterogenous cell population comprising lymphocytes, granulocytes, monocytes and a liquid suspending said cells, wherein at least about 30% of said lymphocyte comprise conventional T cells (Tcons); and administering to the patient over a period of time up to about 180 days a single graft versus host disease (GVHD) prophylactic agent (GVHDPA) comprising tacrolimus (tacrolimus GHVDPA), wherein the tacrolimus GHVDPA is administered to maintain a concentration of tacrolimus in the patient's blood above a threshold level during the period of time; and wherein a risk and/or severity of GHVD associated with the treatment regimen for the hematologic malignancy is significantly reduced.

In cases where aGVHD occurs, responsiveness of aGVHD to GVHD therapeutic agents (e.g., corticosteroids) can be assessed by the criteria of TABLE 5.

TABLE 5

| | |
|---|---|
| Complete | Complete resolution of acute GvHD |
| response | symptoms in all organs, without |
| (CR) | secondary GvHD therapy |
| Partial | Improvement in GvHD stage in all initial |
| response | GvHD target organs without complete |
| (PR) | resolution and without worsening in any other |
| | GvHD target organs, without secondary GvHD therapy |
| Very good | Improvement in GvHD in all initial GvHD |
| partial | target organs, with maximum Stage I |
| response | involvement in one or more organs (except upper |
| (VGPR) | gastrointestinal tract), without secondary GvHD therapy |
| No | Same grade of GvHD or progression of GvHD in |
| response | any organ or death, or the |
| (NR) | addition of secondary GvHD therapy |
| Progression | Worsening GvHD in at least 1 organ with or |
| | without amelioration in any organ |

In some embodiments, in cases where aGVHD occurs in subjects that receive a composition(s) of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a complete response to GVHD therapeutic agents.

In some embodiments, in cases where aGVHD occurs in subjects that receive a composition(s) of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a partial response to GVHD therapeutic agents.

In some embodiments, in cases where aGVHD occurs in subjects that receive a composition(s) of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a very good partial response to GVHD therapeutic agents.

In cases where cGVHD occurs, responsiveness of cGVHD to GVHD therapeutic agents (e.g., corticosteroids) can be assessed by the criteria of TABLE 6. Abbreviations used: ALT, alanine transaminase; FEV1, forced expiratory volume in the first second; OMRS, Oral Mucosa Rating Scale; PFTs, pulmonary function tests; P-ROM, photographic range of motion; ULN, upper limit of normal.

TABLE 6

| Organ | Complete Response | Partial Response | Progression |
|---|---|---|---|
| Skin | NIH Skin Score 0 after | Decrease in NIH Skin | Increase in NIH Skin Score by 1 |
| | previous involvement | Score by 1 or more points | or more points, except 0 to 1 |
| Eyes | NIH Eye Score 0 after | Decrease in NIH Eye Score | Increase in NIH Eye Score by 1 |
| | previous involvement | by 1 or more points | or more points, except 0 to 1 |
| Mouth | NIH Modified OMRS 0 | Decrease in NIH Modified | Increase in NIH Modified |
| | after previous involvement | OMRS of 2 or more points | OMRS of 2 or more points |
| Esophagus | NIH Esophagus Score 0 after | Decrease in NIH | Increase in NIH Esophagus |
| | previous involvement | Esophagus Score by 1 or | Score by 1 or more points, |
| | | more points | except 0 to 1 |
| Upper GI | NIH Upper GI Score 0 after | Decrease in NIH Upper GI | Increase in NIH Upper GI |
| | previous involvement | Score by 1 or more points | Score by 1 or more points, |
| | | | except 0 to 1 |
| Lower GI | NIH Lower GI Score 0 after | Decrease in NIH Lower GI | Increase in NIH Lower GI |
| | previous involvement | Score by 1 or more points | Score by 1 or more points, |
| | | | except from 0 to 1 |
| Liver | Normal ALT, alkaline | Decrease by 50% | Increase by 2 × ULN |
| | phosphatase, and Total | | |
| | bilirubin after previous | | |
| | elevation of 1 or more | | |
| Lungs | Normal % FEV1 after | Increase by 10% | Decrease by 10% predicted |
| | previous involvement | predicted absolute value of | absolute value of % FEV1 |
| | If PFTs not available, | % FEV1 | If PFTs not available, |
| | NIH Lung Symptom | If PFTs not available, | increase in NIH Lung - |
| | Score 0 after previous | decrease in NIH Lung | Symptom Score by 1 or |
| | involvement | Symptom Score by 1 or | more points, except 0 to |
| | | more points | 1 |
| Joints and | Both NIH Joint and Fascia | Decrease in NIH Joint and | Increase in NIH Joint and Fascia |
| fascia | Score 0 and P-ROM score 25 | Fascia Score by 1 or more | Score by 1 or more points or |
| | after previous involvement by | points or increase in P-ROM | decrease in P-ROM score by 1 |
| | at least | score by 1 point for any site | point for any site |
| | 1 measure | | |
| Global | Clinician overall severity | Clinician overall severity | increases by 2 or more points on a |
| | score 0 | score decreases by 2 or more | Clinician overall severity score |
| | | points on a 0-10 scale | 0-10 scale |

In some embodiments, in cases where cGVHD occurs in subjects that receive a composition of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a complete response to GVHD therapeutic agents.

In some embodiments, in cases where cGVHD occurs in subjects that receive a composition of the disclosure, at least about 40%, at least about 50%, at least about 55%, at least about 60%, at least about 65%, at least about 70%, at least about 75%, at least about 80%, at least about 85%, at least about 90%, or at least about 95% of cases exhibit a partial response to GVHD therapeutic agents.

### C. Acute GVHD Incidence

In another aspect, the invention provides methods of transplanting hematopoietic cells such as HSPC's for the treatment one or more diseases or conditions. In many embodiments the hematopoietic cells to be transplanted can be processed from donor blood using embodiments of kits described herein for the preparation of a desired cell population. Such diseases or conditions may include to one or more of stem cell-based cancers (e.g., leukemia and lymphoma) as well autoimmune conditions (e.g., multiple sclerosis, IBD, Crohn's disease, ankylosing spondylitis myasthenia gravis and like diseases). Various embodiments of the disclosure provide therapeutic and related compositions and methods for improving the outcomes associated with hematopoietic stem cell transplantation (HCT), for example, allogeneic hematopoietic stem cell transplantation (alloHCT) for the treatment of one or more conditions such as various stem cell cancers. Many embodiments provide methods of transplanting a conventional T cell (Tcon) population into a human subject without eliciting particular levels of graft versus host disease, stage 2 or higher graft versus host disease (GVHD) response up to 100 days after transplanting.

In another aspect, the invention provides methods of transplanting a conventional T cell (Tcon) population into a human subject without producing selected levels of graft versus host disease (GVHD) wherein the transplant is done using therapeutic compositions comprising cell components/populations produced using embodiments of the kits and associated methods described herein. In specific embodiments the methods of transplanting T con cells can be configured so as to reduce or prevent a stage 2 or higher GVHD response up to 100 days after transplanting.

In some aspects, the invention provides methods of treating a hematologic malignancy in a human subject in need thereof. The method may comprise administering to the subject: a population of hematopoietic stem and progenitor cells (HSPC's); a population of regulatory T cells (Tregs); and a population of conventional T cells (Tcons) one or more of which may be produced using embodiment of the kits and methods described herein. In particular embodiments one or both of the HSPC's and Treg cells may have specific amounts of bound antibody or antigen binding agent. In some embodiments, the population of HSPC's and the population of Tregs may be administered prior to the population of Tcons though, other administration sequences are also contemplated. In some cases, the peripheral blood of the human subject may exhibit an elevated Treg count until 100 days after the administering the populations of cells as compared to a healthy human subject that was not administered the populations of cells. Embodiments of the disclosure generating such elevated Treg counts are particularly useful for reducing or preventing graft versus host disease (GVHD) in bone marrow (e.g., HSPC) transplant patients as the elevated Tregs suppress or reduce GVHD.

In some aspects, embodiments of the disclosure provide methods of transplanting a conventional T cell (Tcon) population into a human subject without eliciting a stage 2 or higher graft versus host disease (GVHD) response up to 100 days after transplantation. The methods may comprise: administering a population of conventional T cells (Tcons) and a population of regulatory T cells (Tregs) which may be generated using embodiments of the kits and associated methods described herein. In some embodiments, the population of Tcons is cryopreserved for at least 4 hours and the population of Tcons and the population of Tregs comprise less than 5 EU/ml endotoxins.

In some aspects, the invention provides methods of transplanting a conventional T cell (Tcon) population into a human subject without eliciting a stage 2 or higher graft versus host disease (GVHD) response up to 100 days after transplanting. The method may comprise: administering a population of hematopoietic stem and progenitor cells (HSPC's); administering a population of conventional T cells (Tcons); and administering a population of regulatory T cells (Tregs). The population of Tcons may be cryopreserved for at least 4 hours. The population of HSPC's and/or the population of Tregs may comprise less than 5% unbound reagents w/w.

In some aspects, described herein are methods of transplanting a conventional T cell (Tcon) population into a human subject without eliciting a stage 2 or higher graft versus host disease (GVHD) response up to 100 days after transplanting. The method may comprise: administering a population of hematopoietic stem and progenitor cells (HSPC's); administering a population of conventional T cells (Tcons); and administering a population of regulatory T cells (Tregs). The population of Tcons may be cryopreserved for at least 4 hours. The population of HSPC's and/or the population of Tregs may comprise less than 1% unbound magnetic reagents w/w.

In some aspects, the invention provides a method of transplanting a conventional T cell (Tcon) population into a human subject without eliciting a stage 2 or higher graft versus host disease (GVHD) response up to 100 days after transplanting. The method may comprise: administering a population of hematopoietic stem and progenitor cells (HSPC's); administering a population of conventional T cells (Tcons); and administering a population of regulatory T cells (Tregs). The HSPC's may correspond to CD34+ cells, the Tregs to CD4+ CD25+ CD127dim cells and the Tcons CD3+ cells. In various embodiments the Tregs may comprise one or more of CD45+, CD4+ CD25+ CD127dim or CD4+ FOXP3+ cells. In particular embodiments, where Tregs comprise CD45+ cells 90% of the cells a population CD45+ cells comprise Tregs.

In some embodiments, the human subject does not develop graft versus host disease (GVHD) within 30 days of the administering of the population of Tcons. In some embodiments, the human subject does not develop graft versus host disease (GVHD) within 100 days of the administering of the population of Tcons. In some embodiments, the human subject does not develop graft versus host disease (GVHD) within 200 days of the administering of the population of Tcons. In some embodiments, the human subject does not develop graft versus host disease (GVHD) within 1 year of the administering of the population of Tcons.

Subjects administered a composition of the disclosure (e.g., a cell component comprising a populations of cells described herein) exhibit a low incidence of ≥grade 1 aGVHD, for example, a lower incidence of ≥grade 1 aGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure develop ≥grade 1 aGVHD, for example, within 30 days post-transplant, 100 days post-transplant, or within another suitable amount of time post-transplant as disclosed herein.

As used herein, an alternate composition lacks one or more cell components and/or prophylactic agent that are disclosed herein and/or recited in the claims. As examples, an alternate composition lacks one or more of a cell component comprising HSPC's, a cell component comprising Tregs, a cell component comprising Tcons, and a prophylactic agent.

In some embodiments, the method may comprise administering to the human subject a graft versus host disease (GVHD) prophylactic agent. In some embodiments, the GVHD prophylactic agent may be tacrolimus or sirolimus.

Subjects administered a composition of the disclosure (e.g., a cell component comprising a populations of cells described herein) exhibit a low incidence of ≥grade 2 aGVHD, for example, a lower incidence of ≥grade 2 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop ≥grade 2 aGVHD, for example, within 30 days post-transplant, 100 days post-transplant, or within another suitable amount of time post-transplant as disclosed herein.

In some embodiments, less than about 20% of subjects that are administered a composition of the disclosure develop ≥grade 2 aGVHD. In some embodiments, less than about 15% of subjects that are administered a composition of the disclosure develop ≥grade 2 aGVHD. In some embodiments, less than about 10% of subjects that are administered a composition of the disclosure develop ≥grade 2 aGVHD. In some embodiments, less than about 8% of subjects that are administered a composition of the disclosure develop ≥grade 2 aGVHD. In some embodiments, less than about 7% of subjects that are administered a composition of the disclosure develop ≥grade 2 aGVHD.

Subjects administered a composition of the disclosure (e.g., a cell component comprising a populations of cells described herein) exhibit a low incidence of ≥grade 3 aGVHD, for example, a lower incidence of ≥grade 3 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop ≥grade 3 aGVHD, for example, within 30 days post-transplant, 100 days post-transplant, or within another suitable amount of time post-transplant as disclosed herein.

In some embodiments, less than about 20% of subjects that are administered a composition of the disclosure develop ≥grade 3 aGVHD. In some embodiments, less than about 15% of subjects that are administered a composition of the disclosure develop ≥grade 3 aGVHD. In some embodiments, less than about 10% of subjects that are administered a composition of the disclosure develop ≥grade 3 aGVHD. In some embodiments, less than about 5% of subjects that are administered a composition of the disclosure develop ≥grade 3 aGVHD. In some embodiments, less than about 3% of subjects that are administered a composition of the disclosure develop ≥grade 3 aGVHD. In some embodiments, less than about 2% of subjects that are administered a composition of the disclosure develop ≥grade 3 aGVHD. In some embodiments, less than about 1% of subjects that are administered a composition of the disclosure develop ≥grade 3 aGVHD.

Subjects administered a composition of the disclosure (e.g., a cell component comprising a populations of cells described herein) exhibit a low incidence of ≥grade 4 aGVHD, for example, a lower incidence of ≥grade 4 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure develop ≥grade 4 aGVHD, for example, within 30 days post-transplant, 100 days post-transplant, or within another suitable amount of time post-transplant as disclosed herein.

In some embodiments, less than about 10% of subjects that are administered a composition of the disclosure develop ≥grade 4 aGVHD. In some embodiments, less than about 5% of subjects that are administered a composition of the disclosure develop ≥grade 4 aGVHD. In some embodiments, less than about 3% of subjects that are administered a composition of the disclosure develop ≥grade 4 aGVHD. In some embodiments, less than about 2% of subjects that are administered a composition of the disclosure develop ≥grade 4 aGVHD. In some embodiments, less than about 1% of subjects that are administered a composition of the disclosure develop ≥grade 4 aGVHD.

The incidence of aGVHD can be assessed after a suitable amount of time elapses post-transplant, for example, about 20 days, about 21 days, about 25 days, about 28 days, about 30 days, about 35 days, about 40 days, about 42 days, about 45 days, about 49 days, about 50 days, about 55 days, about 56 days, about 60 days, about 63 days, about 65 days, about 70 days, about 75 days, about 77 days, about 80 days, about 84 days, about 85 days, about 90 days, about 91 days, about 95 days, about 98 days, about 100 days, about 105 days, about 110 days, about 112 days, about 115 days, about 119 days, about 120 days post-transplant.

The incidence of aGVHD can be calculated based on a population of at least 10, at least at least 11, at least at least 12, at least 13, at least 14, at least 15, at least 16, at least 17, at least 18, at least 19, at least 20, at least 21, at least 22, at least 23, at least 24, at least 25, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, or at least 500 subjects.

### 1. No GVHD Prophylaxis

Subjects administered a composition of the disclosure (e.g., a cell component comprising a populations of cells described herein) in the absence of GVHD prophylactic agents exhibit a low incidence of ≥grade 1 aGVHD, for example, a lower incidence of ≥grade 1 aGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure in the absence of GVHD prophylactic agents develop ≥grade 1 aGVHD, for example, within 30 days post-transplant, 100 days post-transplant, or within another suitable amount of time post-transplant as disclosed herein.

Subjects administered a composition of the disclosure (e.g., a cell component comprising a populations of cells described herein) in the absence of GVHD prophylactic agents exhibit a low incidence of ≥grade 2 aGVHD, for example, a lower incidence of ≥grade 2 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop ≥grade 2 aGVHD, for example, within 30 days post-transplant, 100 days post-transplant, or within another suitable amount of time post-transplant as disclosed herein.

In some embodiments, less than about 50% of subjects that are administered a composition of the disclosure in the absence of GVHD prophylactic agents develop ≥grade 2 aGVHD. In some embodiments, less than about 40% of subjects that are administered a composition of the disclosure in the absence of GVHD prophylactic agents develop ≥grade 2 aGVHD. In some embodiments, less than about 30% of subjects that are administered a composition of the disclosure in the absence of GVHD prophylactic agents develop ≥grade 2 aGVHD. In some embodiments, less than about 25% of subjects that are administered a composition of the disclosure in the absence of GVHD prophylactic agents develop ≥grade 2 aGVHD. In some embodiments, less than about 20% of subjects that are administered a composition of the disclosure in the absence of GVHD prophylactic agents develop ≥grade 2 aGVHD. In some embodiments, less than about 10% of subjects that are administered a composition of the disclosure in the absence of GVHD prophylactic agents develop ≥grade 2 aGVHD.

Subjects administered a composition of the disclosure (e.g., one or more populations of cells described herein) in the absence of GVHD prophylactic agents exhibit a low incidence of ≥grade 3 aGVHD, for example, a lower incidence of ≥grade 3 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop ≥grade 3 aGVHD, for example, within 30 days post-transplant, 100 days post-transplant, or within another suitable amount of time post-transplant as disclosed herein.

In some embodiments, less than about 40% of subjects that are administered a composition of the disclosure in the absence of GVHD prophylactic agents develop ≥grade 3 aGVHD. In some embodiments, less than about 30% of subjects that are administered a composition of the disclosure in the absence of GVHD prophylactic agents develop ≥grade 3 aGVHD. In some embodiments, less than about 20% of subjects that are administered a composition of the disclosure in the absence of GVHD prophylactic agents develop ≥grade 3 aGVHD. In some embodiments, less than about 10% of subjects that are administered a composition of the disclosure in the absence of GVHD prophylactic agents develop ≥grade 3 aGVHD. In some embodiments, less than about 5% of subjects that are administered a composition of the disclosure in the absence of GVHD prophylactic agents develop ≥grade 3 aGVHD. In some embodiments, less than about 3% of subjects that are administered a composition of the disclosure in the absence of GVHD prophylactic agents develop ≥grade 3 aGVHD. In some embodiments, less than about 2% of subjects that are administered a composition of the disclosure in the absence of GVHD prophylactic agents develop ≥grade 3 aGVHD. In some embodiments, less than about 1% of subjects that are administered a composition of the disclosure in the absence of GVHD prophylactic agents develop ≥grade 3 aGVHD.

Subjects administered a composition of the disclosure (e.g., a cell component comprising a populations of cells described herein) in the absence of GVHD prophylactic agents exhibit a low incidence of ≥grade 4 aGVHD, for example, a lower incidence of ≥grade 4 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure in the absence of GVHD prophylactic agents develop ≥grade 4 aGVHD, for example, within 30 days post-transplant, 100 days post-transplant, or within another suitable amount of time post-transplant as disclosed herein.

In some embodiments, less than about 20% of subjects that are administered a composition of the disclosure in the absence of GVHD prophylactic agents develop ≥grade 4 aGVHD. In some embodiments, less than about 15% of subjects that are administered a composition of the disclosure in the absence of GVHD prophylactic agents develop ≥grade 4 aGVHD. In some embodiments, less than about 10% of subjects that are administered a composition of the disclosure in the absence of GVHD prophylactic agents develop ≥grade 4 aGVHD. In some embodiments, less than about 5% of subjects that are administered a composition of the disclosure in the absence of GVHD prophylactic agents develop ≥grade 4 aGVHD. In some embodiments, less than about 3% of subjects that are administered a composition of the disclosure in the absence of GVHD prophylactic agents develop ≥grade 4 aGVHD. In some embodiments, less than about 2% of subjects that are administered a composition of the disclosure in the absence of GVHD prophylactic agents develop ≥grade 4 aGVHD. In some embodiments, less than about 1% of subjects that are administered a composition of the disclosure in the absence of GVHD prophylactic agents develop ≥grade 4 aGVHD.

The absence of GVHD prophylactic agents can refer to cases where no GVHD prophylactic agents are administered to the subject for the first 20 days, 21 days, 25 days, 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100 days, 10⁵ days, 110 days, 112 days, 115 days, 119 days, or 120 days post-transplant.

### 2. Single Agent GVHD Prophylaxis

In some aspects, the methods of treating a human subject for various conditions such as stem cell-based malignancies (e.g., leukemia and lymphoma) whereby the patient receives cell populations are produced using embodiments of the kits and methods described herein and wherein the patient receives a graft versus host disease (GVHD) prophylactic agent. The method may comprise administering no more than one graft versus host disease (GVHD) prophylactic agent for less than 60 days and administering one or more pluralities of populations of cells isolated from donor blood using embodiments of kits described herein. In some embodiments, the plurality of populations of cells comprises: a population of hematopoietic stem and progenitor cells (HSPC's); a population of cells comprising regulatory T cells (Tregs); and a population of conventional T cells (Tcons). In some embodiments, the population of HSPC's comprises less than 2% CD3+ cells

In some embodiments, the method may comprise administering to the human subject a graft versus host disease (GVHD) prophylactic agent. In some embodiments, the GVHD prophylactic agent may be tacrolimus or sirolimus.

Subjects administered a composition of the disclosure (e.g., a cell component comprising a populations of cells described herein) and no more than one GVHD prophylactic agent (for example, a single GVHD prophylactic agent) exhibit a low incidence of ≥grade 1 aGVHD, for example, a lower incidence of grade 1 aGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and no more than one GVHD prophylactic agent (for example, a single GVHD prophylactic agent) develop grade 1 aGVHD, for example, within 30 days post-transplant, 100 days post-transplant, or within another suitable amount of time post-transplant as disclosed herein.

Subjects administered a composition of the disclosure (e.g., a cell component comprising a populations of cells described herein) and no more than one GVHD prophylactic agent (for example, a single GVHD prophylactic agent) exhibit a low incidence of ≥grade 2 aGVHD, for example, a lower incidence of grade 2 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop grade 2 aGVHD, for example, within 30 days post-transplant, 100 days post-transplant, or within another suitable amount of time post-transplant as disclosed herein.

In some embodiments, less than about 20% of subjects that are administered a composition of the disclosure and no more than one GVHD prophylactic agent (for example, a single GVHD prophylactic agent) develop ≥grade 2 aGVHD. In some embodiments, less than about 15% of subjects that are administered a composition of the disclosure and no more than one GVHD prophylactic agent (for example, a single GVHD prophylactic agent) develop ≥grade 2 aGVHD. In some embodiments, less than about 10% of subjects that are administered a composition of the disclosure and no more than one GVHD prophylactic agent (for example, a single GVHD prophylactic agent) develop ≥grade 2 aGVHD. In some embodiments, less than about 8% of subjects that are administered a composition of the disclosure and no more than one GVHD prophylactic agent (for example, a single GVHD prophylactic agent) develop ≥grade 2 aGVHD. In some embodiments, less than about 7% of subjects that are administered a composition of the disclosure and no more than one GVHD prophylactic agent (for example, a single GVHD prophylactic agent) develop ≥grade 2 aGVHD.

Subjects administered a composition of the disclosure (e.g., a cell component comprising a populations of cells described herein) and no more than one GVHD prophylactic agent (for example, a single GVHD prophylactic agent) exhibit a low incidence of ≥grade 3 aGVHD, for example, a lower incidence of ≥grade 3 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop ≥grade 3 aGVHD, for example, within 30 days post-transplant, 100 days post-transplant, or within another suitable amount of time post-transplant as disclosed herein.

In some embodiments, less than about 20% of subjects that are administered a composition of the disclosure and no more than one GVHD prophylactic agent (for example, a single GVHD prophylactic agent) develop ≥grade 3 aGVHD. In some embodiments, less than about 15% of subjects that are administered a composition of the disclosure and no more than one GVHD prophylactic agent (for example, a single GVHD prophylactic agent) develop ≥grade 3 aGVHD. In some embodiments, less than about 10% of subjects that are administered a composition of the disclosure and no more than one GVHD prophylactic agent (for example, a single GVHD prophylactic agent) develop ≥grade 3 aGVHD. In some embodiments, less than about 5% of subjects that are administered a composition of the disclosure and no more than one GVHD prophylactic agent (for example, a single GVHD prophylactic agent) develop ≥grade 3 aGVHD. In some embodiments, less than about 3% of subjects that are administered a composition of the disclosure and no more than one GVHD prophylactic agent (for example, a single GVHD prophylactic agent) develop ≥grade 3 aGVHD. In some embodiments, less than about 2% of subjects that are administered a composition of the disclosure and no more than one GVHD prophylactic agent (for example, a single GVHD prophylactic agent) develop ≥grade 3 aGVHD. In some embodiments, less than about 1% of subjects that are administered a composition of the disclosure and no more than one GVHD prophylactic agent (for example, a single GVHD prophylactic agent) develop ≥grade 3 aGVHD.

Subjects administered a composition of the disclosure (e.g., a cell component comprising a populations of cells described herein) and no more than one GVHD prophylactic agent (for example, a single GVHD prophylactic agent) exhibit a low incidence of ≥grade 4 aGVHD, for example, a lower incidence of ≥grade 4 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure and no more than one GVHD prophylactic agent (for example, a single GVHD prophylactic agent) develop ≥grade 4 aGVHD, for example, within 30 days post-transplant, 100 days post-transplant, or within another suitable amount of time post-transplant as disclosed herein.

In some embodiments, less than about 10% of subjects that are administered a composition of the disclosure and no more than one GVHD prophylactic agent (for example, a single GVHD prophylactic agent) develop ≥grade 4 aGVHD. In some embodiments, less than about 5% of subjects that are administered a composition of the disclosure and no more than one GVHD prophylactic agent (for example, a single GVHD prophylactic agent) develop ≥grade 4 aGVHD. In some embodiments, less than about 3% of subjects that are administered a composition of the disclosure and no more than one GVHD prophylactic agent (for example, a single GVHD prophylactic agent) develop ≥grade 4 aGVHD. In some embodiments, less than about 2% of subjects that are administered a composition of the disclosure and no more than one GVHD prophylactic agent (for example, a single GVHD prophylactic agent) develop ≥grade 4 aGVHD. In some embodiments, less than about 1% of subjects that are administered a composition of the disclosure and no more than one GVHD prophylactic agent (for example, a single GVHD prophylactic agent) develop ≥grade 4 aGVHD.

A single GVHD prophylactic agent can be tacrolimus.

A single GVHD prophylactic agent can be sirolimus.

The no more than one GVHD prophylactic agent (for example, a single GVHD prophylactic agent) can be administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant. In some embodiments, the no more than one GVHD prophylactic agent (for example, a single GVHD prophylactic agent) is administered to the subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years post-transplant.

### 3. Low Dose GVHD Prophylaxis

In some embodiments, the method may comprise administering to the human subject a graft versus host disease (GVHD) prophylactic agent. In some embodiments, the GVHD prophylactic agent may be tacrolimus or sirolimus.

Subjects administered a composition of the disclosure (e.g., a cell component comprising a population of cells described herein) and a low dose of a GVHD prophylactic agent (for example, a single GVHD prophylactic agent at a low dose) exhibit a low incidence of ≥grade 1 aGVHD, for example, a lower incidence of ≥grade 1 aGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and a low dose of a GVHD prophylactic agent (for example, a single GVHD prophylactic agent at a low dose) develop grade 1 aGVHD, for example, within 30 days post-transplant, 100 days post-transplant, or within another suitable amount of time post-transplant as disclosed herein.

Subjects administered a composition of the disclosure (e.g., a cell component comprising a population of cells described herein) and a low dose of a GVHD prophylactic agent (for example, a single GVHD prophylactic agent at a low dose) exhibit a low incidence of ≥grade 2 aGVHD, for example, a lower incidence of ≥grade 2 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop ≥grade 2 aGVHD, for example, within 30 days post-transplant, 100 days post-transplant, or within another suitable amount of time post-transplant as disclosed herein.

In some embodiments, less than about 20% of subjects that are administered a composition of the disclosure and a low dose of a GVHD prophylactic agent (for example, a single GVHD prophylactic agent at a low dose) develop ≥grade 2 aGVHD. In some embodiments, less than about 15% of subjects that are administered a composition of the disclosure and a low dose of a GVHD prophylactic agent (for example, a single GVHD prophylactic agent at a low dose) develop ≥grade 2 aGVHD. In some embodiments, less than about 10% of subjects that are administered a composition of the disclosure and a low dose of a GVHD prophylactic agent (for example, a single GVHD prophylactic agent at a low dose) develop ≥grade 2 aGVHD. In some embodiments, less than about 8% of subjects that are administered a composition of the disclosure and a low dose of a GVHD prophylactic agent (for example, a single GVHD prophylactic agent at a low dose) develop ≥grade 2 aGVHD. In some embodiments, less than about 7% of subjects that are administered a composition of the disclosure and a low dose of a GVHD prophylactic agent (for example, a single GVHD prophylactic agent at a low dose) develop ≥grade 2 aGVHD.

Subjects administered a composition of the disclosure (e.g., a cell component comprising a populations of cells described herein) and a low dose of a GVHD prophylactic agent (for example, a single GVHD prophylactic agent at a low dose) exhibit a low incidence of ≥grade 3 aGVHD, for example, a lower incidence of ≥grade 3 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop ≥grade 3 aGVHD, for example, within 30 days post-transplant, 100 days post-transplant, or within another suitable amount of time post-transplant as disclosed herein.

In some embodiments, less than about 20% of subjects that are administered a composition of the disclosure and a low dose of a GVHD prophylactic agent (for example, a single GVHD prophylactic agent at a low dose) develop ≥grade 3 aGVHD. In some embodiments, less than about 15% of subjects that are administered a composition of the disclosure and a low dose of a GVHD prophylactic agent (for example, a single GVHD prophylactic agent at a low dose) develop ≥grade 3 aGVHD. In some embodiments, less than about 10% of subjects that are administered a composition of the disclosure and a low dose of a GVHD prophylactic agent (for example, a single GVHD prophylactic agent at a low dose) develop ≥grade 3 aGVHD. In some embodiments, less than about 5% of subjects that are administered a composition of the disclosure and a low dose of a GVHD prophylactic agent (for example, a single GVHD prophylactic agent at a low dose) develop ≥grade 3 aGVHD. In some embodiments, less than about 3% of subjects that are administered a composition of the disclosure and a low dose of a GVHD prophylactic agent (for example, a single GVHD prophylactic agent at a low dose) develop ≥grade 3 aGVHD. In some embodiments, less than about 2% of subjects that are administered a composition of the disclosure and a low dose of a GVHD prophylactic agent (for example, a single GVHD prophylactic agent at a low dose) develop ≥grade 3 aGVHD. In some embodiments, less than about 1% of subjects that are administered a composition of the disclosure and a low dose of a GVHD prophylactic agent (for example, a single GVHD prophylactic agent at a low dose) develop ≥grade 3 aGVHD.

Subjects administered a composition of the disclosure (e.g., a cell component comprising a populations of cells described herein) and a low dose of a GVHD prophylactic agent (for example, a single GVHD prophylactic agent at a low dose) exhibit a low incidence of ≥grade 4 aGVHD, for example, a lower incidence of ≥grade 4 aGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure and a low dose of a GVHD prophylactic agent (for example, a single GVHD prophylactic agent at a low dose) develop ≥grade 4 aGVHD, for example, within 30 days post-transplant, 100 days post-transplant, or within another suitable amount of time post-transplant as disclosed herein.

In some embodiments, less than about 10% of subjects that are administered a composition of the disclosure and a low dose of a GVHD prophylactic agent (for example, a single GVHD prophylactic agent at a low dose) develop ≥grade 4 aGVHD. In some embodiments, less than about 5% of subjects that are administered a composition of the disclosure and a low dose of a GVHD prophylactic agent (for example, a single GVHD prophylactic agent at a low dose) develop ≥grade 4 aGVHD. In some embodiments, less than about 3% of subjects that are administered a composition of the disclosure and a low dose of a GVHD prophylactic agent (for example, a single GVHD prophylactic agent at a low dose) develop ≥grade 4 aGVHD. In some embodiments, less than about 2% of subjects that are administered a composition of the disclosure and a low dose of a GVHD prophylactic agent (for example, a single GVHD prophylactic agent at a low dose) develop ≥grade 4 aGVHD. In some embodiments, less than about 1% of subjects that are administered a composition of the disclosure and a low dose of a GVHD prophylactic agent (for example, a single GVHD prophylactic agent at a low dose) develop ≥grade 4 aGVHD.

A low dose of a GVHD prophylactic agent can be, for example a target trough level of less than about 25 ng/mL, less than about 20 ng/mL, less than about 15 ng/mL, less than about 12 ng/mL, less than about 11 ng/mL, less than about 10 ng/mL, less than about 9 ng/mL, less than about 8 ng/mL, less than about 7 ng/mL, less than about 6 ng/mL, less than about 5 ng/mL, less than about 4 ng/mL, less than about 3 ng/mL, less than about 2 ng/mL, or less than about 1 ng/mL.

In some embodiments, a low dose of a GVHD prophylactic is a target trough level of about 1-25 ng/mL, about 1-20 ng/mL, about 1-15 ng/mL, about 1-12 ng/mL, about 1-11 ng/mL, about 1-10 ng/mL, about 1-9 ng/mL, about 1-8 ng/mL, about 1-7 ng/mL, about 1-6 ng/mL, about 1-5 ng/mL, about 1-4 ng/mL, about 1-3 ng/mL, about 1-2 ng/mL, about 2-25 ng/mL, about 2-20 ng/mL, about 2-15 ng/mL, about 2-12 ng/mL, about 2-11 ng/mL, about 2-10 ng/mL, about 2-9 ng/mL, about 2-8 ng/mL, about 2-7 ng/mL, about 2-6 ng/mL, about 2-5 ng/mL, about 2-4 ng/mL, about 2-3 ng/mL, about 3-25 ng/mL, about 3-20 ng/mL, about 3-15 ng/mL, about 3-12 ng/mL, about 3-11 ng/mL, about 3-10 ng/mL, about 3-9 ng/mL, about 3-8 ng/mL, about 3-7 ng/mL, about 3-6 ng/mL, about 3-5 ng/mL, about 3-4 ng/mL, about 4-25 ng/mL, about 4-20 ng/mL, about 4-15 ng/mL, about 4-12 ng/mL, about 4-11 ng/mL, about 4-10 ng/mL, about 4-9 ng/mL, about 4-8 ng/mL, about 4-7 ng/mL, about 4-6 ng/mL, about 4-5 ng/mL, about 5-25 ng/mL, about 5-20 ng/mL, about 5-15 ng/mL, about 5-12 ng/mL, about 5-11 ng/mL, about 5-10 ng/mL, about 5-9 ng/mL, about 5-8 ng/mL, about 5-7 ng/mL, about 5-6 ng/mL, about 6-25 g/mL, about 6-20 ng/mL, about 6-15 ng/mL, about 6-12 ng/mL, about 6-11 ng/mL, about 6-10 ng/mL, about 6-9 ng/mL, about 6-8 ng/mL, about 6-7 ng/mL, about 8-25 ng/mL, about 8-20 ng/mL, about 8-15 ng/mL, about 8-12 ng/mL, about 8-11 ng/mL, about 8-10 ng/mL, about 8-9 ng/mL, about 10-25 ng/mL, about 10-20 ng/mL, about 10-15 ng/mL, about 10-12 ng/mL, or about 10-11 ng/mL.

In some embodiments, a low dose of a GVHD prophylactic agent is tacrolimus with a target trough level of about 5 ng/mL to about 10 ng/mL. In some embodiments, a low dose of a GVHD prophylactic agent is tacrolimus with a target trough level of about 4 ng/mL to about 6 ng/mL.

In some embodiments, a low dose of a GVHD prophylactic agent is sirolimus with a target trough level of about 3 ng/mL to about 8 ng/mL. In some embodiments, a low dose of a GVHD prophylactic agent is sirolimus with a target trough level of about 4 ng/mL to about 8 ng/mL.

The low dose GVHD prophylactic agent (for example, single GVHD prophylactic agent at a low dose) can be administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant. In some embodiments, the low dose GVHD prophylactic agent (for example, single GVHD prophylactic agent at a low dose) is administered to the subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years post-transplant.

### D. Chronic GVHD Incidence

In some embodiments, the human subject does not develop graft versus host disease (GVHD) within 200 days of the administering of the population of Tcons. In some embodiments, the human subject does not develop graft versus host disease (GVHD) within 1 year of the administering of the population of Tcons

Subjects administered a composition of the disclosure (e.g., a cell component comprising a population of cells described herein) exhibit a low incidence of ≥grade 1 cGVHD, for example, a lower incidence of ≥grade 1 cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure develop ≥grade 1 cGVHD, for example, within 365 days post-transplant, two years post-transplant, or within another suitable amount of time post-transplant as disclosed herein.

Subjects administered a composition of the disclosure (e.g., a cell component comprising a population of cells described herein) exhibit a low incidence of ≥grade 2 cGVHD, for example, a lower incidence of ≥grade 2 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop ≥grade 2 cGVHD, for example, within 365 days post-transplant, two years post-transplant, or within another suitable amount of time post-transplant as disclosed herein.

In some embodiments, less than about 50% of subjects that are administered a composition of the disclosure develop ≥grade 2 cGVHD. In some embodiments, less than about 40% of subjects that are administered a composition of the disclosure develop ≥grade 2 cGVHD. In some embodiments, less than about 30% of subjects that are administered a composition of the disclosure develop ≥grade 2 cGVHD. In some embodiments, less than about 20% of subjects that are administered a composition of the disclosure develop ≥grade 2 cGVHD. In some embodiments, less than about 10% of subjects that are administered a composition of the disclosure develop ≥grade 2 cGVHD. In some embodiments, less than about 5% of subjects that are administered a composition of the disclosure develop ≥grade 2 cGVHD.

Subjects administered a composition of the disclosure (e.g., a cell component comprising a populations of cells described herein) exhibit a low incidence of ≥grade 3 cGVHD, for example, a lower incidence of ≥grade 3 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop ≥grade 3 cGVHD, for example, within 365 days post-transplant, two years post-transplant, or within another suitable amount of time post-transplant as disclosed herein.

In some embodiments, less than about 50% of subjects that are administered a composition of the disclosure develop ≥grade 3 cGVHD. In some embodiments, less than about 40% of subjects that are administered a composition of the disclosure develop ≥grade 3 cGVHD. In some embodiments, less than about 30% of subjects that are administered a composition of the disclosure develop ≥grade 3 cGVHD. In some embodiments, less than about 20% of subjects that are administered a composition of the disclosure develop ≥grade 3 cGVHD. In some embodiments, less than about 15% of subjects that are administered a composition of the disclosure develop ≥grade 3 cGVHD. In some embodiments, less than about 10% of subjects that are administered a composition of the disclosure develop ≥grade 3 cGVHD. In some embodiments, less than about 5% of subjects that are administered a composition of the disclosure develop ≥grade 3 cGVHD. In some embodiments, less than about 3% of subjects that are administered a composition of the disclosure develop ≥grade 3 cGVHD. In some embodiments, less than about 2% of subjects that are administered a composition of the disclosure develop ≥grade 3 cGVHD. In some embodiments, less than about 1% of subjects that are administered a composition of the disclosure develop ≥grade 3 cGVHD.

Subjects administered a composition of the disclosure (e.g., a cell component comprising a populations of cells described herein) exhibit a low incidence of ≥grade 4 cGVHD, for example, a lower incidence of ≥grade 4 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure develop ≥grade 4 cGVHD, for example, within 365 days post-transplant, two years post-transplant, or within another suitable amount of time post-transplant as disclosed herein.

In some embodiments, less than about 40% of subjects that are administered a composition of the disclosure develop ≥grade 4 cGVHD. In some embodiments, less than about 30% of subjects that are administered a composition of the disclosure develop ≥grade 4 cGVHD. In some embodiments, less than about 20% of subjects that are administered a composition of the disclosure develop ≥grade 4 cGVHD. In some embodiments, less than about 10% of subjects that are administered a composition of the disclosure develop ≥grade 4 cGVHD. In some embodiments, less than about 5% of subjects that are administered a composition of the disclosure develop ≥grade 4 cGVHD. In some embodiments, less than about 3% of subjects that are administered a composition of the disclosure develop ≥grade 4 cGVHD. In some embodiments, less than about 2% of subjects that are administered a composition of the disclosure develop ≥grade 4 cGVHD. In some embodiments, less than about 1% of subjects that are administered a composition of the disclosure develop ≥grade 4 cGVHD.

Subjects administered a composition of the disclosure (e.g., a cell component comprising a populations of cells described herein) exhibit a low incidence of mild to severe cGVHD, for example, a lower incidence of mild to severe cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure develop mild to severe cGVHD, for example, within 365 days post-transplant, two years post-transplant, or within another suitable amount of time post-transplant as disclosed herein.

Subjects administered a composition of the disclosure (e.g., a cell component comprising a populations of cells described herein) exhibit a low incidence of moderate to severe cGVHD, for example, a lower incidence of moderate to severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop moderate to severe cGVHD, for example, within 365 days post-transplant, two years post-transplant, or within another suitable amount of time post-transplant as disclosed herein.

In some embodiments, less than about 50% of subjects that are administered a composition of the disclosure develop moderate to severe cGVHD. In some embodiments, less than about 40% of subjects that are administered a composition of the disclosure develop moderate to severe cGVHD. In some embodiments, less than about 30% of subjects that are administered a composition of the disclosure develop moderate to severe cGVHD. In some embodiments, less than about 20% of subjects that are administered a composition of the disclosure develop moderate to severe cGVHD. In some embodiments, less than about 10% of subjects that are administered a composition of the disclosure develop moderate to severe cGVHD. In some embodiments, less than about 5% of subjects that are administered a composition of the disclosure develop moderate to severe cGVHD.

Subjects administered a composition of the disclosure (e.g., a cell component comprising a populations of cells described herein) exhibit a low incidence of severe cGVHD, for example, a lower incidence of severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure develop severe cGVHD, for example, within 365 days post-transplant, two years post-transplant, or within another suitable amount of time post-transplant as disclosed herein.

In some embodiments, less than about 50% of subjects that are administered a composition of the disclosure develop severe cGVHD. In some embodiments, less than about 40% of subjects that are administered a composition of the disclosure develop severe cGVHD. In some embodiments, less than about 30% of subjects that are administered a composition of the disclosure develop severe cGVHD. In some embodiments, less than about 20% of subjects that are administered a composition of the disclosure develop severe cGVHD. In some embodiments, less than about 15% of subjects that are administered a composition of the disclosure develop severe cGVHD. In some embodiments, less than about 10% of subjects that are administered a composition of the disclosure develop severe cGVHD. In some embodiments, less than about 5% of subjects that are administered a composition of the disclosure develop severe cGVHD. In some embodiments, less than about 3% of subjects that are administered a composition of the disclosure develop severe cGVHD. In some embodiments, less than about 2% of subjects that are administered a composition of the disclosure develop severe cGVHD. In some embodiments, less than about 1% of subjects that are administered a composition of the disclosure develop severe cGVHD.

The incidence of cGVHD can be assessed after a suitable amount of time elapses post-transplant, for example, about 150 days, about 200 days, about 365 days, about 1.5 years, about 2 years, about 2.5 years, about 3 years, about 3.5 years, about 4 years, about 4.5 years, or about 5 years post-transplant.

The incidence of cGVHD can be calculated based on a population of at least 10, at least at least 11, at least at least 12, at least 13, at least 14, at least 15, at least 16, at least 17, at least 18, at least 19, at least 20, at least 21, at least 22, at least 23, at least 24, at least 25, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, or at least 500 subjects.

4. No GVHD prophylaxis

Subjects administered a composition of the disclosure (e.g., a cell component comprising a population of cells described herein) in the absence of GVHD prophylactic agents exhibit a low incidence of ≥grade 1 cGVHD, for example, a lower incidence of ≥grade 1 cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure in the absence of GVHD prophylactic agents develop ≥grade 1 cGVHD, for example, within 365 days post-transplant, two years post-transplant, or within another suitable amount of time post-transplant as disclosed herein.

Subjects administered a composition of the disclosure (e.g., a cell component comprising a populations of cells described herein) in the absence of GVHD prophylactic agents exhibit a low incidence of ≥grade 2 cGVHD, for example, a lower incidence of ≥grade 2 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop ≥grade 2 cGVHD, for example, within 365 days post-transplant, two years post-transplant, or within another suitable amount of time post-transplant as disclosed herein.

In some embodiments, less than about 50% of subjects that are administered a composition of the disclosure in the absence of GVHD prophylactic agents develop ≥grade 2 cGVHD. In some embodiments, less than about 40% of subjects that are administered a composition of the disclosure in the absence of GVHD prophylactic agents develop ≥grade 2 cGVHD. In some embodiments, less than about 30% of subjects that are administered a composition of the disclosure in the absence of GVHD prophylactic agents develop ≥grade 2 cGVHD. In some embodiments, less than about 25% of subjects that are administered a composition of the disclosure in the absence of GVHD prophylactic agents develop ≥grade 2 cGVHD. In some embodiments, less than about 20% of subjects that are administered a composition of the disclosure in the absence of GVHD prophylactic agents develop ≥grade 2 cGVHD. In some embodiments, less than about 10% of subjects that are administered a composition of the disclosure in the absence of GVHD prophylactic agents develop ≥grade 2 cGVHD. In some embodiments, less than about 5% of subjects that are administered a composition of the disclosure in the absence of GVHD prophylactic agents develop ≥grade 2 cGVHD.

Subjects administered a composition of the disclosure (e.g., a cell component comprising a populations of cells described herein) in the absence of GVHD prophylactic agents exhibit a low incidence of ≥grade 3 cGVHD, for example, a lower incidence of ≥grade 3 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop ≥grade 3 cGVHD, for example, within 365 days post-transplant, two years post-transplant, or within another suitable amount of time post-transplant as disclosed herein.

In some embodiments, less than about 50% of subjects that are administered a composition of the disclosure in the absence of GVHD prophylactic agents develop ≥grade 3 cGVHD. In some embodiments, less than about 40% of subjects that are administered a composition of the disclosure in the absence of GVHD prophylactic agents develop ≥grade 3 cGVHD. In some embodiments, less than about 30% of subjects that are administered a composition of the disclosure in the absence of GVHD prophylactic agents develop ≥grade 3 cGVHD. In some embodiments, less than about 20% of subjects that are administered a composition of the disclosure in the absence of GVHD prophylactic agents develop ≥grade 3 cGVHD. In some embodiments, less than about 10% of subjects that are administered a composition of the disclosure in the absence of GVHD prophylactic agents develop ≥grade 3 cGVHD. In some embodiments, less than about 5% of subjects that are administered a composition of the disclosure in the absence of GVHD prophylactic agents develop ≥grade 3 cGVHD. In some embodiments, less than about 3% of subjects that are administered a composition of the disclosure in the absence of GVHD prophylactic agents develop ≥grade 3 cGVHD. In some embodiments, less than about 2% of subjects that are administered a composition of the disclosure in the absence of GVHD prophylactic agents develop ≥grade 3 cGVHD. In some embodiments, less than about 1% of subjects that are administered a composition of the disclosure in the absence of GVHD prophylactic agents develop ≥grade 3 cGVHD.

Subjects administered a composition of the disclosure (e.g., a cell component comprising a populations of cells described herein) in the absence of GVHD prophylactic agents exhibit a low incidence of ≥grade 4 cGVHD, for example, a lower incidence of ≥grade 4 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, less than about 30%, less than about 40%, or less than about 50% of subjects that are administered a composition of the disclosure in the absence of GVHD prophylactic agents develop ≥grade 4 cGVHD, for example, within 365 days post-transplant, two years post-transplant, or within another suitable amount of time post-transplant as disclosed herein.

In some embodiments, less than about 30% of subjects that are administered a composition of the disclosure in the absence of GVHD prophylactic agents develop ≥grade 4 cGVHD. In some embodiments, less than about 20% of subjects that are administered a composition of the disclosure in the absence of GVHD prophylactic agents develop ≥grade 4 cGVHD. In some embodiments, less than about 15% of subjects that are administered a composition of the disclosure in the absence of GVHD prophylactic agents develop ≥grade 4 cGVHD. In some embodiments, less than about 10% of subjects that are administered a composition of the disclosure in the absence of GVHD prophylactic agents develop ≥grade 4 cGVHD. In some embodiments, less than about 5% of subjects that are administered a composition of the disclosure in the absence of GVHD prophylactic agents develop ≥grade 4 cGVHD. In some embodiments, less than about 3% of subjects that are administered a composition of the disclosure in the absence of GVHD prophylactic agents develop ≥grade 4 cGVHD. In some embodiments, less than about 2% of subjects that are administered a composition of the disclosure in the absence of GVHD prophylactic agents develop ≥grade 4 cGVHD. In some embodiments, less than about 1% of subjects that are administered a composition of the disclosure in the absence of GVHD prophylactic agents develop ≥grade 4 cGVHD.

Subjects administered a composition of the disclosure (e.g., a cell component comprising a populations of cells described herein) in the absence of GVHD prophylactic agents exhibit a low incidence of mild to severe cGVHD, for example, a lower incidence of mild to severe cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure in the absence of GVHD prophylactic agents develop mild to severe cGVHD, for example, within 365 days post-transplant, two years post-transplant, or within another suitable amount of time post-transplant as disclosed herein.

Subjects administered a composition of the disclosure (e.g., a cell component comprising a populations of cells described herein) in the absence of GVHD prophylactic agents exhibit a low incidence of moderate to severe cGVHD, for example, a lower incidence of moderate to severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop moderate to severe cGVHD, for example, within 365 days post-transplant, two years post-transplant, or within another suitable amount of time post-transplant as disclosed herein.

In some embodiments, less than about 50% of subjects that are administered a composition of the disclosure in the absence of GVHD prophylactic agents develop moderate to severe cGVHD. In some embodiments, less than about 40% of subjects that are administered a composition of the disclosure in the absence of GVHD prophylactic agents develop moderate to severe cGVHD. In some embodiments, less than about 30% of subjects that are administered a composition of the disclosure in the absence of GVHD prophylactic agents develop moderate to severe cGVHD. In some embodiments, less than about 25% of subjects that are administered a composition of the disclosure in the absence of GVHD prophylactic agents develop moderate to severe cGVHD. In some embodiments, less than about 20% of subjects that are administered a composition of the disclosure in the absence of GVHD prophylactic agents develop moderate to severe cGVHD. In some embodiments, less than about 10% of subjects that are administered a composition of the disclosure in the absence of GVHD prophylactic agents develop moderate to severe cGVHD. In some embodiments, less than about 5% of subjects that are administered a composition of the disclosure in the absence of GVHD prophylactic agents develop moderate to severe cGVHD.

Subjects administered a composition of the disclosure (e.g., a cell component comprising a populations of cells described herein) in the absence of GVHD prophylactic agents exhibit a low incidence of severe cGVHD, for example, a lower incidence of severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure in the absence of GVHD prophylactic agents develop severe cGVHD, for example, within 365 days post-transplant, two years post-transplant, or within another suitable amount of time post-transplant as disclosed herein.

In some embodiments, less than about 50% of subjects that are administered a composition of the disclosure in the absence of GVHD prophylactic agents develop severe cGVHD. In some embodiments, less than about 40% of subjects that are administered a composition of the disclosure in the absence of GVHD prophylactic agents develop severe cGVHD. In some embodiments, less than about 30% of subjects that are administered a composition of the disclosure in the absence of GVHD prophylactic agents develop severe cGVHD. In some embodiments, less than about 20% of subjects that are administered a composition of the disclosure in the absence of GVHD prophylactic agents develop severe cGVHD. In some embodiments, less than about 10% of subjects that are administered a composition of the disclosure in the absence of GVHD prophylactic agents develop severe cGVHD. In some embodiments, less than about 5% of subjects that are administered a composition of the disclosure in the absence of GVHD prophylactic agents develop severe cGVHD. In some embodiments, less than about 3% of subjects that are administered a composition of the disclosure in the absence of GVHD prophylactic agents develop severe cGVHD. In some embodiments, less than about 2% of subjects that are administered a composition of the disclosure in the absence of GVHD prophylactic agents develop severe cGVHD. In some embodiments, less than about 1% of subjects that are administered a composition of the disclosure in the absence of GVHD prophylactic agents develop severe cGVHD.

The absence of GVHD prophylactic agents can refer to cases where no GVHD prophylactic agents are administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant.

### 5. Single Agent GVHD Prophylaxis

In some aspects, the methods of treating a human subject for various conditions such as stem cell-based malignancies (e.g., leukemia and lymphoma) whereby the patient receives cell populations are produced using embodiments of the kits and methods described herein and wherein the patient receives a graft versus host disease (GVHD) prophylactic agent. The method may comprise administering no more than one graft versus host disease (GVHD) prophylactic agent for less than 60 days and administering one or more pluralities of populations of cells isolated from donor blood using embodiments of kits described herein. In some embodiments, the plurality of populations of cells comprises: a population of hematopoietic stem and progenitor cells (HSPC's); a population of cells comprising regulatory T cells (Tregs); and a population of conventional T cells (Tcons). In some embodiments, the population of HSPC's comprises less than 2% CD3+ cells

In some embodiments, the method may comprise administering to the human subject a graft versus host disease (GVHD) prophylactic agent. In some embodiments, the GVHD prophylactic agent may be tacrolimus or sirolimus.

Subjects administered a composition of the disclosure (e.g., a cell component comprising a populations of cells described herein) and no more than one GVHD prophylactic agent (for example, a single GVHD prophylactic agent) exhibit a low incidence of ≥grade 1 cGVHD, for example, a lower incidence of ≥grade 1 cGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and no more than one GVHD prophylactic agent (for example, a single GVHD prophylactic agent) develop ≥grade 1 cGVHD, for example, within 365 days post-transplant, two years post-transplant, or within another suitable amount of time post-transplant as disclosed herein.

Subjects administered a composition of the disclosure (e.g., a cell component comprising a population of cells described herein) and no more than one GVHD prophylactic agent (for example, a single GVHD prophylactic agent) exhibit a low incidence of ≥grade 2 cGVHD, for example, a lower incidence of ≥grade 2 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop ≥grade 2 cGVHD, for example, within 365 days post-transplant, two years post-transplant, or within another suitable amount of time post-transplant as disclosed herein.

In some embodiments, less than about 50% of subjects that are administered a composition of the disclosure and no more than one GVHD prophylactic agent (for example, a single GVHD prophylactic agent) develop ≥grade 2 cGVHD. In some embodiments, less than about 40% of subjects that are administered a composition of the disclosure and no more than one GVHD prophylactic agent (for example, a single GVHD prophylactic agent) develop ≥grade 2 cGVHD. In some embodiments, less than about 30% of subjects that are administered a composition of the disclosure and no more than one GVHD prophylactic agent (for example, a single GVHD prophylactic agent) develop ≥grade 2 cGVHD. In some embodiments, less than about 20% of subjects that are administered a composition of the disclosure and no more than one GVHD prophylactic agent (for example, a single GVHD prophylactic agent) develop ≥grade 2 cGVHD. In some embodiments, less than about 10% of subjects that are administered a composition of the disclosure and no more than one GVHD prophylactic agent (for example, a single GVHD prophylactic agent) develop ≥grade 2 cGVHD. In some embodiments, less than about 5% of subjects that are administered a composition of the disclosure and no more than one GVHD prophylactic agent (for example, a single GVHD prophylactic agent) develop ≥grade 2 cGVHD.

Subjects administered a composition of the disclosure (e.g., a cell component comprising a populations of cells described herein) and no more than one GVHD prophylactic agent (for example, a single GVHD prophylactic agent) exhibit a low incidence of ≥grade 3 cGVHD, for example, a lower incidence of ≥grade 3 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop ≥grade 3 cGVHD, for example, within 365 days post-transplant, two years post-transplant, or within another suitable amount of time post-transplant as disclosed herein.

In some embodiments, less than about 50% of subjects that are administered a composition of the disclosure and no more than one GVHD prophylactic agent (for example, a single GVHD prophylactic agent) develop ≥grade 3 cGVHD. In some embodiments, less than about 40% of subjects that are administered a composition of the disclosure and no more than one GVHD prophylactic agent (for example, a single GVHD prophylactic agent) develop ≥grade 3 cGVHD. In some embodiments, less than about 30% of subjects that are administered a composition of the disclosure and no more than one GVHD prophylactic agent (for example, a single GVHD prophylactic agent) develop ≥grade 3 cGVHD. In some embodiments, less than about 20% of subjects that are administered a composition of the disclosure and no more than one GVHD prophylactic agent (for example, a single GVHD prophylactic agent) develop ≥grade 3 cGVHD. In some embodiments, less than about 15% of subjects that are administered a composition of the disclosure and no more than one GVHD prophylactic agent (for example, a single GVHD prophylactic agent) develop ≥grade 3 cGVHD. In some embodiments, less than about 10% of subjects that are administered a composition of the disclosure and no more than one GVHD prophylactic agent (for example, a single GVHD prophylactic agent) develop ≥grade 3 cGVHD. In some embodiments, less than about 5% of subjects that are administered a composition of the disclosure and no more than one GVHD prophylactic agent (for example, a single GVHD prophylactic agent) develop ≥grade 3 cGVHD. In some embodiments, less than about 3% of subjects that are administered a composition of the disclosure and no more than one GVHD prophylactic agent (for example, a single GVHD prophylactic agent) develop ≥grade 3 cGVHD. In some embodiments, less than about 2% of subjects that are administered a composition of the disclosure and no more than one GVHD prophylactic agent (for example, a single GVHD prophylactic agent) develop ≥grade 3 cGVHD. In some embodiments, less than about 1% of subjects that are administered a composition of the disclosure and no more than one GVHD prophylactic agent (for example, a single GVHD prophylactic agent) develop ≥grade 3 cGVHD.

Subjects administered a composition of the disclosure (e.g., a cell component comprising a populations of cells described herein) and no more than one GVHD prophylactic agent (for example, a single GVHD prophylactic agent) exhibit a low incidence of ≥grade 4 cGVHD, for example, a lower incidence of ≥grade 4 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure and no more than one GVHD prophylactic agent (for example, a single GVHD prophylactic agent) develop ≥grade 4 cGVHD, for example, within 365 days post-transplant, two years post-transplant, or within another suitable amount of time post-transplant as disclosed herein.

In some embodiments, less than about 40% of subjects that are administered a composition of the disclosure and no more than one GVHD prophylactic agent (for example, a single GVHD prophylactic agent) develop ≥grade 4 cGVHD. In some embodiments, less than about 30% of subjects that are administered a composition of the disclosure and no more than one GVHD prophylactic agent (for example, a single GVHD prophylactic agent) develop ≥grade 4 cGVHD. In some embodiments, less than about 20% of subjects that are administered a composition of the disclosure and no more than one GVHD prophylactic agent (for example, a single GVHD prophylactic agent) develop ≥grade 4 cGVHD. In some embodiments, less than about 10% of subjects that are administered a composition of the disclosure and no more than one GVHD prophylactic agent (for example, a single GVHD prophylactic agent) develop ≥grade 4 cGVHD. In some embodiments, less than about 5% of subjects that are administered a composition of the disclosure and no more than one GVHD prophylactic agent (for example, a single GVHD prophylactic agent) develop ≥grade 4 cGVHD. In some embodiments, less than about 3% of subjects that are administered a composition of the disclosure and no more than one GVHD prophylactic agent (for example, a single GVHD prophylactic agent) develop ≥grade 4 cGVHD. In some embodiments, less than about 2% of subjects that are administered a composition of the disclosure and no more than one GVHD prophylactic agent (for example, a single GVHD prophylactic agent) develop ≥grade 4 cGVHD. In some embodiments, less than about 1% of subjects that are administered a composition of the disclosure and no more than one GVHD prophylactic agent (for example, a single GVHD prophylactic agent) develop ≥grade 4 cGVHD.

Subjects administered a composition of the disclosure (e.g., a cell component comprising a populations of cells described herein) and no more than one GVHD prophylactic agent (for example, a single GVHD prophylactic agent) exhibit a low incidence of mild to severe cGVHD, for example, a lower incidence of mild to severe cGVHD than subjects that are administered an alternate composition. In some embodiments, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and no more than one GVHD prophylactic agent (for example, a single GVHD prophylactic agent) develop mild to severe cGVHD, for example, within 365 days post-transplant, two years post-transplant, or within another suitable amount of time post-transplant as disclosed herein.

Subjects administered a composition of the disclosure (e.g., a cell component comprising a populations of cells described herein) and no more than one GVHD prophylactic agent (for example, a single GVHD prophylactic agent) exhibit a low incidence of moderate to severe cGVHD, for example, a lower incidence of moderate to severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop moderate to severe cGVHD, for example, within 365 days post-transplant, two years post-transplant, or within another suitable amount of time post-transplant as disclosed herein.

In some embodiments, less than about 50% of subjects that are administered a composition of the disclosure and no more than one GVHD prophylactic agent (for example, a single GVHD prophylactic agent) develop moderate to severe cGVHD. In some embodiments, less than about 40% of subjects that are administered a composition of the disclosure and no more than one GVHD prophylactic agent (for example, a single GVHD prophylactic agent) develop moderate to severe cGVHD. In some embodiments, less than about 30% of subjects that are administered a composition of the disclosure and no more than one GVHD prophylactic agent (for example, a single GVHD prophylactic agent) develop moderate to severe cGVHD. In some embodiments, less than about 20% of subjects that are administered a composition of the disclosure and no more than one GVHD prophylactic agent (for example, a single GVHD prophylactic agent) develop moderate to severe cGVHD. In some embodiments, less than about 10% of subjects that are administered a composition of the disclosure and no more than one GVHD prophylactic agent (for example, a single GVHD prophylactic agent) develop moderate to severe cGVHD. In some embodiments, less than about 5% of subjects that are administered a composition of the disclosure and no more than one GVHD prophylactic agent (for example, a single GVHD prophylactic agent) develop moderate to severe cGVHD.

Subjects administered a composition of the disclosure (e.g., a cell component comprising a populations of cells described herein) and no more than one GVHD prophylactic agent (for example, a single GVHD prophylactic agent) exhibit a low incidence of severe cGVHD, for example, a lower incidence of severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and no more than one GVHD prophylactic agent (for example, a single GVHD prophylactic agent) develop severe cGVHD, for example, within 365 days post-transplant, two years post-transplant, or within another suitable amount of time post-transplant as disclosed herein.

In some embodiments, less than about 50% of subjects that are administered a composition of the disclosure and no more than one GVHD prophylactic agent (for example, a single GVHD prophylactic agent) develop severe cGVHD. In some embodiments, less than about 40% of subjects that are administered a composition of the disclosure and no more than one GVHD prophylactic agent (for example, a single GVHD prophylactic agent) develop severe cGVHD. In some embodiments, less than about 30% of subjects that are administered a composition of the disclosure and no more than one GVHD prophylactic agent (for example, a single GVHD prophylactic agent) develop severe cGVHD. In some embodiments, less than about 20% of subjects that are administered a composition of the disclosure and no more than one GVHD prophylactic agent (for example, a single GVHD prophylactic agent) develop severe cGVHD. In some embodiments, less than about 15% of subjects that are administered a composition of the disclosure and no more than one GVHD prophylactic agent (for example, a single GVHD prophylactic agent) develop severe cGVHD. In some embodiments, less than about 10% of subjects that are administered a composition of the disclosure and no more than one GVHD prophylactic agent (for example, a single GVHD prophylactic agent) develop severe cGVHD. In some embodiments, less than about 5% of subjects that are administered a composition of the disclosure and no more than one GVHD prophylactic agent (for example, a single GVHD prophylactic agent) develop severe cGVHD. In some embodiments, less than about 3% of subjects that are administered a composition of the disclosure and no more than one GVHD prophylactic agent (for example, a single GVHD prophylactic agent) develop severe cGVHD. In some embodiments, less than about 2% of subjects that are administered a composition of the disclosure and no more than one GVHD prophylactic agent (for example, a single GVHD prophylactic agent) develop severe cGVHD. In some embodiments, less than about 1% of subjects that are administered a composition of the disclosure and no more than one GVHD prophylactic agent (for example, a single GVHD prophylactic agent) develop severe cGVHD.

A single GVHD prophylactic agent can be tacrolimus.

A single GVHD prophylactic agent can be sirolimus.

The no more than one GVHD prophylactic agent (for example, a single GVHD prophylactic agent) can be administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant. In some embodiments, the no more than one GVHD prophylactic agent (for example, a single GVHD prophylactic agent) is administered to the subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years post-transplant.

### 6. Low Dose GVHD Prophylaxis

In some embodiments, the method may comprise administering to the human subject a graft versus host disease (GVHD) prophylactic agent. In some embodiments, the GVHD prophylactic agent may be tacrolimus or sirolimus.

Subjects administered a composition of the disclosure (e.g., a cell component comprising a populations of cells described herein) and a low dose of a GVHD prophylactic agent (for example, a single GVHD prophylactic agent at a low dose) exhibit a low incidence of ≥grade 1 cGVHD, for example, a lower incidence of ≥grade 1 cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and a low dose of a GVHD prophylactic agent (for example, a single GVHD prophylactic agent at a low dose) develop ≥grade 1 cGVHD, for example, within 365 days post-transplant, two years post-transplant, or within another suitable amount of time post-transplant as disclosed herein.

Subjects administered a composition of the disclosure (e.g., a cell component comprising a populations of cells described herein) and a low dose of a GVHD prophylactic agent (for example, a single GVHD prophylactic agent at a low dose) exhibit a low incidence of ≥grade 2 cGVHD, for example, a lower incidence of ≥grade 2 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop ≥grade 2 cGVHD, for example, within 365 days post-transplant, two years post-transplant, or within another suitable amount of time post-transplant as disclosed herein.

In some embodiments, less than about 50% of subjects that are administered a composition of the disclosure and a low dose of a GVHD prophylactic agent (for example, a single GVHD prophylactic agent at a low dose) develop ≥grade 2 cGVHD. In some embodiments, less than about 40% of subjects that are administered a composition of the disclosure and a low dose of a GVHD prophylactic agent (for example, a single GVHD prophylactic agent at a low dose) develop ≥grade 2 cGVHD. In some embodiments, less than about 30% of subjects that are administered a composition of the disclosure and a low dose of a GVHD prophylactic agent (for example, a single GVHD prophylactic agent at a low dose) develop ≥grade 2 cGVHD. In some embodiments, less than about 20% of subjects that are administered a composition of the disclosure and a low dose of a GVHD prophylactic agent (for example, a single GVHD prophylactic agent at a low dose) develop ≥grade 2 cGVHD. In some embodiments, less than about 15% of subjects that are administered a composition of the disclosure and a low dose of a GVHD prophylactic agent (for example, a single GVHD prophylactic agent at a low dose) develop ≥grade 2 cGVHD. In some embodiments, less than about 10% of subjects that are administered a composition of the disclosure and a low dose of a GVHD prophylactic agent (for example, a single GVHD prophylactic agent at a low dose) develop ≥grade 2 cGVHD. In some embodiments, less than about 5% of subjects that are administered a composition of the disclosure and a low dose of a GVHD prophylactic agent (for example, a single GVHD prophylactic agent at a low dose) develop ≥grade 2 cGVHD.

Subjects administered a composition of the disclosure (e.g., a cell component comprising a populations of cells described herein) and a low dose of a GVHD prophylactic agent (for example, a single GVHD prophylactic agent at a low dose) exhibit a low incidence of ≥grade 3 cGVHD, for example, a lower incidence of ≥grade 3 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop ≥grade 3 cGVHD, for example, within 365 days post-transplant, two years post-transplant, or within another suitable amount of time post-transplant as disclosed herein.

In some embodiments, less than about 50% of subjects that are administered a composition of the disclosure and a low dose of a GVHD prophylactic agent (for example, a single GVHD prophylactic agent at a low dose) develop ≥grade 3 cGVHD. In some embodiments, less than about 40% of subjects that are administered a composition of the disclosure and a low dose of a GVHD prophylactic agent (for example, a single GVHD prophylactic agent at a low dose) develop ≥grade 3 cGVHD. In some embodiments, less than about 30% of subjects that are administered a composition of the disclosure and a low dose of a GVHD prophylactic agent (for example, a single GVHD prophylactic agent at a low dose) develop ≥grade 3 cGVHD. In some embodiments, less than about 20% of subjects that are administered a composition of the disclosure and a low dose of a GVHD prophylactic agent (for example, a single GVHD prophylactic agent at a low dose) develop ≥grade 3 cGVHD. In some embodiments, less than about 15% of subjects that are administered a composition of the disclosure and a low dose of a GVHD prophylactic agent (for example, a single GVHD prophylactic agent at a low dose) develop ≥grade 3 cGVHD. In some embodiments, less than about 10% of subjects that are administered a composition of the disclosure and a low dose of a GVHD prophylactic agent (for example, a single GVHD prophylactic agent at a low dose) develop ≥grade 3 cGVHD. In some embodiments, less than about 5% of subjects that are administered a composition of the disclosure and a low dose of a GVHD prophylactic agent (for example, a single GVHD prophylactic agent at a low dose) develop ≥grade 3 cGVHD. In some embodiments, less than about 3% of subjects that are administered a composition of the disclosure and a low dose of a GVHD prophylactic agent (for example, a single GVHD prophylactic agent at a low dose) develop ≥grade 3 cGVHD. In some embodiments, less than about 2% of subjects that are administered a composition of the disclosure and a low dose of a GVHD prophylactic agent (for example, a single GVHD prophylactic agent at a low dose) develop ≥grade 3 cGVHD. In some embodiments, less than about 1% of subjects that are administered a composition of the disclosure and a low dose of a GVHD prophylactic agent (for example, a single GVHD prophylactic agent at a low dose) develop ≥grade 3 cGVHD.

Subjects administered a composition of the disclosure (e.g., a cell component comprising a populations of cells described herein) and a low dose of a GVHD prophylactic agent (for example, a single GVHD prophylactic agent at a low dose) exhibit a low incidence of ≥grade 4 cGVHD, for example, a lower incidence of ≥grade 4 cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, or less than about 30% of subjects that are administered a composition of the disclosure and a low dose of a GVHD prophylactic agent (for example, a single GVHD prophylactic agent at a low dose) develop ≥grade 4 cGVHD, for example, within 365 days post-transplant, two years post-transplant, or within another suitable amount of time post-transplant as disclosed herein.

In some embodiments, less than about 40% of subjects that are administered a composition of the disclosure and a low dose of a GVHD prophylactic agent (for example, a single GVHD prophylactic agent at a low dose) develop ≥grade 4 cGVHD. In some embodiments, less than about 30% of subjects that are administered a composition of the disclosure and a low dose of a GVHD prophylactic agent (for example, a single GVHD prophylactic agent at a low dose) develop ≥grade 4 cGVHD. In some embodiments, less than about 20% of subjects that are administered a composition of the disclosure and a low dose of a GVHD prophylactic agent (for example, a single GVHD prophylactic agent at a low dose) develop ≥grade 4 cGVHD. In some embodiments, less than about 10% of subjects that are administered a composition of the disclosure and a low dose of a GVHD prophylactic agent (for example, a single GVHD prophylactic agent at a low dose) develop ≥grade 4 cGVHD. In some embodiments, less than about 5% of subjects that are administered a composition of the disclosure and a low dose of a GVHD prophylactic agent (for example, a single GVHD prophylactic agent at a low dose) develop ≥grade 4 cGVHD. In some embodiments, less than about 3% of subjects that are administered a composition of the disclosure and a low dose of a GVHD prophylactic agent (for example, a single GVHD prophylactic agent at a low dose) develop ≥grade 4 cGVHD. In some embodiments, less than about 2% of subjects that are administered a composition of the disclosure and a low dose of a GVHD prophylactic agent (for example, a single GVHD prophylactic agent at a low dose) develop ≥grade 4 cGVHD. In some embodiments, less than about 1% of subjects that are administered a composition of the disclosure and a low dose of a GVHD prophylactic agent (for example, a single GVHD prophylactic agent at a low dose) develop ≥grade 4 cGVHD.

Subjects administered a composition of the disclosure (e.g., a cell component comprising a populations of cells described herein) and a low dose of a GVHD prophylactic agent (for example, a single GVHD prophylactic agent at a low dose) exhibit a low incidence of mild to severe cGVHD, for example, a lower incidence of mild to severe cGVHD than subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure and a low dose of a GVHD prophylactic agent (for example, a single GVHD prophylactic agent at a low dose) develop mild to severe cGVHD, for example, within 365 days post-transplant, two years post-transplant, or within another suitable amount of time post-transplant as disclosed herein.

Subjects administered a composition of the disclosure (e.g., a cell component comprising a populations of cells described herein) and a low dose of a GVHD prophylactic agent (for example, a single GVHD prophylactic agent at a low dose) exhibit a low incidence of moderate to severe cGVHD, for example, a lower incidence of moderate to severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop moderate to severe cGVHD, for example, within 365 days post-transplant, two years post-transplant, or within another suitable amount of time post-transplant as disclosed herein.

In some embodiments, less than about 50% of subjects that are administered a composition of the disclosure and a low dose of a GVHD prophylactic agent (for example, a single GVHD prophylactic agent at a low dose) develop moderate to severe cGVHD. In some embodiments, less than about 40% of subjects that are administered a composition of the disclosure and a low dose of a GVHD prophylactic agent (for example, a single GVHD prophylactic agent at a low dose) develop moderate to severe cGVHD. In some embodiments, less than about 30% of subjects that are administered a composition of the disclosure and a low dose of a GVHD prophylactic agent (for example, a single GVHD prophylactic agent at a low dose) develop moderate to severe cGVHD. In some embodiments, less than about 20% of subjects that are administered a composition of the disclosure and a low dose of a GVHD prophylactic agent (for example, a single GVHD prophylactic agent at a low dose) develop moderate to severe cGVHD. In some embodiments, less than about 15% of subjects that are administered a composition of the disclosure and a low dose of a GVHD prophylactic agent (for example, a single GVHD prophylactic agent at a low dose) develop moderate to severe cGVHD. In some embodiments, less than about 10% of subjects that are administered a composition of the disclosure and a low dose of a GVHD prophylactic agent (for example, a single GVHD prophylactic agent at a low dose) develop moderate to severe cGVHD. In some embodiments, less than about 5% of subjects that are administered a composition of the disclosure and a low dose of a GVHD prophylactic agent (for example, a single GVHD prophylactic agent at a low dose) develop moderate to severe cGVHD.

Subjects administered a composition of the disclosure (e.g., a cell component comprising a populations of cells described herein) and a low dose of a GVHD prophylactic agent (for example, a single GVHD prophylactic agent at a low dose) exhibit a low incidence of severe cGVHD, for example, a lower incidence of severe cGVHD than subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure and a low dose of a GVHD prophylactic agent (for example, a single GVHD prophylactic agent at a low dose) develop severe cGVHD, for example, within 365 days post-transplant, two years post-transplant, or within another suitable amount of time post-transplant as disclosed herein.

In some embodiments, less than about 50% of subjects that are administered a composition of the disclosure and a low dose of a GVHD prophylactic agent (for example, a single GVHD prophylactic agent at a low dose) develop severe cGVHD. In some embodiments, less than about 40% of subjects that are administered a composition of the disclosure and a low dose of a GVHD prophylactic agent (for example, a single GVHD prophylactic agent at a low dose) develop severe cGVHD. In some embodiments, less than about 30% of subjects that are administered a composition of the disclosure and a low dose of a GVHD prophylactic agent (for example, a single GVHD prophylactic agent at a low dose) develop severe cGVHD. In some embodiments, less than about 20% of subjects that are administered a composition of the disclosure and a low dose of a GVHD prophylactic agent (for example, a single GVHD prophylactic agent at a low dose) develop severe cGVHD. In some embodiments, less than about 15% of subjects that are administered a composition of the disclosure and a low dose of a GVHD prophylactic agent (for example, a single GVHD prophylactic agent at a low dose) develop severe cGVHD. In some embodiments, less than about 10% of subjects that are administered a composition of the disclosure and a low dose of a GVHD prophylactic agent (for example, a single GVHD prophylactic agent at a low dose) develop severe cGVHD. In some embodiments, less than about 5% of subjects that are administered a composition of the disclosure and a low dose of a GVHD prophylactic agent (for example, a single GVHD prophylactic agent at a low dose) develop severe cGVHD. In some embodiments, less than about 3% of subjects that are administered a composition of the disclosure and a low dose of a GVHD prophylactic agent (for example, a single GVHD prophylactic agent at a low dose) develop severe cGVHD. In some embodiments, less than about 2% of subjects that are administered a composition of the disclosure and a low dose of a GVHD prophylactic agent (for example, a single GVHD prophylactic agent at a low dose) develop severe cGVHD. In some embodiments, less than about 1% of subjects that are administered a composition of the disclosure and a low dose of a GVHD prophylactic agent (for example, a single GVHD prophylactic agent at a low dose) develop severe cGVHD.

A low dose of a GVHD prophylactic agent can be, for example a target trough level of less than about 25 ng/mL, less than about 20 ng/mL, less than about 15 ng/mL, less than about 12 ng/mL, less than about 11 ng/mL, less than about 10 ng/mL, less than about 9 ng/mL, less than about 8 ng/mL, less than about 7 ng/mL, less than about 6 ng/mL, less than about 5 ng/mL, less than about 4 ng/mL, less than about 3 ng/mL, less than about 2 ng/mL, or less than about 1 ng/mL.

In some embodiments, a low dose of a GVHD prophylactic is a target trough level of about 1-25 ng/mL, about 1-20 ng/mL, about 1-15 ng/mL, about 1-12 ng/mL, about 1-11 ng/mL, about 1-10 ng/mL, about 1-9 ng/mL, about 1-8 ng/mL, about 1-7 ng/mL, about 1-6 ng/mL, about 1-5 ng/mL, about 1-4 ng/mL, about 1-3 ng/mL, about 1-2 ng/mL, about 2-25 ng/mL, about 2-20 ng/mL, about 2-15 ng/mL, about 2-12 ng/mL, about 2-11 ng/mL, about 2-10 ng/mL, about 2-9 ng/mL, about 2-8 ng/mL, about 2-7 ng/mL, about 2-6 ng/mL, about 2-5 ng/mL, about 2-4 ng/mL, about 2-3 ng/mL, about 3-25 ng/mL, about 3-20 ng/mL, about 3-15 ng/mL, about 3-12 ng/mL, about 3-11 ng/mL, about 3-10 ng/mL, about 3-9 ng/mL, about 3-8 ng/mL, about 3-7 ng/mL, about 3-6 ng/mL, about 3-5 ng/mL, about 3-4 ng/mL, about 4-25 ng/mL, about 4-20 ng/mL, about 4-15 ng/mL, about 4-12 ng/mL, about 4-11 ng/mL, about 4-10 ng/mL, about 4-9 ng/mL, about 4-8 ng/mL, about 4-7 ng/mL, about 4-6 ng/mL, about 4-5 ng/mL, about 5-25 ng/mL, about 5-20 ng/mL, about 5-15 ng/mL, about 5-12 ng/mL, about 5-11 ng/mL, about 5-10 ng/mL, about 5-9 ng/mL, about 5-8 ng/mL, about 5-7 ng/mL, about 5-6 ng/mL, about 6-25 g/mL, about 6-20 ng/mL, about 6-15 ng/mL, about 6-12 ng/mL, about 6-11 ng/mL, about 6-10 ng/mL, about 6-9 ng/mL, about 6-8 ng/mL, about 6-7 ng/mL, about 8-25 ng/mL, about 8-20 ng/mL, about 8-15 ng/mL, about 8-12 ng/mL, about 8-11 ng/mL, about 8-10 ng/mL, about 8-9 ng/mL, about 10-25 ng/mL, about 10-20 ng/mL, about 10-15 ng/mL, about 10-12 ng/mL, or about 10-11 ng/mL.

In some embodiments, a low dose of a GVHD prophylactic agent is tacrolimus with a target trough level of about 5 ng/mL to about 10 ng/mL. In some embodiments, a low dose of a GVHD prophylactic agent is tacrolimus with a target trough level of about 4 ng/mL to about 6 ng/mL.

In some embodiments, a low dose of a GVHD prophylactic agent is sirolimus with a target trough level of about 3 ng/mL to about 8 ng/mL. In some embodiments, a low dose of a GVHD prophylactic agent is sirolimus with a target trough level of about 4 ng/mL to about 8 ng/mL.

The low dose GVHD prophylactic agent (for example, single GVHD prophylactic agent at a low dose) can be administered to the subject for the first 20 days, 30 days, 40 days, 50 days, 60 days, 70 days, 80 days, 90 days, 100 days, 110 days, 120 days, 150 days, 200 days, 365 days, 13 months, 14 months, 15 months, 16 months, 17 months, 18 months, 19 months, 20 months, 21 months, 22 months, 23 months, 2 years, 2.5 years, 3 years, 3.5 years, 4 years, 4.5 years, or 5 years post-transplant. In some embodiments, the low dose GVHD prophylactic agent (for example, single GVHD prophylactic agent at a low dose) is administered to the subject for less than about 20 days, less than about 30 days, less than about 40 days, less than about 50 days, less than about 60 days, less than about 70 days, less than about 80 days, less than about 90 days, less than about 100 days, less than about 110 days, less than about 120 days, less than about 150 days, less than about 200 days, less than about 365 days, less than about 13 months, less than about 14 months, less than about 15 months, less than about 16 months, less than about 17 months, less than about 18 months, less than about 19 months, less than about 20 months, less than about 21 months, less than about 22 months, less than about 23 months, less than about 2 years, less than about 2.5 years, less than about 3 years, less than about 3.5 years, less than about 4 years, less than about 4.5 years, or less than about or 5 years post-transplant.

### E. Organ-Specific GVHD Stage Incidence

Subjects administered a composition of the disclosure (e.g., a cell component comprising a populations of cells described herein) exhibit a low incidence of ≥stage 1 GVHD signs for the skin, liver, gut, or a combination thereof, for example, a lower incidence compared to subjects that are administered an alternate composition. In some embodiments, less than about 5%, less than about 10%, less than about 15%, less than about 20%, less than about 25%, less than about 30%, less than about 35%, less than about 40%, less than about 50%, less than about 55%, less than about 60%, less than about 65%, less than about 70%, less than about 75%, less than about 80%, or less than about 85% of subjects that are administered a composition of the disclosure exhibit ≥stage 1 GVHD signs for the skin, liver, gut, or a combination thereof.

Subjects administered a composition of the disclosure (e.g., a cell component comprising a populations of cells described herein) exhibit a low incidence of ≥stage 2 GVHD signs for the skin, liver, gut, or a combination thereof, for example, a lower incidence compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure exhibit ≥stage 1 GVHD signs for the skin, liver, gut, or a combination thereof.

Subjects administered a composition of the disclosure (e.g., a cell component comprising a populations of cells described herein) exhibit a low incidence of ≥stage 3 GVHD signs for the skin, liver, gut, or a combination thereof, for example, a lower incidence compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure exhibit ≥stage 3 GVHD signs for the skin, liver, gut, or a combination thereof.

Subjects administered a composition of the disclosure (e.g., a cell component comprising a populations of cells described herein) exhibit a low incidence of ≥stage 4 GVHD signs for the skin, liver, gut, or a combination thereof, for example, a lower incidence compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, less than about 25%, less than about 26%, less than about 27%, less than about 28%, less than about 29%, less than about 30%, less than about 31%, less than about 32%, less than about 33%, less than about 34%, less than about 35%, less than about 36%, less than about 37%, less than about 38%, less than about 39%, less than about 40%, less than about 41%, less than about 42%, less than about 43%, less than about 44%, less than about 45%, less than about 46%, less than about 47%, less than about 48%, less than about 49%, or less than about 50% of subjects that are administered a composition of the disclosure exhibit ≥stage 4 GVHD signs for the skin, liver, gut, or a combination thereof.

The incidence of the organ-specific GVHD signs can be assessed after a suitable amount of time elapses post-transplant, for example, about 20 days, about 21 days, about 25 days, about 28 days, about 30 days, about 35 days, about 40 days, about 42 days, about 45 days, about 49 days, about 50 days, about 55 days, about 56 days, about 60 days, about 63 days, about 65 days, about 70 days, about 75 days, about 77 days, about 80 days, about 84 days, about 85 days, about 90 days, about 91 days, about 95 days, about 98 days, about 100 days, about 105 days, about 110 days, about 112 days, about 115 days, about 119 days, about 120 days, about 150 days, about 200 days, about 365 days, about 1.5 years, about 2 years, about 2.5 years, about 3 years, about 3.5 years, about 4 years, about 4.5 years, or about 5 years post-transplant.

The incidence of the organ-specific GVHD signs can be calculated based on a population of, for example, at least 10, at least at least 11, at least at least 12, at least 13, at least 14, at least 15, at least 16, at least 17, at least 18, at least 19, at least 20, at least 21, at least 22, at least 23, at least 24, at least 25, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, or at least 500 subjects.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥1.5 when evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

### IV. Other Clinical Outcomes

### A. Survival, Hospitalization, and Relapse

A population of subjects administered a composition of the disclosure (e.g., a cell component comprising a population of cells described herein) exhibit a high overall survival rate, for example, a higher overall survival rate compared to subjects that are administered an alternate composition. In some embodiments, at least about 40%, at least about 50%, at least about 55%, at least about 60%, at least about 65%, at least about 70%, at least about 75%, at least about 80%, at least about 85%, at least about 90%, or at least about 95% of subjects that are administered a composition of the disclosure are alive after about 1 year, about 1.5 years, about 2 years, about 2.5 years, about 3 years, about 3.5 years, about 4 years, about 4.5 years, about 5 years, about 7 years, about 10 years, about 15 years, about 20 years, about 30 years post-transplant.

A population of subjects administered a composition of the disclosure (e.g., a cell component comprising a population of cells described herein) exhibit a low treatment-associated mortality rate, for example, a lower treatment-associated mortality rate compared to subjects that are administered an alternate composition. In some embodiments, a treatment-associated mortality rate of a population of subjects administered a composition of the disclosure is less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 25%, less than about 30%, less than about 35%, less than about 40%, or less than about 50% when evaluated after a suitable amount of time post-transplant, for example at about 6 months, about 1 year, about 1.5 years, about 2 years, about 2.5 years, about 3 years, about 3.5 years, about 4 years, about 4.5 years, or about 5 years post-transplant. In some embodiments a treatment-associated mortality rate of a population of subjects administered a composition of the disclosure is less than about 5% when evaluated at 1-year post-transplant. In some embodiments a treatment-associated mortality rate of a population of subjects administered a composition of the disclosure is less than about 1% when evaluated at 1-year post-transplant.

A population of subjects administered a composition of the disclosure (e.g., a cell component comprising a population of cells described herein) exhibit GVHD-free and relapse-free survival (GRFS) rate, for example, a higher GRFS rate compared to subjects that are administered an alternate composition. In some embodiments, GRFS can refer to survival without relapse or Grade ≥3 aGVHD or extensive (e.g., severe) cGVHD. In some embodiments, GRFS can refer to survival without relapse or Grade ≥2 aGVHD or extensive (e.g., moderate to severe) cGVHD. In some embodiments, GRFS can refer to survival with no GVHD symptoms. In some embodiments, a GRFS rate of a population of subjects administered a composition of the disclosure is at least about 20%, at least about 30%, at least about 40%, at least about 50%, at least about 55%, at least about 60%, at least about 65%, at least about 70%, at least about 75%, at least about 80%, at least about 85%, at least about 90%, or at least about 95% when evaluated after a suitable amount of time post-transplant, for example at about 6 months, about 1 year, about 1.5 years, about 2 years, about 2.5 years, about 3 years, about 3.5 years, about 4 years, about 4.5 years, or about 5 years post-transplant. In some embodiments, a GRFS rate of a population of subjects administered a composition of the disclosure is at least about 60% when evaluated at 1-year post-transplant. In some embodiments, a GRFS rate of a population of subjects administered a composition of the disclosure is at least about 75% when evaluated at 1-year post-transplant. In some embodiments, a GRFS rate of a population of subjects administered a composition of the disclosure is more than 75% when evaluated at 3 years post-transplant. In some embodiments, a GRFS rate of a population of subjects administered a composition of the disclosure is more than 75% when evaluated at 5 years post-transplant. In some embodiments, a GRFS rate of a population of subjects administered a composition of the disclosure is more than 75% when evaluated at 7 years post-transplant. In some embodiments, a GRFS rate of a population of subjects administered a composition of the disclosure is more than 75% when evaluated at 10 years post-transplant.

A population of subjects administered a composition of the disclosure (e.g., a cell component comprising a population of cells described herein) exhibit a short time to discharge from hospital, for example, a shorter time to discharge from hospital compared to subjects that are administered an alternate composition. In some embodiments, the average time to discharge after day 0 of a transplantation regimen is less than about 20 days, less than about 19 days, less than about 18 days, less than about 17 days, less than about 16 days, less than about 15 days, less than about 14 days, less than about 13 days, less than about 12 days, less than about 11 days, less than about 10 days, less than about 9 days, or less than about 8 days. In some embodiments, the average time to discharge after day 0 of a transplantation regimen is less than about 17 days. In some embodiments, the average time to discharge after day 0 of a transplantation regimen is less than about 18 days.

A population of subjects administered a composition of the disclosure (e.g., a cell component comprising a population of cells described herein) exhibit a low relapse rate, for example, a lower relapse rate compared to subjects that are administered an alternate composition. In some embodiments, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 25%, less than about 30%, less than about 35%, less than about 40%, less than about 45%, less than about 50%, less than about 55%, or less than about 60% of subjects that are administered a composition of the disclosure relapse within a suitable amount of time post-transplant, for example at about 90 days, about 100 days, about 120 days, about 6 months, about 1 year, about 1.5 years, about 2 years, about 2.5 years, about 3 years, about 3.5 years, about 4 years, about 4.5 years, or about 5 years post-transplant. In some embodiments, less than about 35% of subjects that are administered a composition of the disclosure relapse within 1-year post-transplant. In some embodiments, less than about 25% of subjects that are administered a composition of the disclosure relapse within 1-year post-transplant.

A population of subjects that do not have active disease when administered a composition of the disclosure (e.g., a cell component comprising a populations of cells described herein) exhibit a low relapse rate, for example, a lower relapse rate compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 25%, less than about 30%, less than about 35%, less than about 40%, less than about 45%, less than about 50%, less than about 55%, or less than about 60% of subjects that do not have active disease when administered a composition of the disclosure relapse within a suitable amount of time post-transplant, for example at about 90 days, about 100 days, about 120 days, about 6 months, about 1 year, about 1.5 years, about 2 years, about 2.5 years, about 3 years, about 3.5 years, about 4 years, about 4.5 years, or about 5 years post-transplant.

A population of subjects that are in complete remission when administered a composition of the disclosure (e.g., a cell component comprising a population of cells described herein) exhibit a low relapse rate, for example, a lower relapse rate compared to subjects that are administered an alternate composition. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 25%, less than about 30%, less than about 35%, or less than about 40% of subjects that are in complete remission when administered a composition of the disclosure relapse within a suitable amount of time post-transplant, for example at about 90 days, about 100 days, about 120 days, about 6 months, about 1 year, about 1.5 years, about 2 years, about 2.5 years, about 3 years, about 3.5 years, about 4 years, about 4.5 years, or about 5 years post-transplant.

### B. Engraftment and Immune Reconstitution

### 7. Graft Failure

A population of subjects that are administered a composition of the disclosure (e.g., a cell component comprising a population of cells described herein) exhibit a low rate of primary graft failure, for example, a lower rate of primary graft failure compared to subjects that are administered an alternate composition. Primary graft failure can be a failure to achieve an absolute neutrophil count of ≥500 cells/μL after Day 30 post-transplant. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 30%, or less than about 40% of subjects administered a composition of the disclosure exhibit primary graft failure. In some embodiments, less than about 1% of subjects administered a composition of the disclosure exhibit primary graft failure. In some embodiments, less than about 3% of subjects administered a composition of the disclosure exhibit primary graft failure.

A population of subjects that are administered a composition of the disclosure (e.g., a cell component comprising a populations of cells described herein) exhibit a low rate of secondary graft failure, for example, a lower rate of secondary graft failure compared to subjects that are administered an alternate composition. Secondary graft failure can be a sustained loss of hematopoiesis after engraftment. In some embodiments, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 20%, less than about 30%, or less than about 40% of subjects administered a composition of the disclosure exhibit secondary graft failure when evaluated a suitable amount of time post-transplant, for example at about 90 days, about 100 days, about 120 days, about 6 months, about 1 year, about 1.5 years, about 2 years, about 2.5 years, about 3 years, about 3.5 years, about 4 years, about 4.5 years, or about 5 years post-transplant. In some embodiments, less than about 1% of subjects administered a composition of the disclosure exhibit secondary graft failure within 1-year post-transplant. In some embodiments, less than about 5% of subjects administered a composition of the disclosure exhibit secondary graft failure within 1-year post-transplant.

### 8. Neutrophil Engraftment

A population of subjects that are administered a composition of the disclosure (e.g., a cell component comprising a populations of cells described herein) exhibit fast neutrophil engraftment, for example, faster neutrophil engraftment compared to subjects that are administered an alternate composition. Neutrophil engraftment can be indicated by a sustained neutrophil count of ≥500 cells/μL in the peripheral blood of the recipient. In some embodiments, a population of subjects administered a composition of the disclosure achieve neutrophil engraftment by a median of about 7 days, about 8 days, about 9 days, about 10 days, about 11 days, about 12 days, about 12 days, about 14 days, or about 15 days post-transplant. In some embodiments, a population of subjects administered a composition of the disclosure achieve neutrophil engraftment by a median of 13 days post-transplant. In some embodiments, a population of subjects administered a composition of the disclosure achieve neutrophil engraftment by a median of 12 days post-transplant. In some embodiments, a population of subjects administered a composition of the disclosure achieve neutrophil engraftment by a median of 11 days post-transplant.

### 9. Platelet Engraftment

A population of subjects that are administered a composition of the disclosure (e.g., a cell component comprising a population of cells described herein) exhibit fast platelet engraftment, for example, faster platelet engraftment compared to subjects that are administered an alternate composition. Platelet engraftment can be indicated by a platelet count ≥20,000/mm³ for 3 consecutive days without platelet transfusion in the peripheral blood of the recipient. In some embodiments, a population of subjects administered a composition of the disclosure achieve platelet engraftment by a median of about 7 days, about 8 days, about 9 days, about 10 days, about 11 days, about 12 days, about 12 days, about 14 days, or about 15 days post-transplant. In some embodiments, a population of subjects administered a composition of the disclosure achieve platelet engraftment by a median of 13 days post-transplant. In some embodiments, a population of subjects administered a composition of the disclosure achieve platelet engraftment by a median of 12 days post-transplant. In some embodiments, a population of subjects administered a composition of the disclosure achieve platelet engraftment by a median of 11 days post-transplant.

### 10. T Cell Engraftment

A population of subjects that are administered a composition of the disclosure (e.g., a cell component comprising a populations of cells described herein) exhibit a high proportion of circulating Tregs, for example, a higher proportion of circulating Tregs compared to subjects that are administered an alternate composition. In some embodiments, an average of at least about 5%, at least about 7.5%, at least about 10%, at least about 12.5%, at least about 15%, at least about 16%, at least about 17%, at least about 18%, at least about 19%, at least about 20%, at least about 21%, at least about 22%, at least about 23%, at least about 24%, or at least about 25% of circulating CD4+ T cells are Tregs when subjects are evaluated a suitable amount of time post-transplant, for example, at about 14 days, 15 days, 20 days, 21 days, 25 days, 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100 days, 110 days, 120 days, 130 days, 140 days, 150 days, 160 days, 170 days, or 180 days post-transplant. In some embodiments, an average of at least about 15% of circulating CD4+ T cells are Tregs when subjects are evaluated 28 days post-transplant.

A population of subjects that are administered a composition of the disclosure (e.g., a cell component comprising a populations of cells described herein) exhibit a normal CD4:CD8 T cell ratio, for example, a higher CD4:CD8 T cell ratio compared to subjects that are administered an alternate composition or normal healthy control subjects.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥0.8 when evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥1 when evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant. In some embodiments, at least 50% of subjects have a CD4:CD8 T cell ratio of ≥1 when evaluated 28 days post-transplant.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥1.2 when evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, at least about 50%, at least about 55%, at least about 60%, at least about 65%, at least about 70%, at least about 75%, at least about 80%, at least about 85%, at least about 90%, or at least about 95% of subjects have a CD4:CD8 T cell ratio of ≥1.5 when evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

A population of subjects that are administered a composition of the disclosure (e.g., a cell component comprising a populations of cells described herein) exhibit a high proportion of donor-derived circulating T cells at an early timepoint after transplant, for example, a higher proportion of donor-derived circulating T cells compared to subjects that are administered an alternate composition.

In some embodiments, more than 50% of circulating T cells are donor-derived in at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, more than 60% of circulating T cells are donor-derived in at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, more than 70% of circulating T cells are donor-derived in at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, more than 80% of circulating T cells are donor-derived in at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, more than 90% of circulating T cells are donor-derived in at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 14 days, 15 days, 20 days, 21 days, 22 days, 23 days, 24 days, 25 days, 26 days, 27 days, 28 days, 29 days, 30 days, 31 days, 32 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, or 100 days post-transplant.

In some embodiments, more than 50% of circulating T cells are donor-derived in at least about at least about 70% of subjects evaluated 30 days post-transplant. In some embodiments, more than 70% of circulating T cells are donor-derived in at least about at least about 60% of subjects evaluated 30 days post-transplant.

### 11. B Cell Engraftment

A population of subjects that are administered a composition of the disclosure (e.g., a cell component comprising a populations of cells described herein) exhibit a high concentration of circulating B cells at an early timepoint after transplant, for example, a higher concentration of circulating B cells compared to subjects that are administered an alternate composition. Achieving a high concentration of circulating B cells at an early timepoint after transplant can be important for immunocompetence, and can allow vaccines to elicit protective immune responses at an earlier timepoint post-transplant.

In some embodiments, more than about 50 B cells per L are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, more than 50 B cells per L are present in the blood of at least 75% of subjects evaluated about 60 days post-transplant.

In some embodiments, more than about 60 B cells per L are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, more than 60 B cells per L are present in the blood of at least 75% of subjects evaluated about 60 days post-transplant.

In some embodiments, more than about 70 B cells per L are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, more than 70 B cells per L are present in the blood of at least 75% of subjects evaluated about 60 days post-transplant.

In some embodiments, more than about 80 B cells per L are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant.

In some embodiments, more than about 90 B cells per L are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, more than 90 B cells per L are present in the blood of at least 75% of subjects evaluated about 180 days post-transplant.

In some embodiments, more than about 100 B cells per L are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, more than 100 B cells per L are present in the blood of at least 75% of subjects evaluated about 180 days post-transplant.

In some embodiments, more than about 110 B cells per L are present in the blood of at least about 50%, at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, more than 110 B cells per L are present in the blood of at least 75% of subjects evaluated about 180 days post-transplant.

According to one or more embodiments, a population of subjects that are administered a composition of the disclosure (e.g., a cell component comprising a populations of cells described herein) exhibit a high proportion of mature B cells at an early timepoint after transplant, for example, a higher proportion of circulating B cells that are mature B cells (e.g., IgD+ and/or CD27+) compared to subjects that are administered an alternate composition. Achieving a high proportion of mature B cells at an early timepoint after transplant can be important for immunocompetence, and can allow vaccines to elicit protective immune responses at an earlier timepoint post-transplant.

In some embodiments, at least about 50% of circulating CD19+ B cells are IgD+ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant.

In some embodiments, at least about 60% of circulating CD19+ B cells are IgD+ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant.

In some embodiments, at least about 70% of circulating CD19+ B cells are IgD+ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant.

In some embodiments, at least about 80% of circulating CD19+ B cells are IgD+ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, at least about 80% of circulating CD19+ B cells are IgD+ in at least about 75% of subjects evaluated about 100 days post-transplant.

In some embodiments, at least about 90% of circulating CD19+ B cells are IgD+ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, at least about 90% of circulating CD19+ B cells are IgD+ in at least about 75% of subjects evaluated about 100 days post-transplant.

In some embodiments, at least about 25% of circulating CD19+ B cells are CD27+ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant.

In some embodiments, at least about 30% of circulating CD19+ B cells are CD27+ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, at least about 30% of circulating CD19+ B cells are CD27+ in at least about 75% of subjects evaluated at 100 days post-transplant.

In some embodiments, at least about 35% of circulating CD19+ B cells are CD27+ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, at least about 35% of circulating CD19+ B cells are CD27+ in at least about 75% of subjects evaluated at 100 days post-transplant.

In some embodiments, at least about 40% of circulating CD19+ B cells are CD27+ in at least about 55%, at least about 60%, at least about 65%, at least about 70%, at least about 75%, at least about 80%, at least about 85%, at least about 90%, or at least about 95% of subjects evaluated after a suitable amount of time post-transplant, for example, at about 28 days, 30 days, 35 days, 40 days, 42 days, 45 days, 49 days, 50 days, 55 days, 56 days, 60 days, 63 days, 65 days, 70 days, 75 days, 77 days, 80 days, 84 days, 85 days, 90 days, 91 days, 95 days, 98 days, 100, 105, 110, 112, 115, 119, 120, 125, 126, 130, 133, or 140 days post-transplant. In some embodiments, at least about 40% of circulating CD19+ B cells are CD27+ in at least about 75% of subjects evaluated at 100 days post-transplant.

Clinical outcomes disclosed herein can be calculated based on a patient population of a number of sizes. For example in various embodiments clinical outcomes can be calculated based on patient population of, at least 10, at least at least 11, at least at least 12, at least 13, at least 14, at least 15, at least 16, at least 17, at least 18, at least 19, at least 20, at least 21, at least 22, at least 23, at least 24, at least 25, at least 25, at least 30, at least 35, at least 40, at least 45, at least 50, at least 60, at least 70, at least 80, at least 90, at least 100, at least 120, at least 140, at least 160, at least 180, at least 200, at least 250, at least 300, at least 350, at least 400, at least 450, or at least 500 subjects.

### V. Embodiments of Kits for the Preparation of Cellular Component of a Therapeutic Composition

Instructions for use (IFU) may accompany the therapeutic kits or therapeutic preparation kits described herein. According to one or more embodiments, the IFU may be written or in electronic form and in the latter case, they may be stored on computer readable media including non-transitory computer readable media such as a flash memory device or they may be assessable through a network and/or the cloud. In one or more embodiments the IFU may include information unique to a particular batch of the therapeutic composition including unique to a particular cell population in the therapeutic composition (e.g., HSPC's). Such information may include one or more of the number of cells (total or per ml), cell purity and levels of impurities (including cells and molecules such as endotoxins) and information on the donor including match characteristics (e.g., human leukocyte antigen, blood type and Rh Factor). For embodiments of the disclosure including kit for the preparation of the therapeutic composition, the IFU may also include information unique to a batch of antigen binding agents used in the kit, the information used by an instrument or device in the preparation of the therapeutic composition to optimize a parameter of composition. The parameter may correspond to one or more of a purity, concentration or dose of cells in the therapeutic composition such as hematopoietic stem and progenitor cells

Various embodiments of the disclosure provide kits for the preparation of cellular components of at least one therapeutic composition (and/or the whole therapeutic composition) that is prepared or otherwise derived from a blood product where the composition is used for the treatment of a disease or condition such as stem cell-based cancer (e.g., leukemia, lymphoma or myeloma), graft versus host disease related to the treatment of the cancer or various autoimmune diseases. Referring now to FIG. 12 to FIG. 16, according to one or more embodiments of the disclosure, a kit 10 for the preparation of cellular components 24 of a therapeutic composition 20 prepared from a blood product 30 comprises a plurality 41 of antigen binging agents 40 that target specified cells in blood product 30 (which will comprise the therapeutic composition(s)), packaging 50 associated with the plurality 41 of antigen binding agents 40 and instructions 60 for use of the kit to prepare the therapeutic composition(s). Typically, the plurality 41 of antigen binding agents 40 comprises at least first and second pluralities 42 and 44 of antigen of antigen binding agents 40. In one or more embodiments, the first plurality 42 of binding agents comprise CD34+ antigen binding agents 43 that target CD34+ cells and the second plurality 44 of antigen binding agents comprise CD25+ binding agents 45 that target CD25+ cells respectively in the blood product 30. Typically, blood product 30 will comprise nucleated cells 31 (e.g., stem cells, T-cells etc.) and will be mobilized blood product 32, that is, blood product obtained from a donor who has received drugs and related treatments to mobilize or otherwise cause transport of their stem cells from their bone marrow into their peripheral blood. However, embodiments using non-mobilized blood product are also contemplated including, for example, use of umbilical cord blood including that of the patient. The therapeutic composition 20 will also typically comprise one or more (also referred to as at least one) therapeutic compositions, for example, at least a first and second cell-based therapeutic compositions 21 and 22 which may be used in a treatment such as HSPC transplantation. In embodiments where the treatment comprises HSPC transplantation, first therapeutic composition 21 may comprise HSPC's 28 and second therapeutic composition 22 may comprise regulatory T-cells 26, also referred to as Treg cells or Tregs.

The antigen-binding agents 40 will typically be conjugated to a particle 70 which allows for the separation of CD 34+ and CD25+ cells or other targeted from the blood product and into the therapeutic composition. In various embodiments, the particle 70 be a nanoparticle 71 and may correspond to one or more of a magnetic particle 72 and/or a fluorophore 73. Magnetic particle 72 may comprise one or more of iron, nickel or cobalt.

In many embodiments, the plurality(ies) 41 of antigen binding agents 40 (e.g., first plurality 42 of CD34+ antigen binding agents 43 and second plurality 44 of CD35+ antigen binding agents 45) can be disposed or otherwise associated with packaging 50 which may correspond to packaging known in the art including boxes, sealed pouches, sealed foil pouches or other sealed foil containers and containers one or more of which may be sterile packaging and/or configured to be sterilized. Also in one or more embodiments, the IFU 60 may also be disposed in or one the packaging 50. In some embodiments, the IFU 60 may be in the form of optical or other scannable or readable (e.g., electronically or magnetically scannable or readable) indicia 62 that are printed or embedded on packaging 50 where the indicia 62 encode information 63 containing the instructions. The indica may be configured to be read by a portable device 64 such as a cell phone, PDA tablet or like device. They may also be configured to be read by a bar code or other optical reader operatively coupled to a computer, processor or machine or instrument used in the selection and sorting of cells from blood product such as a optical or magnetic based cell sorter

In various embodiments, IFU 60 may be written or in electronic form 61 and in the latter case, they may be stored on computer readable media 80 including non-transitory computer readable media 81 such as a flash memory device 83 or other memory device 82 or they may be assessable through a network and/or the cloud. In or more embodiments, the IFU 60 may also include information unique to a batch of antigen binding agents 40b used in the kit, the information used by an instrument or device in the preparation of a therapeutic composition 20 to optimize a parameter of composition. The parameter may correspond to one or more of a purity, concentration or dose of cells in the therapeutic composition such as hematopoietic stem and progenitor cells. As is described below in some embodiments, the information may be encoded in optical or other scannable indicia 62 printed, attached or embedded in instructions 60. In alternative or additional embodiments, the information may also be encoded on scannable indicia 52 printed, attached or embedded on packaging 50 and/or as scannable indicia 94 printed, attached or embedded on container 90.

In various embodiments, the CD34+ and CD25+ antigen-binding agents 43 and 45 may correspond to an antibody or antigen binding portions of the antibody which includes the antigen binding fragment (Fab) of the antibody. In particular embodiments, the antibody may comprise a full-length antibody; a human antibody; a chimeric antibody; a humanized antibody; a single domain antibody, a bi-specific antibody. Also, the antigen binding portion of the antibody may comprise a Fab fragment; an Fab′ fragment; an F(ab′)2; an Fv (variable fragment); or a disulfide linked Fv. Also, in various embodiments, one or both of the CD34+ and CD25+ binding agents may be selected to exclude certain antigen binding agents including for example CD4+ or CD127 antigen binding agents. The antigen binding agent(s) may also be configured and arranged in the kit to allow for separation of the CD34+ and CD25+ cells from mobilized apheresis blood product that is obtained from one donor on a single day. For example, the CD34+ antigen binding agents and the CD25+ binding agents can be packaged in separate packaging within kit in amounts corresponding to that needed to process a typical amount of donated blood from a single donor

In various embodiments, the one or more therapeutic compositions produced using embodiments of the kits 10 described herein, may comprise a first therapeutic composition 21 comprising a first cell population 21p and a second composition 22 comprising a second cell population 22p. Typically, a cell population 24p in therapeutic composition 20 will be contained in a liquid 23 (e.g., first and second liquid volumes, etc.), with the liquid typically corresponding to a buffer 23b (e.g., includes one or more buffering agents). The buffer 23b may comprise a phosphate buffer. Also, the buffer 23b or other liquid 23 may include one or more excipients 23e such as serum albumin (e.g., human) and EDTA in selected amounts for example of about 2.5 volume percent human serum albumin and 1 millimolar EDTA. Other excipients may include viscosity modifying agents and various IV solution excipient known in the art.

Also in various embodiments, the one or more therapeutic compositions may comprise a first therapeutic composition 21 comprising CD34+ cells 27 (which may be suspended in a liquid volume) and a second composition 22 comprising CD25+ cells 25 (which may be suspended in a second volume). In specific embodiments, including those for treatment of stem cell-based cancer using a bone marrow transplant, the CD34+ cells may comprise hematopoietic stem cells as well as progenitor cells and the CD25+ cells may comprise Treg cells which may have a purity between 30 to 90 percent relative to all nucleated cells in the volume of liquid containing the Treg cells. Also in various embodiments, the apheresis blood product or other blood product 30 can be processed (e.g., using the CD34+ and CD25+ antigen binding agents) or otherwise adjusted to achieve a selected dose 24d of CD34+, and/or CD25+ cells or other cells in the one or more therapeutic compositions 20. The dose 24d may correspond to a total dose of cells or dose per unit patient weight, typically in kilograms (kg). In specific embodiments, a dose of CD34+ cells (e.g., HSPC's) in the composition can be greater than about one million cells per kilogram (kg) of the patient's weight and more preferably, greater than about 3.9 million cells per kg patient weight. Also in specific embodiments, a dose of CD25+ cells (e.g., Treg cells) in the composition 20 can be greater than about one million cells per kg of the patient's weight and more preferably greater than about 2.3 million cells per kg patient weight. Also, according to some embodiments, a dose of CD3+ cells in the composition(s) can be greater than about half a million cells per kg patient weight.

In various embodiments, the apheresis blood product can be processed (e.g., using embodiments of the antigen binding agents described herein) so as to have minimal values of various selected cellular components (either totally and/or per kg patient weight). Such selected components also referred to as cell impurities 24i or impurities may correspond to one or more of B-cells, CD56+ (e.g., natural killer cells), granulocytes, monocytes and other cells. For example, in one or more embodiments when the therapeutic composition 20 comprises CD34+ cells (e.g., HSPC's), the non-HSPC cellular components of the composition can comprise less than about 1.3×10⁵ CD56+ cells and B-cells (combined) per kg patient weight, less than about 6.2×10⁵ granulocyte cells per kg patient weight and less than about 2×10⁵ monocyte cells per kg patient weight. Further, in one or more embodiments when the therapeutic composition comprises CD25+ cells (e.g., Treg cells), the non-Treg cellular components of the composition can comprise less than about one or more of the following cells: 1.1×10⁵ non-Treg T cells, less than about 5.4×10⁴ CD56+ cells and B-cells (combined) per kg patient weight, and less than about 3.3×10³ granulocyte and monocytes cells (combined) per kg patient weight.

In various embodiments, including those where the CD34+, CD25 antigen binding agents 43 and 45 are suspended in a buffer 23b or other liquid 23, the antigen binding agents may be disposed in a container 90 such as a bag, vial, tube, test tube, column, or the like. In specific embodiments the container may correspond to a column 91 used in magnetically based cell sorting. In some embodiments, container 90 may correspond to the packaging 50 described herein while in others, the container 90 may be disposed in or on the packaging 50. In various embodiments, container 90 may be sealed, hermetically or otherwise so as to preserve the activity of the antigen binding agents so kit 10 has an extended shelf life (e.g., six months, nine months a year or longer). In specific embodiment the interior 92 of the container may include an inert or inert likes gas 93 such as nitrogen or argon so as to further extend the shelf life of the kit including antigen binding agents.

According to one or more embodiments, container 90 may have indicia 94 encoding various information 96 including for example information unique to a batch 40b of antigen binding agents 40 included in kit 10 where the information is used by an instrument or device in preparation of at least one therapeutic composition to optimize a parameter of the composition. The indicia 94 may correspond to electronic, magnetic or optically readable indicia 95 (e.g., a bar code) or other form of readable/scannable indicia known in the art. The parameter may correspond to one or more of a purity, concentration or dose of cells in the therapeutic composition (either total number of cells or number of cells per kg patient weight). For embodiments where the container corresponds to a bag, the bag may be made of a polymer bag such as polyvinyl chloride (PVC) and/or polymers known in the art which are used for blood bag.

In some embodiments the container 90 may comprise a column 91 fluidically (e.g., by IV tubing known in the art) or otherwise coupled to a bag 97 (e.g., a PVC or blood bag known in the art) such that the column can be used to select and separate cells which are then directed into the blood bag 97. In specific embodiments, kit 10 can include a container set 99 comprising a column 91 for magnetic or other form of cell separation, a PVC or other blood bag 97 and tubing or tubing set 98 which connects or is connectable to the column 91 and the blood bag 97 so as to allow cells to be sorted in/by the column and then directed into the blood bag for administration to the patient. In these and related embodiments, the antigen binding agents 40 can be preloaded into the column 91 or alternatively, they may be kept in separate container with an apparatus or means (e.g., a custom syringe) for allowing a user to load the antigen binding agents into the column 91.

In various embodiments, the amounts of one or both of the CD34+ and CD25+ antigen binding agents 43 and 44 (or other antigen binding agents 40) in the kit 10 can be selected to refine or otherwise process selected amounts of blood (e.g., apheresis blood product, cord blood etc.). For example, according to one embodiment, the amounts of CD34+ and CD25+ antigen binding agents 43 and 45 in kit 10 can be selected to process at least about eight liters of donor blood. In another embodiment, the amount of CD34+ and CD25+ antigen binding agents in the kit can be selected to process at least about 15 liters of donor blood with larger amounts contemplated as well. In the above and related embodiments, the instructions and/or packaging used in the kit can specify the amount of blood that can be processed by the kit. In specific embodiments, the packaging 50 or IFU 60 may include indicia 52 or 62 (e.g., optical, magnetic, RF or computer readable indica) encoding information 53 or 63 on the specific amount of blood that can be processed by the kit. The optical indicia may correspond to a bar code (e.g., a Code 129, Code 38 or QR code format) or other optical pattern while the RF indicia may correspond to an RF signal (e.g., which may be in BLUETOOTH format) transmitted by an RF ID tag or like device. After reading such information by a reading/scanning device 65 such as a portable device 64 (e.g., by a cell phone, bar code reader, RF receiver) or other optical or RF information reading device), the information can then be signaled to an instrument, device or machine 66 (which includes processor or logic resources 67) which performs one or more steps of the blood processing such as a magnetic or optical based cell sorting device including for example fluorescence-activated cell sorter (FACS). For embodiments of the kit including a container set 99 (e.g., a column, blood bag and tubing set (as described above), one or more of these items may include optical or other indicia encoding information indicating that these items are all in a set (i.e., the same set). Such information may be scanned and read by a user such as a lab technician who is using the kit including the set to generate a selected population of cells (e.g., HSPC's) for one or more therapeutic compositions describe herein.

In some embodiments, the kit may also include non-transitory computer readable media 81 which stores or contains computer executable instructions, algorithms and the like (herein referred to as software module or module 84) for performing processing steps or operations on a device, instrument or machine 66 to create the at least one therapeutic composition 2—using the anti-CD34+ and CD 25+ antigen-binding agents 43 and 45. The device, instrument or machine 66 may correspond to one or more of an optical cell sorter such as a fluorescence-activated cell sorter (FACS) or a magnetic-based cell sorter. According to one or more embodiments, computer readable storage media 80 may correspond to CD-ROM, or a memory device 83 such as a flash memory device 84. In related or additional embodiments, the non-transitory computer readable storage media 81 may also encode information 85 containing the IFU and information unique to a batch 40b of antigen binding agents 40 (e.g., to optimize processing of blood product by the antigen binding agents) which can be uploaded to a computer or other logic resources so as to be read by a doctor or other medical or lab personnel processing the blood product into the desired cellular components (e.g., HSPC's and Tregs). Similarly, information 63 contained in the IFU 60 can be configured to be uploaded to a device or instrument involved in the processing of the blood product into the respective cellular components. In such embodiments, the uploaded information can be used by that device or instrument to facilitate or optimize processing of the blood product into the respective cellular component.

Embodiments of the disclosure also provide methods for preparation of at least one therapeutic composition 20 for treatment of a disease or condition in a human subject in need thereof using one or more embodiments of kits 10 described herein. The disease or condition to be treated may include stem cell-based malignancy (e.g., leukemia, lymphoma, myeloma) or other cancer, graft versus host disease related to the treatment of the cancer or various autoimmune diseases. According to one embodiment, a method 200 for preparation of at least one therapeutic composition 20 from a blood product 30 for treatment of a disease or condition in a human subject in need thereof comprises providing a kit 10 (in a step 210) which includes a first plurality 42 of antigen-binding agents 40 that target CD34+ cells in the blood product (CD34+ antigen-binding agents 43) and a second plurality 44 of antigen binding agents 40 that target CD25+ cells in the blood product (CD25+ antigen-binding agents 45). As described above, typically, the CD34+ and CD25+ antigen-binding agents 43 and 45 are conjugated to a particle which allows for separation of targeted CD34+ and CD25+ cells from the blood product and into the at least one therapeutic composition 20. In these and related embodiments, the first plurality of antigen binding agents 42 are used to separate CD34+ cells 27 from the blood product 30 in a first separation step 220 so as to produce an enriched mixture 27em of CD34+ cells (e.g., HSPC's); and the second plurality of antigen binding agents 44 can be used to separate CD25+ cells 25 from the blood product 30 in second separation step 230 so as to produce an enriched mixture 25em of CD25+ cells. Typically, one or both of enriched cells mixture 25em and 27em comprises the cells 24 (also referred to as cell components 24) and a liquid solution 23 such as a buffer solution 23b (e.g., a phosphate buffer) in which the cells are suspended. The mobilized apheresis blood product 31 will usually be obtained from one donor on a single day but also may obtained over the course of several days. Also, typically, the blood product 30 used to prepare the at least one therapeutic composition 20 will comprise mobilized blood product 31, that is, blood product obtained from a donor who has received drugs treatments to mobilize or otherwise cause transport of their stem cells from their bone marrow into peripheral blood. The therapeutic composition 20 typically comprises one or more therapeutic compositions for example at least a first and second cell based therapeutic composition 21, 22 and which may be used in a treatment such as HSPC transplantation. The antigen-binding agents 40 will typically be conjugated to a particle 70 which allows for the separation of targeted CD34+ and CD25+ cells from the blood product and into the therapeutic composition. In one or more embodiments, the particle conjugated to the anti The CD34+ and CD25+ antigen-binding agents may be a nanoparticle and may correspond to one or more of a magnetic particle and/or a fluorophore. The magnetic particle may comprise one or more of iron, nickel or cobalt.

In many embodiments, the method may include an analysis step 240 to analyze at least one of the enriched mixtures of 27em and 25em CD34+ or CD25+ cells for a cell purity, of the respective cells in the mixture with other analyses also contemplated such as for cell concentration, total number of cells or cell impurities in the respective enriched mixtures. The analysis can be performed using various cell analytical equipment known in the art including flow cytometry devices, FACS devices, cell counters and the like. Having performed the analysis, a determination can be made (in a step 250) as to whether a cell purity of at least one of the enriched mixtures of CD34+ or CD25+ cell meets a predetermined threshold. In one or more embodiments, the determination 250 be done using a computer program or algorithm including for example a machine learning based algorithm implemented one or more processors or other computing devices. If the predetermined threshold is not met, additional processing (done in step 260) can be done of at least one of the enriched CD34+ or CD25+ cell mixtures 27em and 25em. In one or more embodiments, the additional processing may comprise performing antigen-binding agent-based cell separations (e.g., optical or magnetic) of at least one of the enriched CD34+ or CD25+ cell mixtures.

Also in one or more embodiments, at least one of the enriched CD34+ or CD25+ cell mixtures can be titrated or otherwise adjusted in a step 270 to achieve a selected patient specific dose of CD34+ or CD25+ cells (expressed as total number of cells or number of cells per kg patient weight). Such adjustment(s) can include either diluting the cell mixture or centrifuging a volume of the cell mixture and resuspending the centrifuged cells into a selected volume of fluid (e.g., a buffer solution). The patient specific dose of cells can be relative to number of cells per kilogram patient weight or a total number of cells (e.g., a million, ten million etc.).

According to one or more embodiments, the method may also include exchanging the buffer (in a buffer exchange step 280) in which at least one of the enriched CD34+ or CD25+ cell mixtures were suspended with another buffer which is used to suspend the cells in for administration to the patient. Similar to the adjustment made for dosing, the buffer exchange 280 step may include centrifuging one or both of the cell mixtures and resuspending the cells in another buffer (e.g., phosphate buffered saline, and/or ringers lactate). Also, before or after buffer exchange, one or more excipients 23e can be added to at least one of the enriched mixtures of CD34+ or CD25+ cells. The excipient may correspond to one or more of a buffering agent, an osmotic modifying agent, a viscosity modifying agent, a cell nutrient, a preservative, or one or more immunoglobulins such as albumin or one or more antibodies. In particular embodiments, the one or more antibodies may comprise those found in intravenous IG therapy (also known as IVIg) such as Gamma-Guard® manufactured by the Baxalta Corporation.

Another aspect provides a kit that comprises a solution comprising a first container comprising a first population of CD45+ cells, a second container comprising a solution comprising a second population of CD45+ cells, and a third container comprising a solution comprising a population of cells enriched for regulatory T cells (Tregs). In this aspect, the solution comprising the first population of CD45+ cells, the solution comprising the second population of CD45+ cells, and the solution comprising the population of cells enriched for Tregs are as defined according to any herein disclosed multi-component pharmaceutical treatment or method. In various embodiments, the kit further comprises a fourth container comprising the GVHD prophylactic agent. In some cases, the further comprising instructions for performing any herein-disclosed method.

A further aspect provides a kit comprising: (a) one or more reagents to sort CD34+ cells from a mobilized peripheral blood composition; (b) one or more reagents to sort regulatory T cells (Tregs) from the mobilized peripheral blood composition; (c) one or more reagents to detect a number of CD3+ conventional T cells in the mobilized peripheral blood; and (d) a solution comprising one or more doses of a graft vs host disease (GVHD) prophylactic agent. In some embodiments, the kit further comprises instructions for performing any herein-disclosed method.

Various embodiments provide a kit comprising one or more reagents for sorting HSPCs, for instance, a kit may comprise reagents to enrich a CD34+ cell population from a mobilized peripheral blood donation.

Some embodiments provide a kit comprising one or more reagents for sorting Tregs, for instance, a kit may comprise reagents to enrich a Treg cell population (using markers as described elsewhere herein) from a mobilized peripheral blood donation.

Embodiments provides a kit comprising one or more conditioning reagents for a conditioning regimen, for instance, a kit may comprise reagents for myeloablation or myeloreduction of a recipient.

Some embodiments provide a kit comprising one or more GVHD prophylactic agents.

In any herein-disclosed method, the method further comprises providing instructions for use (IFU), the IFU including instructions for administering the cell populations to the patient. In some cases, the IFU also include instructions for administering one or more pharmaceutical agents or compositions to the patient.

In some embodiments, kits may comprise instructions for use in preparation of a therapeutic composition. The kit may comprise instructions detailing any of the methods described herein. The kits described herein may comprise one or more of the compositions described herein.

In some embodiments, the kit may comprise instructions to isolate Tregs using an anti-human CD25 affinity reagent such that less than 85% of CD25 polypeptides expressed on the surface of the Tregs in the donor cell sample are occupied by the anti-human CD25 affinity reagent. In some embodiments, the instructions include directions to isolate Tregs such that less than 80% of CD25 polypeptides expressed on the surface of the Tregs in the donor cell sample are occupied by the anti-human CD25 affinity reagent. In some embodiments, the instructions include directions to isolate Tregs such that less than 75% of CD25 polypeptides expressed on the surface of the Tregs in the donor cell sample are occupied by the anti-human CD25 affinity reagent. In some embodiments, the instructions include directions to isolate Tregs such that at least 30% of CD25 polypeptides expressed on the surface of the Tregs in the donor cell sample are occupied by the anti-human CD25 affinity reagent. In some embodiments, the instructions include directions to isolate Tregs such that at least 40% of CD25 polypeptides expressed on the surface of the Tregs in the donor cell sample are occupied by the anti-human CD25 affinity reagent. In some embodiments, the instructions include directions to isolate Tregs such that from about 30% to 80% of CD25 polypeptides expressed on the surface of the Tregs in the donor cell sample are occupied by the anti-human CD25 affinity reagent. In some embodiments, the instructions include directions to isolate Tregs such that from 50% to 75% of CD25 polypeptides expressed on the surface of the Tregs in the donor cell sample are occupied by the anti-human CD25 affinity reagent.

### Definitions

In the present disclosure, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as size, number, concentration, percentage, ratio, or thickness, are to be understood to include any integer within the recited range, unless otherwise indicated. As used herein, the term “about” and its grammatical equivalents means±20% of the indicated range, value, or structure, more preferably ±10 and still more preferably ±5 of the indicated range, value, or structure. All numerical values or numerical ranges are understood to include numbers or ranges that are “about” the stated numbers or ranges. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

As used herein, the term “patient weight” may be either the patient's “actual weight” or the patient's “ideal weight”.

As used herein, the term “therapeutic cell” refers to a cell that is selected or administered to a subject based on the ability of the cell to offer a therapeutic benefit to a subject. Exemplary therapeutic cells include hematopoietic stem and progenitor cells, memory T cells, regulatory T cells, and invariant natural killer T cells. A population of therapeutic cells can include more than one type of therapeutic cell, e.g., HSPC, Tmem, Treg, iNKT or any combination thereof. A population of therapeutic cells can comprise essentially a single therapeutic cell type. In the context of a population of therapeutic cells, a percentage (%) of therapeutic cells refers to the percent of a cell-type that is included in a combination or composition of therapeutic cells in which the total number of therapeutic cells adds up to 100% and the specific therapeutic cell type represents a portion of the total number of therapeutic cells. For example, a population of therapeutic cells comprising 30% HSPC indicates that approximately 30% of the total population of therapeutic cells is HSPC. A population of therapeutic cells can exist within a larger population of cells that have a neutral effect on the subject and the neutral cells are not included in the calculation of total therapeutic cells.

As used herein, the term “hematopoietic stem and progenitor cells” or “HSPC” refer to hematopoietic stem cells and/or hematopoietic progenitor cells that express increased levels of phenotypic markers CD34, CD133, CD90, or any combination thereof, relative to other types of hematopoietic cells (e.g., the cells are positive for expression of the phenotypic marker as determined by flow cytometry, western blot, or other methods known in the art). Also, the HSPC can be negative for an expressed marker relative to other types of hematopoietic cells. For example, such markers include CD19, TCRα, TCRβ, CD45RA, Lin, CD38, or any combination thereof. Preferably, the HSPC are CD34.⁺ cells and/or CD19⁻ TCRα.⁻ HSPC can self-renew or can differentiate into (i) myeloid progenitor cells, which ultimately give rise to monocytes and macrophages, neutrophils, basophils, eosinophils, erythrocytes, megakaryocytes/platelets, or dendritic cells; or (ii) lymphoid progenitor cells which ultimately give rise to T-cells, B-cells, and lymphocyte-like cells called natural killer cells (NK-cells). For a general discussion of hematopoiesis and HSPC differentiation, see Rowe et al. Cell Stem Cell. 2016. 18:707-720, the contents of which is incorporated herein by reference in its entirety.

As used herein, the term “naïve conventional αβ-T cells” or “naïve Tcon” refers to a non-antigen experienced T cell that expresses the phenotypic markers TCRα/, CD45RA, and expresses medium to high levels of CD127 (CD127⁺), and does not express or has low expression of CD45RO and CD25. In some embodiments, naïve Tcon are characterized by the expression of phenotypic markers of naïve Tcon including TCRα, TCRβ, CD3, CD4 or CD8, CD62L, CCR7, CD28, CD127, and CD45RA. In some embodiments a naïve Tcon is CD3⁺ CD25⁻ CD45RA⁺ and comprises a polymorphic TCRα, and a polymorphic TCRβ. In some embodiments, naïve Tcon are not naïve T regulatory cells, as defined herein. Naïve Tcon cells do not express the Vα24Jα18 TCR found on iNKT cells.

As used herein, the term “regulatory T cell” or “Treg” refers to a subclass of T cell that is capable of suppressing autoimmune reactions and expresses the phenotypic markers CD4, CD25, and has no or low expression of CD127. Treg also express FOXP3, however, CD127 expression has been demonstrated to correlate inversely with FOXP3 expression on CD4⁺CD25⁺ cells, and the CD4⁺CD25⁺CD127⁻/low phenotype is considered to be an acceptable surrogate marker for Tregs and a practical alternative to intracellular staining for FOXP3 (Cozzo C, et al. J. Immunol. 2003 Dec. 1; 171:5678-82; Liu W, et al. J Exp. Med. 2006. 203(7):1701-1711; Seddiki N, et al. J Exp. Med. 2006; 203(7):1693-1700; the contents of each which is incorporated herein by reference in its entirety). Tregs can include at least two subclasses referred to herein as naïve Tregs and memory Tregs.

As used herein, the term “naïve Treg” is a non-antigen experienced regulatory T cell that expresses the phenotypic markers CD4, CD25, and CD45RA as a primary cell, and does not express or has low expression of CD45RO and CD127. Naïve Tregs are advantageous because the cells have higher plasticity for responding to antigens than antigen experienced Tregs. In addition, naïve Tregs have increased longevity compared to antigen experienced Tregs.

As used herein, the term “memory Treg” is an antigen experienced regulatory T cell that is capable of providing suppressive effects on autoimmunity and expresses the phenotypic markers CD4, CD25, and CD45RO and does not express or has low expression of CD127 and CD45RA.

As used herein, the term “memory T cell” or “Tmem” refers to antigen experienced T cells that express the phenotypic markers TCRα, TCRβ, CD3, CD4 or CD8, CD95, and IL-2Rβ. Memory T cells provide immunity and are capable of persisting for a long period of time in an inactive state. Memory T cells are able to rapidly acquire effector functions upon re-challenge with antigen. A population of memory T cells can include the any combination of the subclasses T central memory cells (T_{CM}) and T effector memory cells (TEM).

As used herein, “T central memory cell” or “T_{CM}” refers to an antigen experienced T cell that expresses the phenotypic markers CD4 or CD8, CD62L, CD45RO, CCR7, IL-2Rβ, CD28, CD127, and CD95 and does not express or has low expression of CD45RA as compared to naïve Tcon cells. Central memory T cells can differentiate into TEM cells following antigen re-challenge.

As used herein, “T effector memory cell” or “TEM” refers to an antigen experienced T cell that expresses the phenotypic markers CD4 or CD8, CD45RO, CD127, IL-2Rβ, and CD95, and does not express or has low expression of CD45RA, CD62L, CCR7, and CD28. T effector memory cell are terminally differentiated and acquire effector function after re-stimulation by antigen.

As used herein, “T stem central memory cell” or “T._{SCM}” refers to an antigen experienced T cell that expresses the phenotypic markers CD4 or CD8, CD45RA, CD62L, CD95, IL-2Rβ, CCR7, CXCR3, CD122, and LFA-1. T_{SCM} cells possess memory T cell capability of rapid acquisition of effector function following antigen re-challenge, but have enhanced stem cell-like qualities such as long-term persistence compared to T_{CM} cells. T_{SCM} cells can generate central memory, effector memory, and effector T cell subsets.

As used herein, “invariant Natural Killer T cells” or “iNKT” is a subclass of CD1d-restricted Natural Killer T (NKT) cells that express a highly conserved αβ-T cell receptor that comprises of Vα24Jα18 TCRα chain in humans (referred to herein as “Vα24Jα18⁺”). iNKT cells can be identified by binding with CD1d-multimers like that are loaded with α-galactosylceramide (GalCer), PBS-57, PBS-44 or other natural or synthetic glycolipids, and can be found as tetramers, dendrimers, and other structures, Fc fusions, or any combination thereof. Another method of identification is an antibody or combination of antibodies that specifically recognize the Vα24Jα18 region. Examples include a Vα24 antibody, a Jα18 antibody, or the monoclonal antibody clone 6B11 which binds specifically to a unique region of the Vα24Jα18 TCR and can be used to identify iNKT cells (Montoya et al. Immunology. 2007. 122(1):1-14, the contents of which is incorporated herein by reference in its entirety). In some embodiments, iNKT cells are CD1d-tetramer glycolipid loaded⁺ (CD1d-tet⁺), 6B11⁺, or both. iNKT cells may be interchangeably referred to herein as CD1d-tet.sup⁺ 6B11⁺ or Vα24Jα18⁺ cells. Without wishing to be limited to a particular mechanism, it is thought that iNKT cells promote/accelerate the activity of Treg and HSPC.

As referred to herein, “lineage positive” or “Lin.⁺” cells express the phenotypic markers such as CD19, CD11c, CD66B, CD14, CD20, or any combination thereof. As referred to herein, “lineage negative” or “Lin.⁺” cells do not express or have low expression of the phenotypic markers CD19, CD11c, CD66B, CD14, CD20, or any combination thereof compared to HSPC, Treg, Tmem, or iNKT cells. Lin.⁺ cells express phenotypic markers that are present on mature erythroid cells, granulocytes, macrophages, natural killer cells (NK) cells, and B and T lymphocytes.

As used herein, “sample” refers to a cell source (e.g. biological tissue) from which a population of cells may be isolated, enriched, or depleted. In some embodiments, a sample has generally not been previously processed or has been minimally processed. For example, the sample may be mobilized peripheral blood, mobilized apheresis product, bone marrow, umbilical cord blood, non-mobilized blood, non-mobilized apheresis product, or any combination thereof. In some embodiments, the sample is prepared or minimally processed by processing with a density gradient, Ficoll, Percoll, red blood cell hypotonic lysis, Ammonium-Chloride-Potassium (ACK) buffer, washed into a pH balanced isotonic buffer, or any combination thereof. In some embodiments, the sample is provided by a single tissue harvest. In some embodiments, the sample is provided by one or more tissue harvests.

As used herein, “donor” refers to one or more individuals (typically human) from which a sample, i.e., a donor cell sample, is obtained. For example, a donor may refer to an human leukocyte antigen (HLA) matched sibling, an HLA matched unrelated donor, a partially matched unrelated donor, a haploidentical related donor, autologous donor, an HLA unmatched allogeneic donor, a pool of donors, or any combination thereof. In some embodiments a donor may be a subject. “Donor tissue” refers to tissue harvested from a donor. Donor tissue can be a sample. The donor tissue is generally the same species as the subject.

As used herein, “subject” or “recipient” or “patient” refers to one or more individuals that are in need of receiving treatment, therapy, or cellular graft disclosed herein. Subjects that can be treated by the present disclosure are, in general, human. However, additional subjects include a non-human primate, cow, horse, sheep, goat, pig, dog, cat, mouse, rabbit, rat, or Guinea pig. The subjects can be male or female and can be any suitable age, including infant, juvenile, adolescent, adult, and geriatric subjects. During and following the treatment, a subject becomes a recipient or graft recipient.

As used herein, “tissue harvest” refers a process of collecting a donor tissue or a donor sample from a donor. Non-limiting examples of a tissue harvest include collecting bone marrow, peripheral blood, umbilical cord blood, etc. from a donor. A tissue harvest may be performed by any method which known in the art.

As used herein, “enriched” with respect to a population of a cells or cell-types in a mixture refers a population of cells that has been processed to increase the relative amount or ratio of the enriched cell-type relative to other cells (e.g., accounting cell-types) in the mixture. Thus, depending upon the source of the original population of cells subjected to the enriching process, a mixture or composition can contain 30%, 35%, 40%, 45%, 50%, 55%, 60%, 65%, 70%, 75%, 80%, 85%, 90%, 91%, 92%, 93%, 94%, 95%, 96%, 97%, 98%, or 99% or more (in number or count) of the “enriched” population of cells relative to other cells in the mixture. In some embodiments, the enrichment process can result in a 1.5-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 50-fold, 100-fold, 200-fold, 300-fold, 400-fold, 500-fold, 600-fold, 700-fold, 800-fold, 900-fold, 1,000-fold, 5,000-fold, 10,000-fold or more of the “enriched’ population of cells relative to other cells in the mixture. For example, in some embodiments, a mixture of cells that is enriched for iNKT cells may comprise about 0.03 to 1% iNKT cells, 0.05% to 0.5% iNKT cells, 0.1% to 1% iNKT cells, or any combination thereof. Exemplary methods of enriching a cell population include magnetic activated cell sorting (MACS) and fluorescence activated cell sorting (FACS).

As used herein, “depleted” with respect to a population of a cells or cell-types in a mixture refers a population of cells that has been processed to decrease the relative amount or ratio of the depleted cell-type relative to other cells (e.g., accounting cell-types) in the mixture. In some embodiments, cells subjected to a depleting process can result in a mixture or composition containing 50%, 45%, 40%, 35%, 30%, 25%, 20%, 15%, 10%, 9%, 8%, 7%, 6%, 5%, 4%, 3%, 2%, 1%, or 0.1%, 0.01%, 0.001%, 0.0001%, 0.00001%, 0.000001%, 0.0000001%, 0.00000001% or less (in number or count) of the “depleted” population of cells. In some embodiments, cells subjected to a depleting process can results in a mixture or composition containing 10-fold, 100-fold, 1,000-fold, 10,000-fold, 100,000-fold, 1,000,000-fold, 10,000,000-fold, or less of the depleted population relative to the unprocessed sample. In some embodiments, the depleted cell-type is no longer detectable using conventional methods following the processing step that depletes the cell-type.

In certain embodiments, amounts of a certain cell types in a mixture of cell types (such as those found in blood product) can be enriched and amounts of a different cell type are depleted. For example a mixture of cells can be enriched for CD34+ cells and depleted CD34− cells. Such an approach serves to increase the purity of a selected cell type in a mixture while reducing the number of unwanted cells in the mixture. This in turn increases the desired clinical effects from the selected cell types (e.g., production of healthy cells by HSPC's) while reducing adverse effects from the unwanted cells (e.g., GVHD caused by CD8+ cytotoxic T-cells).

As used herein a cell population “positive” for a marker refers to uniform staining of the cell population above the levels found on an isotype control. In some embodiments, a decrease in or low expression of one or more markers refers to a loss of or measure of at least 1 log₁₀ in the mean fluorescence intensity (MFI) less than a reference control. In some embodiments, an increase in or high expression of one or more markers refers to an increase or measure of MFI at least 1 log₁₀ higher than an isotype control or reference control. In some embodiments, an at least 2-fold increase in the MFI relative to the reference population indicates the cells are positive for expression of the marker. For example, a cell population that is positive for a marker can demonstrate a 2-fold to 4 fold, 4 fold to 10 fold, 10 fold to 100 fold, and 100 fold to 1,000 fold, 1,000 fold to 10,000 fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 50-fold, 100-fold, 200-fold, 300-fold, 400-fold, 500-fold, 600-fold, 700-fold, 800-fold, 900-fold, 1,000-fold, 5,000-fold, 10,000-fold or more higher MFI compared to an isotype control. In some embodiments, a cell population positive for of one or more markers refers to a percentage of cells that exhibit the marker, which can be at least 50% of the cells, 55% of the cells, 60% of the cells, 65% of the cells, 70% of the cells, 75% of the cells, 80% of the cells, 85% of the cells, 90% of the cells, 95% of the cells, and 100% of the cells and any % between 50% and 100% when compared to a reference cell population.

As used herein a cell population “negative” for a marker refers to the absence of significant staining of the cell population with the specific antibody above an isotype control. In some embodiments, an at least 2-fold decrease in the MFI relative to the reference population indicates the cells are negative for expression of the marker. For example, a cell population that is negative for a marker can demonstrate a 2-fold to 4 fold, 4 fold to 10 fold, 10 fold to 100 fold, and 100 fold to 1,000 fold, 1,000 fold to 10,000 fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 50-fold, 100-fold, 200-fold, 300-fold, 400-fold, 500-fold, 600-fold, 700-fold, 800-fold, 900-fold, 1,000-fold, 5,000-fold, 10,000-fold or more lower MFI compared to a positive control. In some embodiments, a decrease in or low expression of one or more markers refers to a percentage decrease of cells that exhibit the marker in a population of cells of at least 20% of the cells, 25% of the cells, 30% of the cells, 35% of the cells, 40% of the cells, 45% of the cells, 50% of the cells, 55% of the cells, 60% of the cells, 65% of the cells, 70% of the cells, 75% of the cells, 80% of the cells, 85% of the cells, 90% of the cell, 95% of the cells, and 100% of the cells and any % between 20% and 100% when compared to a reference cell population.

As used herein, “percent purity” or “% purity” refers to the number of target cells multiplied by 100 and then divided by the number of cellular events counted, as measured on a flow cytometer, hemocytometer, coulter counter, microscopy, or other cell counting method (# of target cells.times.100/# of cellular events).

As used herein, “overall percent yield” or “overall % yield” refers to the number of target cells after a processing step times 100 and then divided by number of target cells in the original population (# of target cells after a processing step.times.100/# of target cells in the original population). The “percent yield of a processing step” or “% yield of a processing step” refers to the number of target cells after a processing step times 100 and then divided by number of target cells in the preprocessed population (# of target cells after a processing step.times.100/# of target cells in the preprocessed population)

As used herein, “rough sort” refers to a method of enriching or depleting a population of cells wherein depending upon the source of the original population of cells subjected to the rough sort, the resulting population can contain at least 30%, 35%, 40%, 45%, 50%, 55%, 60%, 65%, 70%, 75%, 80%, 85%, 90%, 95%, or greater of a particular cell population compared to the starting mixture of cells. Methods of performing a rough sort a well-known in the art and can include density separation, apheresis/leukapheresis, tetrameric antibody complex mediated enrichment/depletion, and magnetic activated cell sorting (MACS), such as CLINIMACS®, PRODIGY®, or EASYSEP™/ROBOSEP™

As used herein, “fine sort” refers to a method of enriching or depleting a population of cells wherein the resulting population can contain at least 90%, 91%, 92%, 93%, 94%, 95%, 96%, 97%, 98%, 99%, 99.9%, or greater of a particular cell population or populations compared to the starting mixture of cells. Methods of performing a fine sort as well-known in the art can include multi-parameter fluorescence based molecular phenotypes such as fluorescence-activated cell sorting (FACS) and microfluidics based sorting. Additional methods of performing a fine sort are provided, for example, in PCT/US17/61414, which is hereby incorporated by reference in its entirety.

As used herein, the term “binding molecule” may be any of a large number of different molecules, or aggregates, and the terms are used interchangeably. Proteins, polypeptides, peptides, nucleic acids (nucleotides, oligonucleotides and polynucleotides), antibodies, saccharides, polysaccharides, lipids, receptors, test compounds (particularly those produced by combinatorial chemistry), may each or in combination (when bound to each other alone or via the target ligand) be a binding molecule.

As used herein, “specifically binds” or “specific for” refers to an association or union of a binding molecule (e.g., antibody) or a binding domain to a target (molecule or complex) with an affinity or Ka (i.e., an equilibrium association constant of a particular binding interaction with units of 1/M) equal to or greater than 10⁵M⁻¹ (which equals the ratio of the on-rate [kₒₙ] to the off-rate [k_{off}] for this association reaction), while not significantly associating or uniting with any other molecules or components in a sample. Binding molecules or binding domains may be classified as “high affinity” binding molecules or binding domains or as “low affinity” binding molecules or binding domains. “High affinity” binding molecules or binding domains refer to those binding molecules or binding domains having a Kₐ of at least 10⁷M⁻¹, at least 10⁸M⁻¹, at least 10⁹M⁻¹, at least 10¹⁰M⁻¹, at least 10¹¹M⁻¹, at least 10¹² M⁻¹, or at least 10¹³M⁻¹. “Low affinity” binding molecules or binding domains refer to those binding molecules or binding domains having a Kₐ of up to 10⁷ M⁻¹, up to 10⁶M⁻¹, up to 10⁵M⁻¹. Alternatively, affinity may be defined as an equilibrium dissociation constant (K_{d}) of a particular binding interaction with units of M (e.g., 10⁻5 M to 10.⁻513 M

As used herein, a “sculpted cellular graft” or “sculpted graft” refers to population of cells that has been processed from a starting population of cells to provide numbers of specific cell-types within a specified range and to reduce or remove undesired cell-types to a specified range. Ranges are typically provided as a number of cells of a particular variety per kg of patient body weight, but they may also be represented as a total number of cells within the graft. A sculpted cellular graft can comprise a mixture of cells that include target cell to accounting cell ratios that do not occur in nature or percentage representations that do not occur in nature.

As used herein, a “unit dose” of refers to specified minimum numbers, specified numbers, or ranges of populations of therapeutic cells for each kilogram (kg) of body weight of a subject (e.g., patient) receiving a sculpted cellular graft. It is recognized that the number of unit doses varies depending on the weight and/or size of the subject. A unit dose may be divided into a fraction of a unit dose depending on the weight of the subject. In some embodiments, the therapeutic cell populations (e.g., HSPC, Tmem, Treg, iNKT, etc.) may be divided into separate containers for administration to a subject.

### VI. EXAMPLES

The following examples are included for illustrative purposes only and are not intended to limit the scope of the disclosure.

### Example 1: Clinical Study

### C. Study Design

A clinical study was conducted in subjects with advanced hematologic malignancies undergoing myeloablative allogeneic hematopoietic cell transplantation (alloHCT).

The graft composition and manufacturing processes are detailed below.

Primary endpoints of the study include the incidence of primary graft failure; and the incidence, severity, and timing of Grade III-V acute GVHD.

Secondary endpoints for all groups include neutrophil engraftment, platelet engraftment, incidence of secondary graft failure, incidence and severity of treatment-emergent adverse events (TEAEs), incidence and severity of steroid-refractory acute GVHD, for example, grade 3-4 steroid-refractory acute GVHD, incidence and severity of chronic GVHD, incidence of post-transplant lymphoproliferative disorder (PTLD), non-relapse mortality (NRM), disease relapse (Arms I & III), relapse free survival, GVHD and relapse free survival (GRFS), overall survival, incidence of serious infections, and T cell immunity reconstitution parameters.

### D. Study Populations

Subjects were eligible to receive a composition of the disclosure if they met all the following criteria:
(1) Age ≥18 and ≤65 years at the time of enrollment.
(2) Diagnosed with the any of the following histopathologically-confirmed diseases: (a) Acute myeloid, lymphoid or mixed phenotype leukemia in complete remission (CR) or CR with incomplete hematologic recovery (CRi) without the presence of known minimal residual disease; or (b) Acute myeloid, lymphoid or mixed phenotype leukemia that is either: (i) not in morphologic CR with bone marrow infiltration by leukemic blasts of ≤10%, or (ii) in morphologic CR with evidence of minimal residual positivity by either multiparameter flow cytometric analysis or by a nucleic acid-based technique; (c) High or Very High-risk Myelodysplastic syndromes; (d) Myelofibrosis (MF) that is eligible for transplant per National Comprehensive Cancer Network Guidelines. Specifically, patients should be diagnosed with MF that is either: (i) intermediate-2- or high-risk according to the IPSS, DIPSS or DIPSS-plus scoring systems; or (ii) intermediate-1-risk disease associated with high-risk features such as high symptoms burden, low platelet counts, or complex cytogenetics; per NCCN guidelines and Investigator judgement (e) myeloproliferative syndromes; (f) Non-Hodgkin lymphoma with poor risk features not suitable for autologous HCT.
(3) Planning to undergo myeloablative allogeneic hematopoietic cell transplantation (MA-alloHCT) including a suitable myeloablative conditioning regimen.
(4) Matched to a donor that is a: (a) Matched sibling donor who is an 8/8 match for HLA-A, -B, -C, and -DRB1, all typed using DNA-based high resolution methods; or (b) Matched unrelated donor who is a 8/8 match at HLA-A, -B, -C, and -DRB1, all typed using DNA-based high resolution methods.
(5) Estimated glomerular filtration rate (eGFR) >30 mL/minute; or >50 mL/minute for patients for whom GVHD prophylaxis with tacrolimus is planned.
(6) Cardiac ejection fraction at rest ≥45% or shortening fraction of ≥27% by echocardiogram or radionuclide scan (MUGA).
(7) Diffusing capacity of the lung for carbon monoxide (DLCO) (adjusted for hemoglobin) ≥50%.
(8) Negative serum or urine beta-HCG test in females of childbearing potential within 3 weeks of registration.
(9) Total bilirubin <2 times upper limit of normal (ULN) (patients with Gilbert's syndrome may be included where hemolysis has been excluded and with approval of the medical monitor).
(10) ALT/AST <3 times upper limit of normal (ULN).

Subjects were ineligible to receive the composition of the disclosure if they met any of the following exclusion criteria:
(1) Received a prior allogeneic HCT.
(2) Candidate for autologous transplant.
(3) Currently receiving corticosteroids or other immunosuppressive therapy. Topical corticosteroids or oral systemic corticosteroid doses less than or equal to 10 mg/day are allowed.
(4) Planned donor lymphocyte infusion (DLI) recipient.
(5) Planned recipient of pharmaceutical in vivo or ex vivo T cell depletion, e.g., post-transplant cyclophosphamide (Cy), peri-transplant anti-thymocyte globulin (ATG), or alemtuzumab. For patients that have previously been exposed to a T cell-depleting agent, a 5 half-life washout of the agent must occur prior to planned Day 0 (day of infusion of the Treg and HSPC components of the graft).
(6) Positive for anti-donor HLA antibodies against a mismatched allele in the selected donor as determined by either: (a) A positive crossmatch test of any titer; or (b) The presence of anti-donor HLA antibody to any HLA locus.
(7) Karnofsky performance score <70%.
(8) Hematopoietic cell transplantation-specific Comorbidity Index (HCT-CI) >4.
(9) Uncontrolled bacterial, viral or fungal infections (currently taking antimicrobial therapy and with progression or no clinical improvement) at time of enrollment.
(10) Seropositive for HIV-1 or -2, HTLV-1 or -2, Hepatitis B sAg, or Hepatitis C antibody,
(11) Known allergy or hypersensitivity to, or intolerance of, tacrolimus (or tacrolimus and sirolimus if eligible for single-agent prophylaxis with either)
(12) Documented allergy or hypersensitivity to iron dextran or bovine, murine, algal or Streptomyces avidinii proteins.
(13) Any uncontrolled autoimmune disease requiring active immunosuppressive treatment.
(14) Concurrent malignancies or active disease within 1 year, except non-melanoma skin cancers that have been curatively resected.
(15) Psychosocial circumstances that preclude the patient being able to go through transplant or participate responsibly in follow up care.
(16) Women who are pregnant or breastfeeding.
(17) Women of childbearing potential (WOCBP) or men who have sexual contact with WOCBP unwilling to use effective forms of birth control or abstinence for one year after transplantation.
(18) Any serious medical condition or abnormality in clinical laboratory tests that, in the investigator's or Medical Monitor's judgment, precludes the recipient's safe participation in and completion of the study, or which could affect compliance with the protocol or interpretation of results.

Subjects were part of the following groups:

Arm 1: subjects planning to undergo myeloablative allogeneic hematopoietic cell transplantation (MA-alloHCT) for the treatment of either acute myeloid, lymphoid or mixed phenotype leukemia in complete remission (CR) or CR with incomplete hematologic recovery (CRi) with no known minimal residual disease positivity, planning to MA-alloHCT.

Arm 2: subjects planning to undergo MA-alloHCT for acute myeloid, lymphoid or mixed phenotype leukemia that is either: (i) not in morphologic CR with bone marrow infiltration by leukemic blasts of ≤10%, or (ii) in morphologic CR with evidence of minimal residual positivity by either multiparameter flow cytometric analysis or by a nucleic acid-based technique.

Arm 3: subjects planning to undergo MA-alloHCT for high or very high-risk myelodysplasic syndrome (MDS) myelodysplastic syndromes or for myelofibrosis (primary myelofibrosis or myelofibrosis evolved from other myeloproliferative neoplasms).

Subjects with sensitivity to iron dextran, chemical products derived from cyanine dyes, and proteins products derived from murine, bovine, algal, and Streptomyces avidinii sources are excluded.

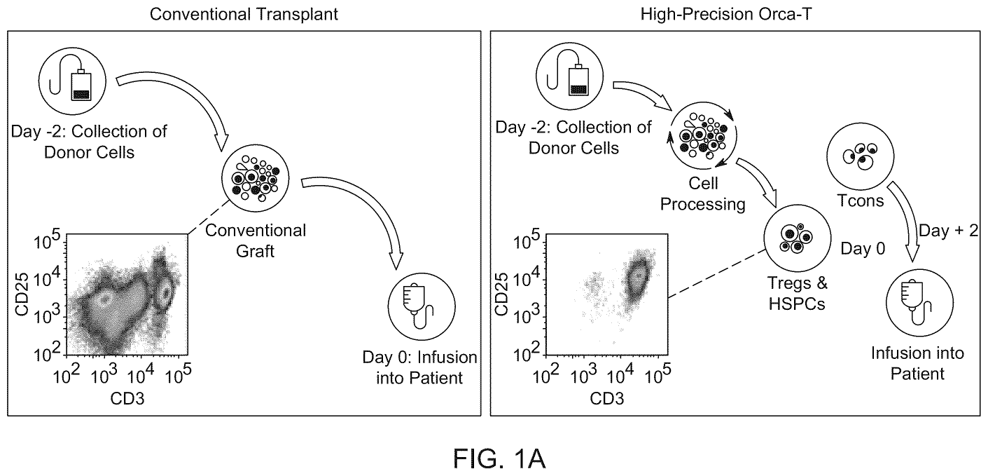

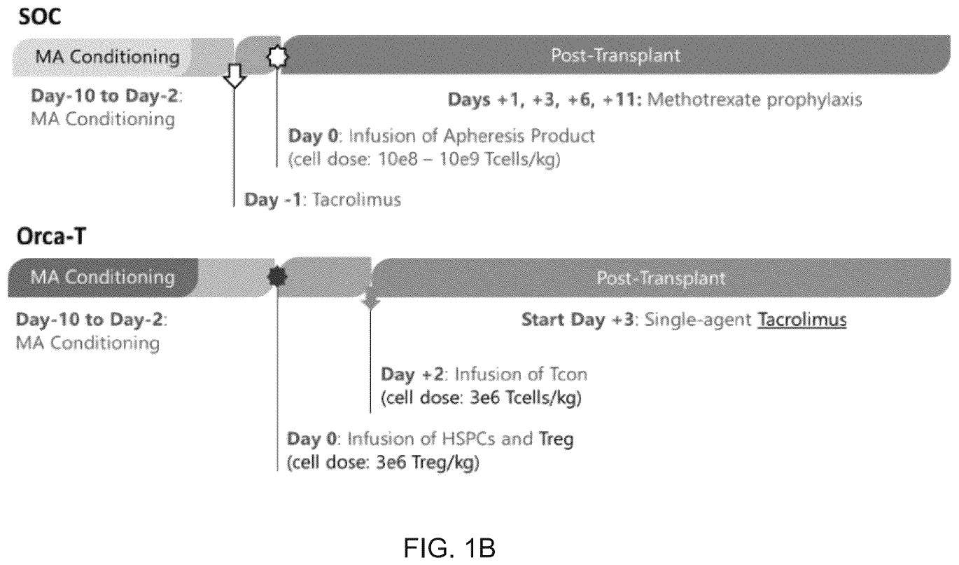

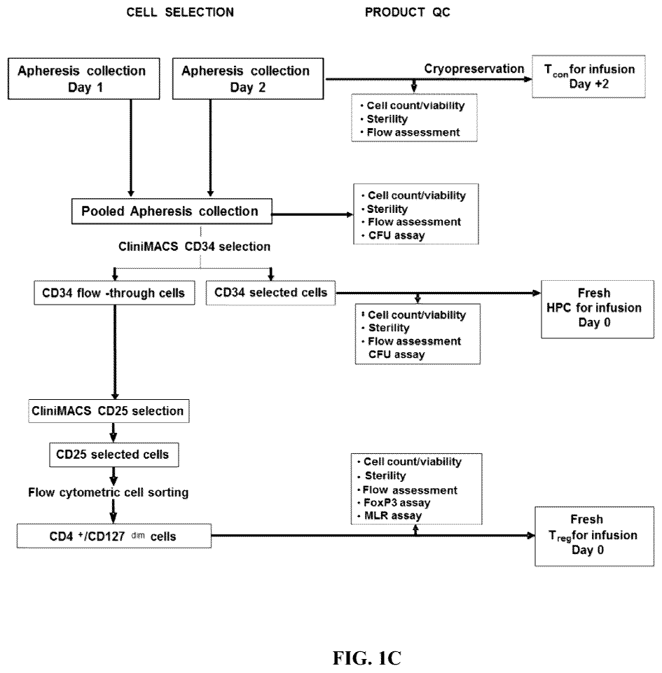

FIGS. 1A-1B illustrate the schematics of the transplant according to the present study (identified as High-Precision Orca-T or OrcaT) and the differences compared to a standard of care (SOC) cohort (identified as Conventional Transplant or SOC). FIG. 1C illustrates a schematic of graft production and administration.

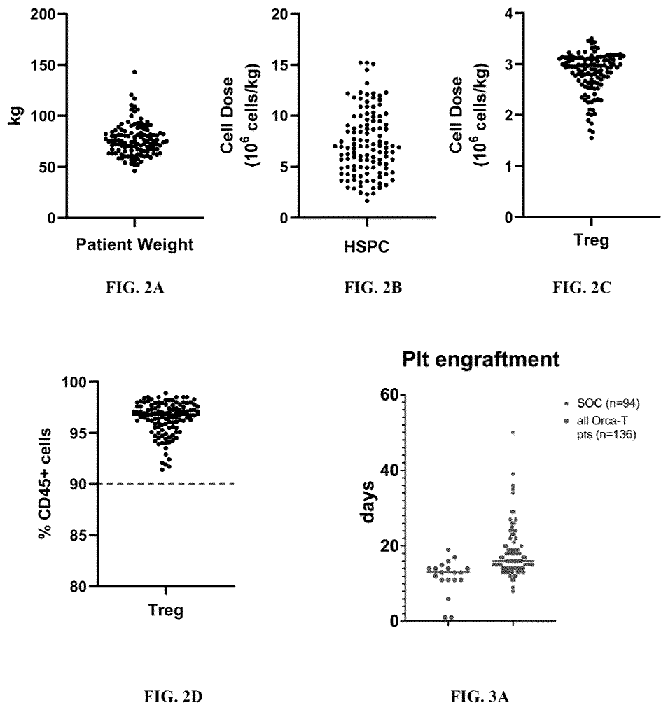

FIG. 2A illustrates the weights of patients enrolled in the study. Table 7 shows the shows the demographics, primary disease, features determining disease risk status, disease status at transplant, and transplant details for a representative subset of the subjects. Abbreviations: DLBCL, diffuse large B cell lymphoma; AML, acute myeloid leukemia; ALL, acute lymphocytic leukemia; MDS, myelodysplastic syndrome; MF, myelofibrosis; MPAL, mixed phenotype acute leukemia; CML, chronic myeloid leukemia; CR1, 1st complete remission; CR2, 2nd complete remission; active, active disease (not in CR as defined for disease entity); MRD+, minimal residual disease positive; MRD, matched related donor; URD, unrelated donor; MF, myelofibrosis. FTBI, fractionated total body irradiation; Cy, cyclophosphamide; VP-16, etoposide; Bu, busulfan; FLT3+, FMS-like tyrosine kinase 3; c-kit, ASXL1, additional sex combs like 1. Days of follow up denotes the number of days since transplant Day 0 for each patient at the time of reporting.

TABLE 7

| | | | High-risk | Disease status | Donor | Conditioning | GVHD | Days of |
|---|---|---|---|---|---|---|---|---|
| Age | Sex | Disease | Features | at transplant | type | Regimen | prophylaxis | follow up |
| 49 | M | BLBCL | refractory | active | MRD | BCNU/VP16/Cy | Sirolimus | 1219 |
| 42 | M | AML | FLT3+ | CR2 | MRD | BU/Cy | Sirolimus to | 1177 |
| | | | | | | | Tacrolimus | |
| 34 | M | ALL | refractory | active | MRD | TBI/Cy/VP16 | Sirolimus | 1107 |
| 20 | M | ALL | MRD+ | CR2 | MRD | BU/Cy | Sirolimus | 1079 |
| 54 | M | CML | refractory | active | MRD | BU/Cy | Sirolimus | 1058 |
| 53 | M | AML | AML CR2 | CR2 | MRD | BU/Cy | Sirolimus | 981 |
| 56 | M | MF | refractory | active | MRD | BU/Cy | Tacrolimus | 932 |
| 40 | F | AML | refractory | CR1 | MRD | BU/Cy | Tacrolimus | 260 |
| 59 | M | PTCL | refractory | active | MRD | BCNU/VP16/Cy | Sirolimus | 225 |
| 42 | F | AML | refractory | CR1 | MRD | BU/Cy | None | 211 |
| 40 | M | AML | refractory | CR1 | MRD | BU/Cy | None | 204 |
| 48 | M | MDS | Monosomy 7 | active | MRD | BU/Cy | None | 190 |
| 35 | M | ALL | MRD+ | CR1 | MRD | FTBI/VP16/Cy | Sirolimus | 176 |
| 28 | M | ALL | CR2 | CR2 | MRD | BU/Cy | None | 127 |
| 56 | F | MF | n/a | active | URD | BU/Cy | Tacrolimus | 119 |
| 39 | M | MPAL | MPAL | CR1 | MRD | FTBI/VP16/Cy | Sirolimus | 120 |
| 41 | F | AML | TP53 and c- | CR1 | MRD | BU/Cy | Tacrolimus | 99 |
| | | | Kit mutated | | | | | |
| 39 | M | ALL | Ph-like | CR1 | MRD | FTBI/VP16/Cy | None | 43 |
| 34 | M | MPAL | Complex | CR1 | MRD | FTBI/VP16/Cy | Sirolimus | 36 |
| | | | karyotype, | | | | | |
| | | | MPAL | | | | | |
| 26 | F | AML | AML t(6:9) | CR1 | MRD | BU/Cy | Tacrolimus | 34 |
| 52 | M | AML | ASXL1 | CR1 | MRD | BU/Cy | Tacrolimus | 22 |
| | | | mutation | | | | | |

### Standard of Care Control Cohort

For comparison of clinical outcomes, a standard of care (SOC) comparator cohort was identified retrospectively from subjects who received a SOC myeloablative alloHCT at one of the same clinical sites during the same period; a schematic of the protocol followed for SOC patients is also shown in FIGS. 1A-1B. To be included in the SOC cohort, subjects had to meet all the following criteria: (a) they were diagnosed with a hematologic malignancy eligible for treatment with an alloHCT; (b) they received an allograft of mobilized peripheral blood (i.e., not a bone marrow-derived graft); (c) their donor was a fully HLA-matched related donor (unrelated donors were not included due to the limited number in the treatment cohort to date); (d) they received a myeloablative conditioning regimen; and (e) they were not enrolled on an investigative protocol. Personnel who identified subjects for the SOC cohort were blinded as to their clinical outcomes.

All patients in the SOC cohort received myeloablative conditioning regimens and dual-agent GVHD prophylaxis with tacrolimus and methotrexate.

Clinical characteristics of subjects in the SOC cohort are provided in TABLE 8.

TABLE 8: characteristics of a representative subset of subjects in the standard of care (SOC) cohort. Abbreviations used: DLBCL, diffuse large B cell lymphoma; AML, acute myeloid leukemia; ALL, acute myeloid leukemia; MDS, myelodysplastic syndrome; MF, myelofibrosis; MPAL, mixed phenotype acute leukemia; CML, chronic myeloid leukemia; CR1, 1st complete remission; CR2, 2nd complete remission; “active”, active disease (not CR as defined for each disease entity); MRD, minimal residual disease; MRD, matched related donor; MF, myelofibrosis. FTBI, fractionated total body irradiation; Cy, cyclophosphamide; VP-16, etoposide; Bu, busulfan; FLT3+, FMS-like tyrosine kinase 3; Tac, tacrolimus; MTX, methotrexate.

TABLE 8

| | | | Disease | | | |
|---|---|---|---|---|---|---|
| | | | Status at | Donor | Conditioning | GVHD |
| Age | Sex | Disease | Transplant | Type | regimen | Prophylaxis |
| 46 | F | ALL | CR 1 | MRD | FTBI/VP16 | FK/MTX |
| 49 | F | ALL | CR 1 | MRD | FTBI/VP16 | FK/MTX |
| 56 | F | AML | CR 1 | MRD | BU/CY | FK/MTX |
| 58 | M | AML | CR 1 | MRD | BU/CY | FK/MTX |
| 44 | M | ALL | CR 1 | MRD | FTBI/VP16 | FK/MTX |
| 45 | M | AML | CR 2 | MRD | FTBI/VP16 | FK/MTX |
| 52 | M | ALL | CR 1 | MRD | BU/CY | FK/MTX |
| 56 | M | AML | CR 1 | MRD | BU/CY | FK/MTX |
| 32 | F | ALL | CR 1 | MRD | FTBI/VP16 | FK/MTX |
| 23 | M | AML | CR 2 | MRD | BU/CY | FK/MTX |
| 59 | F | ALL | CR 1 | MRD | BU/CY | FK/MTX |
| 50 | F | AML | CR 2 | MRD | BU/CY | FK/MTX |
| 50 | M | ALL | CR 1 | MRD | FTBI/VP16 | FK/MTX |
| 31 | F | AML | CR 1 | MRD | BU/CY | FK/MTX |
| 57 | F | AML | CR 1 | MRD | BU/CY | FK/MTX |
| 21 | M | AML | CR 1 | MRD | BU/CY | FK/MTX |
| 27 | M | OAL | CR 2 | MRD | BU/CY | FK/MTX |
| 51 | F | ALL | CR 1 | MRD | FTBI/VP16 | FK/MTX |
| 38 | M | ALL | CR 1 | MRD | FTBI/VP16 | FK/MTX |
| 44 | F | CML | CP1 | MRD | BU/CY | FK/MTX |
| 19 | M | ALL | CR 1 | MRD | FTBI/VP16 | FK/MTX |
| 58 | M | ALL | CR 1 | MRD | BU/CY | FK/MTX |
| 55 | M | ALL | CR 1 | MRD | BU/CY | FK/MTX |
| 39 | M | AML | active | MRD | BU/CY | FK/MTX |
| 25 | M | AML | CR 2 | MRD | BU/CY | FK/MTX |
| 33 | M | NHL | CR 1 | MRD | BU/CY | FK/MTX |
| 32 | M | AML | CR 2 | MRD | BU/CY | FK/MTX |
| 59 | M | AML | CR 1 | MRD | BU/CY | FK/MTX |
| 47 | M | AML | CR 1 | MRD | BU/CY | FK/MTX |
| 47 | M | ALL | CR 1 | MRD | FTBI/VP16 | FK/MTX |
| 43 | M | AML | CR 1 | MRD | BU/CY | FK/MTX |
| 59 | F | ALL | CR 2 | MRD | BU/CY | FK/MTX |
| 27 | M | ALL | CR 1 | MRD | FTBI/VP16 | FK/MTX |
| 40 | M | AML | CR 1 | MRD | BU/CY | FK/MTX |
| 24 | F | ALL | CR 1 | MRD | FTBI/VP16 | FK/MTX |
| 27 | F | NHL | active | MRD | BU/CY | FK/MTX |
| 34 | M | ALL | CR 1 | MRD | FTBI/VP16 | FK/MTX |
| 58 | M | AML | CR 1 | MRD | BU/CY | FK/MTX |
| 41 | M | CML | active | MRD | BU/CY | FK/MTX |
| 58 | M | ALL | CR 1 | MRD | BU/CY | FK/MTX |
| 52 | F | AML | active | MRD | BU/CY | FK/MTX |
| 62 | M | MDS | active | MRD | BU/CY | FK/MTX |
| 62 | M | MDS | active | MRD | BU/CY | FK/MTX |
| 40 | M | AML | CR 1 | MRD | BU/CY | FK/MTX |
| 44 | F | AML | CR 1 | MRD | FTBI/VP16 | FK/MTX |
| 54 | M | ALL | CR 1 | MRD | BU/CY | FK/MTX |
| 36 | M | AML | CR 1 | MRD | BU/CY | FK/MTX |
| 46 | F | CML | CR 2 | MRD | FTBI/VP16 | FK/MTX |
| 41 | M | MDS | active | MRD | BU/CY | FK/MTX |
| 43 | F | ALL | CR 2 | MRD | FTBI/VP16 | FK/MTX |
| 51 | F | MDS | active | MRD | BU/CY | FK/MTX |
| 54 | M | MDS | active | MRD | BU/CY | FK/MTX |
| 52 | F | CML | CR1 | MRD | BU/CY | FK/MTX |
| 56 | F | MDS | active | MRD | BU/CY | FK/MTX |
| 60 | M | AML | CR 1 | MRD | BU/CY | FK/MTX |
| 56 | M | MDS | active | MRD | BU/CY | FK/MTX |
| 57 | F | MDS | active | MRD | BU/CY | FK/MTX |
| 49 | F | ALL | CR 1 | MRD | FTBI/VP16 | FK/MTX |
| 34 | M | AML | CR 1 | MRD | BU/CY | FK/MTX |
| 59 | F | ALL | CR 1 | MRD | BU/CY | FK/MTX |
| 60 | M | MDS | active | MRD | BU/CY | FK/MTX |
| 32 | M | AML | CR 1 | MRD | FTBI/VP16 | FK/MTX |
| 57 | F | OAL | CR | MRD | BU/CY | FK/MTX |
| 43 | F | AML | CR 1 | MRD | BU/CY | FK/MTX |
| 47 | M | ALL | CR 1 | MRD | BU/CY | FK/MTX |
| 60 | M | MDS | active | MRD | BU/CY | FK/MTX |
| 63 | M | AML | CR 1 | MRD | BU/CY | FK/MTX |
| 49 | M | AML | CR 1 | MRD | BU/CY | FK/MTX |
| 51 | F | AML | CR 2 | MRD | BU/CY | FK/MTX |
| 49 | M | AML | CR 2 | MRD | BU/CY | FK/MTX |
| 40 | F | ALL | CR 1 | MRD | BU/CY | FK/MTX |
| 48 | M | NHL | CR 2 | MRD | BU/CY | FK/MTX |
| 52 | M | AML | CR 2 | MRD | BU/CY | FK/MTX |
| 36 | M | OAL | CR 1 | MRD | FTBI/VP16 | FK/MTX |
| 27 | M | NHL | active | MRD | FTBI/VP16 | FK/MTX |
| 57 | M | AML | CR 1 | MRD | BU/CY | FK/MTX |
| 56 | F | NHL | active | MRD | BU/CY | FK/MTX |
| 52 | F | AML | CR 1 | MRD | BU/CY | FK/MTX |
| 39 | M | AML | active | MRD | FTBI/VP16 | FK/MTX |
| 57 | F | MDS | active | MRD | BU/CY | FK/MTX |
| 26 | M | ALL | CR 1 | MRD | FTBI/VP16 | FK/MTX |
| 21 | F | ALL | CR3+ | MRD | FTBI/VP16 | FK/MTX |
| 55 | M | NHL | active | MRD | BU/CY | FK/MTX |
| 25 | M | ALL | CR 1 | MRD | FTBI/VP16 | FK/MTX |
| 34 | F | MDS | active | MRD | BU/CY | FK/MTX |
| 40 | F | AML | CR 1 | MRD | BU/CY | FK/MTX |
| 59 | M | ALL | CR 1 | MRD | BU/CY | FK/MTX |
| 42 | F | NHL | active | MRD | BU/CY | FK/MTX |
| 29 | M | ALL | CR 1 | MRD | FTBI/VP16 | FK/MTX |
| 26 | F | ALL | CR | MRD | FTBI/VP16 | FK/MTX |
| 40 | M | CML | CP1 | MRD | BU/CY | FK/MTX |
| 58 | F | MDS | CR | MRD | BU/CY | FK/MTX |
| 53 | M | ALL | CR 1 | MRD | BU/CY | FK/MTX |
| 21 | M | ALL | CR 2 | MRD | BU/CY | FK/MTX |

### E. Donors

HLA-identical related or unrelated donors were used.

Donors were used that met all of the following inclusion criteria:
(1) Age ≥16 and ≤75 years at time of enrollment
(2) Matched to the patient as follows: Either one of: (i) matched related donor who is an 8/8 match for HLA-A, -B, -C, and -DRB1, all typed using DNA-based high resolution methods; (ii) matched unrelated donor who is an 8/8 match at HLA-A, -B, -C, and -DRB1, all typed using DNA-based high resolution methods.
(3) Able to donate at a site that will employ a Spectra Optia Apheresis System for post-mobilization apheresis;
(4) Meets federal eligibility criteria for donors of viable, leukocyte-rich cells or tissues as defined by 21 CFR § 1271 2018 and all relevant FDA Guidance for Industry (Eligibility Determination for Donors of Human Cells, Tissues, and Cellular and Tissue-Based Products, 2007; Use of Donor Screening Tests to Test Donors of Human Cells, Tissues and Cellular and Tissue-Based Products for Infection with Treponema pallidum (Syphilis), 2015; Use of Nucleic Acid Tests to Reduce the Risk of Transmission of Hepatitis B Virus from Donors of Human Cells, Tissues, and Cellular and Tissue-Based Products, 2016; Use of Nucleic Acid Tests to Reduce the Risk of Transmission of West Nile Virus from Living Donors of Human Cells, Tissues, and Cellular and Tissue-Based Products (HCT/Ps), 2016; Donor Screening Recommendations to Reduce the Risk of Transmission of Zika Virus by Human Cells, Tissues, and Cellular and Tissue-Based Products, 2018).
(5) Meets any other criteria for donation as specified by standard NMDP guidelines (NMDP donors) or institutional standards (non-NMDP donors).
(6) Female donors of child-bearing potential must have a negative serum or urine beta HCG test within 3 weeks of mobilization.
(7) Capable of undergoing leukapheresis, have adequate venous access, and be willing to undergo insertion of a central catheter should leukapheresis via peripheral vein be inadequate.

Donors determined to be ineligible, based on the results of required testing and/or screening, were nonetheless included if either apply, as per 21 CFR § 1271.65 2018: (a) the donor is a first-degree or second-degree blood relative of the recipient; or (b) Urgent medical need, meaning no comparable human cell product was available and the recipient is likely to suffer death or serious morbidity without the human cell product, as attested by the Investigator.

Donors meeting any of the following exclusion criteria were not eligible:
(1) Evidence of active infection
(2) Seropositive for HIV-1 or -2, HTLV-1 or -2
(3) Positive for anti-hepatitis C (HCV) antibody or HCV NAT.
(4) Positive serologic or PCR test results indicating acute or chronic HBV infection. Donors whose HBV infection status cannot be determined conclusively by serologic test results must be negative for HBV by PCR to be eligible for study participation.
(5) Potential for Zika virus infection as defined as any of the following: (i) Medical diagnosis of Zika virus infection in the past 6 months; (ii) Residence in, or travel to, an area with active Zika virus transmission within the past 6 months; (iii) Unprotected sex within the past 6 months with a person who is known to have either of the risk factors (i) or (ii). Donors determined to be ineligible based on the results of Zika virus screening may be determined to be eligible if: (a) the donor has no signs or symptoms consistent with active Zika virus infection; and (b) The donor is a first-degree or second-degree blood relative of the recipient, or ii) in cases of urgent medical need, meaning no comparable human cell product is available and the recipient is likely to suffer death or serious morbidity without the human cell product, as attested by the Investigator.
(6) Women who are pregnant or breastfeeding.

### F. Generation of Cell Components

Donors received mobilization therapy with daily G-CSF. The recommended dose was 10 μg/kg/day SQ (rounded off to the nearest vial size of either 300 or 480 pg). The Mobilization Phase started on the first day of administration of G-CSF and continued until the final day of leukapheresis. A schematic of graft production and administration for the protocol is provided in FIG. 1C.

Large volume apheresis started on the 4th day of G-CSF administration, which was generally day −3 relative to CD34-enriched (HSPC) and Treg product infusions into the subject (defined as Day 0). Apheresis commenced with a target of ≥3×10⁶ CD34⁺ cells/kg recipient body weight post-selection. In order to have sufficient cells available for CD34 enrichment and Treg sorting procedures, donors underwent apheresis collections on 2 consecutive days (e.g., Days −3 and −2). To the degree possible, the 1st day's apheresis (Day −3) collection was scheduled for afternoon hours and the 2nd day's (Day −2) for early morning, thereby limiting the time from the end of the first collection to the infusion of the cellular products to less than 72 hours. Plerixafor (e.g., 0.24 mg/kg SC, once) was available prior to the second apheresis if recommended by the investigator and/or attending physician to achieve the HSPC dose target.

The CD34 reduced (flow-through) fractions were retained and used for isolation of donor Treg. For cell selection, clinical grade reagents were used under Good Manufacturing Practice (GMP) Conditions within the BMT Cellular Therapy Facility.

CD25+ cells were then selected from the CD34-depleted fraction using bead purification (Miltenyi). Tcons were obtained from the negative fraction and the positive fraction was used for Treg purification. CD4+CD25+CD127dim cells underwent further selection by FACS using a BD Influx cell sorter (BD Biosciences, San Jose CA). Enrichment of Tregs was following depletion of CD34+ cells by immunomagnetic selection, selection of CD25+ by immunomagnetic selection and purification by FACS sorting of CD4+CD127lowCD25+ cells. High purity of Tregs were obtained. These cells were highly suppressive in a mixed lymphocyte reaction (MLR).

When a lower than intended Treg dose level was achieved, e.g., a final Treg yield of <2×10⁶/kg body weight of the recipient, the recipient received a dose of 1-2×10⁶ Treg/kg if that dose could be achieved, and a reduced Tcon dose such that the ratio of administered Treg to Tcon was 1:1.

### G. Treatments

Subjects received the cell components indicated in TABLE 9

TABLE 9

| | | Cell Population |
|---|---|---|
| | | Target Dose Range, |
| | | per Recipient |
| Graft Component: | Dose Regimen | kg |
| HSPC (also described herein as | IV, Once, | ≥3 × 10⁶ |
| a “first composition of CD45+ | on Day 0 | |
| cells) | | |
| Treg (also described herein as a | IV, Once, | 2-3 × 10⁶ |
| “cell composition enriched for | on Day 0 | |
| Tregs) | | |
| Tcon (also described herein as a | IV, Once, | 3 × 10⁶ |
| “second composition of CD45+ | on Day +2 | |
| cells) | | |

In the Examples, term “HSPC” as a transplant, as a graft, as a cell dose, as a product, as a drug product, as a component, or as a graft component, and the like corresponds to the “first composition of CD45+ cells” and the like as disclosed elsewhere in the application, including the claims; the term “Treg” as a transplant, as a graft, as a cell dose, as a product, as a drug product, as a component, or as a graft component, and the like corresponds to the “cell composition enriched for Tregs) and the like as disclosed elsewhere in the application, including the claims; and the term “Tcon” as a transplant, as a graft, as a cell dose, as a product, as a drug product, as a component, or as a graft component, and the like corresponds to the “second composition of CD45+ cells) and the like as disclosed elsewhere in the application, including the claims.

Patients received Treg cells with purity higher than 90%, with a median Treg purity of 93.8%+/−3.1%. FIGS. 2B-2D illustrates the cell dose of HSPCs and Treg cells administered to patients. Table 10 below illustrates an analysis of the HSPC drug product (e.g., the first population of CD45+ cells) from a representative subset of 20 subjects enrolled in this study. Table 11 below illustrates an analysis of the Treg drug product (e.g., the population of cells enriched for Tregs) from a representative subset of 20 subjects enrolled in this study. Cell components were provided as single dose transfer bags, with an approximate fill volume of 100 mL each for both the Treg and the HSPC components. For each one of Tables 10-11, samples from 20 subjects from the multicenter clinical trial were analyzed. Means and standard deviations (SD) shown in each table. The mean (SD) patient weight was 71.8 kg (11.2 kg). Impurity cell doses were calculated by multiplying the % impurity by the reported cell dose for each patient

The Tcon component (e.g., the second population of CD45+ cells) was provided frozen, after storage in a vapor phase liquid nitrogen tank, in an approximate volume of 15 mL. The pooled apheresis product was assessed for CD3+ Tcon cells and a volume of the apheresis frozen product comprising 3.0E+06 Tcons was calculated and administered to subjects. Table 12 below illustrates an analysis of the Tcon drug product from a representative subset of 20 subjects enrolled in this study. Analysis of the Tcon drug products from 20 grafts in the multicenter clinical trial (N=20). Means and standard deviations (SD) shown. Cell doses were calculated for the cell populations based on the targeted CD3+ T cell dose of 3M/kg. Biomarker data (BM), manufacturing data (mfg); mean (SD) patient weight was 71.8 kg (11.2 kg).

Each cellular therapy product comprises cells, Plasma-Lyte A, and human serum albumin, at a pH of approximately 7.4. The bags and/or primary bag containers had labels bearing the appropriate label text as required by governing regulatory agencies.

The amount of time from when the cell product was received from the donor to when it was administered to the recipient was under 60 hours as is shown for a subset of patients in TABLE 13.

Dosing was based on a subject's actual body weight rounded to the nearest tenth of a kilogram, as assessed during screening, or based on adjusted body weight (ABW) if the subject's actual weight is greater than 120% of the ideal body weight (IBW), calculated as ABW [in kg]=[(actual weight −IBW)×0.40]+IBW.

TABLE 14 illustrates endotoxin levels in each cell population for a subset of patients.

TABLE 10
HSPC product analysis

| | % of Live | | | |
|---|---|---|---|---|
| Cell population | CD45+ cells | SD | Cell Dose/kg | SD |
| CD45+ live cells | 100 | 0.0 | 8.2E+06 | 3.6E+06 |
| HSPCs (purity) | 93.3 | 11.5 | 7.7E+06 | 3.8E+06 |
| Non-HSPCs (impurity) | 6.7 | 11.5 | 4.2E+05 | 7.3E+05 |
| Granulocytes | 3.2 | 6.8 | 1.9E+05 | 4.3E+05 |
| Monocytes | 1.2 | 2.2 | 7.2E+04 | 1.3E+05 |
| Lymphocytes | 1.1 | 0.8 | 7.8E+04 | 5.7E+04 |
| Non-T lymphs | 1.0 | 0.8 | 7.2E+04 | 5.6E+04 |
| T cells | 0.1 | 0.1 | 6.1E+03 | 9.5E+03 |

TABLE 11
Treg Product analysis

| | % of Live | | | |
|---|---|---|---|---|
| Cell population | CD45+ cells | SD | Cell Dose/kg | SD |
| CD45+ live cells | 100.0 | 0.0 | 2.8E+06 | 3.6E+05 |
| Tregs (purity) | 96.5 | 2.2 | 2.7E+06 | 3.7E+05 |
| Non-Tregs (impurity) | 3.5 | 2.2 | 9.3E+04 | 5.4E+04 |
| Grans and Monos | 0.1 | 0.1 | 1.9E+03 | 1.4E+03 |
| Lymphocytes | 3.2 | 2.1 | 8.6E+04 | 5.2E+04 |
| T cells | 2.4 | 1.9 | 6.3E+04 | 4.6E+04 |
| CD4+ T cells | 2.3 | 1.9 | 6.2E+04 | 4.6E+04 |
| Non CD4+ T cells | 0.0 | 0.1 | 1.4E+03 | 1.6E+03 |
| Non-T cell lymphs | 0.8 | 1.2 | 2.2E+04 | 3.2E+04 |

TABLE 12
Tcon product analysis

| | % of Live | | | |
|---|---|---|---|---|
| Cell population | CD45+ cells | SD | Cell Dose/kg | SD |
| CD45+ live cells (mfg) | 100.0 | 0.0 | 1.0E+07 | 2.4E+06 |
| CD45+ cell (BM) | 100.0 | 0.0 | 1.9E+07 | 7.1E+06 |
| Grans + Monos (mfg) | 52.2 | 8.1 | 6.2E+06 | 2.3E+06 |
| Grans + Monos (BM) | 56.7 | 8.8 | 1.1E+07 | 5.8E+06 |
| Granulocytes (BM) | 27.6 | 9.9 | 5.6E+06 | 3.9E+06 |
| Monocytes (BM) | 25.7 | 6.6 | 4.9E+06 | 2.1E+06 |
| Lymphocytes (mfg) | 41.1 | 7.8 | 4.6E+06 | 5.7E+05 |
| Lymphocytes (BM) | 29.8 | 9.4 | 5.1E+06 | 6.8E+05 |
| CD3+ (T) cells reported | 30.1 | 6.4 | 3.0E+06 | 0.0E+00 |
| CD3+ (T) cells (BM) | 17.7 | 5.7 | 3.0E+06 | 0.0E+00 |
| CD4+T cells (BM) | 8.8 | 3.5 | 1.5E+06 | 3.2E+05 |
| CD8+ T cells (BM) | 7.4 | 3.2 | 1.2E+06 | 3.0E+05 |
| B cells (BM) | 5.2 | 2.5 | 9.1E+05 | 3.7E+05 |
| NK cells (BM) | 3.6 | 1.9 | 6.4E+05 | 3.3E+05 |
| HSPCs (mfg) | 0.8 | 0.4 | 9.0E+04 | 5.8E+04 |
| Tregs (BM) | 0.5 | 0.2 | 8.2E+04 | 3.8E+04 |

TABLE 13
Time duration between collection and administration

| Category | Activity | N | Time (hrs) |
|---|---|---|---|
| Transportation | Local collection site to | N = 17 | 2.8 +/− 1.3 |
| | sorting facility | | |
| Transportation | Out of State collection | N = 7 | 11.2 +/− 2.6 |
| | facility to sorting | | |
| | facility | | |
| Manufacturing | Product dwell time at | N = 24 | 25.6 +/− 5.0 |
| | sorting facility | | |
| Total | Vein-to-vein | N = 24 | 60.5 +/− 10.9 |

TABLE 14
Endotoxin levels

| | Endotoxin (EU/ml) | Endotoxin (EU/ml) | Endotoxin (EU/ml) |
|---|---|---|---|
| | in HSPC drug | in Treg drug | in Tcon drug |
| Patients | product | product | product |
| 1 | <0.025 | <0.05 | <0.5 |
| 2 | <0.025 | <0.05 | <0.5 |
| 3 | 0.043 | <0.061 | <0.5 |
| 4 | <0.025 | <0.061 | <0.5 |
| 5 | <0.026 | <0.05 | <0.5 |
| 6 | <0.025 | <0.05 | <0.5 |
| 7 | <0.025 | <0.05 | <0.5 |
| 8 | <0.025 | <0.05 | <0.5 |
| 9 | <0.025 | <0.05 | <0.5 |
| 10 | <0.025 | <0.05 | <0.717 |
| 11 | <0.025 | <0.05 | <0.5 |
| 12 | <0.025 | <0.05 | <0.5 |
| 13 | <0.025 | <0.05 | <0.5 |
| 14 | <0.025 | <0.05 | <0.5 |
| 15 | <0.025 | <0.05 | <0.5 |
| 16 | <0.025 | <0.05 | <0.5 |
| 17 | <0.025 | <0.05 | <0.5 |
| 18 | <0.025 | <0.05 | <0.5 |
| 19 | <0.025 | <0.05 | <0.5 |
| 20 | <0.025 | <0.05 | <0.5 |
| 21 | <0.025 | <0.05 | <0.5 |
| 22 | <0.025 | <0.05 | <0.5 |
| 23 | <0.025 | <0.05 | <0.5 |
| 24 | <0.025 | <0.05 | <0.5 |
| 25 | <0.025 | <0.05 | <0.5 |
| 26 | <0.025 | <0.05 | <0.5 |
| 27 | <0.025 | <0.05 | <0.5 |
| 28 | <0.025 | <0.05 | <0.5 |
| 29 | <0.5 | <0.5 | <0.5 |
| 30 | <0.025 | <0.05 | <0.5 |
| 31 | <0.025 | <0.05 | <0.5 |
| 32 | <0.025 | <0.05 | <0.5 |
| 33 | <0.025 | <0.05 | <0.5 |
| 34 | <0.025 | <0.063 | <0.5 |
| 35 | <0.025 | <0.05 | <0.5 |
| 36 | <0.025 | <0.05 | <0.5 |
| 37 | <0.025 | <0.05 | <0.5 |
| 38 | <0.025 | <0.05 | <0.5 |
| 39 | <0.025 | <0.05 | <0.5 |
| 40 | <0.025 | <0.05 | <0.5 |
| 41 | <0.025 | <0.05 | <0.5 |
| 42 | <0.025 | <0.05 | <0.5 |
| 43 | <0.025 | <0.05 | <0.5 |
| 44 | <0.025 | 0.066 | <0.636 |
| 45 | <0.025 | 0.05 | <0.5 |
| 46 | <0.025 | 0.05 | <0.5 |
| 47 | <0.025 | <0.05 | <0.5 |
| 48 | <0.025 | <0.025 | <0.5 |
| 49 | <0.025 | 0.075 | <0.5 |
| 50 | <0.025 | <0.05 | <0.5 |
| 51 | <0.025 | <0.05 | <0.5 |
| 52 | <0.025 | <0.05 | <0.5 |
| 53 | <0.025 | <0.05 | <0.5 |
| 54 | <0.0391 | <0.05 | <0.5 |
| 55 | <0.025 | <0.05 | <0.5 |
| 56 | <0.025 | <0.05 | <0.5 |
| 57 | <0.025 | <0.05 | <0.5 |
| 58 | <0.025 | <0.05 | <0.809 |
| 59 | <0.0294 | <0.05 | <0.5 |
| 60 | 0.0457 | <0.05 | <0.5 |
| 61 | 0.0427 | <0.063 | <0.5 |
| 62 | <0.025 | <0.05 | <0.5 |

Recipients had appropriate long-term central venous access placed prior to initiation of the conditioning regimen. Unless contraindicated, subjects were administered acetaminophen or paracetamol (e.g., 500-1000 mg) and diphenhydramine (e.g., 25-50 mg) prior to administration of each cell component.

The CD34+ HSPC cell component (e.g., the first population of CD45+ cells), then the Treg cell component (e.g., the population of cells enriched for Tregs), were intravenously (IV) through a central venous catheter on study Day 0.

Subjects received a myeloablative conditioning (MA) regimen prior to administration of the cell components. Examples of MA regimens are provided in TABLE 15. Busulfan could also be dosed to maintain an average daily AUC of a 4,800-6,000 μM-min.

TABLE 15

| MA regimen | Drugs and doses |
|---|---|
| Total Body Irradiation/Cyclophosphamide/ | TBI (1320-1400 cGy) |
| Thiotepa (TBI/Cy/Thiotepa) | Cyclophosphamide (120 mg/kg) |
| | Thiotepa (10 mg/kg) |
| Busulfan/ Melphalan/ Fludarabine (Bu/Mel/Flu) | Busulfan (9.6 mg/kg IV) |
| | Fludarabine (125 mg/m²) |
| | Melphalan (140 mg/m²) |
| Busulfan/ Fludarabine (Bu/Flu) | Busulfan (16 mg/kg PO or 12.8 mg/kg IV) |
| | Fludarabine (160 mg/m²) or 120-180 mg/m² |
| | administered over 4 days |
| Busulfan/ Cyclophosphamide (Bu/Cy) | Busulfan (16 mg/kg PO or 12.8 mg/kg IV) |
| | Cyclophosphamide (100 mg/kg) |
| Cyclophosphamide/Total Body Irradiation | Cyclophosphamide (100 mg/kg) |
| (Cy/TBI) | TBI (1200-1420 cGy) |
| Total Body Irradiation/Etoposide (TBI/Etoposide) | TBI (1200-1320 cGy) |
| | Etoposide (60 mg/kg) |
| Total Body Irradiation/Etoposide/ | TBI (1320 cGy) |
| Cyclophosphamide (TBI/Etoposide/Cy) | Etoposide (60 mg/kg, 1 dose) |
| | Cyclophosphamide (60 mg/kg, 1 dose) |
| Bu/Flu/TTP | Thiotepa 5 mg/kg × 2 days (days −7 and −6) |
| | Busulfan 3.2 mg/kg × 3 days (days −5 to −3), total |
| | dose 9.6 mg/kg |
| | Fludarabine 50/m² (meters squared − body surface |
| | area) × 3 days (days −5 to −3) |
| TBI/Flu/TTP | Total body irradiation: HFTBI administered in 11 |
| | fractions of 125 cGy (centigray) over 4 days, to a |
| | total dose of 1375 cGy. |
| | Thiotepa 5 mg/kg × 2 days (days −7 and −6) |
| | Fludarabine 25/m² × 5 days |
| TBI/Flu/TTP/Bu | Thiotepa 5 mg/kg × 2 days (days −7 and −6) |
| | Busulfan 3.2 mg/kg × 3 days (days −5 to −3), total |
| | dose 9.6 mg/kg |
| | Fludarabine 50/m² (meters squared − body surface |
| | area) × 3 days (days −5 to −3) |
| | Total body irradiation: HFTBI administered in 11 |
| | fractions of 125 cGy (centigray) over 4 days, to a |
| | total dose of 1375 cGy |

Subjects received either tacrolimus or sirolimus as a single-agent GVHD prophylaxis beginning on the day following Tcon infusion (typically Day +3), e.g., of the second population of CD45+ cells.

Tacrolimus was initiated at 0.03 mg/kg/day IV, with a target trough blood level of 5-10 ng/mL. Per os (PO) administration was permissible if the patient was able to tolerate food.

For subjects that underwent MA with TBI/Cy/Thiotepa, Cy/TBI, TBI/Etoposide, TBI/Etoposide/Cy (i.e., regimens that do not include busulfan), sirolimus was initiated as a single loading dose of 6 mg PO, followed by 2 mg daily for a target blood level of 3-8 ng/mL.

For subjects that did not show signs of ≥grade 2 acute GVHD prior to Day +60, the GVHD prophylaxis could be reduced, e.g., by approximately 20% of the dose per month. For subjects that showed signs of ≥grade 2 acute GVHD, GVHD prophylaxis could be tapered after no signs of GVHD were observed for a suitable period of time (e.g., a suitable period of time after ceasing administration of any GVHD therapeutic agents, and not observing ≥grade 2 GVHD).

### H. Clinical Outcomes

The following paragraphs provide data and explanations related to clinical outcomes from the clinical studies described herein. As an introductory matter in the explanation of the data, it is to be recognized that graft failure can be a complication of HCT, and can be associated with significant mortality.

Neutrophil engraftment through Day +28: Neutrophil engraftment was defined as achieving an absolute neutrophil count (ANC) ≥500/mm³ for 3 consecutive days, by Day +28. The first of the three days was designated the day of engraftment. If ANC never dropped below 500/mm³, Day +1 was assigned as the day of engraftment. Study group patients showed earlier neutrophil (median of 11 days vs. 14 days, p<0.0001 by Mann-Whitney U).

Platelet engraftment through Day +50: Platelet engraftment was defined as achieving a platelet count >20,000/mm³ for 3 consecutive days without platelet transfusion in the preceding 7 days, by Day +50. The first of the three days was designated the day of engraftment. If platelet count never dropped below 20,000/mm³, Day +1 was assigned as the day of engraftment. Study group patients showed earlier platelet engraftment (11 vs 17 days, p<0.0001).

Secondary graft failure through Day +100: Secondary graft failure was defined as neutrophil engraftment followed by subsequent decline in absolute neutrophil counts <500 cells/μL, unresponsive to growth factor therapy, by Day +100.

A failure to achieve an absolute neutrophil count of >500 cells/μL after Day +30 can indicate primary graft failure. No subjects in the study group experienced primary graft failure.

A sustained loss of hematopoiesis after engraftment has occurred can indicate secondary graft failure. No subjects in the study group experienced secondary graft failure.

Peripheral blood samples from subjects were also analyzed for chimerism as part of the follow-up procedures. The blood samples were processed to select for various cell populations using standard markers for each cell population. Blood was drawn from subjects that received the composition of the disclosure on day +28, day +56, day +100, day +180 and day +365 post-transplant, and the frequency of peripheral blood cells were quantified by flow cytometry (Markers used for analysis are described in Table 16).

Achieving a platelet count ≥20,000/mm³ for 3 consecutive days without platelet transfusion can indicate platelet engraftment. As shown in FIG. 3A, subjects in the study group exhibited more rapid platelet engraftment than subjects in the SOC cohort, achieving platelet engraftment after a median of 11 days compared to 17 days for the SOC cohort (p<0.0001). FIG. 3B illustrates the platelet counts in donors before and after mobilization and in receiving patients before transplant and after transplants. Boxplots where shown: boxes show the 75^{th}, 50^{th}, and 25^{th} percentiles; whiskers show the 90^{th} and 10^{th} percentiles. X-axes nomenclature: the leading number (e.g. 01, 02, 025, . . . ) are mentioned for ordering; following the underscore, Dscrn=healthy donor pre-G-CSF mobilization, Rscrn=recipient within 1 month prior to conditioning, apher=healthy donor blood draw at the time of apheresis, d028=recipient day 28 post transplant, d056-d365=recipient days post transplant. N's shown indicate the sample sizes for each timepoint. Symbols indicate values for individual measurements.

Achieving a sustained neutrophil count of >500 cells/μL can indicate neutrophil engraftment. FIG. 3C illustrates the platelet counts in donors before and after mobilization and in receiving patients before transplant and after transplants. Figure legends are similar to the legend described in FIG. 3B.

FIGS. 3D-E illustrate monocyte and lymphocyte engraftment in the patients. FIGS. 3M and N illustrate a comparison of lymphocyte and monocyte counts in representative patients compared to a representative SOC cohort.

FIG. 3F illustrates that B cells engraft in recipients of the composition of the disclosure, and that mature B cells are present by day +100 post-transplant. Superior engraftment and/or earlier functionality of engrafted B cells may represent a significant advantage over standard of care grafts, for example, enhancing immunity and allowing for vaccination post-transplant.

FIG. 3G illustrates CD3+ T cell engraftment; FIG. 3H demonstrates NK cell engraftment, FIG. 3I illustrates CD4+ T cell engraftment and FIG. 3J illustrates CD8+ T cell engraftment in recipients of the composition of the disclosure. FIG. 3K illustrates a ratio of CD4:CD8 T cells in the recipients.

FIG. 3L provides a comparison of the proportion of CD3+CD4+ T cells that were Tregs in healthy donors, compared to graft recipients on several days post-transplant. These data show that recipients of the composition of the disclosure exhibit high frequencies of circulating CD4+ Tregs. Without wishing to be bound by theory, a lower Treg frequency may contribute to prevention of GVHD, reduced risk of developing GVHD, reduced incidence of GVHD, reduced severity of GVHD, or a combination thereof.

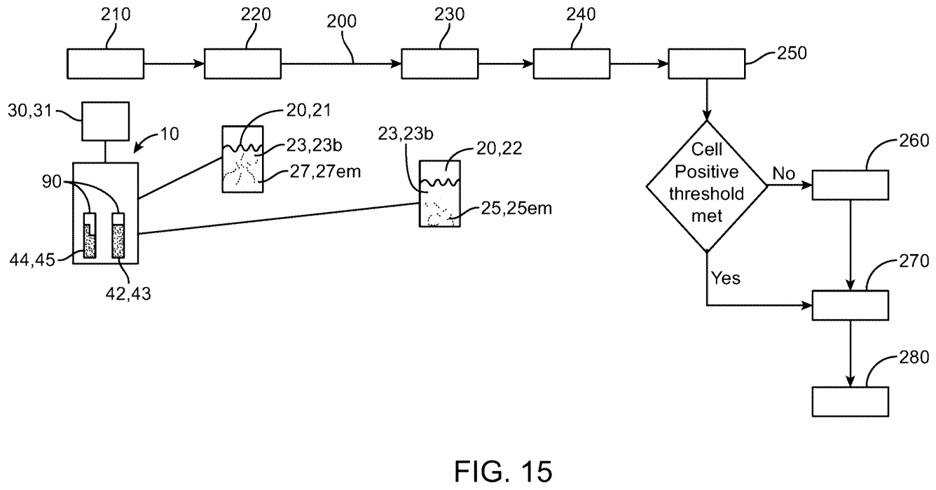

FIG. 30 shows representative data from two subjects compared to a healthy control. In the healthy control, 3.72% of circulating CD3+CD4+ T cells were Tregs (CD25+CD127dim). In the two graft recipients, 28.1% and 23.7% of CD3+CD4+ T cells were Tregs on day +28, 32.3% and 17.8% on day +56, and 19.2% and 20.7% on day +100.

Blood drawn from a graft recipient on day +100 post-transplant the sample was processed for flow cytometric evaluation of various B cell markers including CD19, CD20, IgD, and CD27. FIG. 3P compares scatterplots from the graft recipient to a healthy control. In all cases, the Y axis is for CD19+ staining. The left panels show gating of lymphocytes to identify B cells (CD19+) and T cells (CD3+). 13.4% of lymphocytes in the graft recipient were B cells, compared to 9.84% in the healthy control. The following panels show that 98.3-100% of cells gates as CD19+ were also CD20+. The panels second from the right show the fraction of B cells that are IgD+, which can be used to identify mature B cells. 92.1% of B cells in the graft recipient were IgD+, and 89.5% in the healthy control. The right-most panels show staining for CD27, which can be used to identify memory B cells, late plasmablasts, and plasma cells, for example. 43.6% of B cells in the graft recipient were CD27+, and 67.1% in the healthy control.

These results demonstrate that B cells engraft in recipients of the composition of the disclosure, and that mature B cells are present by day +100 post-transplant. Superior engraftment and/or earlier functionality of engrafted B cells may represent a significant advantage over standard of care grafts, for example, enhancing immunity and allowing for vaccination post-transplant.

### I. GVHD Evaluation

Acute GVHD was staged and graded per MAGIC Standardization criteria.

Chronic GVHD was diagnosed, staged and graded per the International NIH Chronic GVHD Diagnosis and Staging Consensus Working Group criteria.

Clinically significant manifestations of both acute and chronic GVHD were treated first by local, topical, and/or systemic corticosteroids (e.g. prednisone). Any patients who were refractory or resistant to, dependent upon, or intolerant of corticosteroids, per BMT-NIH-CIBMTR Task Force definition, were to be considered for second line therapy.

Treatment-emergent adverse events (TEAEs): TEAEs were categorized by System Organ Class and preferred term using MedDRA version 21.0 and were graded according to the CTCAE version 5.0.

Acute GVHD: Acute GVHD (aGVHD) is a significant driver of morbidity and mortality associated with alloHCT, reducing the severity and incidence of aGVHD has the potential to greatly benefit graft recipients. Acute GVHD was staged and graded per Mount Sinai Acute GvHD International Consortium (MAGIC) Standardization criteria.

Subjects were considered evaluable for aGVHD if they developed aGVHD symptoms before day +100 (100 days post-transplant), or were beyond day +100 without exhibiting aGVHD symptoms. Using these criteria, 17 patients are evaluable for aGVHD at the time of reporting.

The Grade ≥2 aGVHD rate observed was 16% at the time of reporting. This compares favorably to published rates of in similar populations, and was lower than patients in the SOC cohort. The onset of grade ≥2 aGVHD compared to the SOC cohort is shown in FIG. 4A. One subject developed aGVHD of the upper gastrointestinal (GI) tract, manifesting as nausea and cachexia. This subject's symptoms resolved with a short course of corticosteroids, which were subsequently weened.

Severe (Grade 3-4) aGVHD is a major contributor to non-relapse mortality post-alloHCT and can be observed in 10-20% of patients following an HLA-matched, related donor transplants. 5% of the patients developed grade 3-4 aGVHD in the study group, whereas 20% of patients in the SOC cohort developed Grade 3-4 aGVHD. The onset of grade ≥3 aGVHD in through Day +120 compared to the SOC cohort is shown in FIG. 4B.

As noted in TABLE 7, four patients in the study group did not receive any GVHD prophylaxis. Of those patients, only one has developed aGVHD symptoms (the case of upper GI tract aGVHD noted above). Results to the time of reporting suggest that the composition of the disclosure may be safely administered to patients without GVHD prophylaxis or with minimal prophylaxis. These strategies could significantly benefit patients due to, for example, increased graft versus tumor (GVT), increased graft versus infection (GVI), and reduced adverse effects that can be associated with immunosuppressive agents (e.g., renal toxicity and hepatotoxicity).

These results suggest a composition of the disclosure can reduce the incidence and severity of aGVHD in recipients compared to standard of care, for example, even in the absence of GVHD prophylactics or with reduced GVHD prophylaxis.

Steroid-refractory acute GVHD: Steroid-refractory acute GVHD was defined as per the EBMT-NIH-CIBMTR Task Force position statement.

Chronic GVHD: As chronic GVHD (cGVHD) is associated with significant morbidity and with decreased survival, reducing the severity or incidence of cGVHD has the potential to greatly benefit graft recipients. Chronic GVHD was diagnosed per 2014 International NIH Chronic GVHD Diagnosis and Staging Consensus Working Group criteria.

At the time of reporting, no subjects in the study group had developed moderate or severe cGVHD. One subject in the study group developed transient, steroid-responsive mild cGVHD of the skin.

Analysis was conducted using a cutoff of 365 days (i.e. cGVHD events that occur after Day +365 were not included). As shown in FIG. 4C, Subjects in the study group experienced significantly fewer moderate or severe cGVHD events than patients in the SOC cohort (5% vs. 43%, respectively; p<0.0001). Despite receiving less GVHD prophylaxis or no GVHD prophylaxis, data showed that a composition of the disclosure can significantly reduce the incidence of cGVHD compared to subjects in the SOC cohort. Additionally, several subjects in the study group have undergone greater than 2.5 years of follow-up with no cGVHD symptoms reported.

As cGVHD is associated with decreased overall survival and significant long-term morbidity, these results suggest that a composition of the disclosure can improve long-term survival and quality of life in recipient subjects compared to standard of care.

Post-Transplant Lymphoproliferative Disorder (PTLD): PTLD was defined as a biopsy consistent with the 2017 World Health Organization (WHO) classification of PTLD (nondestructive [plasmacytic hyperplasia, infectious mononucleosis-like, and florid follicular hyperplasia], polymorphic, monomorphic or Hodgkin lymphoma-like), along with lymphoma type-appropriate staging procedures such as computed tomography (CT) with or without 18F-fluorodeoxyglucose positron-emission tomography (FDG-PET).

Incidence of non-relapse mortality (NRM). NRM was defined as death without evidence of disease recurrence. Disease relapse/progression was considered a competing event.

Incidence of disease relapse: For acute leukemias, relapse was defined as any of the following (MRD+ alone was insufficient): (i) ≥50% blasts in the bone marrow or peripheral blood; or (ii) Reappearance of pre-transplant cytogenetic abnormality; or (iii) new evidence or redevelopment of extramedullary disease. For MDS, relapse was defined as any of the following: (i) satisfying criteria for evolution into acute leukemia; (ii) reappearance of pre-transplant morphologic abnormalities, detected in bone marrow specimens; or, (iii) reappearance of pre-transplant cytogenetic abnormality in at least one metaphase on each of two separate consecutive examinations at least one month apart, regardless of the number of metaphases analyzed.

Treatment related mortality includes deaths from complications or toxicities associated with therapy, such as infection, GVHD, or organ failure. At the time of reporting, the treatment-related mortality rate in the study group was 5% for one-year post-transplant, compared to 13% for the SOC cohort, as shown in FIG. 4D. No subjects in the study group had died from complications other than disease relapse.

Subjects were monitored for survival and relapse. For comparison of clinical outcomes, a standard of care (SOC) comparator cohort was identified retrospectively from subjects who received a SOC myeloablative alloHCT of mobilized peripheral blood from a fully HLA-matched donor at one of the same clinical sites during the same period. All subjects in the SOC cohort received myeloablative conditioning regimens and dual-agent GVHD prophylaxis with tacrolimus and methotrexate.

FIGS. 4E-4H illustrate the relapse, GVHD and relapse free survival rates, chronic GVHD free survival rates and overall survival rates of subjects that were recipients of standard of care grafts compared to subjects that received grafts described in this example. These data suggest that the compositions described herein improve relapse-free survival in subjects undergoing myeloablative alloHCT.

GVHD and relapse-free survival is a composite readout that can refer to survival without relapse or Grade ≥3 acute or extensive chronic GVHD. At the time of reporting, the percent of GVHD-free and relapse-free survival to one-year post-transplant was 74% for the study group, compared to 34% for the SOC cohort, as shown in FIG. 4F. The difference was statistically significant (p=0.0001 by log-rank test).

Overall survival: OS was defined as the time from the date of transplant to the date of death from any cause or, for surviving patients, to the date of last follow-up. At the time of reporting, the percent of overall survival to one-year post-transplant was 90% for the study group, compared to 78% for the SOC cohort, as shown in FIG. 4H. The difference was statistically significant (p=0.0242 by log-rank test).

### H. Hospitalization Time

Standard of care HCT can require a lengthy inpatient stay. Compared to patients in the SOC cohort, the median time from transplant (Day 0) to hospital discharge for patients in the study group was 2.5 days shorter (from 18.5 to 16 days, p<0.01 by Mann-Whitney U test). FIG. 4I illustrates hospitalization times for a representative subset of patients.

I. Disease Status after Therapy

Subjects in the study were first evaluated for relapse on day +90 post-transplant. At the time of reporting, only 16% patients relapsed compared to 19% patients in the SOC cohort.

TABLE 16: subject status for a representative subset of subjects past day +90. Abbreviations used: DLBCL, diffuse large B cell lymphoma; AML, acute myeloid leukemia; ALL, acute myeloid leukemia; MDS, myelodysplastic syndrome; MF, myelofibrosis; MPAL, mixed phenotype acute leukemia; CML, chronic myeloid leukemia; CR: complete remission; CR1, 1st complete remission; CR2, 2nd complete remission; active, active disease (not in CR as defined for given disease entity).

TABLE 16

| Disease | Disease status at transplant | Most recent follow up and status |
|---|---|---|
| DLBCL | Active | Relapsed, day +113 |
| AML | CR2 | Relapsed, day +367 |
| ALL | Active | Alive, in remission, day +1107 |
| ALL | CR2 | Alive, in remission, day +1079 |
| CML | Active | Relapsed, day +76, then responded to donor lymphocyte |
| | | infusion; Alive, in remission, day +1058 |
| AML | CR2 | Alive, in remission, day +981 |
| MF | Active | Alive, in remission, day +932 |
| AML | CR1 | Alive, in remission, day +260 |
| PTCL | Active | Alive, in remission, day +225 |
| AML | CR1 | Alive, in remission, day +211 |
| AML | CR1 | Alive, in remission, day +204 |
| MDS | Active | Alive, in remission, day +190 |
| ALL | CR1 | Relapsed, day +119 |
| ALL | CR2 | Alive, in remission, day +127 |
| MF | Active | Alive, in remission, day +119 |
| MPAL | CR1 | Alive, in remission, day +120 |
| AML | CR1 | Alive, in remission, day +99 |

The risk of relapse can be associated with disease status at the time of transplant. For example, the prognosis of AML or ALL subjects can be significantly worse if the subjects are not in complete remission at time of transplant (i.e., prognosis is worse if the subjects have active leukemia or detectable minimal residual disease at the time of transplant). In another example, prognosis is worse in subjects that have detectable minimal residual disease versus patients who do not have detectable minimal residual disease. Subjects with active disease or with minimal residual disease can represent a critical unmet medical need.

FIG. 5 summarizes the disease status of a subset of subjects in the study group before transplant and at day +90, +180, and +356 post-transplant. Of the 10 subjects that received the composition of the disclosure, 7 achieved durable remissions, demonstrating a graft-versus tumor effect. Two of the subjects that relapsed had active disease at the time of transplant, and one had detectable minimal residual disease (MRD) at the time of transplant.

Prevention of relapse was robust with the treated patients with relapse-free survival of 81% at both 1 year and 18 months in recipients. MRD status was determined for 77 patients with acute leukemia by multicolor flow cytometry; of these patients, 31% were MRD+ when they received Orca-T. Amongst MRD-patients (n=53), RFS with Orca-T was 90% at both 1 year as compared to 66% in the CIBMTR cohort (n=324 MRD-patients). Amongst MRD+ patients (n=24), RFS was 68% at one year with the transplant patients as compared to 48% in the comparator cohort (n=104).

Relapse prevention with the treatment appeared to be enhanced further with a conditioning regimen consisting of busulfan, fludarabine, and thiotepa (“BFT”, n=56 patients, median f/u 342 days); RFS was 90% at 12 months in this group. This included patients with MDS (n=6, 100% RFS at 1 yr), MRD+ acute leukemia (n=14, RFS 73% at 1 yr), MRD-acute leukemia (n=26, RFS 96% at 1 year), and acute leukemia with unknown MRD status (n=11, 91% RFS at 1 yr) (FIG. 10).

As with relapse, severe infections were low following treatment with 11% of patients developing Grade 3 infections per the BMT-CTN grading scale.

Median times to neutrophil and platelet engraftment were rapid with treatment at 13 and 16 days, respectively; graft failure was rare at 1.6%. Grade ≥3 aGVHD and mod/severe cGVHD rates were low with Orca-T at 5% and 6%, respectively, through 1-year post-transplant. Non-relapse mortality was low at 5% at 1 year; NRM with BFT was 0%. Overall, treatment shows GRFS of 76% and 69% and 1 year and 18 months post-transplant, respectively; OS was 91% and 86% at these timepoints post-transplant. No formal comparison to the CIBMTR cohort was performed.

TABLE 17

| | CIBMTR | |
|---|---|---|
| parameter | Control | Orca-T |

| | | |
|---|---|---|
| n | 375 | 126 |

| | | | | |
|---|---|---|---|---|
| Median follow-up | 31 | (4-50) | 13 | (1-69) |

| in months (range) | | | |
|---|---|---|---|
| Relapse-free survival @ 12 | n/a | 81% | (74-88) |
| months (95% CI) | | | |
| Relapse-free survival @ 12 | n/a | 92% | (84-100) |

months (95% CI) - BFT
conditioning

| | | | | |
|---|---|---|---|---|
| Relapse-free survival @ 12 | 48% | (39-58) | 72% | (53-91) |

months (95% CI) - MRD+
acute leukemia

| | | | | |
|---|---|---|---|---|
| Relapse-free survival @ 12 | 66% | (61-72) | 90% | (82-98) |

months (95% CI) - MRD
neg acute leukemia

| | | | | |
|---|---|---|---|---|
| Grade ≥3 aGVHD through | 16% | (2-19) | 5% | (1-9) |

Day +180* (95% CI)

| | | | | |
|---|---|---|---|---|
| Moderateto | 38% | (33-44) | 6% | (0-12) |

Severe cGVHD through
Day +365** (95% CI)

| | | | | |
|---|---|---|---|---|
| Non-relapse mortality @ | 10% | (7-13) | 5% | (1-9) |

| 1 year (95% CI) | | | |
|---|---|---|---|
| Non-relapse mortality @ | n/a | 0% | (n/a) |

1 year (95% CI) - BFT
conditioning

| | | | | |
|---|---|---|---|---|
| GVHD and Relapse-Free | 34% | (30-39) | 75% | (67-83) |

Survival at 1 year (95% CI)

| | | | | |
|---|---|---|---|---|
| Overall survival at | 68% | (63-73) | 89% | (83-95) |

1 year (95% CI)

### J. Characteristics of Transplanted Cells

A schematic of graft production and administration for the sorting protocol is provided in FIG. 1A. Cell product from apheresis collection Day 2 was given an assessment and a portion of the apheresis product comprising a heterogenous cell component (e.g., a second population of CD45+ cells) consisting of 3×10⁶ Tcon cells was administered to the subjects. A portion of the heterogenous cell component was analyzed for different cell components after staining for various cell populations as described in Table 18.

TABLE 18

| Cell population | Immunophenotype ((+) positive staining, (−) negative staining) |
|---|---|
| Granulocytes | CD45+, high side scatter, CD14− |
| Monocytes | CD45+, moderate side scatter, high forward scatter, CD14+ |
| Lymphocytes | CD45+, low forward and side scatter |
| PBMC | Defined as the total population of lymphocytes and monocytes |
| Lymphocyte subsets | |
| T cells | CD3+ lymphocytes |
| CD4+ T cells | CD4+, CD8− T cells |
| CD8+ T cells | CD8+, CD4− T cells |
| Naïve T cells | CD45RA+CD45RO− T cells (can be subdivided based on CD4 and CD8 |
| | expression) |
| Memory T cells | CD45RA−CD45RO+ T cells (can be subdivided based on CD4 and CD8 |
| | expression) |
| Tregs | CD25+, CD127dim CD4+ T cells |
| NKT cells | CD56+ T cells |
| B cells | CD19+, CD3− lymphocytes |
| NK cells | CD56+, CD3− lymphocytes |
| CD34+ cells (HSPCs) | CD45dim, CD34+, CD3− cells that fall within the general lymphocyte |
| | scatter region |
| PBMC subsets | |
| Plasmacytoid dendritic cells | CD3−, CD19−, CD56−, CD14− (Lin−) cells that are CD4+, CD11b-, and |
| (pDCs) | BDCA2+ |
| Myeloid dendritic cells | CD3−, CD19−, CD56−, CD14− (Lin−) cells that are CD4+, CD11b+, and |
| (mDCs) | variably express CD16 |

Table 19 below illustrates data collected from a few patients from the study and identifies the various cell populations transplanted into a subject as part of the heterogenous cell component comprising 3×10⁶ Tcon cells. Longitudinal peripheral blood counts of platelets, WBCs, neutrophils, lymphocytes, monocytes, T cells, B cells, and NK cells are presented in Table 20. T cell and B cell counts were readily observed at days 28 and 56 respectively, and increased with each subsequent time point. Median NK cell levels were observed to be in the normal range at all post-transplant time points. CD4+ T cell and Treg cell counts exhibited similar post-transplant patterns with both being appreciably present at d28 and increasing with each subsequent time point. Strikingly, relative to the level measured in 75 corresponding PBSC donors, the Treg frequency among CD4+ T cells was significantly elevated at all time points post-transplant (FIG. 3L). Median CD8+ T cell counts increased for the first 6 months post-transplant and then plateaued in the normal range. Upon PCA, very few significant differences were observed in 2-group comparisons of recipient sex and donor relation.

TABLE 19
Heterogenous cell component

| | | | Standard | | | | | |
|---|---|---|---|---|---|---|---|---|
| Cell subset | Parent gate | Mean | Deviation | Median | Range | Minimum | Maximum | Count |
| Tcons | 45+ Lymph | 60.05 | 8.67 | 58.6 | 33.4 | 47.6 | 81 | 22 |
| CD4+ T cells | T cells | 48.59 | 10.07 | 46.55 | 36.1 | 32.7 | 68.8 | 22 |
| CD8+ T cells | T cells | 42.45 | 10.27 | 42.1 | 32.7 | 24.7 | 57.4 | 22 |
| B-cells | Lymph | 18.62 | 6.37 | 19.25 | 25.09 | 5.71 | 30.8 | 22 |
| NK cells | 45+ Lymph | 11.81 | 5.5 | 11.4 | 23.5 | 4.2 | 27.7 | 22 |
| NKT cells | T cells | 10.02 | 5.38 | 9.01 | 20.98 | 3.32 | 24.3 | 22 |
| Treg cells | T4 | 6.01 | 2.1 | 5.84 | 7.49 | 2.71 | 10.2 | 21 |
| Naïve T cells | T cells | 50.25 | 8.57 | 49.5 | 32.8 | 34.8 | 67.6 | 21 |
| DRp38pT4 | T4 | 3.81 | 2.64 | 2.99 | 10.81 | 1.09 | 11.9 | 22 |
| DRp38pT8 | T8 | 7.39 | 4.67 | 5.72 | 18.24 | 1.96 | 20.2 | 22 |
| CD34+ HSPCs | 45+ Lymph | 1.62 | 0.64 | 1.66 | 2.33 | 0.31 | 2.64 | 22 |
| Granulocytes | 45+ | 58.48 | 9.12 | 58.7 | 34.8 | 38.3 | 73.1 | 20 |
| and Monocytes | | | | | | | | |
| Lymphocytes | 45+ | 28.64 | 10.02 | 28.75 | 34.4 | 10.4 | 44.8 | 20 |
| CD14+ | Total | 26.28 | 6.59 | 26.5 | 25 | 16.4 | 41.4 | 20 |
| pDC | Lin− | 6.66 | 5.05 | 4.43 | 17.53 | 1.97 | 19.5 | 12 |
| mDC | Lin− | 14.15 | 9.2 | 10.95 | 27.7 | 3.3 | 31 | 12 |

TABLE 20
Cell populations in patients

| | Donors | d + 28 | d + 56 | d + 100 | d + 180 | d + 365 |
|---|---|---|---|---|---|---|
| | N = 86 | N = 96 | N = 93 | N = 90 | N = 82 | N = 50 |
| Platelets | 268* | 94 | 122 | 150 | 174 | 199 |
| (×10³/ml) | (227-307) | (58-147) | (83-158) | (107-197) | (132-211) | (161-241) |
| WBCs | 6.2 | 4.2 | 4.1 | 4.2 | 4.4 | 5.2 |
| (×10³/ml) | (5.2-7.7) | (2.7-6) | (3.1-5.7) | (3.3-5.6) | (3.3-5.8) | (3.7-6.6) |
| Neutrophils | 3.5 | 2.6 | 2.4 | 2.7 | 2.5 | 3 |
| (×10³/ml) | (2.9-4.4) | (1.5-3.9) | (1.7-3.9) | (1.9-3.5) | (1.7-3.6) | (2-4.4) |
| Lymphocytes | 2 | 0.5 | 0.6 | 0.6 | 1.1 | 1.3 |
| (×10³/ml) | (1.7-2.3) | (0.3-0.8) | (0.4-0.8) | (0.4-1.1) | (0.6-1.6) | (0.8-2.2) |
| Monocytes | 0.4 | 0.6 | 0.4 | 0.5 | 0.4 | 0.4 |
| (×10³/ml) | (0.4-0.6) | (0.5-0.9) | (0.3-0.6) | (0.3-0.6) | (0.3-0.6) | (0.3-0.5) |
| T cells (/ml) | 1244 | 99 | 182 | 221 | 561 | 643 |
| | (1047-1510) | (54-226) | (81-471) | (108-523) | (199-976) | (312-1134) |
| CD4+ T cells | 646 | 32 | 66 | 78 | 148 | 203 |
| (/ml) | (518-852) | (16-56) | (30-125) | (45-119) | (80-204) | (87-319) |
| CD8+ T cells | 461 | 49 | 88 | 132 | 374 | 381 |
| (/ml) | (327-625) | (20-164) | (30-389) | (47-364) | (87-747) | (183-822) |
| Tregs (/ml) | 37 | 3 | 5 | 7 | 8 | 13 |
| | (29-48) | (1-7) | (3-10) | (4-10) | (4-13) | (8-22) |
| Tregs (% of | 5.7 | 10.7 | 8.5 | 7.8 | 6.7 | 6.8 |
| CD4+ T cells) | (4.5-7.2) | (7.3-17.8) | (5.2-14.1) | (5.1-12.2) | (4.7-10) | (5.3-9.9) |
| B cells (/ml) | 177 | 1 | 43 | 63 | 104 | 233 |
| | (119-253) | (0-2) | (5-114) | (17-150) | (48-236) | (92-493) |
| NK cells (/ml) | 287 | 245 | 182 | 182 | 160 | 189 |
| | (193-404) | (139-391) | (119-292) | (109-274) | (103-236) | (121-271) |

### Example 2: Analysis of Patients Receiving Different Conditioning Regimens

The data was further quantified for clinical outcomes in subjects that received different conditioning regimens. FIG. 6A compares acute GVHD in patients that received a Bu/Cy conditioning (see Table 14) vs a conditioning regimen comprising Thiotepa (BFT—busulfan, fludarabine and thiotepa; regimen described in Table 14). The acute GVHD rates were similar for the two regimens but were lower in patients that received thiotepa. A higher difference was seen in patients that received thiotepa in chronic GVHD (FIG. 6B). Surprisingly, the relapse rate in patients dropped significantly (p<0.03) in patients with the BFT regimen where no patients who received the BFT regimen relapsed (see FIGS. 6C and D). The GVHD and Relapse Free survival rates (GRFS) were also significantly lower in the BFT regimen patients (see FIG. 6E). The overall survival was also improved in the BFT-receiving patients (see FIG. 6F). These data demonstrate that conditioning regimens comprising thiotepa provide a clear and surprising advantage.

### Example 3: Analysis of Patients Receiving Different GVHD Prophylactic Agent Regimens

The data from the clinical trial was further quantified for clinical outcomes in subjects that received different GVHD prophylactic agents—sirolimus or tacrolimus. FIG. 7A compares acute GVHD in patients that received sirolimus only or tacrolimus only versus standard of care (SOC) patients that received a combination of methotrexate and tacrolimus (see FIG. 1B for SOC regimen). The patients that received only tacrolimus showed higher GVHD rates (FIGS. 7A-7C) than patients who received sirolimus. However, the survival (FIG. 7E), relapse rates (FIG. 7F), GRFS rates (FIG. 7G) and overall survival rates (FIG. 7H) were improved in tacrolimus-only patients relative to the sirolimus-only patients. As FIG. 7A shows, multiple drugs can be used as GHVD prophylaxis with significantly lower rates of acute GVHD versus standard of care.

Upon further analysis, it was observed that patients had different serum trough levels. Serum tacrolimus levels had direct effects on the clinical outcome in the patient populations as is shown in FIGS. 8A-9G). As illustrated in FIG. 8A, patients who maintained an average serum tacrolimus trough level higher than 4 ng/ml for the first 30 days post-transplant had lower GVHD rates. FIGS. 8B-8C are derived from the data in FIG. 8A and further present data for acute GVHD and chronic GVHD rates. They illustrate a calculation of the probability of developing GVHD (y axis) if tacrolimus levels fall below the threshold value (x axis).

FIGS. 9A-9G illustrate direct comparison of clinical outcomes in patients that had a serum tacrolimus trough level higher or lower than 4 ng/ml. Each figure legend describes the number of patients and the time periods where their serum tacrolimus trough levels were higher or lower than 4 ng/ml. Acute GVHD rates showed significant improvement with higher tacrolimus levels (FIG. 9A) and chronic GVHD rates also showed improvements (FIG. 9B).

Patients who received the same conditioning regimen (Bu/Cy for FIGS. 9C-9D; TBI/BFT for FIGS. 9E-9G) and but had different serum tacrolimus trough levels (higher or lower than 4 ng/ml) were also analyzed for GVHD rates and results are illustrated in FIGS. 9C-9G. In all instances, a higher tacrolimus trough level showed improvement in GVHD rates.

When T cell chimerism was compared to the average trough tacrolimus level through day +30 post-transplant, a significant correlation was found; lower trough tacrolimus levels were associated with increased engraftment of donor T cells (p=0.0011). FIG. 9H shows the average trough tacrolimus level through day +30 post-transplant for a small subset of patients, plotted against the proportion of CD3+ cells of donor origin at day +30 (except that chimerism data is from day 90 where indicated by “D90”). These data suggest that maintaining lower circulating levels of tacrolimus can contribute to improved engraftment of donor T cells and improved donor T cell chimerism, which may contribute to improved relapse-free survival in allogeneic hematopoietic stem cell transplant recipients. Without wishing to be bound by a theory, it can be hypothesized that an improved chimerism due to higher tacrolimus levels may have led to the improved GVHD and survival outcomes in patients.

### Example 4: Receptor Occupancy

PBMCs were isolated from a donor cell sample. A negative selection CD4 kit was used to isolate bulk CD4+ T cells. CD4 T cells were split into 5 groups and stained with the indicated CD25 clones conjugated to PE fluorophore. Receptor saturation was confirmed empirically for each antibody. Tregs were FACS sorted and then transferred to 5% Human Serum/i % penstrep/XVIVO-15 and stimulated with the IL-2 (10 U/mL or 100 U/mL) for 30 minutes at 37° C. Cells were fixed in methanol and stained for pSTAT5. Cells gated on CD45/CD3/CD4 were detected for pSTAT5 expression; results are provided in FIG. 11.

In one experiment, a CD25 antibody that binds competitively to the affinity reagent was employed as a conjugate to a fluorophore (Detection reagent). A portion of the cellular material was analyzed without staining with the detection antibody to and the MFI in the fluorophore channel was recorded as MFI-0. A portion of the cellular material (not having been exposed to the affinity reagent) was stained with an adequate amount of detection reagent to effect at least 90% of the saturation value and the MFI was noted (MFI-sat). Cellular material stained with the affinity reagent was stained under the same conditions, and the MFI was recorded (MFI-x). % Receptor Occupancy=100×(MFI-x−MFI-0)/(MFI-sat−MFI-0). Table 21 provides a small-scale titration of CD25 microbeads.

Table 21 provides relative concentrations of antibodies used to occupy the CD25 polypeptides expressed on the surface of the Tregs. Exact antibody concentrations are not provided in this illustrative experiment as antibody concentrations can vary due to several factors, some of which include: the production lot, specific antibody clone used, binding efficiency, binding conditions, and cell health and viability etc.

A person skilled in the art can measure the concentration of the antibody to be used (to maintain receptor occupancy) as described herein or by other routine methods. The concentration of antibody can be dependent on purity of the antibody or cells, a desired purity or yield level of the desired cells. Concentration of the antibody can also be modified by measuring a desired level of pSTAT5 activity.

TABLE 21
Small scale titration of CD25 microbeads

| | | Based on MFI | |
|---|---|---|---|
| | Treg MFI | % Vacancy | % Occupancy |
| 8X Target Conc. | 494 | 0% | 100% |
| 4X Target Conc. | 549 | 1.4% | 98.4% |
| 2X Target Conc. | 717 | 5.5% | 94.5% |
| Target Conc | 1518 | 25.3% | 74.7% |
| 0.5X Target Conc. | 2398 | 47.1% | 52.9% |
| 0.25X Target Conc. | 3117 | 64.8% | 35.2% |
| 0.125X Target Conc. | 3707 | 79.4% | 20.6% |
| 0.0625X Target Conc. | 4276 | 93.5% | 6.5% |
| 0X Target Conc. | 4541 | 100% | 0% |

In another experiment, varying amounts of anti-CD25 iron-dextran conjugates can be incubated against a constant number of GCSF-mobilized peripheral blood stem cells to generate an experimental series. A set portion of the sample may be removed to assess % receptor occupancy using a CD25− fluorophore conjugate that binds competitively with the anti-CD25 iron dextran conjugate. A portion of each of the experimental series may be transferred to culture media and stimulated with IL-2 (either 10 U/mL or 100 U per mL) for 15-30 minutes at 37° C. The cells can then be fixed in cold methanol and stained for Treg cell surface markers (CD45, CD3, CD4, CD25, CD127) and phospho-STAT5 (pSTAT5) as a marker of activation. The extent of pSTAT5 (MFI) and % positive pSTAT5 on Treg cells may then be quantified by flow cytometry. The pSTAT5 stimulation is expected to reduce as the % receptor occupancy increases.

### CONCLUSION

While various embodiments of the present disclosure have been shown and described herein, it will be obvious to those skilled in the art that such embodiments are provided by way of example only. Numerous variations, changes, and substitutions will be apparent to those skilled in the art without departing from the disclosure. For example, various embodiments of therapeutic compositions, methods of can be adapted for various pediatric, geriatric and veterinary applications, the latter including one or more of felines, canines and equines. Specific adaptions for such can include one or more of the cell types in the therapeutic composition administered to the patient and the dose of cells in the therapeutic composition. They can also be adapted for treatment of any number of stem cell-based cancers including leukemia, lymphoma, myeloma, non-malignant hematologic conditions such as sickle cell anemia, as well as a number of t-cell mediated and other autoimmune diseases including one more of multiple sclerosis, IBD, Celiac Disease. Crohn's disease. ulcerative colitis, ankylosing spondylosis, myasthenia gravis and diabetes. It should be understood that various alternatives to the embodiments of the disclosure described herein may be employed in practicing the disclosure.

Elements, characteristics, or acts from one embodiment can be readily recombined or substituted with one or more elements, characteristics or acts from other embodiments to form numerous additional embodiments within the scope of the disclosure. Moreover, elements that are shown or described as being combined with other elements, characteristics, steps or acts, can, in various embodiments, exist as stand-alone elements, characteristics, steps or acts. Further, various embodiments expressly contemplate the negative recitation of any element, characteristic, step or act etc. that is/are shown or described in one or more embodiments. Hence, the scope of the present disclosure is not limited to the specifics of the described embodiments, but is instead limited solely by the appended claims.

### Additional Embodiments

Embodiment A1: A therapeutic composition for hematopoietic cell transplantation to a patient in need thereof, the therapeutic composition comprising: a first population of isolated CD45+ cells wherein at least a portion of the CD45+ cells have an antibody bound to a marker on the cell surface which is used to separate CD34+ cells from a mixture of nucleated cells from a volume of blood from a human donor wherein the mixture of nucleated cells comprise at least about 70% CD34+ cells, less than about 5% CD3+ cells, and less about 20% granulocytes; and a second population of isolated CD45+ cells wherein at least about 50% of the isolated CD45+ cells are regulatory T (Treg) cells.

Embodiment A2: The composition of Embodiment A1, wherein the first population comprises at least about 90% CD34+ cells.

Embodiment A3: The composition of Embodiment A1, wherein at least about 70% of the isolated CD45+ cells in the second population of isolated CD45+ cells are Treg cells.

Embodiment A4: The composition of Embodiment A1, wherein the first population of isolated CD45+ cells comprises less than about 6.2×10⁵ granulocyte cells per kg patient weight.

Embodiment A5: The composition of Embodiment A1, wherein the first population of isolated CD45+ cells comprises less than about 2×10⁵ monocyte cells per kg patient weight.

Embodiment A6: The composition of Embodiment A1, wherein the first population of isolated CD45+ cells comprises less than about 1.3×10⁵ B-cells and natural killer (NK) cells in combination per kg patient weight.

Embodiment A7: The composition of Embodiment A1, wherein at least a portion of the CD45+ cells in the second population of cells has cells have an antibody bound to a marker on the cell surface which is used to separate CD45+ cells from the mixture of nucleated cells from the human donor blood volume.

Embodiment A8: The composition of Embodiment A7, wherein the antibody is an anti-CD25+ antibody.

Embodiment A9: The composition of Embodiment A8, wherein at least a portion of the Treg cells in the second cell population have about 500 to 10,000 of antibodies bound to each cell.

Embodiment A10: The composition of Embodiment A9, wherein at least a portion of the Treg cells in the second cell population have about 5000 to 10,000 of antibodies bound to each cell.

Embodiment A11: The composition of Embodiment A7, wherein at least about 70% of the Treg cells in the second cell population have bound antibody.

Embodiment A12: The composition of Embodiment A11, wherein at least about 90% of the Treg cells have bound antibody.

Embodiment A13: The composition of Embodiment A1, wherein the marker is a receptor.

Embodiment A14: The composition of Embodiment A1, where the bound antibody is conjugated to a particle.

Embodiment A15: The composition of Embodiment A14, wherein the particle is a magnetic particle.

Embodiment A16: The composition of Embodiment A15, wherein the magnetic particle comprises iron, nickel or cobalt.

Embodiment A17: The composition of Embodiment A14, wherein the particle is a fluorophore particle.

Embodiment A18: A therapeutic composition for hematopoietic stem cell transplantation to a patient in need thereof, the therapeutic composition comprising: a population of isolated T regulatory (Treg) cells, wherein at least a portion of the Treg cells have a particle bound to a receptor on the cell surface which is used to separate Treg cells from a mixture of nucleated cells from a mobilized blood donor, the antibody conjugated to a particle.

Embodiment A19: A therapeutic composition for hematopoietic stem cell transplantation to a patient in need thereof, the therapeutic composition comprising: a population of isolated T cells wherein at least a portion of the T cells have an anti-CD4+ antibody bound to a marker on the cell surface which is used to separate T cells from a mixture of nucleated cells from a mobilized blood donor, the antibody conjugated to a particle.

Embodiment A20: The composition of Embodiment A19, wherein the T cells are regulatory T (Treg) cells

Embodiment A21: The composition of Embodiment A19, wherein the at least a portion of the Treg cells have about 1000 to 400,000 of the anti-CD4+ antibodies bound to each cell.

Embodiment A22: The composition of Embodiment A21, wherein the at least a portion of the Treg cells have about 50,000 to 400,000 of the anti-CD4+ antibodies bound to each cell.

Embodiment A23: The composition of Embodiment A19, wherein at least about 70% of the Treg cells have bound antibody.

Embodiment A24: The composition of Embodiment A19, wherein at least about 90% of the Treg cells have bound antibody.

Embodiment A25: The composition of Embodiment A19, wherein the marker is a receptor.

Embodiment A26: The composition of Embodiment A19, wherein the particle is a magnetic particle.

Embodiment A27: The composition of Embodiment A26, wherein the magnetic particle comprises iron, nickel or cobalt.

Embodiment A28: The composition of Embodiment A19, wherein the particle is a fluorophore particle.

Embodiment A29: A therapeutic composition for hematopoietic cell transplantation to a human subject in need thereof, the therapeutic composition comprising: a population of isolated hematopoietic stem and progenitor cells (HSPC) wherein at least a portion of the HSPC's have an antibody bound to a marker on the cell surface which is used to separate HSPC's from a mixture of nucleated cells from a mobilized blood donor, the antibody conjugated to a particle.

Embodiment A30: The composition of Embodiment A29, wherein the antibody is an anti-CD34+ antibody.

Embodiment A31: The composition of Embodiment A29, wherein the at least a portion of HSPC's have about 500 to 10,000 of conjugated antibodies bound to each cell.

Embodiment A32: The composition of Embodiment A31, wherein the at least a portion of HSPC's have about 5000 to 10,000 of conjugated antibodies bound to each cell.

Embodiment A33: The composition of Embodiment A31, wherein at least about 70% of the HSPC's have bound antibody.

Embodiment A34: The composition of Embodiment A33, wherein at least about 90% of the HSPC's have bound antibody.

Embodiment A35: The composition of Embodiment A31, wherein the marker is a receptor.

Embodiment A36: The composition of Embodiment A31, wherein the particle is a magnetic particle.

Embodiment A37: The composition of Embodiment A36, wherein the magnetic particle comprises iron, nickel or cobalt.

Embodiment A38: The composition of Embodiment A29, wherein the particle is a fluorophore particle.

Embodiment B1: A kit for preparation of at least one therapeutic composition for the treatment of a disease or condition, the at least one therapeutic composition having cellular components and prepared from mobilized apheresis blood product, the kit comprising: a first plurality of antigen-binding agents that target CD34+ cells in the blood product; a second plurality of antigen-binding agents that target CD25+ cells in the blood product; and instructions for use of the kit to prepare the at least one therapeutic composition.

Embodiment B2: A kit for preparation of at least one therapeutic composition for treatment of a disease or condition in a human subject in need thereof, the at least one therapeutic composition having cellular components and prepared from mobilized apheresis blood product, the kit comprising: a first plurality of antigen-binding agents that target CD34+ cells in the blood product; a second plurality of antigen-binding agents that target CD25+ cells in the blood product, wherein the CD34+ and CD25+ antigen-binding agents are conjugated to a particle which allows for separation of targeted CD34+ and CD25+ cells from the blood product and into the at least one therapeutic composition; packaging associated with said antigen binding agents, and instructions for use of the kit to prepare the at least one therapeutic composition.

Embodiment B3: The kit of Embodiment B2, wherein the CD34+ and CD25+ antigen-binding agents are disposed in the packaging.

Embodiment B4: The kit of Embodiment B2, wherein the disease or condition is graft versus host disease, a malignancy or an autoimmune condition.

Embodiment B5: The kit of Embodiment B2, wherein the CD34+ and CD25+ antigen-binding agents are configured and arranged to allow for separation of the CD34+ and CD25+ cells from mobilized apheresis blood product that is obtained from one donor on a single day.

Embodiment B6: The kit of Embodiment B2, wherein the treatment comprises hematopoietic stem cell transplantation.

Embodiment B7: The kit of Embodiment B2, wherein the antigen binding agent comprises an antibody or an antigen binding fragment.

Embodiment B8: The kit of Embodiment B2, wherein the at least one therapeutic composition comprises hematopoietic stem and progenitor cells (HSPC), the composition having more than about one million HSPCs per kg patient weight.

Embodiment B9: The kit of Embodiment B8, wherein the at least one therapeutic composition comprises more than about 3.9×10⁶ HSPCs per kg patient weight.

Embodiment B10: The kit of Embodiment B8, wherein the at least one therapeutic composition comprises less than about 1.2×10⁶ non-HSPCs per kg patient weight.

Embodiment B11: The kit of Embodiment B8, wherein the non-HSPC cellular components of the at least one therapeutic composition comprise:
granulocyte cells less than about 6.2×10⁵ cells per kg patient weight; monocyte cells less than about. 2×10⁵ cells per kg patient weight; and B-cells and natural killer cells less than about. 1.3×10⁵ cells per kg patient weight.

Embodiment B12: The kit of Embodiment B2, wherein the at least one therapeutic composition comprises regulatory T-cells (Treg), the composition having more than about one million Treg cells per kg patient weight.

Embodiment B13: The kit of Embodiment B12, wherein the at least one therapeutic composition comprises more than about 2.3×10⁶ Treg per kg patient weight.

Embodiment B14: The kit of Embodiment B12, wherein the at least one therapeutic composition comprises less than about 1.1×10⁵ non-Treg T-cells per kg patient weight.

Embodiment B15: The kit of Embodiment B12, wherein the at least one therapeutic composition comprises less than about 1.4×10⁵ non-Treg cells per kg patient weight.

Embodiment B16: The kit of Embodiment B12, wherein the non-Treg cellular components of the at least one therapeutic composition comprise: granulocyte and monocytes cells less than about 3.3×10³ cells per kg patient weight.

Embodiment B17: The kit of Embodiment B12, wherein the non-Treg cellular components of the at least one therapeutic composition comprise: B-cells and natural killer cells less than about. 5.4×10⁴ cells per kg patient weight.

Embodiment B18: The kit of Embodiment B2, wherein the cellular components of the at least one therapeutic composition comprise: CD34+ cells greater than about one million cells per kg patient weight; and CD25+ cells greater than about one million cells per kg patient weight.

Embodiment B19: The kit of Embodiment B2, wherein the cellular components of the at least one therapeutic composition comprise: CD3+ cells greater than about half a million cells per kg patient weight.

Embodiment B20: The kit of Embodiment B2, wherein the treatment comprises solid organ transplantation.

Embodiment B21: The kit of Embodiment B2, wherein the at least one therapeutic composition comprises first and second cell populations contained in first and second liquid volumes.

Embodiment B22: The kit of Embodiment B21, wherein first cell population comprises CD34+ hematopoietic stem cells.

Embodiment B23: The kit of Embodiment B22, wherein the CD34+ hematopoietic stem cells have a purity of at least about 80% relative to all nucleated cells in the first cell population.

Embodiment B24: The kit of Embodiment B22, wherein the first liquid volume has at least about one million CD34+ hematopoietic stem cells per kg patient weight.

Embodiment B25: The kit of Embodiment B21, wherein the at least one therapeutic composition has fewer than about 50,000 CD3+ T-cells per kg patient weight.

Embodiment B26: The kit of Embodiment B21, wherein second cell population comprises T regulatory (Treg) cells.

Embodiment B27: The kit of Embodiment B26, wherein the Treg cells that have a purity between about 30 to 90 percent relative to all nucleated cells in the second volume.

Embodiment B28: The kit of Embodiment B2, wherein the kit does not contain CD8+ or CD19+ antigen binding agents.

Embodiment B29: The kit of Embodiment B2, wherein all cellular components of the at least one therapeutic composition are obtained from a mobilized blood product.

Embodiment B30: The kit of Embodiment B2, wherein the kit does not contain CD4+ or CD127+ antigen binding agents.

Embodiment B31: The kit of Embodiment B2, further comprising a plurality of anti-CD4+ antigen binding agents conjugated to a fluorophore for optical sorting of CD4+ T-cells.

Embodiment B32: The kit of Embodiment B2, further comprising a plurality of anti-CD127+ antigen binding agents conjugated to a fluorophore for optical sorting of CD4+ T-cells.

Embodiment B33: The kit of Embodiment B2, wherein an amount of antigen-binding agents in the kit is selected to process greater than about 8 liters of donor blood.

Embodiment B34: The kit of Embodiment B2, wherein an amount of antigen-binding agents in the kit is selected to process greater than about 15 liters of donor blood.

Embodiment B35: The kit or method of Embodiment B34, wherein the at least one therapeutic composition includes a CD3+ fraction.

Embodiment B36: The kit or method of Embodiment B35, wherein the CD3+ fraction is derived from blood that has a T-regulatory cell content of about 0.5 to 5% of CD45+ cells.

Embodiment B37: The kit or method of Embodiment B35, wherein the CD3+ fraction is derived from a volume of blood that is set aside from the apheresis blood product and/or not derived from apheresis blood product.

Embodiment B38: The kit of Embodiment B2, wherein the instructions for use are stored on electronic media or non-transitory computer readable media or non-volatile memory.

Embodiment B39: The kit of Embodiment B2, wherein the particle is a magnetic particle.

Embodiment B40: The kit of Embodiment B39, wherein the magnetic particle comprises iron, nickel or cobalt.

Embodiment B41: The kit of Embodiment B2, wherein the particle is a fluorophore.

Embodiment B42: The kit of Embodiment B2, wherein the packaging comprises a container in which the antigen-binding agents are disposed.

Embodiment B43: The kit of Embodiment B42, wherein the container includes an aqueous solution or a buffer solution in which the antigen binding agents are suspended.

Embodiment B44: The kit of Embodiment B2, wherein the instructions for use are stored on a network, a computer network, the Internet or the cloud.

Embodiment B45: The kit of Embodiment B2, wherein the instructions for use include information unique to a batch of antigen-binding agents used in the kit, the information used by an instrument or device in the preparation of the at least one therapeutic composition to optimize a parameter of the at least one therapeutic composition.

Embodiment B46: The kit of Embodiment B45, wherein the parameter is a purity, concentration or dose of HSC in the at least one therapeutic composition.

Embodiment B47: The kit of Embodiment B45, wherein the information comprises optically or electronically readable indicia.

Embodiment B48: The kit of Embodiment B2, further comprising: a buffer solution in which the antigen-binding agents are suspended; and

a container for the buffer solution.

Embodiment B49: The kit of Embodiment B48, wherein the container comprises a bag, a polymer bag or a polyvinyl chloride bag.

Embodiment B50: The kit of Embodiment B48, wherein the container includes indicia encoding information unique to a batch of antigen binding agents used in the kit, the information used by an instrument or device in the preparation of the at least one therapeutic composition to optimize a parameter of the at least one therapeutic composition.

Embodiment B51: The kit of Embodiment B50, wherein the indicia are optically or electronically readable indicia.

Embodiment B52: The kit of Embodiment B48, wherein the buffer solution contains about 2.5 volume percent human serum albumin and 1 millimolar EDTA.

Embodiment B53: The kit of Embodiment B48, wherein the buffer solution is a phosphate buffer.

Embodiment B54: The kit of Embodiment B48, wherein the buffer solution has a pH of about 7.2.

Embodiment B55: The kit of Embodiment B48, further comprising a plurality of immunoglobulin molecules suspended in the buffer solution.

Embodiment B56: The kit of Embodiment B55, wherein the plurality of immunoglobulin molecules comprises IVgG.

Embodiment B57: A kit for preparation of a therapeutic composition comprising cellular components for treatment of a disease or condition in a human subject in need thereof, the at least one therapeutic composition prepared from a mixture of nucleated cells from a mobilized blood donor, the kit comprising: a buffer solution; a plurality of anti-CD34+ antigen-binding agents and plurality of anti-CD 25+ antigen-binding agents suspended within the buffer solution, said antigen binding agents conjugated to a magnetic particle for magnetically separating CD34+ and CD25+ cells from the blood product and into the at least one therapeutic composition; non-transitory computer readable storage media containing computer executable instructions for performing operations on a device or instrument to create the at least one therapeutic composition using the anti-CD34+ and CD 25+ antigen-binding agents; and instructions for use of the kit to prepare the at least one therapeutic composition.

Embodiment B58: The kit of Embodiment B57, wherein the disease or condition is graft versus host disease, a malignancy or an autoimmune condition.

Embodiment B59: The kit of Embodiment B57, wherein the treatment comprises hematopoietic stem cell transplantation.

Embodiment B60: The kit of Embodiment B57, wherein the antigen binding agent comprises an antibody or an antigen binding fragment.

Embodiment B61: The kit of Embodiment B57, wherein the non-transitory computer readable media comprises a memory device, a non-volatile memory device or a flash memory device.

Embodiment B62: The kit of Embodiment B57, wherein the device or instrument is a cell sorter.

Embodiment B63: The kit of Embodiment B57, wherein the cell sorter is an optical cell sorter or a fluorescence-activated cell sorter.

Embodiment B64: The kit of Embodiment B57, wherein the cell sorter is a magnetic-based cell sorter.

Embodiment B65: The kit of Embodiment B57, wherein the non-transitory computer readable storage media encodes information unique to a batch of antigen-binding agents used in the kit, the information used by an instrument or device in the preparation of the at least one therapeutic composition to optimize a parameter of the at least one therapeutic composition.

Embodiment B66: A kit for preparation of a therapeutic composition comprising cellular components for treatment of a disease or condition in a human subject in need thereof, wherein the at least one therapeutic composition is prepared from a mixture of nucleated cells from a mobilized blood donor, the kit comprising: a buffer solution; a plurality of anti-CD34+ antigen-binding agents and a plurality of anti-CD25+ antigen-binding agents suspended within the buffer solution, said antigen binding agents conjugated to a magnetic particle for magnetically separating CD34+ and CD25+ cells from the mixture of nucleated cells; and a container holding the buffer antigen binding agent solution, wherein the container includes indicia encoding information unique to a batch of antigen-binding agents used in the kit, the information used by an instrument or device in the preparation of the at least one therapeutic composition to optimize a parameter of the at least one therapeutic composition.; and instructions for use of the kit to prepare the at least one therapeutic composition by magnetic separation.

Embodiment B67: A kit for preparation of a therapeutic composition for hematopoietic stem cell (HSC) transplantation to a human subject in need thereof, wherein the therapeutic composition is prepared from a mixture of nucleated cells from a mobilized blood donor, the kit comprising: a buffer solution; a plurality of anti-CD34+ antigen-binding agents thereof suspended within the buffer solution, said antigen binding agents conjugated to a magnetic particle for magnetically separating HSCs from the mixture of nucleated cells from a mobilized blood donor; and instructions for use of the kit to prepare the therapeutic composition by magnetic separation of HSCs from the mixture of nucleated cells from the mobilized blood donor, wherein the instructions for use include information unique to a batch of anti-CD34+ antigen-binding agents used in the kit the information used by an instrument or device in the preparation of the one therapeutic composition to optimize a parameter of the at least one therapeutic composition.

Embodiment B68: The kit of Embodiment B67, wherein the information comprises optically or electronically readable indicia associated with the instructions.

Embodiment B69: A kit for preparation of a therapeutic composition for hematopoietic stem cell (HSC) transplantation to a human subject in need thereof, wherein the at least one therapeutic composition is prepared from a mixture of nucleated cells from a mobilized blood donor, the kit comprising: a buffer solution; a plurality of anti-TCR alpha beta antigen-binding agents and a plurality of CD25+ antigen binding agents suspended within the buffer solution, said antigen binding agents are conjugated to a magnetic particle for magnetically separating HSCs from the mixture of nucleated cells from a mobilized blood donor; and instructions for use of the kit to prepare the at least one therapeutic composition by magnetic separation of HSCs from the mixture of nucleated cells from the mobilized blood donor.

Embodiment B70: A kit for preparation of a therapeutic composition for organ transplantation to a human subject in need thereof, wherein the therapeutic composition is prepared from a mixture of nucleated cells from a mobilized blood donor, the kit comprising: a buffer solution; a plurality of anti-CD25+ antigen-binding agents suspended within the buffer solution, said antigen binding agents conjugated to a magnetic particle for magnetically separating CD25+ T-cells from the mixture of nucleated cells from the mobilized blood donor; and instructions for use of the kit to prepare the at least one therapeutic composition by magnetic separation of CD25+ T-cells from the mixture of nucleated cells from the mobilized blood donor, wherein the instructions for use include information unique to a batch of CD25+ T+ antigen-binding agents used in the kit, the information used by an instrument or device in the preparation of the at least one therapeutic composition to optimize a parameter of the at least one therapeutic composition.

Embodiment B71: A method for preparation of at least one therapeutic composition for treatment of a disease or condition in a human subject in need thereof, the at least one therapeutic composition having cellular components and prepared from mobilized apheresis blood product, the method comprising: providing a first plurality of antigen-binding agents that target CD34+ cells in the blood product and a second plurality of antigen binding agents that target CD25+ cells in the blood product; wherein the CD34+ and CD25+ antigen-binding agents are conjugated to a particle which allows for separation of targeted CD34+ and CD25+ cells from the blood product and into the at least one therapeutic composition; using the first plurality of antigen binding agents to separate CD34+ cells from the blood product so as to produce an enriched mixture of CD34+ cells; using the second plurality of antigen binding agents to separate CD25+ cells from the blood product so as to produce an enriched mixture of CD25+ cells; and wherein the mobilized apheresis blood product is obtained from one donor on a single day.

Embodiment B72: The method of Embodiment B71, further comprising analyzing at least one of the enriched mixtures of CD34+ or CD25+ cells for a cell purity of the respective cells in the mixture.

Embodiment B73: The method of Embodiment B72, further comprising determining whether a cell purity of at least one of the enriched CD34+ or CD25+ cell mixtures meets a predetermined threshold.

Embodiment B74: The method of Embodiment B73, further comprising performing additional processing of at least one of the enriched CD34+ or CD25+ cell mixtures if the predetermined threshold is not met.

Embodiment B75: The method of Embodiment B74, wherein the additional processing comprises performing antigen-binding agent-based cell separations of at least of the enriched CD34+ or CD25+ cell mixtures.

Embodiment B76: The method of Embodiment B71, further comprising adjusting at least one of the enriched CD34+ or CD25+ cell mixtures to achieve a selected patient specific dose of CD34+ or CD25+ cells.

Embodiment B77: The method of Embodiment B76, wherein the patient specific dose is relative to number of cells per kilogram patient weight.

Embodiment B78: The method of Embodiment B71, further comprising exchanging buffer of least one of the enriched CD34+ or CD25+ cell mixtures with another buffer which is used to suspend the cells in for administration to the patient.

Embodiment B79: The method of Embodiment B71, further comprising adding an excipient to at least one of the enriched mixtures of CD34+ or CD25+ cells.

Embodiment B80: The method of Embodiment B79, wherein the excipient is at least one of a buffering agent, an antibody or IgG.

Embodiment B81: A method for preparation of at least one therapeutic composition for treatment of a disease or condition in a human subject in need thereof, the at least one therapeutic composition having cellular components and prepared from mobilized apheresis blood product, the method comprising: providing a first plurality of antigen-binding agents that target CD34+ cells in the blood product and a second plurality of antigen binding agents that target CD25+ cells in the blood product; wherein the CD34+ and CD25+ antigen-binding agents are conjugated to a particle which allows for separation of targeted CD34+ and CD25+ cells from the blood product and into the at least one therapeutic composition; using the first plurality of antigen binding agents to separate CD34+ cells from the blood product so as to produce an enriched mixture of CD34+ cells; using the second plurality of antigen binding agents to separate CD25+ cells from the blood product so as to produce an enriched mixture of CD25+ cells; analyzing at least one of the enriched mixtures of CD34+ or CD25+ cells for a cell purity of the respective cells in the mixture; and wherein the mobilized apheresis blood product is obtained from one donor on a single day.

Embodiment B82: The method of Embodiment B81, further comprising determining whether a cell purity of at least one of the enriched CD34+ or CD25+ cell mixtures meets a predetermined threshold.

Embodiment B83: The method of Embodiment B82, further comprising performing additional processing of at least one of the enriched CD34+ or CD25+ cell mixtures if the predetermined threshold is not met.

Embodiment B84: The method of Embodiment B83, wherein the additional processing comprises performing antigen-binding agent-based cell separations of at least of the enriched CD34+ or CD25+ cell mixtures.

Embodiment B85: The method of Embodiment B81, wherein the disease or condition is graft versus host disease, a malignancy or an autoimmune condition.

Embodiment B86: The method of Embodiment B81, wherein the CD34+ and CD25+ antigen-binding agents are configured and arranged to allow for separation of the CD34+ and CD25+ cells from mobilized apheresis blood product that is obtained from one donor on a single day.

Embodiment B87: The method of Embodiment B81, wherein the treatment comprises hematopoietic stem cell transplantation.

Embodiment B88: The method of Embodiment B81, wherein the antigen binding agent comprises an antibody or an antigen binding fragment.

Embodiment B89: The method of Embodiment B81, wherein the cellular components of the at least one therapeutic composition comprise: CD34+ cells greater than about one million cells/kg patient weight; and CD25+ cells greater than about one million cells/kg patient weight.

Embodiment B90: The method of Embodiment B89, wherein the cellular components further comprise: CD3+ cells greater than about half a million cells/kg patient weight.

Embodiment B91: The method of Embodiment B81, wherein the cellular components of the at least one therapeutic composition comprise: CD3+ cells greater than about half a million cells/kg patient weight.

Embodiment B92: The method of Embodiment B81, wherein the treatment comprises solid organ transplantation.

Embodiment B93: The method of Embodiment B81, wherein the at least one therapeutic composition comprises first and second cell populations contained in first and second liquid volumes.

Embodiment B94: The method of Embodiment B93, wherein first cell population comprises CD34+ hematopoietic stem cells.

Embodiment B95: The method of Embodiment B94, wherein the CD34+ hematopoietic stem cells have a purity of at least about 80% relative to all nucleated cells in the first cell population.

Embodiment B96: The method of Embodiment B94, wherein the first liquid volume has at least about one million CD34+ hematopoietic stem cells per kg patient weight.

Embodiment B97: The method of Embodiment B93, wherein the at least one therapeutic composition has fewer than about 50,000 CD3+ T-cells per kg patient weight.

Embodiment B98: The method of Embodiment B93, wherein second cell population comprises T regulatory (Treg) cells.

Embodiment B99: The method of Embodiment B98, wherein the Treg cells that have a purity between about 30 to 90 percent relative to all nucleated cells in the first volume.

Embodiment B100: The method of Embodiment B94, wherein the CD34+ and CD25+ antigen-binding agents do not contain CD8+ or CD19+ antigen binding agents.

Embodiment B101: The method of Embodiment B94, wherein all cellular components of the at least one therapeutic composition are obtained from a mobilized blood product.

Embodiment B102: The method of Embodiment B94, wherein the CD34+ and CD25+ antigen-binding agents do not contain CD4+ or CD127+ antigen binding agents.

Embodiment B103: The method of Embodiment B81, wherein an amount of antigen-binding agent in the first or second plurality is selected to process greater than about 8 liters of donor blood.

Embodiment B104: The method of Embodiment B81, wherein an amount of antigen-binding agent in the first or second plurality selected to process greater than about 15 liters of donor blood.

Embodiment B105: The method or method of Embodiment B81, wherein the at least one therapeutic composition includes a CD3+ fraction.

Embodiment B106: The method or method of Embodiment B105, wherein the CD3+ fraction is derived from blood that has a T-regulatory cell content of about 0.5 to 5% of CD45+ cells.

Embodiment B107: The method or method of Embodiment B105, wherein the CD3+ fraction is derived from a volume of blood that is set aside from the apheresis blood product and/or not derived from apheresis blood product.

Embodiment B108: The method of Embodiment B81, wherein the particle is a magnetic particle.

Embodiment B109: The method of Embodiment B108, wherein the magnetic particle comprises iron, nickel or cobalt.

Embodiment B110: The method of Embodiment B81, wherein the particle is a fluorophore.

Embodiment B111: The method of Embodiment B81, the antigen-binding agents are suspended in a buffer.

What is claimed is:

1. A method of treating a disease or condition in a human subject, the method comprising:
(a) obtaining isolated regulatory T cells (Tregs); wherein the obtaining Tregs comprises:
(i) contacting a donor cell sample comprising Tregs with an amount of an anti-human CD25 affinity reagent such that less than 85% of the CD25 polypeptides expressed on the surface of the Tregs in the donor cell sample are occupied by the anti-human CD25 affinity reagent;
(ii) isolating Tregs from (a)(i) occupied by the anti-human CD25 affinity reagent;
thereby obtaining the isolated Tregs; and
(b) administering the isolated Tregs to the human subject;
wherein the isolated Tregs exhibit pSTAT5 activity according to an in vitro assay.

2. The method of claim 1, wherein from 30% to 80% of the CD25 polypeptides on the Tregs are occupied by the anti-human CD25 affinity reagent.

3. The method of claim 1, wherein the isolated Tregs exhibit from 10% to 50% of the pSTAT5 activity in an in vitro assay as compared to Tregs that do not have CD25 polypeptides occupied by the anti-human CD25 affinity reagent.

4. The method of claim 1, wherein the method further comprises isolating hematopoietic stem cells (HSCs), hematopoietic stem and progenitor cells (HSPCs), or both HSCs and HSPCs from the donor cell sample.

5. The method of claim 4, wherein the isolating of the isolated HSCs or the isolated HSPCs comprises contacting the donor cell sample with an anti-human CD34 affinity reagent and isolating cells that have CD34s occupied by the anti-human CD34 affinity reagent, thereby obtaining the isolated HSCs, the isolated HSPCs, or both the isolated HSCs and the isolated HSPCs.

6. The method of claim 4, wherein the method further comprises administering the isolated HSCs, the HSPCs, or both the isolated HSCs and the isolated HSPCs to the human subject, wherein from 5×10⁵ to 2×10⁷ HSCs or from 5×10⁵ to 2×10⁷ HSPCs per kilogram of the human subject's actual body weight or ideal body weight are administered to the human subject.

7. The method of claim 6, wherein from 5×10⁵ to 5×10⁶ Tregs per kilogram of the human subject's actual body weight or ideal body weight are administered to the human subject.

8. The method of claim 1, further comprising contacting the donor cell sample with an anti-human CD3 affinity reagent and quantifying an amount of CD3 positive conventional T cells (Tcons) in the donor cell sample by identifying the presence of CD3 polypeptides on Tcons that are occupied by the anti-human CD3 affinity reagent.

9. The method of claim 8, wherein the human subject is further administered a cell population comprising from 5×10⁵ to 5×10⁶ Tcons per kilogram of actual body weight or ideal body weight of said human subject.

10. The method of claim 1, further comprising administering a single GVHD prophylactic agent to the human subject, wherein the single GVHD prophylactic agent is tacrolimus or sirolimus.

11. The method of claim 1, further comprising administering a conditioning regimen to the human subject.

12. The method of claim 1, wherein the donor cell sample is a blood sample.

13. The method of claim 1, wherein the contacting comprises determining that less than 85% of the CD25 polypeptides expressed on the surface of the Tregs in the donor cell sample are occupied by the anti-human CD25 affinity reagent according to an in vitro assay; wherein the in vitro assay comprises:
(1) measuring the amount of background fluorescence from a first sample of a cell population, wherein the background fluorescence is emitted at a wavelength corresponding to a first fluorophore and wherein a Mean Fluorescence Intensity Zero (MFI-0) value is assigned the measured amount of background fluorescence;
(2) contacting a second sample of a cell population with an anti-CD25 detection antibody which is conjugated to the first fluorophore, wherein the contacting occurs at a concentration, temperature, and time sufficient to achieve at least 90% saturation;
(3) measuring the amount of fluorescence emitted from the cells of step (2), wherein a Mean Fluorescence Intensity Saturation (MFI-sat) value is assigned the measured amount of fluorescence;
(4) contacting simultaneously a third sample of a cell population with the anti-CD25 detection antibody conjugated to the first fluorophore and the anti-human CD25 affinity reagent which lacks a fluorophore and under the contacting conditions used in step (2);
(5) measuring the amount of fluorescence emitted from the cells of step (4), wherein a Mean Fluorescence Intensity X (MFI-x) value is assigned the measured amount of fluorescence; and
(6) quantifying the percentage of receptor occupancy by the anti-human CD25 affinity reagent according to the following equation:
% Receptor Occupancy=100%−[(MFI-x−MFI-0)/(MFI-sat−MFI-0)×100%].

14. The method of claim 1, wherein the in vitro assay to assess pSTAT5 activity comprises an intracellular cytokine staining comprising:
(1) obtaining a cell having been exposed to the anti-human CD25 affinity reagent;
(2) contacting the cell with IL-2 at a temperature and time sufficient for the IL-2 to interact with its cognate receptor on the cell and activate IL-2 signaling by the cell;
(3) contacting the cell with each of:
(a) a first antibody conjugated with a first fluorophore and directed against CD3,
(b) a second antibody conjugated with a second fluorophore and directed against CD4,
(c) a third antibody conjugated with a third fluorophore and directed against CD25,
(d) a fourth antibody conjugated with a fourth fluorophore and directed against CD127, and
(e) a fifth antibody conjugated with a fifth fluorophore and directed against pSTAT5, wherein each of the first, second, third, fourth, and fifth fluorophore are different fluorophores;
(4) subjecting the cell of step (3) to flow cytometry which is gated for CD3⁺CD4⁺CD25⁺CD127− and collecting the CD3⁺CD4⁺CD25⁺CD127− cells:
(5) detecting the amount of fluorescence from the fifth fluorophores and detecting the amount of florescence from the first, the second, and/or the third fluorophores in the collected cells of step (5); and
(6) calculating the percentage of the collected cells which have fluorescence from the first, the second, and/or the third fluorophores and which have fluorescence from the fifth fluorophore, wherein this percentage represents the fraction of cells having receptors occupied by the anti-human CD25 affinity reagent.

15. The method of claim 14, wherein the fifth antibody directed against pSTAT5 is directed against pSTAT5 phosphorylated at Tyrosine 694.

16. The method of claim 1, wherein the pSTAT5 activity is assessed by determining MFI and/or percentage positive of the isolated Treg bound by an antibody directed against STAT5 phosphorylated at Tyrosine 694 as quantified by flow cytometry.

17. The method of claim 1, wherein the in vitro assay to assess pSTAT5 activity comprises determining the IL2 signaling capacity of the isolated Tregs.

18. The method of claim 1, wherein the anti-human CD25 affinity reagent is capable of blocking the pSTAT5 activity and/or blocking IL2 signaling in the isolated Tregs.

19. A method of treating a disease or condition in a human subject, the method comprising:
(a) obtaining isolated regulatory T cells (Tregs); wherein the obtaining Tregs comprises:
(i) contacting a donor cell sample comprising Tregs with an amount of an anti-human CD25 affinity reagent such that more than 85% of the CD25 polypeptides expressed on the surface of the Tregs in the donor cell sample are occupied by the anti-human CD25 affinity reagent;
(ii) isolating Tregs from (a)(i) obtained by the anti-human CD25 affinity reagent;
thereby obtaining the isolated Tregs; and
(b) administering the isolated Tregs to the human subject;
wherein the isolated Tregs exhibit pSTAT5 activity according to an in vitro assay;
wherein the in vitro assay to assess pSTAT5 activity comprises determining the IL2 signaling capacity of the isolated Tregs; and
wherein the anti-human CD25 affinity reagent does not block IL2 signaling in the Tregs.

20. A kit for use in preparation of a therapeutic composition, the kit comprising:
(a) an anti-human CD25 affinity reagent; and
(b) instructions for use of (a) to isolate regulatory T cells (Tregs) from a donor cell sample;
wherein the instructions include directions to isolate the Tregs which have the anti-human CD25 affinity reagent such that less than 85% of CD25 polypeptides expressed on the surface of the Tregs in the donor cell sample are occupied by the anti-human CD25 affinity reagent, and
optionally wherein the kit further comprises an anti-human CD34 affinity reagent and instructions to isolate a cell population of CD34 positive hematopoietic stem and progenitor cells (HSPCs); a means to isolate CD25 positive cells and/or CD34 positive cells; and an anti-human CD3 affinity reagent and instructions for quantifying an amount of conventional T cells (Tcons) in the donor cell sample, optionally wherein the means comprises a sorting column, optionally wherein the sorting column is a magnetized column, and optionally wherein the instructions include directions to detect pSTAT5 activity in the Tregs in an in vitro assay.
